(19) United States
(12) Patent Application Publication
(10) Pub. No.: US 2022/0411514 A1
(43) Pub. Date: Dec. 29, 2022

(54) ANTI-GAL3 ANTIBODIES AND METHODS OF USE FOR INSULIN RESISTANCE

(71) Applicant: TrueBinding, Inc., Foster City, CA (US)

(72) Inventors: Dongxu Sun, Palo Alto, CA (US); Ragadeepthi Tunduguru, Foster City, CA (US); Dalya Rivka Rosner, Redwood City, CA (US); Ksenya Shchors, San Mateo, CA (US); Suhail Rasool, Foster City, CA (US)

(21) Appl. No.: 17/834,703

(22) Filed: Jun. 7, 2022

Related U.S. Application Data
(60) Provisional application No. 63/202,373, filed on Jun. 8, 2021.

(51) Int. Cl. C07K 16/28; A61P 3/10

(52) U.S. Cl. CPC C07K 16/2851; A61P 3/10; C07K 2317/76; C07K 2317/34; A61K 2039/505

(57) ABSTRACT

Disclosed herein are methods and compositions for disrupting an interaction between Galectin-3 and insulin receptor or glucose transporters. Further disclosed herein are methods and compositions for the treatment of a disease or a disorder in a subject, such as the treatment of diabetes mellitus, insulin resistance, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular diseases, cardiac hypertrophy, myocardial ischemia, hypertension, pancreatic cancer associated diabetes (PCDM), rhabdomyosarcoma, or cancers.

### CROSS-REFERENCE TO RELATED APPLICATIONS

This application claims the benefit of priority of U.S. Provisional Patent Application 63/202,373, filed Jun. 8, 2021, which is hereby expressly incorporated by reference in its entirety.

### REFERENCE TO SEQUENCE LISTING

The present application is being filed along with a Sequence Listing in electronic format. The Sequence Listing is provided as a file entitled SeqListingIMMUT029A.TXT, which was created and last modified on Jun. 7, 2022, which is 1,984,217 bytes in size. The information in the electronic Sequence Listing is hereby incorporated by reference in its entirety.

### FIELD

Aspects of the present disclosure relate generally to antibodies or binding fragments thereof that block or disrupt the interaction between Galectin-3 (Gal3) and insulin receptor (INSR) or a glucose transporter, such as glucose transporter 1 (GLUT1) or glucose transporter 4 (GLUT4).

### BACKGROUND

Galectin-3 (Gal3, GAL3) is a lectin, or a carbohydrate-binding protein, with specificity towards beta-galactosides. In human cells, Gal3 is expressed and can be found in the nucleus, cytoplasm, cell surface, and in the extracellular space. Gal3 recognizes and interacts with beta-galactose conjugates on various proteins.

### SUMMARY

Aspects of the present disclosure relate generally to antibodies or binding fragments thereof that block or disrupt the interaction between Galectin-3 (Gal3) and insulin receptor (INSR) or a glucose transporter, such as glucose transporter 1 (GLUT1) or glucose transporter 4 (GLUT4). Further disclosed herein are methods and compositions for the treatment of diseases or disorders, such as (but not limited to) diabetes mellitus, insulin resistance, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular diseases, cardiac hypertrophy, myocardial ischemia, hypertension, pancreatic cancer associated diabetes (PCDM), rhabdomyosarcoma, or cancers, which can be associated with INSR and/or GLUT dysfunction.

Galectin-3 (Gal3) has been implicated to have immunomodulatory activity. An example of this is the interaction between Gal3 and T-cell immunoglobulin and mucin-domain containing-3 (TIM-3), which causes suppression of immune responses such as T cell activation and may enable cancer cells to evade immune clearance. This phenomenon and methods to inhibit the same are exemplified in WO 2019/023247 and WO 2020/160156, each of which is hereby expressly incorporated by reference in its entirety.

Disclosed herein are anti-Gal3 antibodies or binding fragments thereof. In some embodiments, the antibodies or binding fragments thereof comprise a heavy chain variable region comprising a V_{H}-CDR1, a V_{H}-CDR2, and a V_{H}-CDR3; and a light chain variable region comprising a V_{L}-CDR1, a V_{L}-CDR2, and a V_{L}-CDR3. In some embodiments, the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 32, 37, or 66. In some embodiments, the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 801, 951, 952, 77, or 108. In some embodiments, the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 953, 954, 802, 118, or 164. In some embodiments, the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 171, 178, or 215. In some embodiments, the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 222, 229, or 225. In some embodiments, the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 257, 256, or 291.

In some embodiments, the heavy chain variable region of the anti-Gal3 antibody or binding fragment thereof comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 806-820, 955-968, 1067-1109, or 1415-1439. In some embodiments, the light chain variable region of the anti-Gal3 antibody or binding fragment thereof comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 821-835, 969-982, 1110-1152, or 1440-1464. In some embodiments, the anti-Gal3 antibody or binding fragment thereof comprises a heavy chain, wherein the heavy chain comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 836-850, 983-996, 1411, 1153-1195, or 1465-1489. In some embodiments, the anti-Gal3 antibody or binding fragment thereof comprises a light chain, wherein the light chain comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 851-865, 997-1010, 1196-1238, 1412 or 1490-1514.

Also disclosed herein are nucleic acids comprising a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the nucleic acid sequences of SEQ ID NOs: 866-925, 1011-1066, 1239-1410, 1413-1414, or 1515-1614.

Also disclosed herein are methods of enhancing glucose transporter (GLUT) translocation in a cell. In some embodiments, the methods may comprise contacting the cell with an anti-Gal3 antibody or binding fragment thereof. In some embodiments, the glucose transporter is glucose transporter 1 (GLUT1) and/or glucose transporter 4 (GLUT4). In some embodiments, binding of the anti-Gal3 antibody or binding fragment thereof to Gal3 in the cell inhibits Gal3-mediated blocking of GLUT translocation. In some embodiments, the method is performed in vitro or in vivo. In some embodiments, GLUT translocation in the cell is enhanced by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% after contacting with the anti-Gal3 antibody or binding fragment thereof relative to a cell that is not contacted with the anti-Gal3 antibody or binding fragment thereof. In some embodiments, the anti-Gal3 antibody or binding fragment is any one or more of the anti-Gal3 antibodies or binding fragments thereof, or any portion or component of any one or more of the anti-Gal3 antibodies or binding fragments thereof disclosed herein, including but not limited to 1, 2, 3, 4, 5, or 6 CDRs, heavy chain variable regions, light chain variable regions, heavy chains, or light chains.

Also disclosed herein are methods of enhancing glucose transporter (GLUT) translocation in a cell. In some embodiments, the methods may comprise contacting the cell with a preincubated complex of Gal3 and an anti-Gal3 antibody or binding fragment thereof. In some embodiments, the glucose transporter is glucose transporter 1 (GLUT1) and/or glucose transporter 4 (GLUT4). In some embodiments, the method is performed in vitro or in vivo. In some embodiments, GLUT translocation in the cell is enhanced by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% after contacting with the preincubated complex of Gal3 and the anti-Gal3 antibody or binding fragment thereof relative to a cell that is not contacted with the preincubated complex of Gal3 and the anti-Gal3 antibody or binding fragment thereof. In some embodiments, the anti-Gal3 antibody or binding fragment is any one or more of the anti-Gal3 antibodies or binding fragments thereof, or any portion or component of any one or more of the anti-Gal3 antibodies or binding fragments thereof disclosed herein, including but not limited to 1, 2, 3, 4, 5, or 6 CDRs, heavy chain variable regions, light chain variable regions, heavy chains, or light chains.

Also disclosed herein are methods of improving insulin sensitivity in a subject in need thereof. In some embodiments, the methods may comprise administering to the subject an anti-Gal3 antibody or binding fragment thereof. In some embodiments, binding of the anti-Gal3 antibody or binding fragment thereof to Gal3 in the subject inhibits Gal3-mediated blocking of GLUT translocation in the subject, thereby improving insulin sensitivity in the subject. In some embodiments, the GLUT is glucose transporter 1 (GLUT1) and/or glucose transporter 4 (GLUT4). In some embodiments, the methods further comprise identifying the subject as needing improvement in insulin sensitivity prior to the administering step. In some embodiments, the methods further comprise detecting an improvement in insulin sensitivity in the subject following the administering step. In some embodiments, detecting the improvement in insulin sensitivity in the subject is done by measuring blood sugar levels, measuring blood insulin levels, glucose tolerance testing, or hyperinsulinemic euglycemic clamp. In some embodiments, the insulin sensitivity in the subject is improved by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% relative to the insulin sensitivity of the subject prior to the administering step. In some embodiments, the anti-Gal3 antibody or binding fragment is any one or more of the anti-Gal3 antibodies or binding fragments thereof, or any portion or component of any one or more of the anti-Gal3 antibodies or binding fragments thereof disclosed herein, including but not limited to 1, 2, 3, 4, 5, or 6 CDRs, heavy chain variable regions, light chain variable regions, heavy chains, or light chains.

Also disclosed herein are methods of improving insulin sensitivity in a subject in need thereof. In some embodiments, the methods may comprise administering to the subject a preincubated complex of Gal3 and an anti-Gal3 antibody or binding fragment thereof, thereby improving insulin sensitivity in the subject. In some embodiments, the GLUT is glucose transporter 1 (GLUT1) and/or glucose transporter 4 (GLUT4). In some embodiments, the methods further comprise identifying the subject as needing improvement in insulin sensitivity prior to the administering step. In some embodiments, the methods further comprise detecting an improvement in insulin sensitivity in the subject following the administering step. In some embodiments, detecting the improvement in insulin sensitivity in the subject is done by measuring blood sugar levels, measuring blood insulin levels, glucose tolerance testing, or hyperinsulinemic euglycemic clamp. In some embodiments, the insulin sensitivity in the subject is improved by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% relative to the insulin sensitivity of the subject prior to the administering step. In some embodiments, the anti-Gal3 antibody or binding fragment is any one or more of the anti-Gal3 antibodies or binding fragments thereof, or any portion or component of any one or more of the anti-Gal3 antibodies or binding fragments thereof disclosed herein, including but not limited to 1, 2, 3, 4, 5, or 6 CDRs, heavy chain variable regions, light chain variable regions, heavy chains, or light chains.

Also disclosed herein are methods of treating a disease associated with insulin resistance in a subject in need thereof. In some embodiments, the methods may comprise administering to the subject an anti-Gal3 antibody or binding fragment thereof, thereby treating the disease associated with insulin resistance in the subject. In some embodiments, the disease associated with insulin resistance comprises diabetes mellitus, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular disease, cardiac hypertrophy, myocardial ischemia, hypertension, pancreatic cancer associated diabetes (PCDM), or cancer. In some embodiments, the methods further comprise identifying the subject as needing treatment of the disease associated with insulin resistance prior to the administering step. In some embodiments, the methods further comprise detecting an improvement in the disease associated with insulin resistance following the administering step. In some embodiments, detecting an improvement in the disease associated with insulin resistance comprises detecting an improvement in insulin sensitivity in the subject. In some embodiments, detecting the improvement in insulin sensitivity in the subject is done by measuring blood sugar levels, measuring blood insulin levels, glucose tolerance testing, or hyperinsulinemic euglycemic clamp. In some embodiments, the insulin sensitivity in the subject is improved by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% relative to the insulin sensitivity of the subject prior to the administering step. In some embodiments, the anti-Gal3 antibody or binding fragment is any one or more of the anti-Gal3 antibodies or binding fragments thereof, or any portion or component of any one or more of the anti-Gal3 antibodies or binding fragments thereof disclosed herein, including but not limited to 1, 2, 3, 4, 5, or 6 CDRs, heavy chain variable regions, light chain variable regions, heavy chains, or light chains.

Also disclosed herein are methods of treating a disease associated with insulin resistance in a subject in need thereof. In some embodiments, the methods may comprise administering to the subject a preincubated complex of Gal3 and an anti-Gal3 antibody or binding fragment thereof, thereby treating the disease associated with insulin resistance in the subject. In some embodiments, the disease associated with insulin resistance comprises diabetes mellitus, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular disease, cardiac hypertrophy, myocardial ischemia, hypertension, pancreatic cancer associated diabetes (PCDM), or cancer. In some embodiments, the methods further comprise identifying the subject as needing treatment of the disease associated with insulin resistance prior to the administering step. In some embodiments, the methods further comprise detecting an improvement in the disease associated with insulin resistance following the administering step. In some embodiments, detecting an improvement in the disease associated with insulin resistance comprises detecting an improvement in insulin sensitivity in the subject. In some embodiments, detecting the improvement in insulin sensitivity in the subject is done by measuring blood sugar levels, measuring blood insulin levels, glucose tolerance testing, or hyperinsulinemic euglycemic clamp. In some embodiments, the insulin sensitivity in the subject is improved by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% relative to the insulin sensitivity of the subject prior to the administering step. In some embodiments, the anti-Gal3 antibody or binding fragment is any one or more of the anti-Gal3 antibodies or binding fragments thereof, or any portion or component of any one or more of the anti-Gal3 antibodies or binding fragments thereof disclosed herein, including but not limited to 1, 2, 3, 4, 5, or 6 CDRs, heavy chain variable regions, light chain variable regions, heavy chains, or light chains.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 demonstrates the ability of galectin-3 targeted antibodies to block the binding of Gal3 and Insulin Receptor (INSR) as measured by enzyme-linked immunosorbent assay (ELISA) at 10, 3, and 1 μg/mL. Bars represent mean+/−standard deviation.

FIG. 2 depicts titrations of a limited series of galectin-3 targeted antibodies to block Gal3 and Insulin Receptor (INSR) binding as measured by enzyme-linked immunosorbent assay (ELISA). Bars represent mean+/−standard deviation.

FIG. 3A depicts a summary of properties for exemplary anti-Gal3 antibodies.

FIG. 3B depicts the identification of Gal3-binding antibody bins by antibody competition. Values represent inhibition as assessed by biolayer interferometry.

FIG. 4 demonstrates the reduction of weight gain in mice fed with a normal diet or with 60% high fat diet (HFD) for 8 weeks and dosed with Isotype control antibody HuIgG4, or with Gal3-targeted TB001 (IMT001-4). Left panel depicts mean absolute values, and right panel depicts mean percent change per animal+/−standard error.

FIG. 5 depicts glucose tolerance in mice treated as in FIG. 4. Left panel depicts serum mean glucose levels after glucose bolus; right panel depicts mean area under curve (AUC) from data as represented in the left panel+/1 standard error.

FIG. 6 depicts insulin resistance in mice as treated in FIG. 4. Left panel depicts serum mean glucose levels after insulin bolus; right panel depicts mean area over curve (AOC) from data as represented in the left panel+/1 standard error.

FIG. 7 depicts hematoxylin and eosin staining of formalin fixed paraffin embedded liver sections from mice as treated in FIG. 4. Note the evidence of steatosis in HFD-fed mice treated with control IgG4 and absence thereof in those treated with IMT001-4 (TB001).

FIG. 8 depicts serum liver enzyme ALT levels in mice treated as in FIG. 4. Bars represent mean+/−standard error.

FIG. 9 depicts the amounts of circulating Gal3 in Bks-db or C57BL6/J control mice.

FIG. 10 depicts a Kaplan-Meier curve of healthy C57BL6/J mice and db/db mice treated with the anti-Gal3 antibody mTB001, a PBS negative control, or a semaglutide positive control.

FIG. 11 depicts the change in levels of fasting blood glucose of healthy C57BL6/J mice or db/db mice treated with either mTB001 or PBS.

FIG. 12 depicts the mean survival of NOD/ShiLtJ mice treated with mTB001 compared to untreated control.

FIG. 13A depicts the change in levels of fasting blood glucose of NOD/ShiLtJ mice treated with mTB001 compared to untreated control.

FIG. 13B depicts the circulating levels of C-peptide in NOD/ShiLtJ mice treated with mTB001 compared to untreated control, and in normal control mice.

FIG. 14 depicts primers used for RT-qPCR for quantifying inflammatory cytokines in an inflammatory bowel disease (IBD) mouse model.

FIG. 15 depicts the measured colon length in DSS-induced IBD mice treated with mTB001 or PBS compared to normal mice.

FIG. 16 depicts the quantification of circulating IFN-γ in DSS-induced IBD mice treated with mTB001 (10 mg/kg and 1 mg/kg) or PBS compared to normal mice.

FIG. 17 depicts protein sequences of Gal3, insulin receptor (INSR), glucose transporter 4 (GLUT4), and glucose transporter 1 (GLUT1).

FIG. 18 depicts peptide sequences of Gal3 used to generate and analyze antibodies.

FIG. 19A depicts exemplary variable heavy chain complementarity-determining region (CDR) 1 for anti-Gal3 antibodies disclosed herein. In some embodiments, any of the compositions or methods provided herein can include one or more of the variable heavy chain CDR1 provided herein.

FIG. 19B depicts exemplary variable heavy chain CDR2 for anti-Gal3 antibodies disclosed herein. In some embodiments, any of the compositions or methods provided herein can include one or more of the variable heavy chain CDR2 provided herein.

FIG. 19C depicts exemplary variable heavy chain CDR3 for anti-Gal3 antibodies disclosed herein. In some embodiments, any of the compositions or methods provided herein can include one or more of the variable heavy chain CDR3 provided herein.

FIG. 20A depicts exemplary variable light chain CDR1 for anti-Gal3 antibodies disclosed herein. In some embodiments, any of the compositions or methods provided herein can include one or more of the variable light chain CDR1 provided herein.

FIG. 20B depicts exemplary variable light chain CDR2 for anti-Gal3 antibodies disclosed herein. In some embodiments, any of the compositions or methods provided herein can include one or more of the variable light chain CDR2 provided herein.

FIG. 20C depicts exemplary variable light chain CDR3 for anti-Gal3 antibodies disclosed herein. In some embodiments, any of the compositions or methods provided herein can include one or more of the variable light chain CDR3 provided herein.

FIG. 21 depicts exemplary heavy chain variable region (VH) sequences for anti-Gal3 antibodies disclosed herein. In some embodiments, any of the compositions or methods provided herein can include one or more of the VH sequences provided herein.

FIG. 22 depicts exemplary light chain variable region (VL) sequences for anti-Gal3 antibodies disclosed herein. In some embodiments, any of the compositions or methods provided herein can include one or more of the VL sequences provided herein.

FIG. 23 depicts exemplary combinations of heavy and light chain CDRs (CDR1, CDR2, and CDR3) of exemplary anti-Gal3 antibodies disclosed herein. In some embodiments, any of the compositions or methods provided herein can include one or more of the heavy and light chain CDR combinations provided herein.

FIG. 24 depicts exemplary combinations of heavy and light chain variable regions of exemplary anti-Gal3 antibodies disclosed herein. In some embodiments, any of the compositions or methods provided herein can include one or more of the heavy and light chain variable region combinations provided herein.

FIG. 25 depicts exemplary heavy chain (HC) sequences and light chain (LC) sequences, and possible pairings for exemplary anti-Gal3 antibodies disclosed herein. In some embodiments, any of the compositions or methods provided herein can include one or more of the HC or LC, or pairs of HC and LC sequences provided herein.

FIG. 26 depicts peptides that were found to bind to exemplary anti-Gal3 antibodies disclosed herein (according to the peptide nomenclature depicted in FIG. 18 as discussed herein) and binning of these exemplary antibodies.

FIG. 27 depicts KD (M) values of Gal3 binding for exemplary anti-Gal3 antibodies disclosed herein.

FIG. 28 depicts antibody affinities (K_{D}) of anti-Gal3 humanized antibodies IMT001 and IMT006a for human, cynomolgus, and mouse Gal3.

FIG. 29 depicts antibody names used throughout the present disclosure refer to the same antibody (with exemplary peptide and nucleic acid sequences provided elsewhere in the disclosure and appropriately attributed to at least one of the depicted names) and may be used interchangeably. The names shown in a column correspond to the same antibody.

FIG. 30 depicts a graphical representation of relative GLUT4 translocation determined as a fold change between insulin-stimulated and insulin-unstimulated (basal) cells±Gal3 when contacted with various exemplary anti-Gal3 antibodies (or no antibody control), as measured by immunocytochemistry based assay. Antibody 2D10 here refers to 2D10-VH0-VL0.

FIG. 31A-B depict graphical representations of relative GLUT4 translocation determined as a fold change between insulin-stimulated and insulin-unstimulated (basal) cells±Gal3 when contacted with variants of 20H5 (FIG. 31A) or 2D10-VH0-VL0 (2D10) (FIG. 31B) (or no antibody control) as measured by immunocytochemistry based assay.

FIG. 32 depicts an alignment of hinge and constant heavy chain domain 2 (CH2) domain amino acid sequences of wild-type human immunoglobulin G1 (IgG1), IgG2 and IgG4 as well as their sigma variants. The alignment above uses EU numbering. Residues identical to wild-type IgG1 are indicated as dots; gaps are indicated with hyphens. Sequence is given explicitly if it differs from wild-type IgG1 or from the parental subtype for a variants. Open boxes beneath the alignment correspond to International Immunogenetics Information System (IMGT) strand definitions. Boxes beneath the alignment correspond to the strand and helix secondary structure assignment for wild-type IgG1. Residues 267-273 form the BC loop and 322-332 form the FG loop. Also provided are exemplary constant regions for human IgG4 heavy (S228P mutant) and light (kappa) chains (SEQ ID NOs: 945-946), murine IgG2A (LALAPG and LALA mutants) (SEQ ID NOs: 947-948), and human IgG1 (KEM, REM, and LALAPGv2 mutants) (SEQ ID NOs: 1615-1617). In some embodiments, any one or more of the VH/VL and/or CDRs provided in the other figures or otherwise disclosed herein can be paired with any one or more of the exemplary constant regions provided herein.

FIG. 33 depicts nucleic acid sequences that encode for exemplary heavy chain variable regions of anti-Gal3 antibodies disclosed herein. In some embodiments, any of the compositions or methods provided herein can include one or more of the heavy chain variable regions encoded by the nucleic acids provided herein.

FIG. 34 depicts nucleic acid sequences that encode for exemplary light chain variable regions of anti-Gal3 antibodies disclosed herein. In some embodiments, any of the compositions or methods provided herein can include one or more of the light chain variable regions encoded by the nucleic acids provided herein.

FIG. 35 depicts nucleic acid sequences that encode for exemplary heavy chains of anti-Gal3 antibodies disclosed herein. In some embodiments, any of the compositions or methods provided herein can include one or more of the heavy chains encoded by the nucleic acids provided herein.

FIG. 36 depicts nucleic acid sequences that encode for exemplary light chains of anti-Gal3 antibodies disclosed herein. In some embodiments, any of the compositions or methods provided herein can include one or more of the light chains encoded by the nucleic acids provided herein.

FIG. 37A-B depicts an exemplary alignment for the heavy chain CDRs (FIG. 37A) and light chain CDRs (FIG. 37B) for the exemplary anti-Gal3 antibodies disclosed herein.

FIG. 38A-D each depict a table with a subset of anti-Gal3 antibodies disclosed herein. In some embodiments, sequences, including heavy chain variable region CDRs, light chain variable region CDRs, heavy chain variable regions, light chain variable regions, heavy chains, and light chains, or combinations thereof, can be selected from any one or more of the anti-Gal3 antibodies provided as subsets in each table.

FIG. 39A-E each depict a table with a subset of anti-Gal3 antibodies disclosed herein. In some embodiments, sequences, including heavy chain variable region CDRs, light chain variable region CDRs, heavy chain variable regions, light chain variable regions, heavy chains, and light chains, or combinations thereof, can be selected from any one or more of the anti-Gal3 antibodies provided as subsets in each table.

FIG. 40A-C depict the quantification of affinity (KD) of TB006 (FIG. 40A), TB006 (FIG. 40B), and a control antibody Synagis (FIG. 40C) to human Gal3.

FIG. 41A-C depict the quantification of affinity (KD) of TB006 (FIG. 41A), TB006 (FIG. 41B), and a control antibody Synagis (FIG. 41C) to mouse Gal3.

FIG. 42A-B depict the quantification of affinity (KD) of TB006 (FIG. 42A, left panel), TB001 (FIG. 42B, right panel), and a control antibody Synagis (FIG. 42B) to cynomolgus Gal3.

FIG. 43A-C depict the quantification of affinity (KD) of TB006 (FIG. 43A), TB001 (FIG. 43B), and a control antibody Synagis (FIG. 43C) to rat Gal3.

FIG. 44A-E depict the quantification of insulin-independent GLUT4 translocation mediated by Gal3 complexed with exemplary anti-Gal3 antibodies TB006 (FIG. 44A), 2D10 variants (FIG. 44B), 20H5 variants (FIG. 44C), 21H6 variants (FIG. 44D), and other exemplary anti-Gal3 antibodies disclosed herein (FIG. 44E).

FIG. 45 depicts the quantification of insulin-independent GLUT4 translocated mediated by different mutants of Gal3 complexed with exemplary anti-Gal3 antibody TB006.

FIG. 46A-D depict the quantification of Gal3 binding to GLUT1 or GLUT4 by ELISA (FIG. 46A), and the ability of exemplary anti-Gal3 antibodies TB006 and 2D10-VH0-VL0 to disrupt the binding of Gal3 to GLUT1 (FIG. 46B) or GLUT4 (FIG. 46C). FIG. 46D depicts the IC50 of antibody-mediated disruption of the Gal3 binding to GLUT1 or GLUT4 as measured in FIG. 46B-C.

FIG. 47 depicts a hydrogen-deuterium mass spectrometric heat map of hGal3 complexed with GLUT4, showing the putative regions of Gal3 responsible for binding to GLUT4. On the heat map, darker regions have less deuterium uptake after binding.

FIG. 48 depicts fluorescent microscopy images of L6 myoblasts treated with exemplary anti-Gal3 antibodies TB006 or 2D10 with or without Gal3, indicating that the anti-Gal3 antibodies are internalized into the myoblasts only in the presence of Gal3.

FIG. 49A-B depict quantification of inhibition of Gal3-mediated glucose tolerance by co-treatment with anti-Gal3 antibodies in vivo. FIG. 49A shows an exemplary schematic of the study. FIG. 49B shows glucose levels in a time series of blood samples taken after administration of Gal3 with or without the exemplary anti-Gal3 antibody TB001 and subsequent administration of glucose. Mice injected with Gal3 only exhibited increased blood glucose levels, while mice injected with a mixture of Gal3 and anti-Gal3 antibody exhibited blood glucose levels similar to PBS injected control mice.

### DETAILED DESCRIPTION OF THE DISCLOSURE

Diabetes mellitus is a group of metabolic diseases characterized by a high blood sugar level over a prolonged period of time. There are three main types of diabetes mellitus: Type I diabetes, Type II diabetes, and gestational diabetes. Type I diabetes is caused by the failure of the pancreas to produce enough insulin due to the loss of insulin-producing beta cells. Type II diabetes is characterized by insulin resistance and may be due to a variety of lifestyle, dietary, and genetic factors, including obesity, poor diet, and stress. Gestational diabetes occurs when a pregnant woman without a prior history of diabetes develops high blood sugar levels.

In addition to diabetes, insulin resistance is associated with other diseases. Insulin resistance arises when cells of a subject become less sensitive to insulin, resulting in additional secretion of insulin by the pancreas. If the pancreas is unable to keep up with the necessary production of insulin to keep blood sugar levels at a safe level, the subject may progress into prediabetes and diabetes. Hyperinsulinemia (abnormally high levels of insulin in the blood) may also arise. Insulin resistance has also been attributed to other diseases including dysmetabolic syndrome, type A insulin resistance syndrome, type B insulin resistance syndrome, acanthosis nigricans, polycystic ovary syndrome (PCOS). Obesity and cardiovascular disease is also linked to insulin resistance and prediabetes/diabetes.

Galectin-3 (Gal3, GAL3) is known to play an important role in cell proliferation, adhesion, differentiation, angiogenesis, and apoptosis. This activity is, at least in part, due to immunomodulatory properties and binding affinity towards other immune regulatory proteins, signaling proteins, and other cell surface markers. Gal3 functions by distinct N-terminal and C-terminal domains. The N-terminal domain (isoform 1: amino acids 1-111, isoform 3: amino acids 1-125) comprise a tandem repeat domain (TRD, isoform 1: amino acids 36-109, isoform 3: amino acids 50-123) and is largely responsible for oligomerization of Gal3. The C-terminal domain (isoform 1: amino acids 112-250, isoform 3: amino acids 126-264) comprise a carbohydrate-recognition-binding domain (CRD), which binds to β-galactosides. An exemplary sequence for isoform 1 of human Gal3 (NCBI Reference No. NP_002297.2) is shown in SEQ ID NO: 1. An exemplary sequence for isoform 3 of human Gal3 (NCBI Reference No. NP_001344607.1) is shown in SEQ ID NO: 2.

Further, Gal3 has been shown to be elevated in obese humans and is believed to cause insulin resistance and glucose intolerance in these subjects. Gal3 has been shown to bind directly to insulin receptor and to inhibit downstream signaling. Thus, Gal3 may contribute to obesity-induced insulin resistance and chronic tissue inflammation.

In some embodiments, anti-Gal3 antibodies or binding fragments thereof or compositions comprising anti-Gal3 antibodies or binding fragments thereof are provided. In some embodiments, the anti-Gal3 antibodies or binding fragments thereof bind to the N-terminal domain, the N-terminus and/or the TRD of Gal3. In some embodiments, the anti-Gal3 antibodies or binding fragments thereof bind to the C-terminal domain, the C-terminus and/or the CRD of Gal3. In some embodiments, the anti-Gal3 antibodies or binding fragments thereof do not bind to the N-terminal domain, the N-terminus and/or the TRD of Gal3. In some embodiments, the anti-Gal3 antibodies or binding fragments thereof do not bind to the C-terminal domain, the C-terminus and/or the CRD of Gal3.

In some embodiments provided herein, it is shown that Gal3 inhibits glucose transporter (GLUT) translocation in biological cells. In some embodiments, the glucose transporter is GLUT1 and/or GLUT4. This inhibition may lead to insulin resistance and diseases or disorders associated with insulin resistance.

Disclosed herein, in some embodiments, are methods of enhancing GLUT translocation in a cell by contacting the cell with an anti-Gal3 antibody or binding fragment thereof. In some embodiments, the anti-Gal3 antibody or binding fragment thereof blocks or disrupts an interaction between Gal3 and GLUT, or another protein associated with GLUT translocation. In some embodiments, the GLUT is GLUT1 and/or GLUT4. This contacting may be done in vitro or in vivo.

Also disclosed herein, in some embodiments, are methods of enhancing GLUT translocation in a cell by contacting the cell with a preincubated complex of Gal3 and an anti-Gal3 antibody or binding fragment thereof. In some embodiments, the GLUT is GLUT1 and/or GLUT4. This contacting may be done in vitro or in vivo.

Also disclosed herein, in some embodiments, are methods of improving insulin sensitivity in a subject in need thereof by administering an anti-Gal3 antibody or binding fragment thereof to the subject. In some embodiments, the anti-Gal3 antibody or binding fragment thereof blocks or disrupts an interaction between Gal3 and GLUT, or another protein associated with GLUT translocation, thereby improving insulin sensitivity in the subject. In some embodiments, the GLUT is GLUT1 and/or GLUT4.

Also disclosed herein, in some embodiments, are methods of improving insulin sensitivity in a subject in need thereof by administering a preincubated complex of Gal3 and an anti-Gal3 antibody or binding fragment thereof to the subject, thereby improving insulin sensitivity in the subject. In some embodiments, the GLUT is GLUT1 and/or GLUT4.

Also disclosed herein, in some embodiments, are methods of treating a disease associated with insulin resistance in a subject in need thereof by administering an anti-Gal3 antibody or binding fragment thereof to the subject, thereby treating the disease associated with insulin resistance in the subject. In some embodiments, the disease associated with insulin resistance comprises diabetes mellitus, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular disease, cardiac hypertrophy, myocardial ischemia, hypertension, pancreatic cancer associated diabetes (PCDM) or cancer. In some embodiments, administration of the anti-Gal3 antibody or binding fragment thereof to the subject treats the disease, or insulin resistance associated with the disease. In some embodiments, the method involves administering any antibody or variant thereof as provided herein, in a therapeutically effective amount, sufficient to interfere with the interaction between GAL3 and GLUT (e.g. GLUT1 and/or GLUT4), so as to treat (either in response to a subject having and/or to reduce the risk of) one or more of: diabetes mellitus, insulin resistance, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular diseases, cardiac hypertrophy, myocardial ischemia, hypertension, pancreatic cancer associated diabetes (PCDM), or cancers.

Also disclosed herein, in some embodiments, are methods of treating a disease associated with insulin resistance in a subject in need thereof by administering a preincubated complex of Gal3 and an anti-Gal3 antibody or binding fragment thereof to the subject, thereby treating the disease associated with insulin resistance in the subject. In some embodiments, the disease associated with insulin resistance comprises diabetes mellitus, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular disease, cardiac hypertrophy, myocardial ischemia, hypertension, pancreatic cancer associated diabetes (PCDM) or cancer. In some embodiments, administration of the anti-Gal3 antibody or binding fragment thereof to the subject treats the disease, or insulin resistance associated with the disease. In some embodiments, the method involves administering any antibody or variant thereof as provided herein, in a therapeutically effective amount, sufficient to interfere with the interaction between GAL3 and GLUT (e.g. GLUT1 and/or GLUT4), so as to treat (either in response to a subject having and/or to reduce the risk of) one or more of: diabetes mellitus, insulin resistance, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular diseases, cardiac hypertrophy, myocardial ischemia, hypertension, pancreatic cancer associated diabetes (PCDM), or cancers.

In some embodiments, administering any one of the anti-Gal3 antibodies or binding fragments thereof disclosed herein can reduce insulin resistance in a subject.

### Definitions

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

The section headings used herein are for organizational purposes only and are not to be construed as limiting the subject matter described.

In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “polypeptide”, “peptide”, and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component.

As used herein the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence.

As used herein, the term “antibody” denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope. Antibodies may be polyclonal antibodies, although monoclonal antibodies may be preferred because they may be reproduced by cell culture or recombinantly and can be modified to reduce their antigenicity.

In addition to entire immunoglobulins (or their recombinant counterparts), immunoglobulin fragments or “binding fragments” comprising the epitope binding site (e.g., Fab′, F(ab′)2, single-chain variable fragment (scFv), diabody, minibody, nanobody, single-domain antibody (sdAb), or other fragments) are useful as antibody moieties in the present invention. Such antibody fragments may be generated from whole immunoglobulins by ricin, pepsin, papain, or other protease cleavage. Minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g. pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (e.g. U.S. Pat. No. 5,985,660, hereby expressly incorporated by reference in its entirety).

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As known in the art, a “constant region” of an antibody refers to the constant region of the antibody light chain or the constant region of the antibody heavy chain, either alone or in combination.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the IMGT approach (Lefranc et al., 2003) Dev Comp Immunol. 27:55-77), computational programs such as Paratome (Kunik et al., 2012, Nucl Acids Res. W521-4), the AbM definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, IMGT, Paratome, AbM, and/or conformational definitions, or a combination of any of the foregoing.

As disclosed herein, sequences having a % identity to any of the sequences disclosed herein are envisioned and may be used. The terms “% identity” refer to the percentage of units (i.e. amino acids or nucleotides) that are the same between two or more sequences relative to the length of the sequence. When the two or more sequences being compared are the same length, the % identity will be respective that length. When two or more sequences being compared are different lengths, deletions and/or insertions may be introduced to obtain the best alignment. In some embodiments, these sequences may include peptide sequences, nucleic acid sequences, CDR sequences, variable region sequences, or heavy or light chain sequences. In some embodiments, any sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 substitutions, deletions, or additions relative to any of the sequences disclosed herein may be used. The changes in sequences may apply to, for example, single amino acids, single nucleic acid bases, or nucleic acid codons; however, differences in longer stretches of sequences are also envisioned. As applied to antibody sequences, these differences in sequences may apply to antigen-binding regions (e.g., CDRs) or regions that do not bind to antigens or are only secondary to antigen binding (e.g., framework regions).

As disclosed herein, sequences having a % homology to any of the sequences disclosed herein are envisioned and may be used. The term “% homology” refers to the degree of conservation between two sequences when considering their three-dimensional structure. For example, homology between two protein sequences may be dependent on structural motifs, such as beta strands, alpha helices, and other folds, as well as their distribution throughout the sequence. Homology may be determined through structural determination, either empirically or in silico. In some embodiments, any sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence homology to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 substitutions, deletions, or additions relative to any of the sequences disclosed herein, which may or may not affect the overall % homology, may be used.

As applied herein, sequences having a certain % similarity to any of the sequence disclosed herein are envisioned and may be used. In some embodiments, these sequences may include peptide sequences, nucleic acid sequences, CDR sequences, variable region sequences, or heavy or light chain sequences. As understood in the art with respect to peptide sequences, “similarity” refers to the comparison of amino acids based on their properties, including but not limited to size, polarity, charge, pK, aromaticity, hydrogen bonding properties, or presence of functional groups (e.g. hydroxyl, thiol, amine, carboxyl, and the like). The term “% similarity” refers to the percentage of units (i.e. amino acids) that are the same between two or more sequences relative to the length of the sequence. When the two or more sequences being compared are the same length, the % similarity will be respective that length. When two or more sequences being compared are different lengths, deletions and/or insertions may be introduced to obtain the best alignment. The similarity of two amino acids may dictate whether a certain substitution is conservative or non-conservative. Methods of determining the conservativeness of an amino acid substitution are generally known in the art and may involve substitution matrices. Commonly used substitution matrices include BLOSUM45, BLOSUM62, BLOSUM80, PAM100, PAM120, PAM160, PAM200, PAM250, but other substitution matrices or approaches may be used as considered appropriate by the skilled person. A certain substitution matrix may be preferential over the others when considering aspects such as stringency, conservation and/or divergence of related sequences (e.g. within the same species or broader), and length of the sequences in question. As used herein, a peptide sequence having a certain % similarity to another sequence will have up to that % of amino acids that are either identical or an acceptable substitution as governed by the method of similarity determination used. In some embodiments, a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 similar substitutions relative to any of the sequences disclosed herein may be used. As applied to antibody sequences, these similar substitutions may apply to antigen-binding regions (i.e. CDRs) or regions that do not bind to antigens or are only secondary to antigen binding (i.e. framework regions).

The term “consensus sequence” as used herein with regard to sequences refers to the generalized sequence representing all of the different combinations of permissible amino acids at each location of a group of sequences. A consensus sequence may provide insight into the conserved regions of related sequences where the unit (e.g. amino acid or nucleotide) is the same in most or all of the sequences, and regions that exhibit divergence between sequences. In the case of antibodies, the consensus sequence of a CDR may indicate amino acids that are important or dispensable for antigen binding. It is envisioned that consensus sequences may be prepared with any of the sequences provided herein, and the resultant various sequences derived from the consensus sequence can be validated to have similar effects as the template sequences.

The term “compete,” as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, and/or more rapidly, and/or with greater duration and/or with greater affinity with a particular cell or substance than it does with alternative cells or substances. An antibody “specifically binds” or “preferentially binds” to a target if it binds with greater affinity, and/or avidity, and/or more readily, and/or with greater duration than it binds to other substances. For example, an antibody that specifically or preferentially binds to a target epitope is an antibody that binds this epitope with greater affinity, and/or avidity, and/or more readily, and/or with greater duration than it binds to other target epitopes or non-target epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

The term “block” or “disrupt” as used herein with regard to an antibody refers to the ability of an antibody to interfere with a biological process, including but not limited to activity of an enzyme, binding of two or more biological molecules (e.g. two or more proteins, peptides, nucleic acids, lipids, and the like), or advancement of a signaling cascade. Generally, interference with a biological process will involve the antibody binding to its target or an epitope thereof, thereby interfering with the normal function of said target, such as occluding an active site of the target, occluding another region of the target important for its function, or altering the localization and/or transport of the target. The blocking or disruption activity of an antibody may be quantified in terms of the reduction of the biological process in question relative to a control condition where the biological process is not disrupted. In other cases, the blocking or disruption activity of an antibody may be quantified in terms of a modulation in another biological process known to be associated with the target biological process, whether it be directly related or inversely related. In some embodiments, the blocking or disruption activity may cause a change of at least 5%, 10%, 20%, 30%, 40%, 50%, 60%, 70%, 80%, 90%, or 100%, or any percentage within a range defined by any two of the aforementioned values, relative to a control condition. In some embodiments provided herein, an interaction between Gal3 and GLUT (e.g., GLUT1 and/or GLUT4) is a biological process that can be disrupted by an anti-Gal3 antibody. It is envisioned that the interaction between Gal3 and GLUT may or may not be a direct interaction between Gal3 and GLUT, and the anti-Gal3 antibody may interfere with some other aspect of the activity of Gal3 and/or GLUT.

As used herein, the term “antigen binding molecule” refers to a molecule that comprises an antigen binding portion that binds to an antigen and, optionally, a scaffold or framework portion that allows the antigen binding portion to adopt a conformation that promotes binding of the antigen binding portion or provides some additional properties to the antigen binding molecule. In some embodiments, the antigen is Gal3. In some embodiments, the antigen binding portion comprises at least one CDR from an antibody that binds to the antigen. In some embodiments, the antigen binding portion comprises all three CDRs from a heavy chain of an antibody that binds to the antigen or from a light chain of an antibody that binds to the antigen. In some embodiments, the antigen binding portion comprises all six CDRs from an antibody that binds to the antigen (three from the heavy chain and three from the light chain). In some embodiments, the antigen binding portion is an antibody fragment.

Nonlimiting examples of antigen binding molecules include antibodies, antibody fragments (e.g., an antigen binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, pig, dog, cat, horse, donkey, guinea pig, goat, or camelid. Antibody fragments may compete for binding of a target antigen with an intact antibody and the fragments may be produced by the modification of intact antibodies (e.g. enzymatic or chemical cleavage) or synthesized de novo using recombinant DNA technologies or peptide synthesis. The antigen binding molecule can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding molecule as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronectin components as a scaffold.

An antigen binding molecule can also include a protein comprising one or more antibody fragments incorporated into a single polypeptide chain or into multiple polypeptide chains. For instance, antigen binding molecule can include, but are not limited to, a diabody (see, e.g., EP 404,097; WO 93/11161; and Hollinger et al., Proc. Natl. Acad. Sci. USA, Vol. 90:6444-6448, 1993); an intrabody; a domain antibody (single VL or VH domain or two or more VH domains joined by a peptide linker; see Ward et al., Nature, Vol. 341:544-546, 1989); a maxibody (2 scFvs fused to Fc region, see Fredericks et al., Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al., Protein Eng Des Sel., Vol. 17:315-23, 2004); a peptibody (one or more peptides attached to an Fc region, see WO 00/24782); a linear antibody (a pair of tandem Fd segments (VH-CH1-VH-CH1) which, together with complementary light chain polypeptides, form a pair of antigen binding regions, see Zapata et al., Protein Eng., Vol. 8:1057-1062, 1995); a small modular immunopharmaceutical (see U.S. Patent Publication No. 20030133939); and immunoglobulin fusion proteins (e.g. IgG-scFv, IgG-Fab, 2scFv-IgG, 4scFv-IgG, VH-IgG, IgG-VH, and Fab-scFv-Fc).

In certain embodiments, an antigen binding molecule can have, for example, the structure of an immunoglobulin. An “immunoglobulin” is a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Unless otherwise specified, the complementarity defining regions disclosed herein follow the IMGT definition. In some embodiments, any of the CDRs disclosed herein can instead be interpreted by Kabat, Chothia, or other definitions accepted by those of skill in the art.

The term “humanized” as applies to a non-human (e.g. rodent or primate) antibodies are hybrid immunoglobulins, immunoglobulin chains or fragments thereof which contain minimal sequence derived from non-human immunoglobulin.

As used herein, the term “cytokine” refers to small proteins, polypeptides, or peptides that are involved in inflammatory signaling or proteins released by one cell population that act on another cell as intercellular mediators or have an autocrine effect on the cells producing the proteins. Cytokines include but are not limited to chemokines, interferons, interleukins, lymphokines, monokines, tumor necrosis factors, CCL1, CCl2, CCL3, CCL4, CCL5, CCL6, CCL7, CCL8, CCL9, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CX3CL1, XCL1, XCL2, INFα, INFβ, INFγ, IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-17A-F, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, adesleukin, GM-CSF, TNFα, TNFβ, TNFγ, TGF-I-3 TNFSF4, TNFSF5, TNFSF6, TNFSF7, TNFSF8, TNFSF9, TNFSF10, TNFSF11, TNFSF12, TNFSF13, TNFSF13B, TNFSF14, TNFSF15, TNFSF18, or TNFSF19, leukemia inhibitor factor (LIF), ciliary neurotrophic factor (CNTF), CNTF-like cytokine (CLC), cardiotrophin (CT), Kit ligand (KL), or any combination thereof.

As used herein, the terms “treating” or “treatment” (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The terms “effective amount” or “effective dose” as used herein have their plain and ordinary meaning as understood in light of the specification, and refer to that amount of a recited composition or compound that results in an observable designated effect. Actual dosage levels of active ingredients in an active composition of the presently disclosed subject matter can be varied so as to administer an amount of the active composition or compound that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

In some non-limiting embodiments, an effective amount or effective dose of a composition or compound may relate to the amount or dose that provides a significant, measurable, or sufficient therapeutic effect towards the treatment of any one or more of the diseases provided herein, such as insulin resistance, diabetes mellitus, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular disease, cardiac hypertrophy, myocardial ischemia, hypertension, pancreatic cancer associated diabetes (PCDM) or cancers. In some embodiments, the effective amount or effective dose of a composition or compound may treat, ameliorate, or prevent the progression of symptoms of any one or more of the diseases provided herein.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical formulation is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include sugars, starch, glucose, fructose, lactose, sucrose, maltose, gelatin, malt, rice, flour, chalk, silica gel, sodium stearate, glycerol monostearate, talc, salts, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as glycerol, erythritol, threitol, arabitol, xylitol, ribitol, mannitol, sorbitol, galactitol, fucitol, iditol, inositol, isomalt, maltitol, or lactitol, salt-forming counterions such as sodium, and nonionic surfactants such as TWEEN®, polyethylene glycol (PEG), and PLURONICS®. The formulation, if desired, can also contain minor amounts of wetting, bulking, emulsifying agents, or pH buffering agents. These formulations can take the form of solutions, suspensions, emulsion, sustained release formulations and the like. The formulation should suit the mode of administration.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

As used herein, a “carrier” refers to a compound, particle, solid, semi-solid, liquid, or diluent that facilitates the passage, delivery and/or incorporation of a compound to cells, tissues and/or bodily organs. For example, without limitation, a lipid nanoparticle (LNP) is a type of carrier that can encapsulate an oligonucleotide to thereby protect the oligonucleotide from degradation during passage through the bloodstream and/or to facilitate delivery to a desired organ, such as to the lungs.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “excipient” has its ordinary meaning as understood in light of the specification, and refers to inert substances, compounds, or materials added to a pharmaceutical composition to provide, without limitation, bulk, consistency, stability, binding ability, lubrication, disintegrating ability etc., to the composition. Excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, dextran, fructose, mannose, lactose, galactose, sucrose, sorbitol, cellulose, methyl cellulose, hydroxypropyl methyl cellulose (hypromellose), glycerin, polyvinyl alcohol, povidone, propylene glycol, serum, amino acids, polyethylene glycol, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. The amount of the excipient may be found in a pharmaceutical composition at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), tris(hydroxymethyl)aminomethane (Tris), citric acid, ascorbic acid, acetic acid, salts, phosphates, citrates, acetates, succinates, chlorides, bicarbonates, borates, sulfates, sodium chloride, sodium bicarbonate, sodium phosphate, sodium borate, sodium citrate, potassium chloride, potassium phosphate, magnesium sulfate sugars, dextrose, dextran 40, fructose, mannose, lactose, trehalose, galactose, sucrose, sorbitol, mannitol, cellulose, serum, amino acids, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, polysorbate 20, polysorbate 40, polysorbate, 60, polysorbate 80, poloxamer, poloxamer 188, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, 0-propiolactone, gelatin, cell debris, nucleic acids, peptides, amino acids, or growth medium components or any combination thereof. The amount of the excipient may be found in the formulation at a percentage that is at least 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy.

As used herein, the term “insulin resistance” is understood in the art and refers to the phenomenon where the cells of a subject become less sensitive to insulin, whether it is insulin produced endogenously by the pancreas of the subject, or administered exogenously for treatments. Insulin is necessary to signal glucose uptake by cells to use as a source of energy. Excessive blood sugar levels and/or increased blood levels of insulin (which may also arise due to the pancreas responding to high blood sugar levels) may lead to cells becoming less responsive to normal levels of insulin release. This can notably lead to prediabetes and type 2 diabetes, but insulin resistance has been associated with other diseases and disorders, such as dysmetabolic syndrome, obesity, muscle wasting, hyperinsulinemia, cardiovascular disease, cardiac hypertrophy, myocardial ischemia, hypertension, type A insulin resistance syndrome, type B insulin resistance syndrome, gestational diabetes, acanthosis nigricans, polycystic ovary syndrome, pancreatic cancer associated diabetes (PCDM) and cancer. Furthermore, while insulin resistance is not generally the main cause of type 1 diabetes, type 1 diabetics can also develop insulin resistance.

Also provided herein are methods for “improving insulin sensitivity”, such as in a cell or a patient, which refers to the amelioration of insulin resistance and increasing the relative response of the cell or patient to normal insulin levels. Insulin sensitivity is the ability of insulin to clear excess glucose from the bloodstream by inducing the uptake of glucose by peripheral tissues such as skeletal muscle and adipose tissue. Diabetes is a metabolic disorder which occurs due to the defect in insulin secretion and insulin action (in other terms, insulin sensitivity in insulin responsive tissues which leads to glucose clearance). An improvement in insulin sensitivity may be measured through approaches generally known in the art, such as measuring blood sugar levels or blood insulin levels of a patient, glucose tolerance testing, or hyperinsulinemic euglycemic clamp. For in vitro studies, an improvement in insulin sensitivity can be indirectly measured according to a glucose transporter 1 (GLUT1) or glucose transporter 4 (GLUT4) translocation insulin stimulation index. Information regarding this measurement is known to one of skill in the art, as shown in, for example: Sun Y, Chiu T T, Foley K P, Bilan P J, Klip A. (2014) “Myosin Va mediates Rab8A-regulated GLUT4 vesicle exocytosis in insulin-stimulated muscle cells”. Mol Biol Cell. 25: 1159-70 and Tunduguru R, Zhang J, Aslamy A, Salunkhe V A, Brozinick J T, Elmendorf J S, Thurmond D C. “The actin-related p41ARC subunit contributes to p21-activated kinase-1 (PAK1)-mediated glucose uptake into skeletal muscle cells”. J Biol Chem. 2017 September 25. pii: jbc.M117.801340, each of which is hereby expressly incorporated by reference in its entirety. Improvement in insulin sensitivity, for example with an anti-Gal3 antibody or binding fragment thereof, can be compared to other treatment regimens, such as dietary changes, weight loss, exercise, metformin, and thiazolidinediones, indicated for insulin resistant patients.

As used herein, the term “glucose transporter 1 (GLUT1)” refers to a major insulin-independent glucose transporter that is highly conserved among mammals and is found prevalently in different tissues. Dysfunction in GLUT1 is associated with GLUT1 deficiency syndromes (De Vivo disease), idiopathic generalized epilepsy 12, dystonia 9, and stomatin-deficient cryohydrocytosis. An exemplary sequence for human GLUT1 is provided as SEQ ID NO: 1618. GLUT1 is also known as SLC2A1.

As used herein, the term “glucose transporter 4 (GLUT4)” refers to the insulin-responsive glucose transporter expressed by cells. An exemplary sequence for human GLUT4 is provided as SEQ ID NO: 950. GLUT4 is a transmembrane protein that facilitates exchange of glucose between cells and the blood stream. GLUT4 stored in intracellular vesicles are translocated to the plasma membrane following a signaling cascade induced by insulin. For insulin resistant cells, this translocation process is inhibited, resulting in less GLUT4 being transported to the plasma membrane and abnormal glucose intake. GLUT4 is also known as SLC2A4.

Also provided herein are methods directed to “enhancing GLUT translocation”. This term refers to the inducement of improved GLUT transport and activity within a cell, such as an insulin resistant cell, which may have originally exhibited reduced GLUT translocation compared to normal cells. In some embodiments, the GLUT is GLUT 1 and/or GLUT4. Enhancing GLUT translocation may also apply to the reversal of dysfunctional GLUT translocation caused by a deleterious cellular function. In some embodiments, as shown herein, GLUT translocation is inhibited by Gal3. Application of a Gal3 inhibitor, such as an anti-Gal3 antibody, may improve GLUT translocation, thereby reversing the activity of Gal3. In some embodiments, the enhancement may be by at least 5%, 10%, 20%, 30%, 40%, 50%, 60%, 70%, 80%, 90%, or 100%, or any percentage that is within a range defined by any two of the aforementioned values.

As used herein, the term “inhibit” refers to the reduction or decrease in an expected activity, such as a cellular activity. The reduction or decrease may be by at least 5%, 10%, 20%, 30%, 40%, 50%, 60%, 70%, 80%, 90%, or 100%, or any percentage that is within a range defined by any two of the aforementioned values, where a reduction or decrease of 100% indicates a complete inhibition and any lower percentage indicates a partial inhibition. The reduction or decrease of the expected activity may be observed in a direct or indirect way. For example, as disclosed herein, it is demonstrated that Gal3 inhibits GLUT (e.g., GLUT1 and/or GLUT4) translocation within the cell. This inhibition of GLUT translocation (as well as a potential reversal of said inhibition, e.g. with one or more of the proteins disclosed herein) can be observed through the direct observation of GLUT movement or abundance within the cell, or observing indirect properties such as glucose intake.

The term “half maximal inhibitory concentration” often abbreviated as “IC50” refers to the concentration of a substance (e.g., a compound or antibody) that results in 50% inhibition of a biological process, or a component of the process (e.g. protein binding).

The term “% w/w” or “% wt/wt” means a percentage expressed in terms of the weight of the ingredient or agent over the total weight of the composition multiplied by 100.

It is understood that an antibody with an antibody name described herein can be referred using a shortened version of the antibody name, as long as there are no conflicts with another antibody described herein. For example, F846C.1B2 can also be referred to as 846C.1B2, or 846.1B2. This can also refer to fragments of the antibody (e.g., with the same 1, 3, or 6 CDRs).

### Exemplary Anti-Gal3 Antibodies and Binding Fragments Thereof

Disclosed herein and as applicable to any of the methods or uses disclosed herein are anti-Gal3 antibodies or binding fragments thereof. In some embodiments, the anti-Gal3 antibody or binding fragment thereof binds to the N-terminal domain of Gal3, N-terminus of Gal3, or the tandem repeat domain (TRD) of Gal3. In some embodiments, the anti-Gal3 antibody or binding fragment thereof does not bind to the N-terminus of Gal3, the N-terminal domain of Gal3, or the TRD of Gal3. In some embodiments, the anti-Gal3 antibody or binding fragment thereof binds to the C-terminus of Gal3, the C-terminal domain of Gal3, or the CRD of Gal3. In some embodiments, the anti-Gal3 antibody or binding fragment thereof does not bind to the C-terminus of Gal3, the C-terminal domain of Gal3, or the CRD of Gal3. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the constructs provided herein are provided in a subcutaneous formulation or an intravenous formulation. In some embodiments, the constructs provided herein are provided in a subcutaneous formulation.

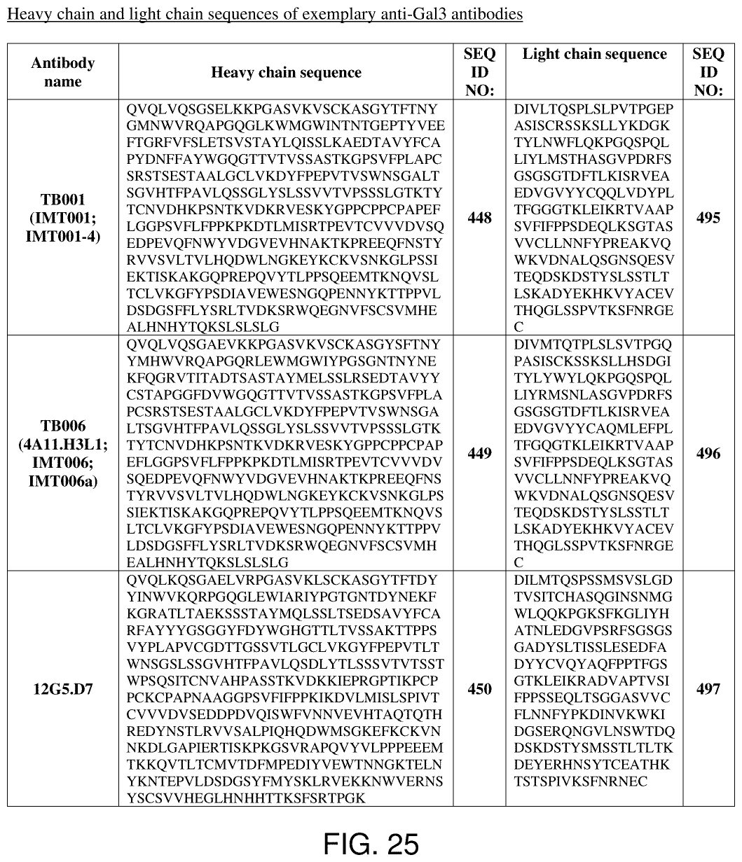

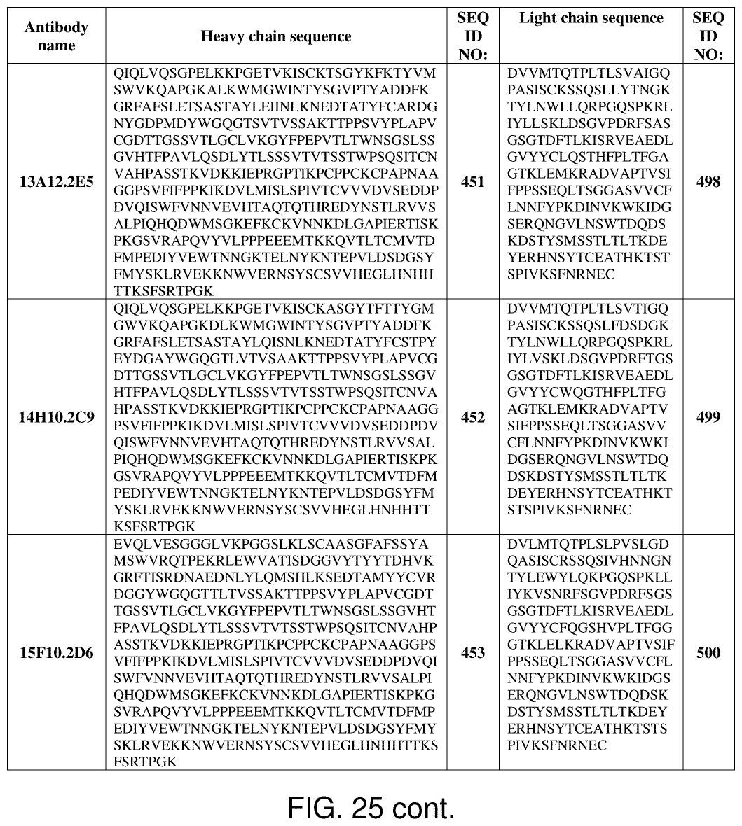

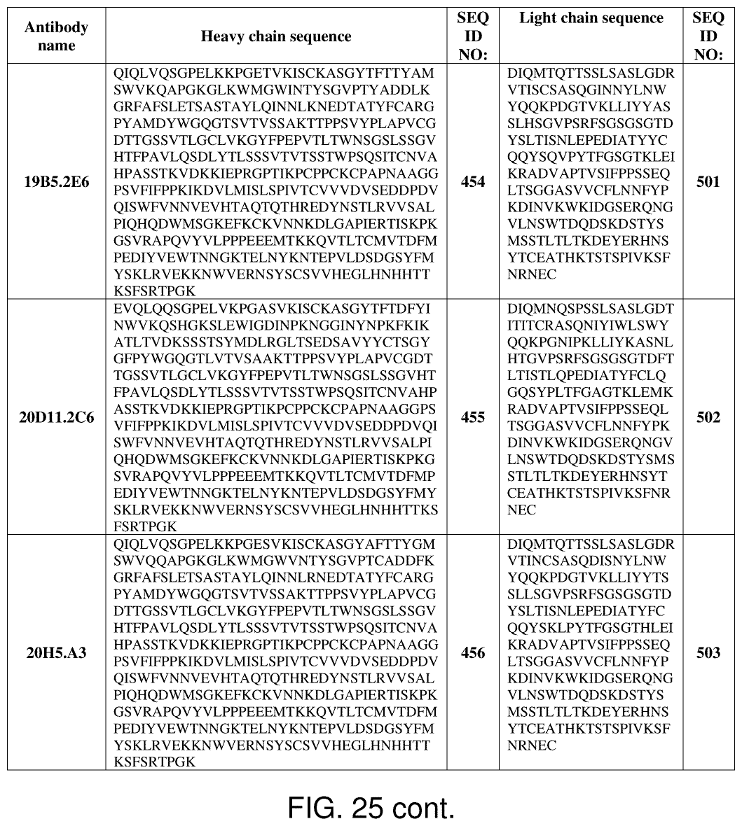

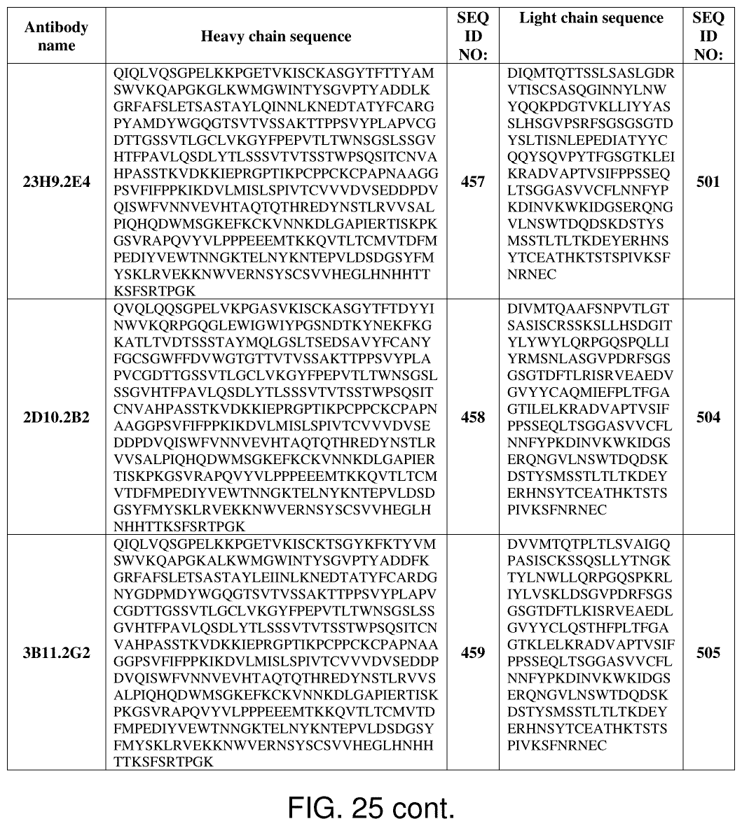

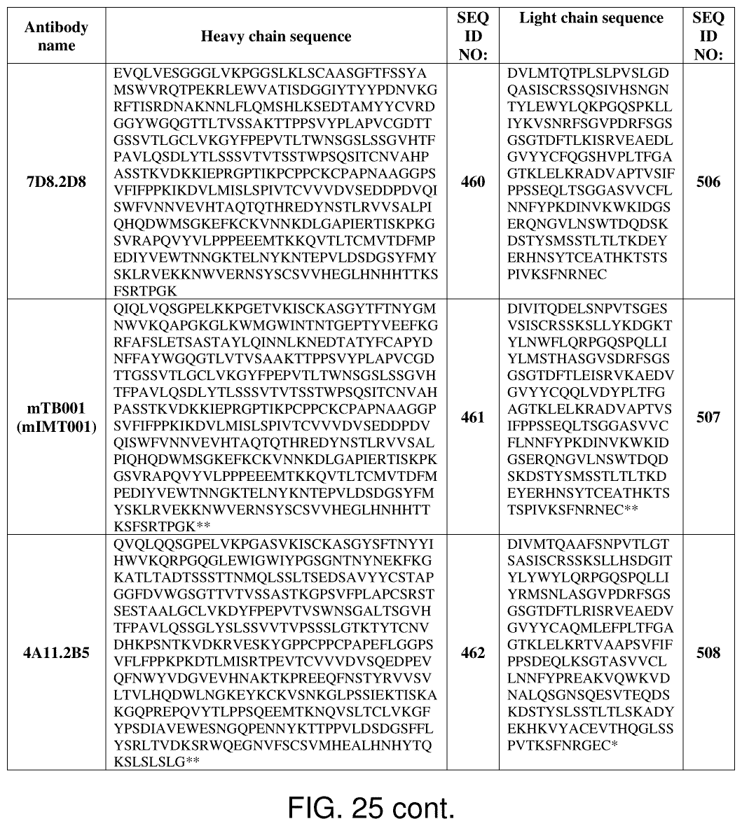

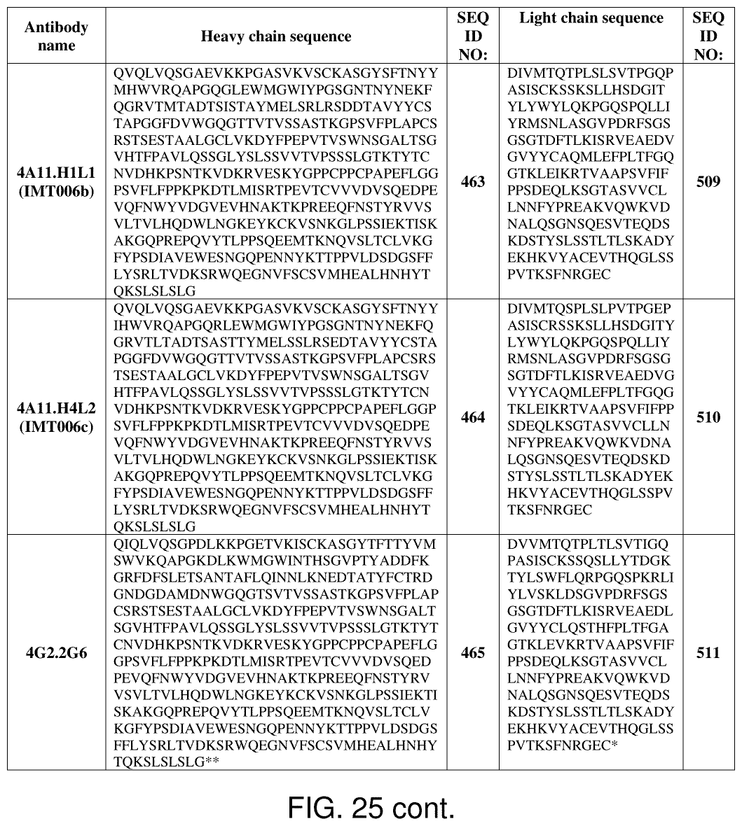

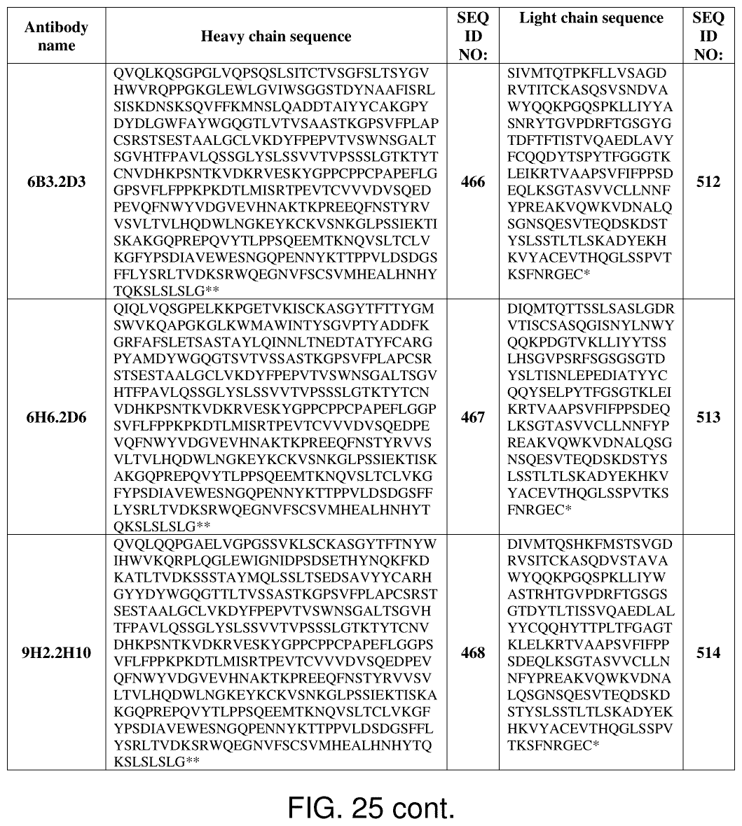

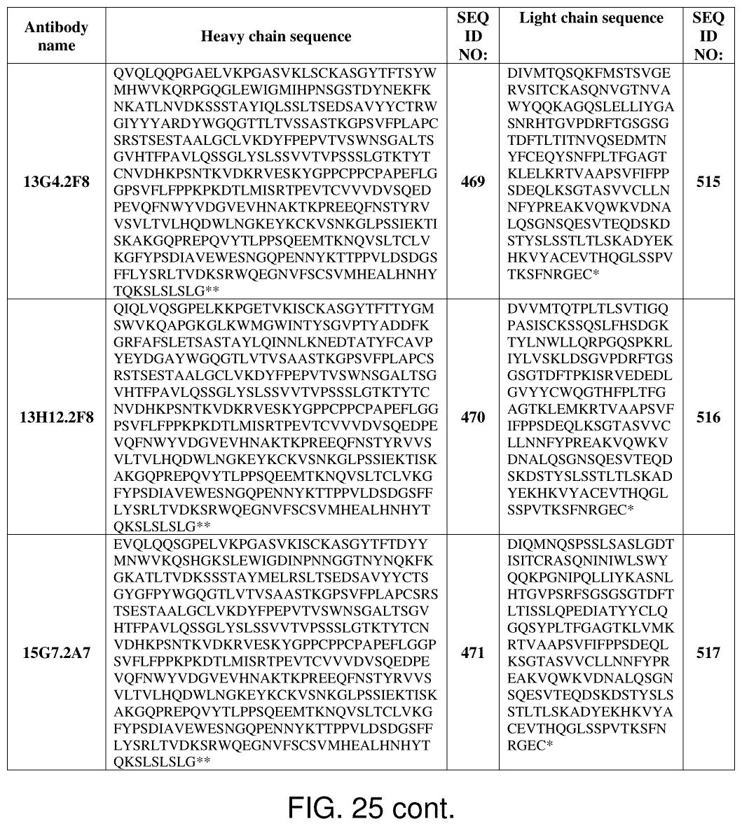

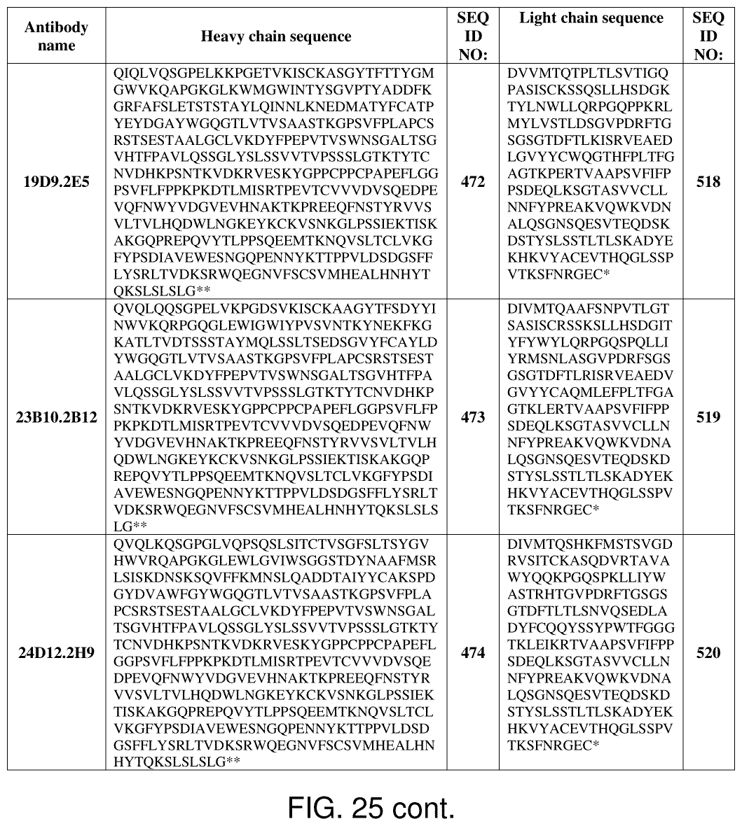

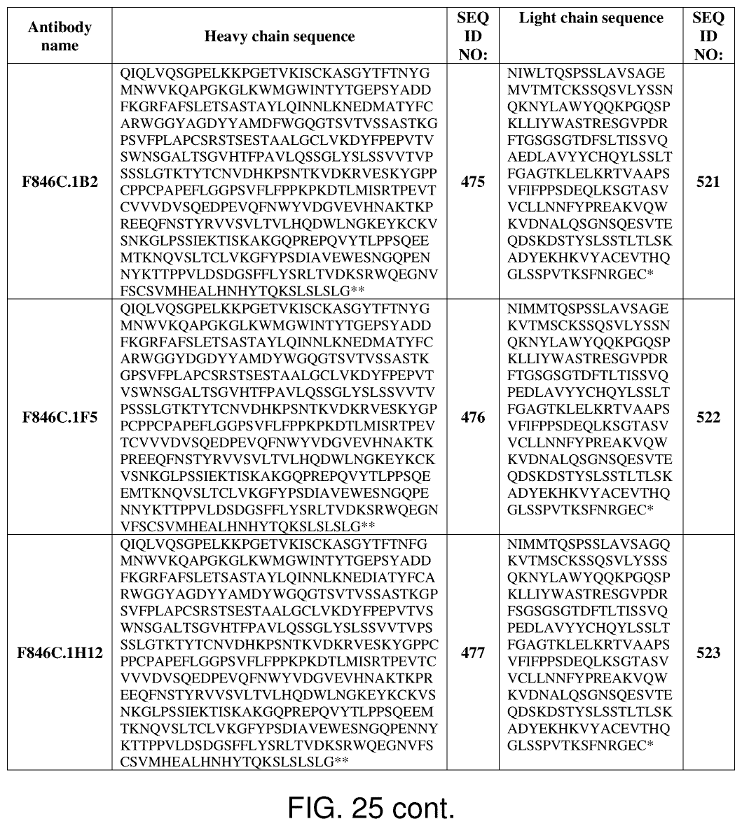

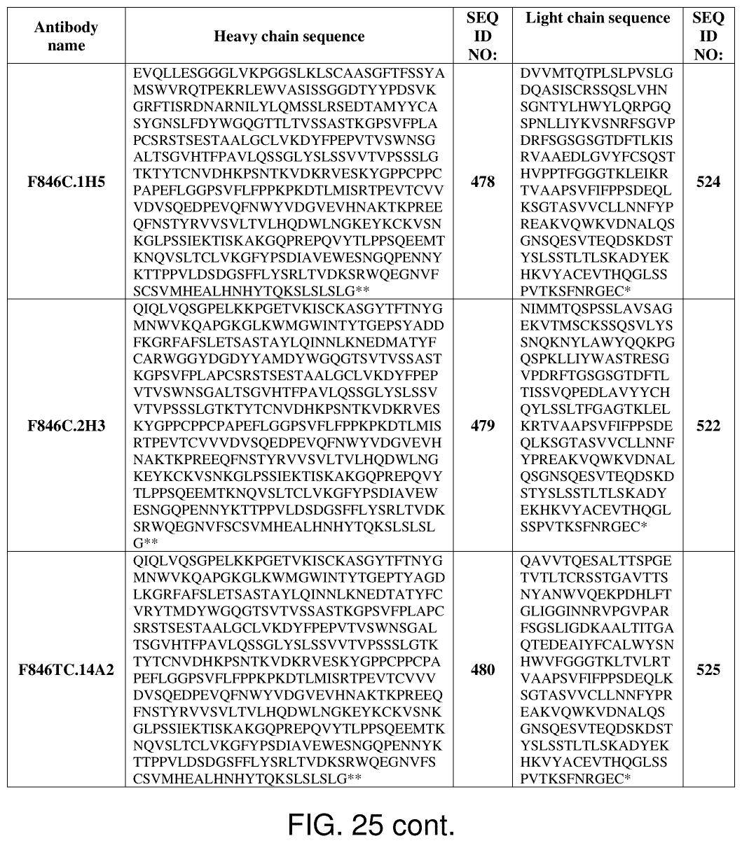

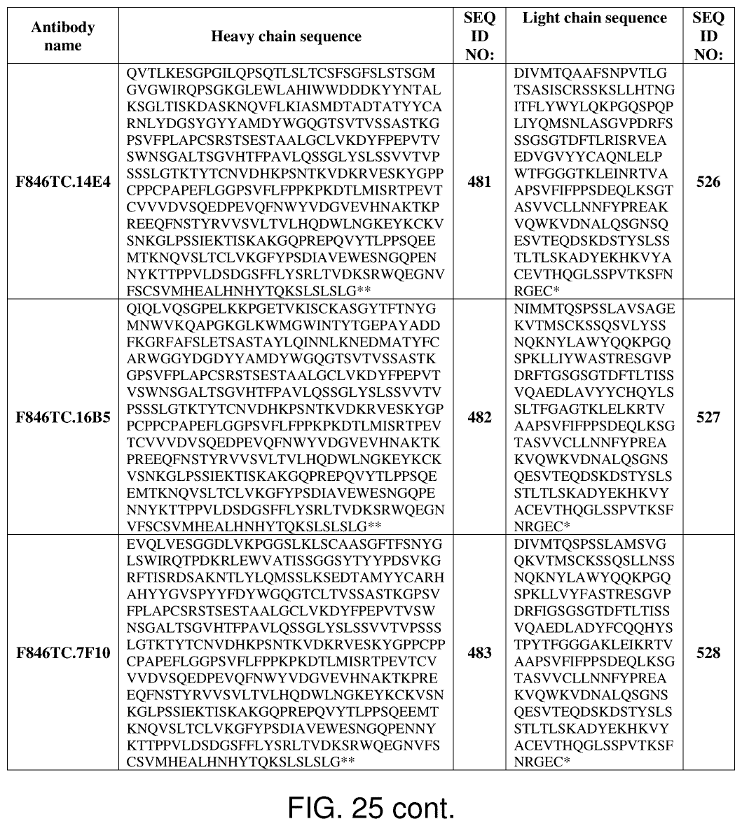

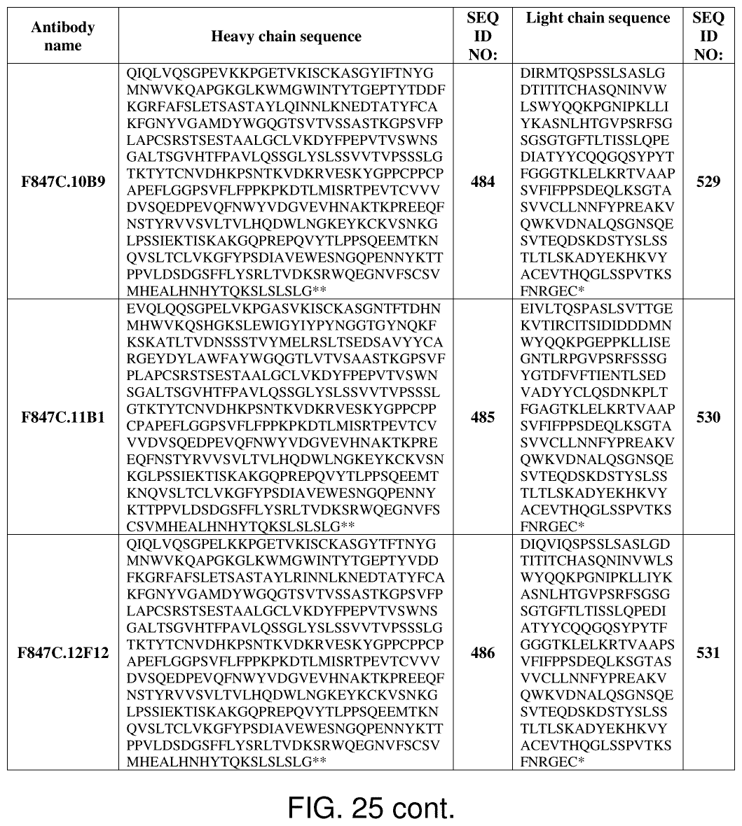

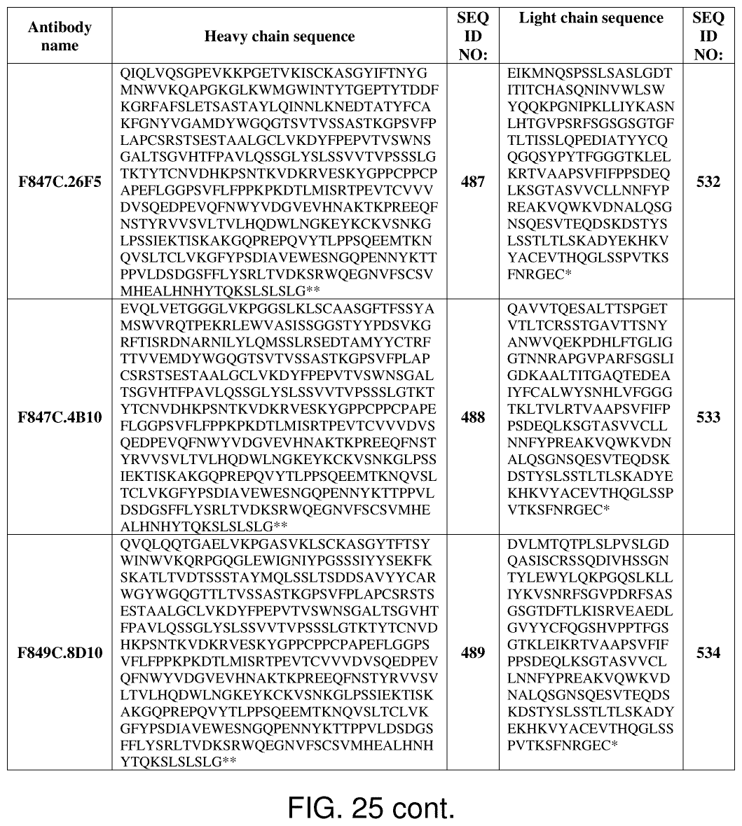

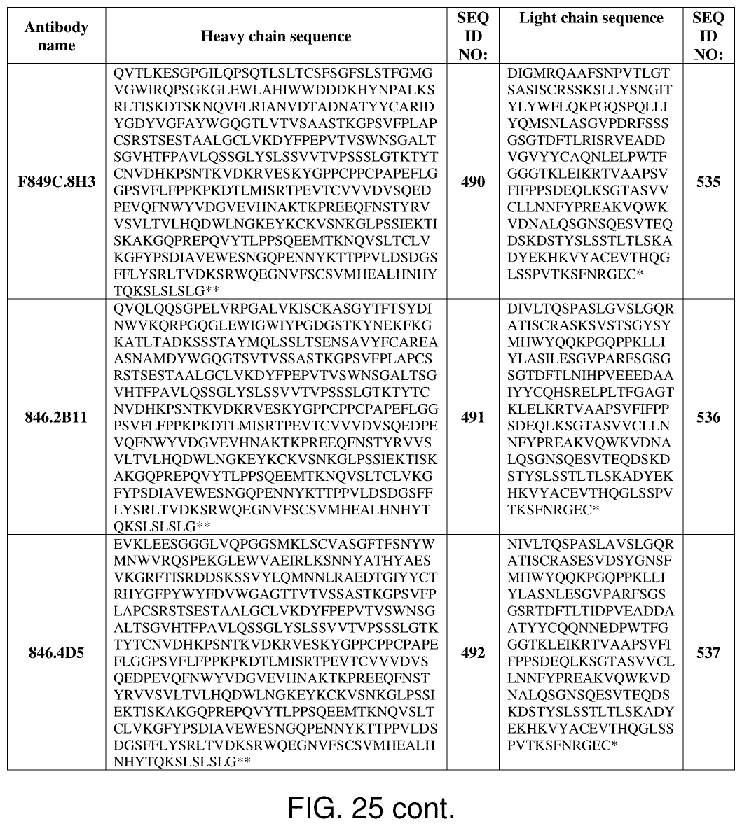

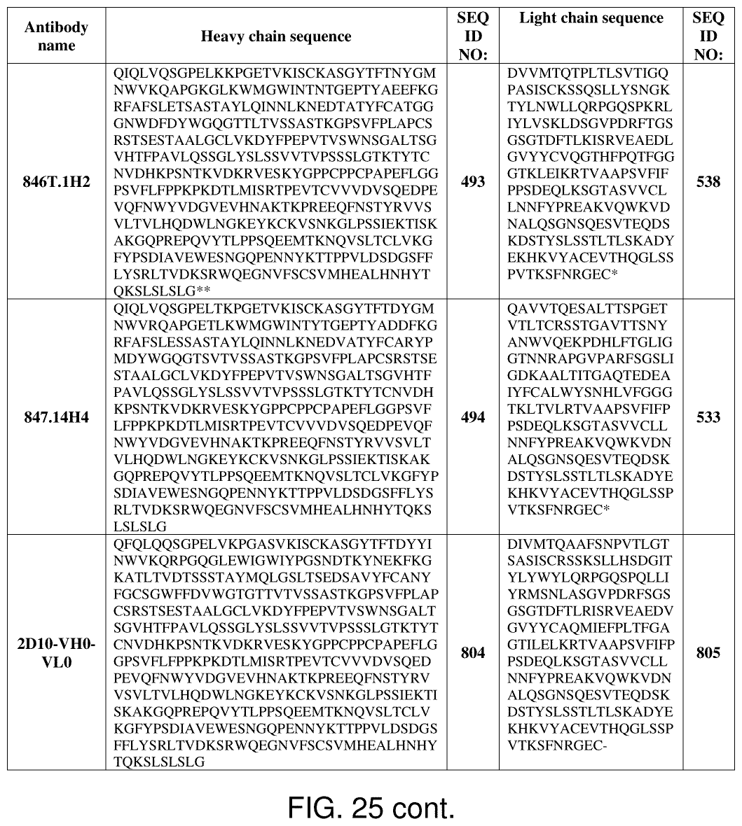

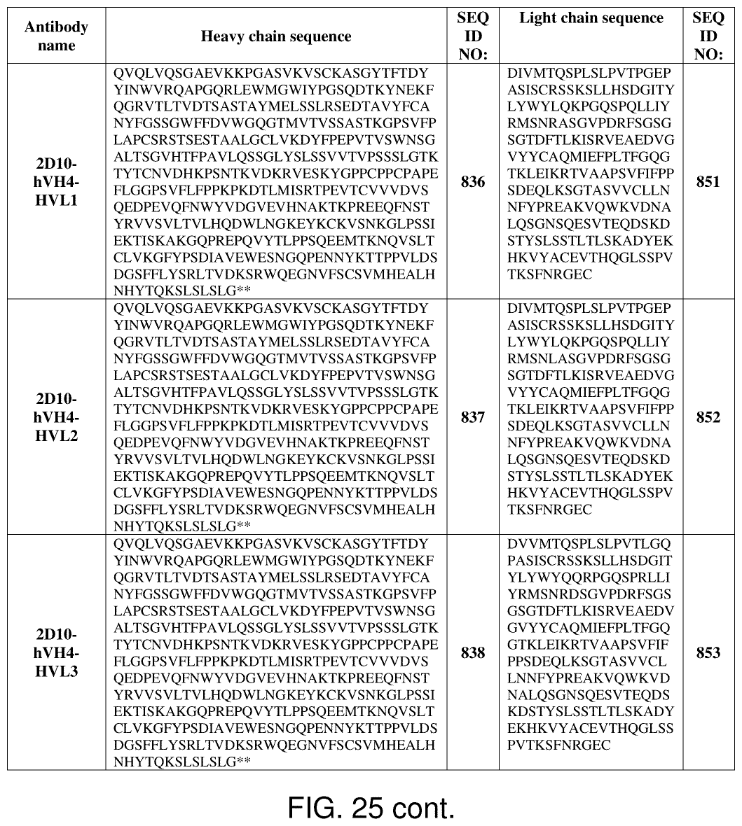

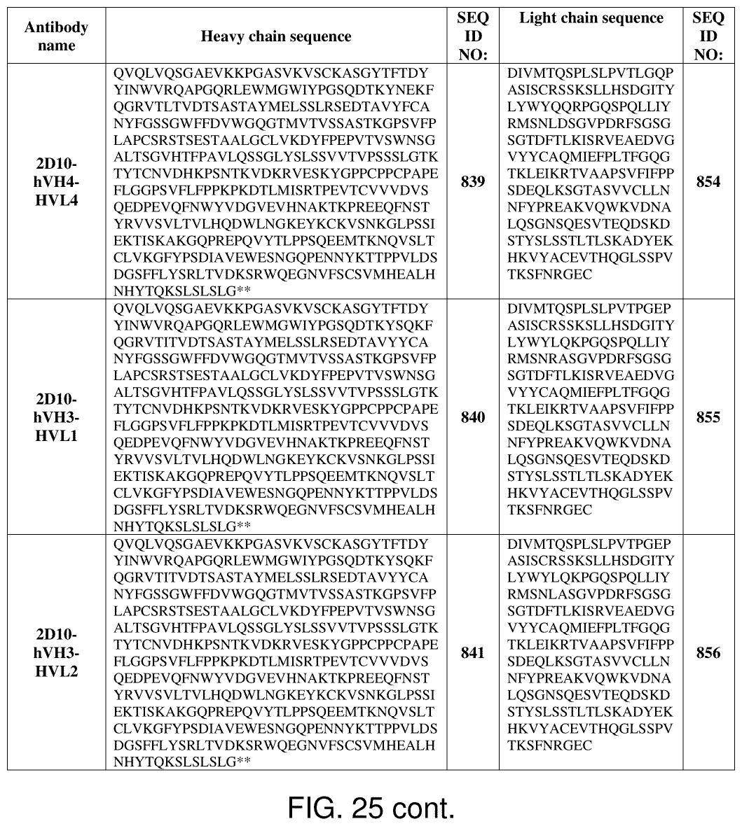

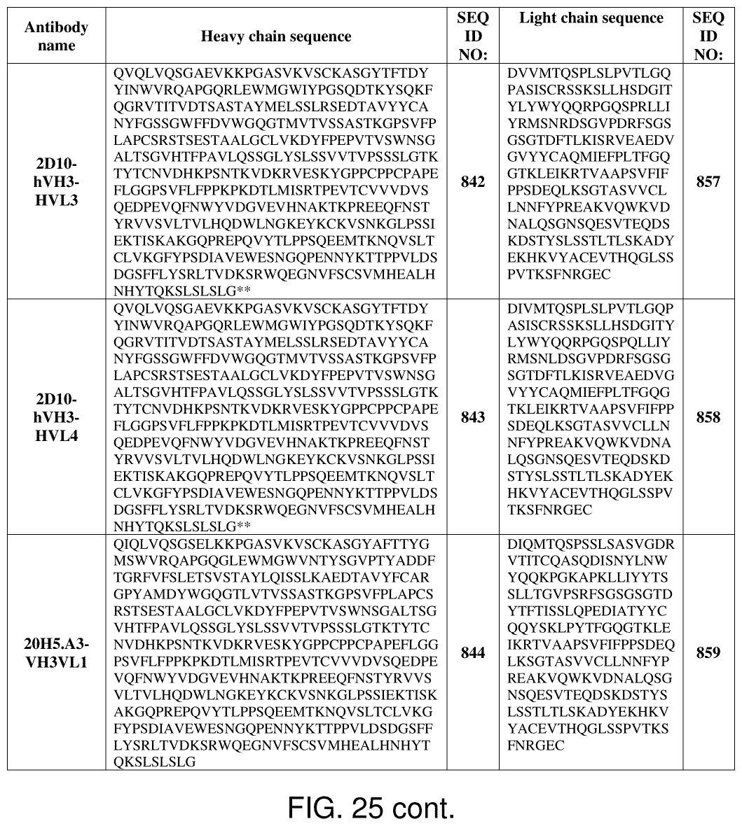

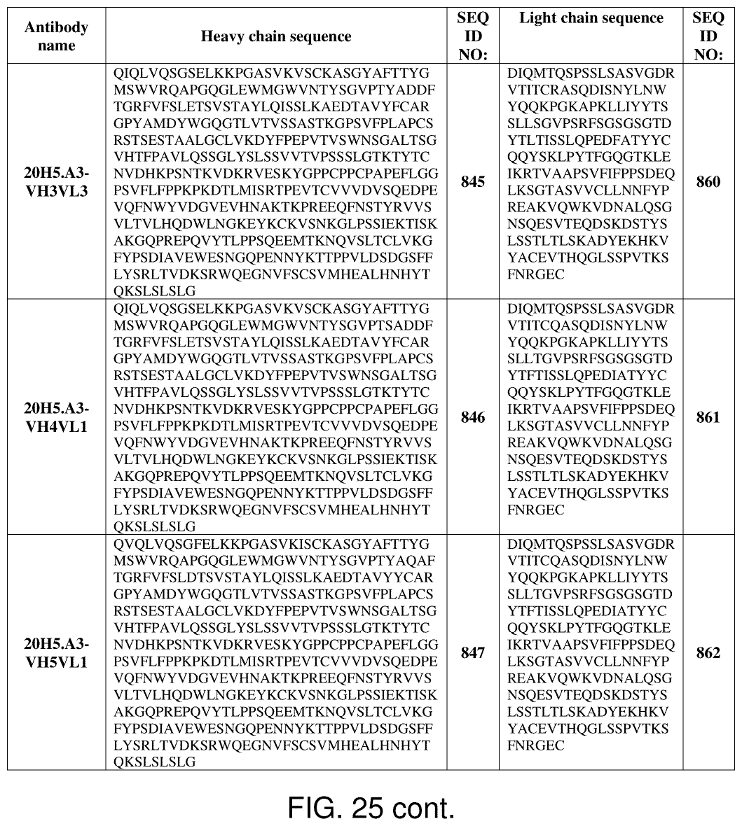

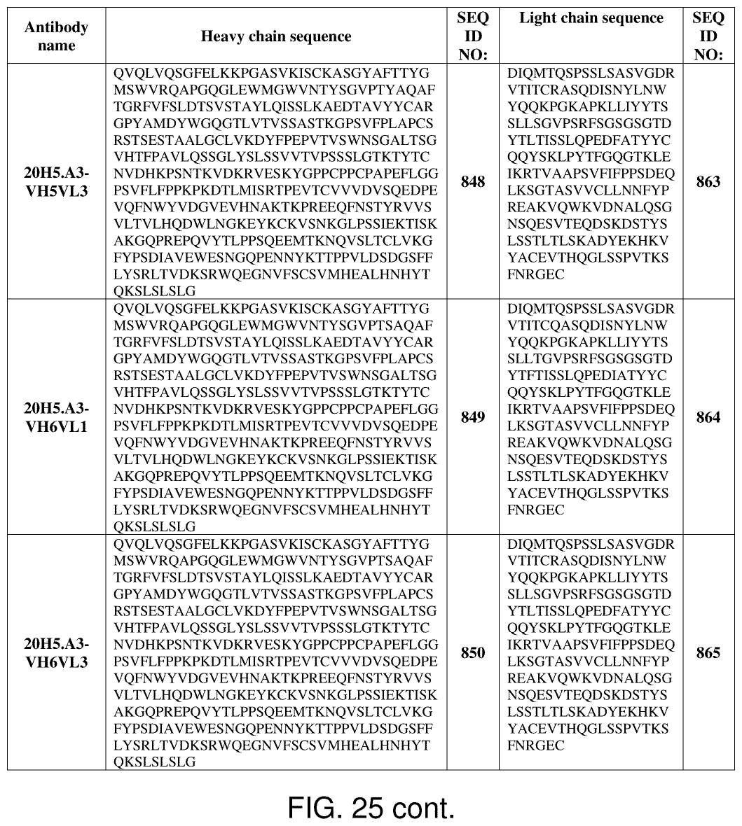

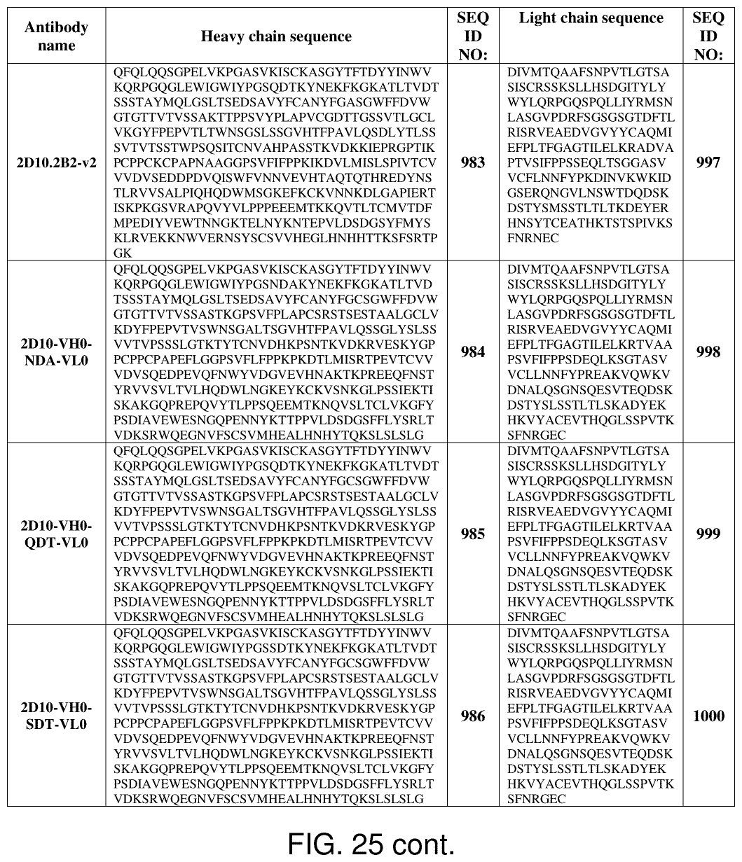

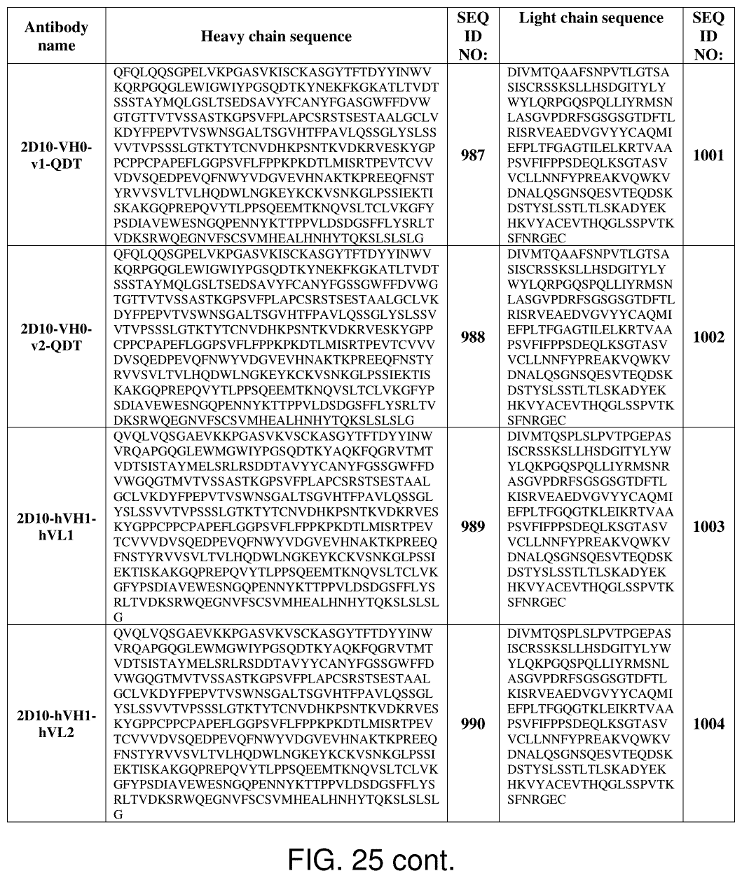

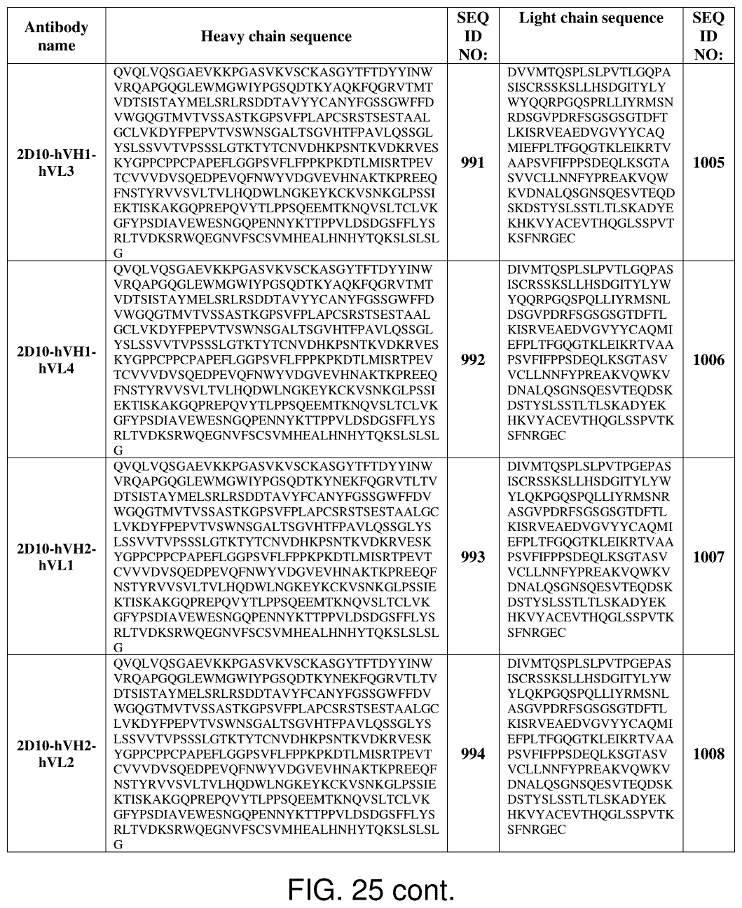

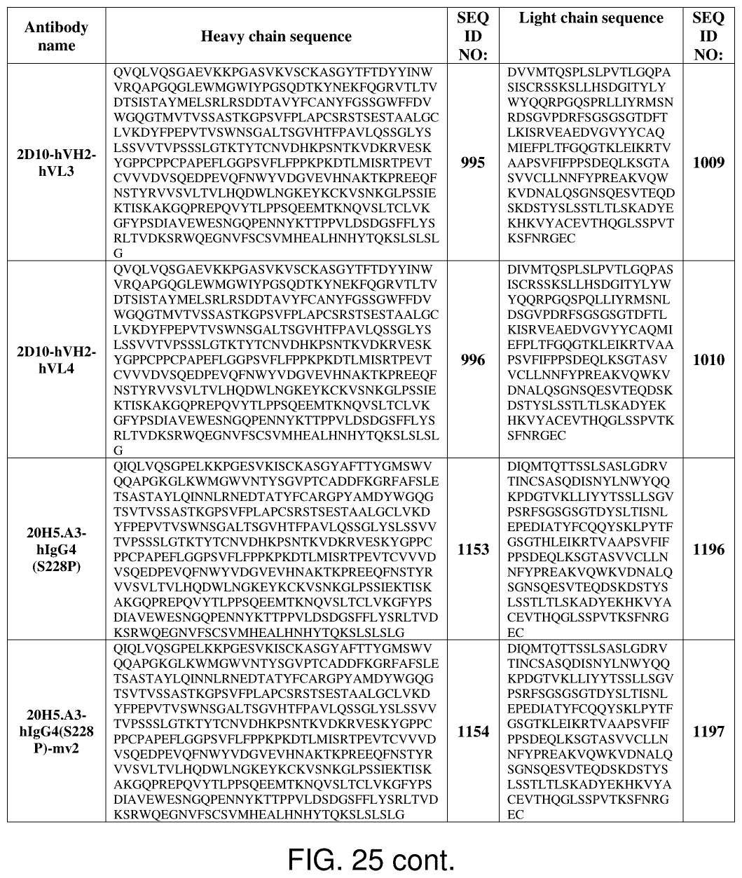

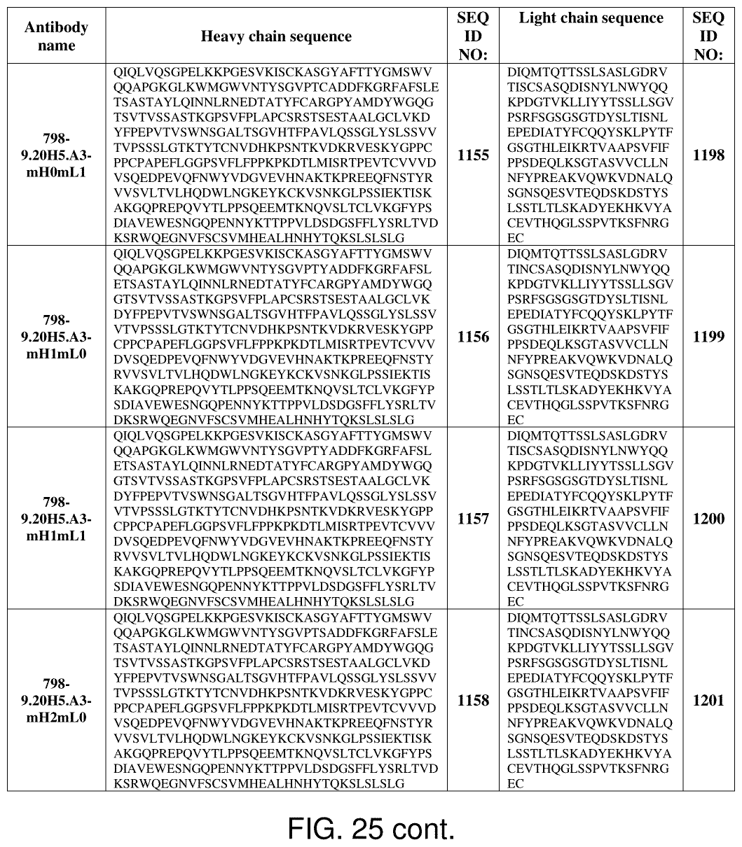

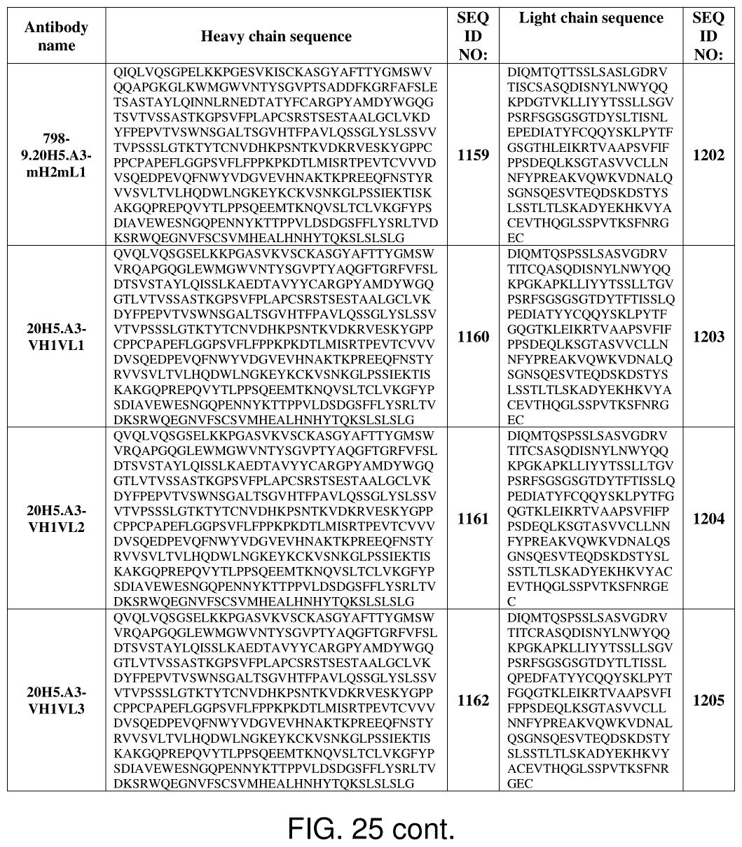

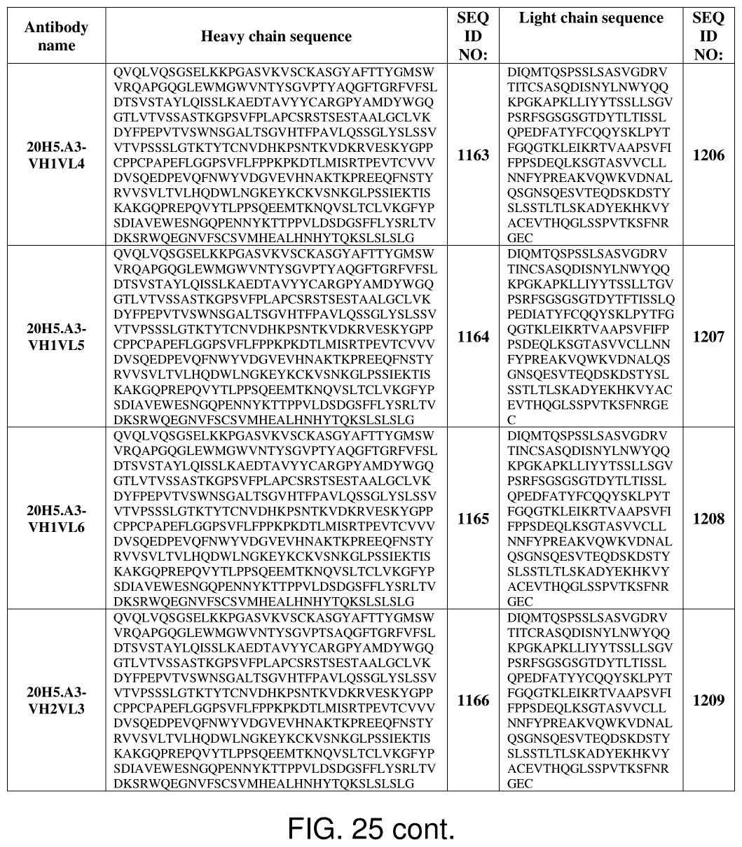

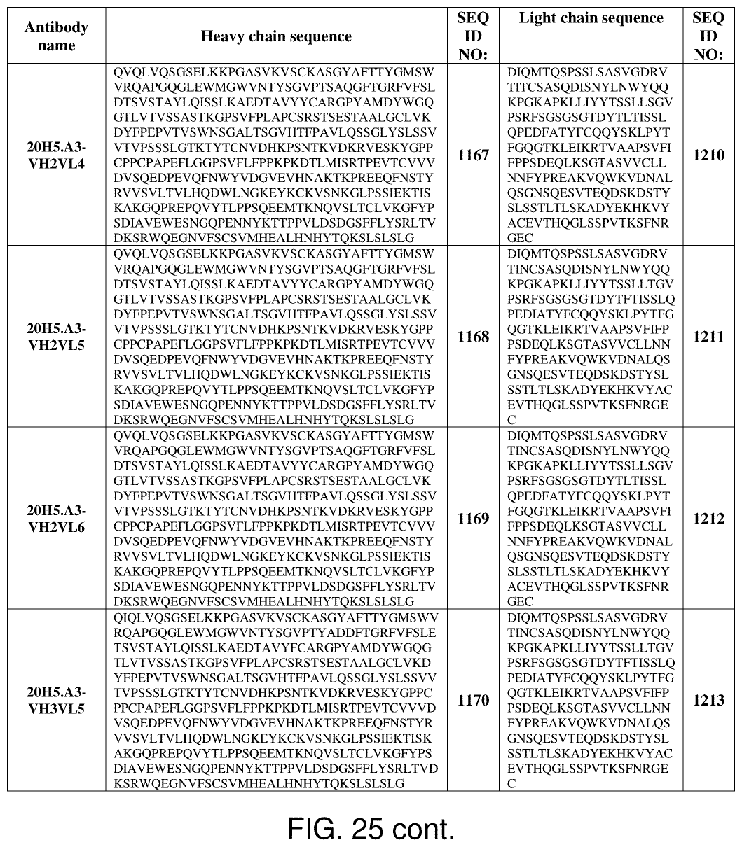

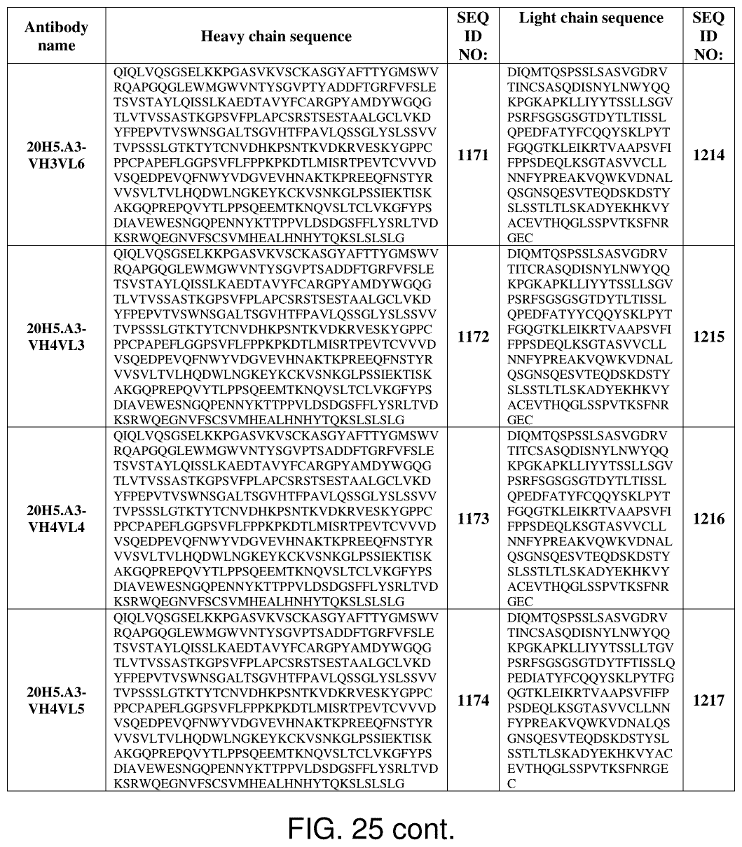

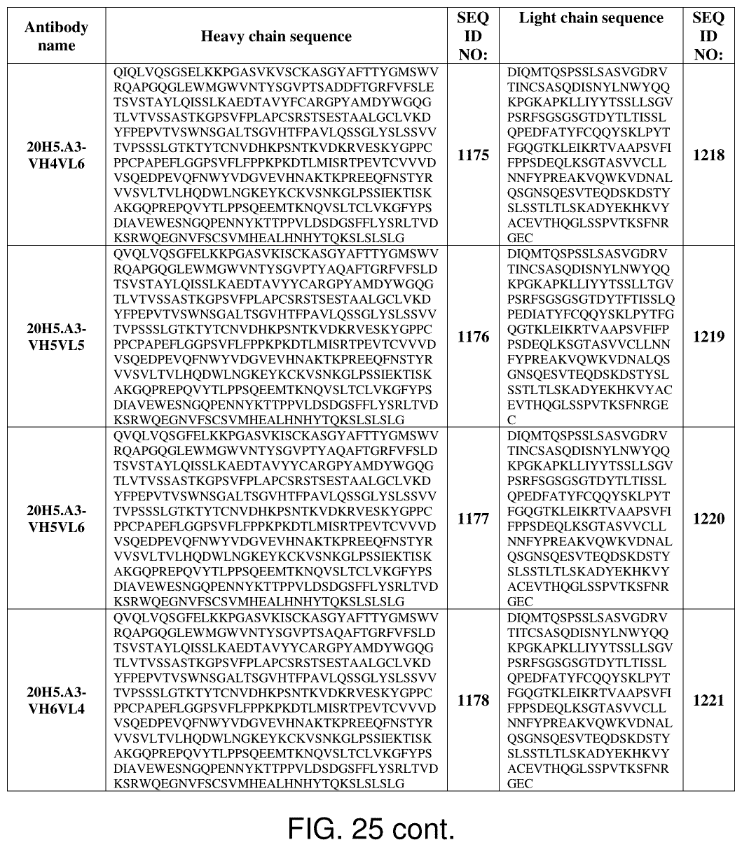

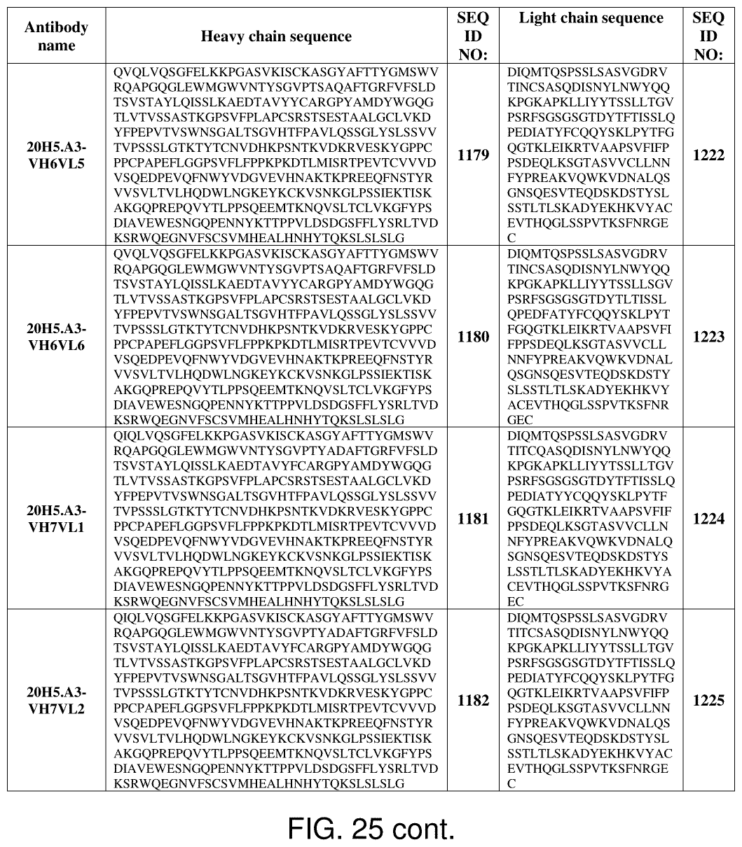

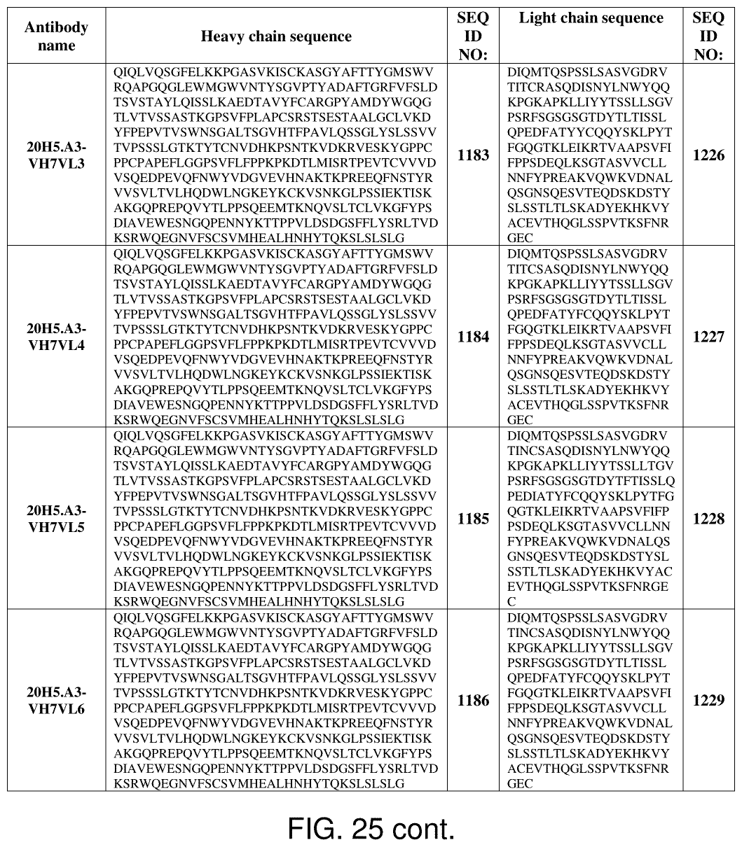

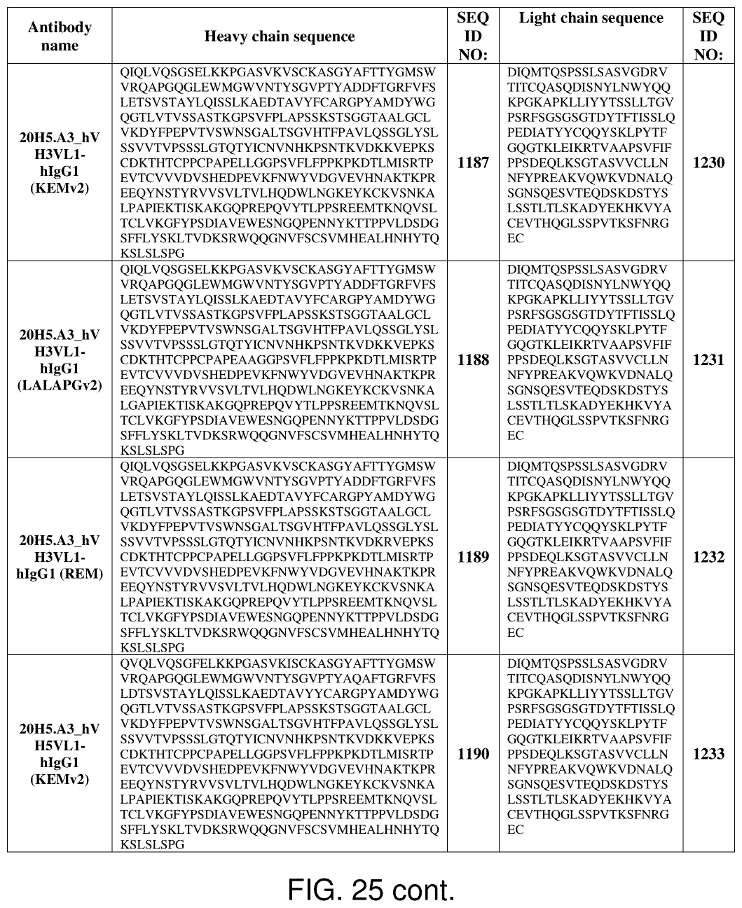

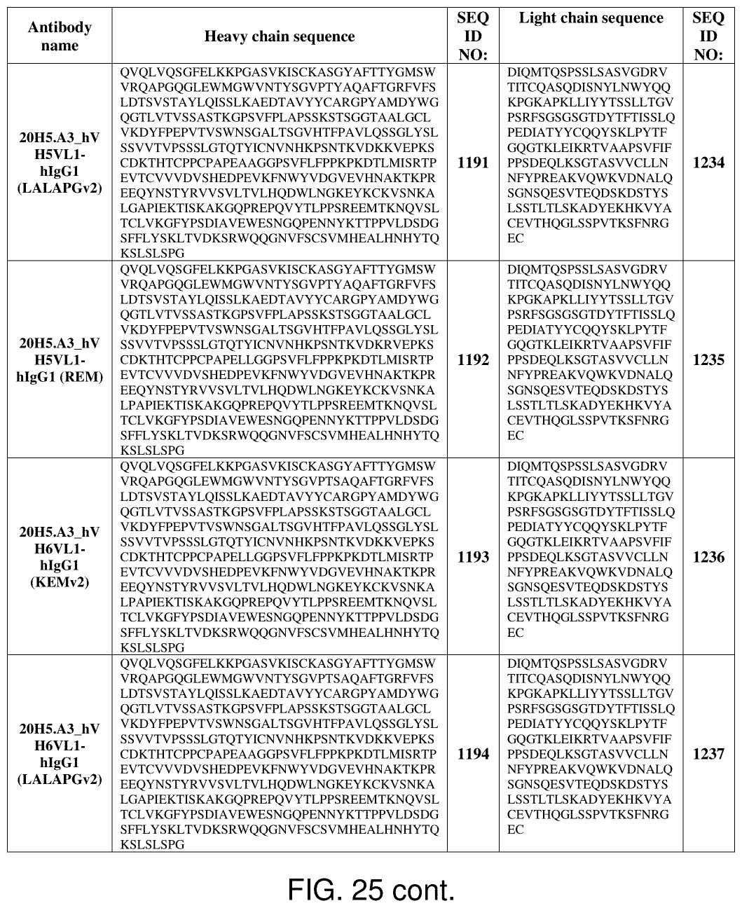

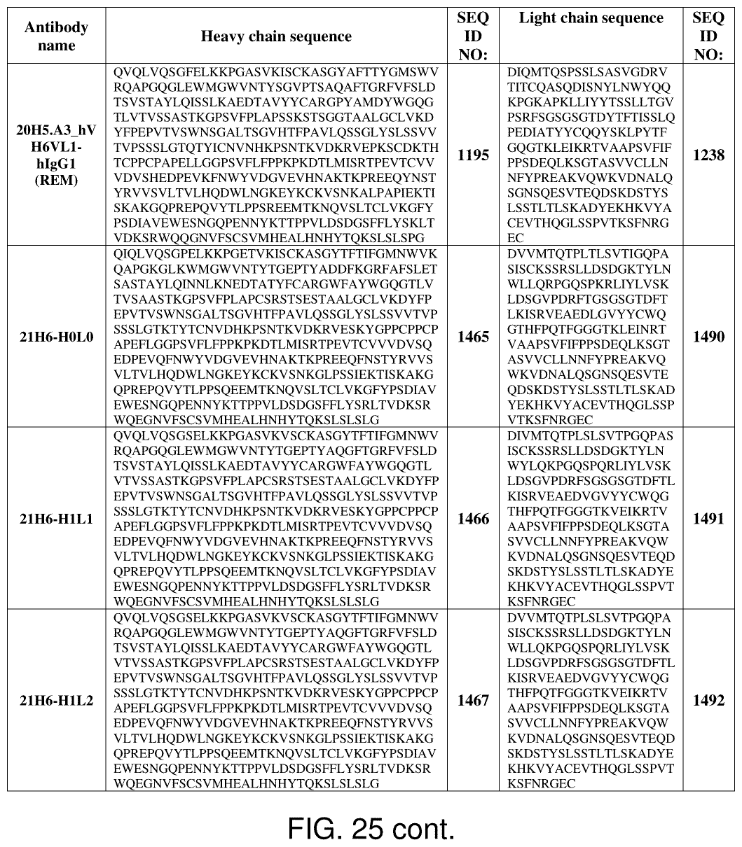

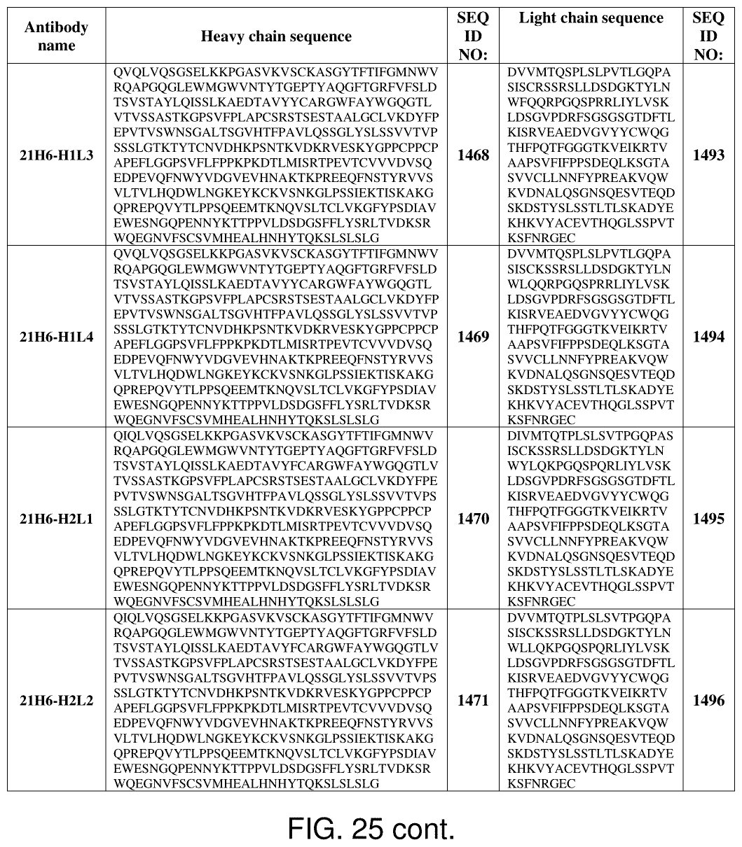

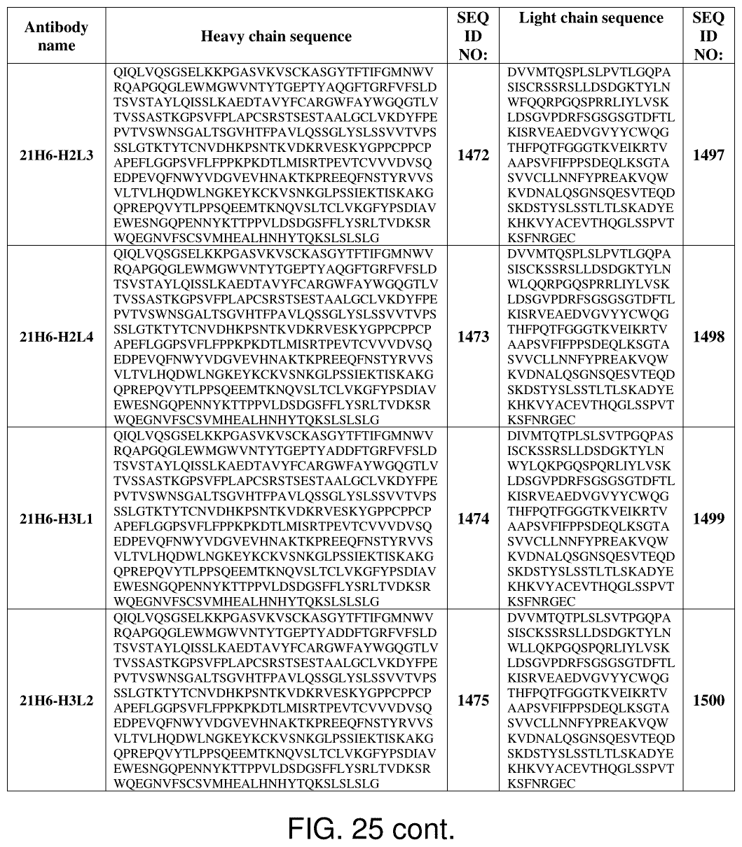

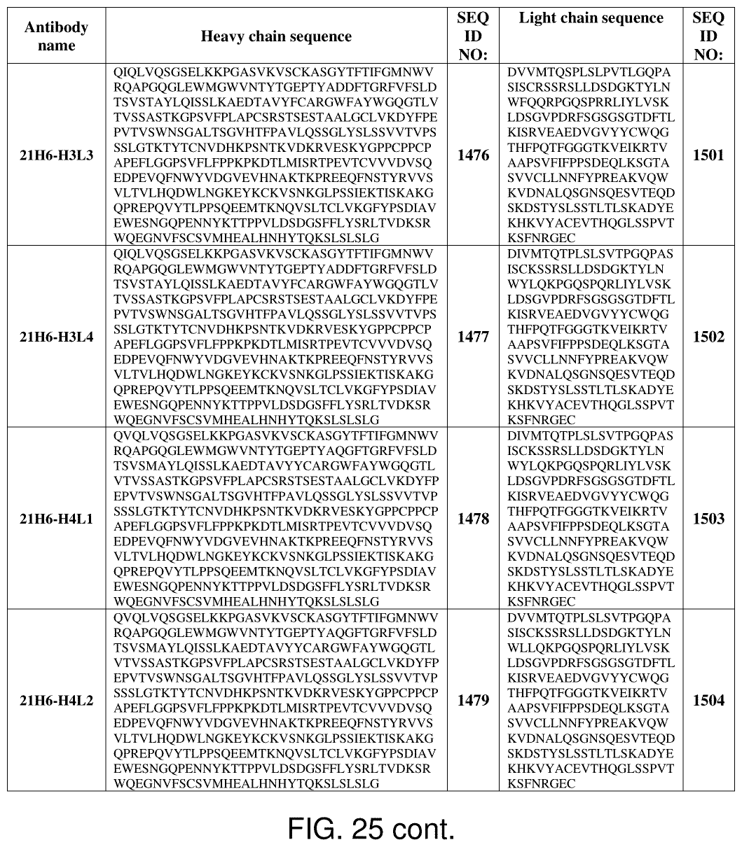

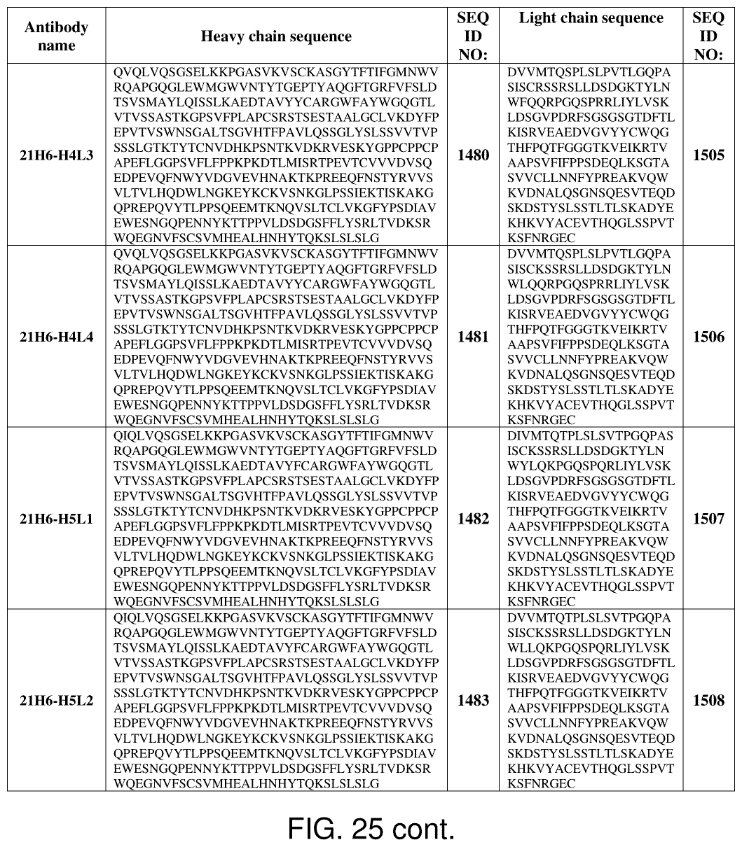

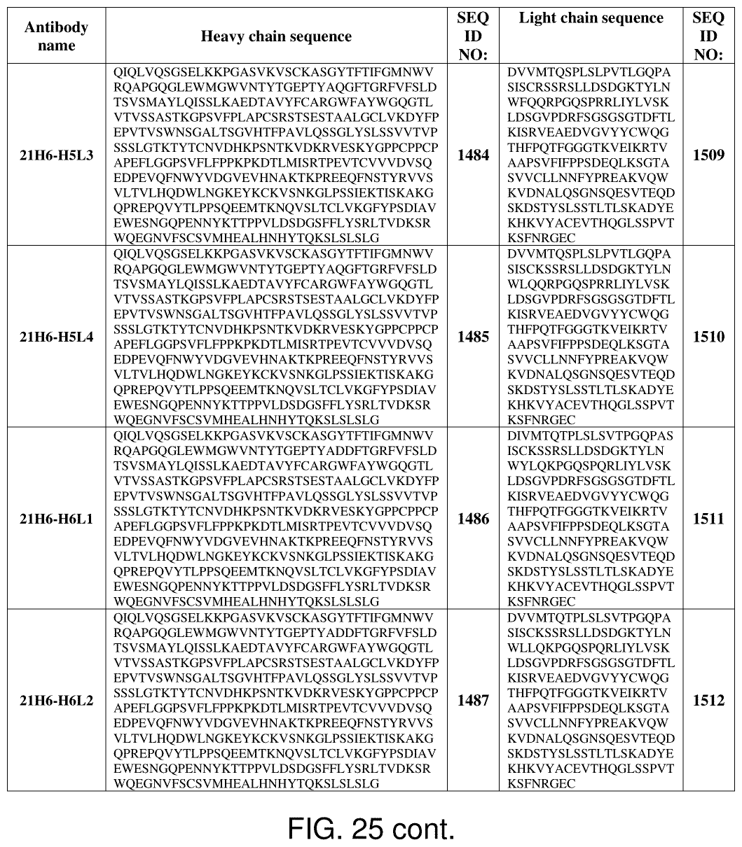

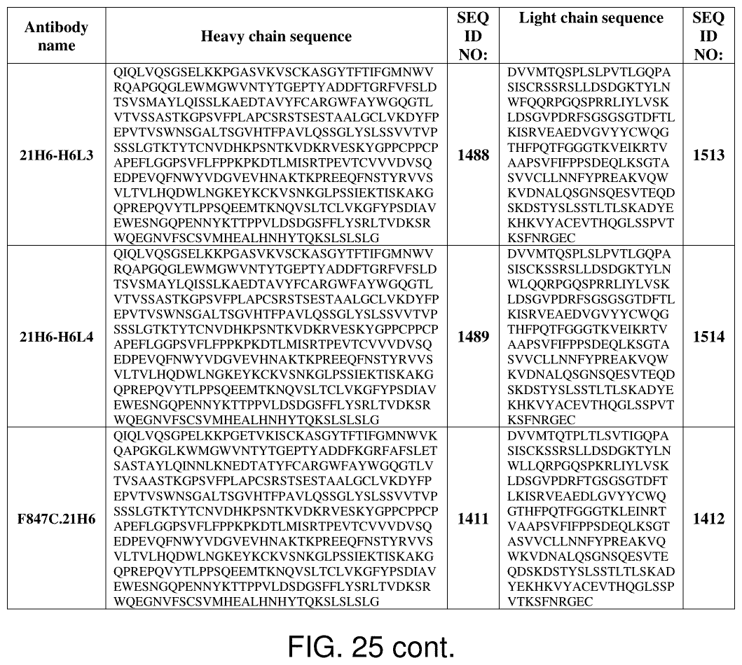

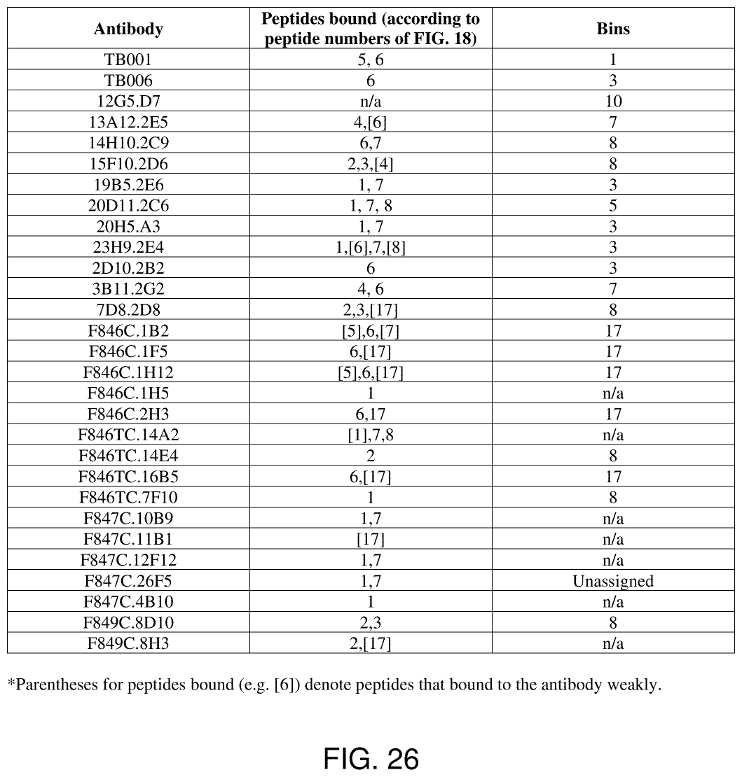

In some embodiments, antibodies or binding fragments thereof are provided. In some embodiments, the antibodies are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 27-70. In some embodiments, the V_{H}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 71-111, 801, 951, 952. In some embodiments, the V_{H}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 112-169, 802, 953, 954. In some embodiments, the V_{L}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 170-220. In some embodiments, the V_{L}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 221-247. In some embodiments, the V_{L}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 248-296. In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a VL sequence, a VH sequence, a VL/VH pairing, and/or V_{H}-CDR1, V_{H}-CDR2, V_{H}-CDR3, V_{L}-CDR1, V_{L}-CDR2, V_{L}-CDR3 (including 1, 2, 3, 4, or 5 amino acid substitutions of any one or more of these CDRs) set from the heavy chain and light chain sequences as depicted in FIG. 25.

In some embodiments, antibodies or binding fragments thereof are provided. In some embodiments, the antibodies are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 27-70. In some embodiments, the V_{H}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 71-111, 801, 951, 952. In some embodiments, the V_{H}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 112-169, 802, 953, 954. In some embodiments, the V_{L}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 170-220. In some embodiments, the V_{L}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 221-247. In some embodiments, the V_{L}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 248-296. In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to a VL sequence, a VH sequence, a VL/VH pairing, and/or V_{H}-CDR1, V_{H}-CDR2, V_{H}-CDR3, V_{L}-CDR1, V_{L}-CDR2, V_{L}-CDR3 (including 1, 2, 3, 4, or 5 amino acid substitutions of any one or more of these CDRs) set from the heavy chain and light chain sequences as depicted in FIG. 25.

In some embodiments, antibodies or binding fragments thereof are provided. In some embodiments, the antibodies or binding fragments thereof are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a VL-CDR3. In some embodiments, the V_{H}-CDR1 comprises an amino acid sequence having at least 0, 1, 2, 3, 4, 5, or 6 substitutions relative to any amino acid sequence according to SEQ ID NOs: 27-70. In some embodiments, the V_{H}-CDR2 comprises an amino acid sequence having at least 0, 1, 2, 3, 4, 5, or 6 substitutions relative to any amino acid sequence according to SEQ ID NOs: 71-111, 801, 951, 952. In some embodiments, the V_{H}-CDR3 comprises an amino acid sequence having at least 0, 1, 2, 3, 4, 5, or 6 substitutions relative to any amino acid sequence according to SEQ ID NOs: 112-169, 802, 953, 954. In some embodiments, the V_{L}-CDR1 comprises an amino acid sequence having at least 0, 1, 2, 3, 4, 5, or 6 substitutions relative to any amino acid sequence according to SEQ ID NOs: 170-220. In some embodiments, the V_{L}-CDR2 comprises an amino acid sequence having at least 0, 1, 2, 3, 4, 5, or 6 substitutions relative to any amino acid sequence according to SEQ ID NOs: 221-247. In some embodiments, the V_{L}-CDR3 comprises an amino acid sequence having at least 0, 1, 2, 3, 4, 5, or 6 substitutions relative to any amino acid sequence according to SEQ ID NOs: 248-296.

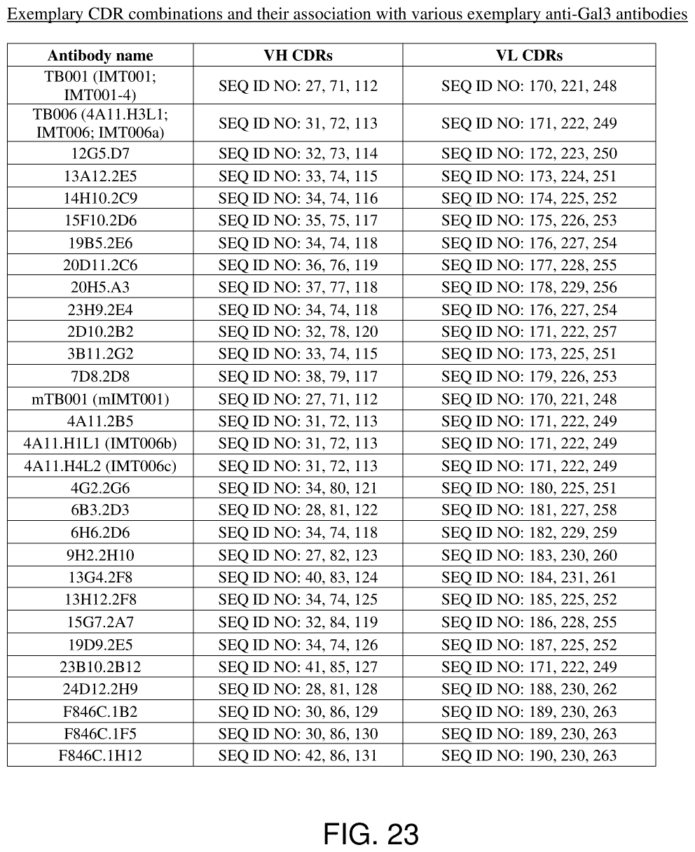

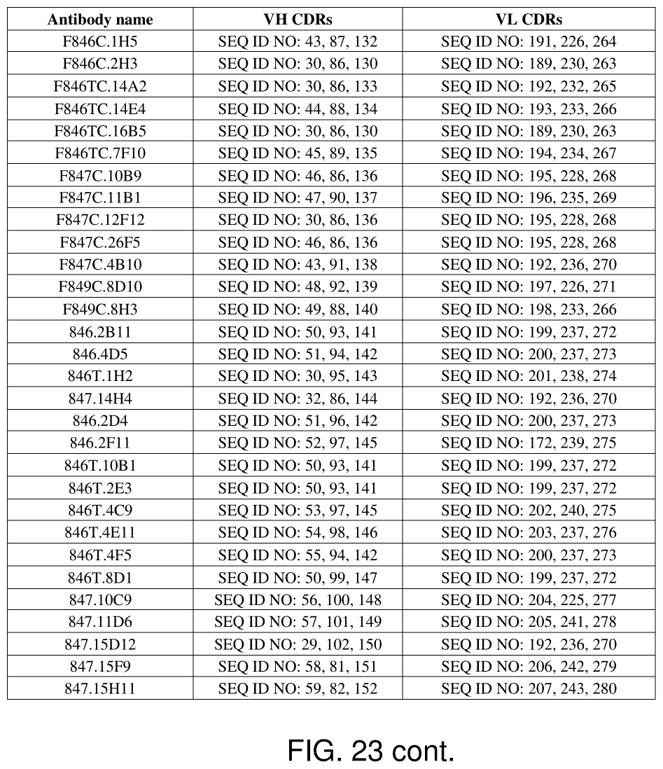

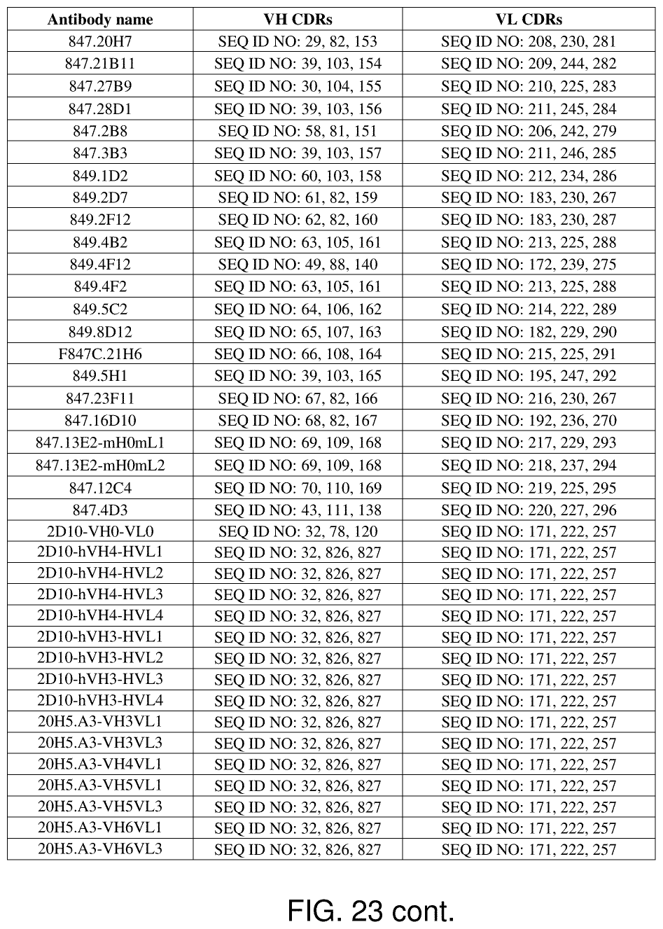

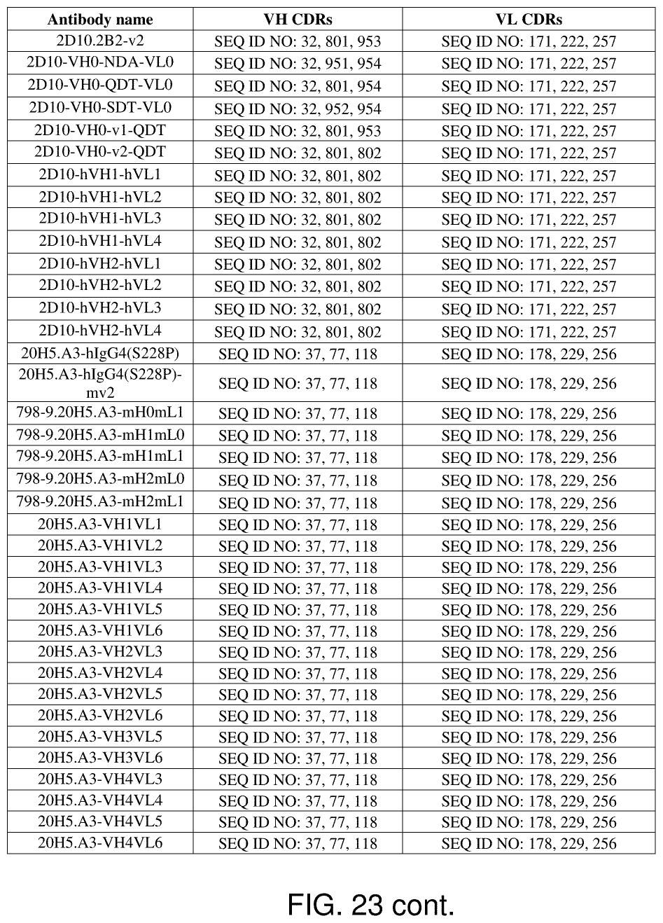

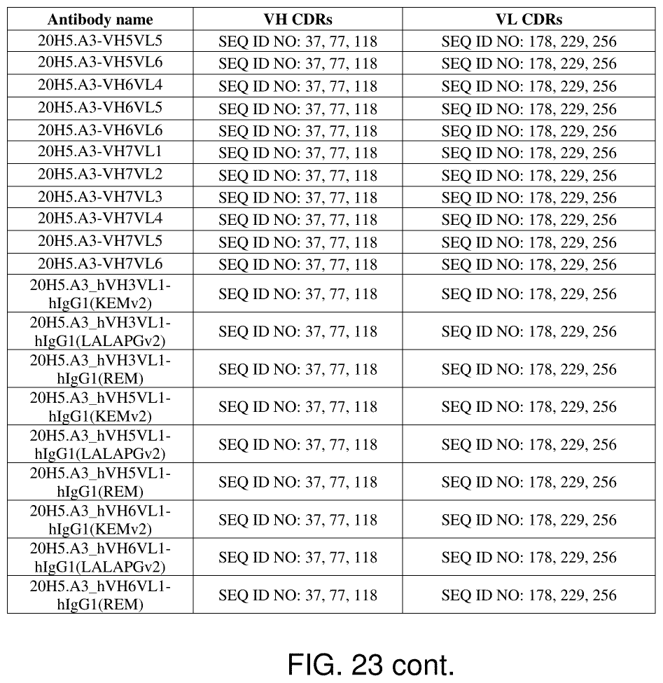

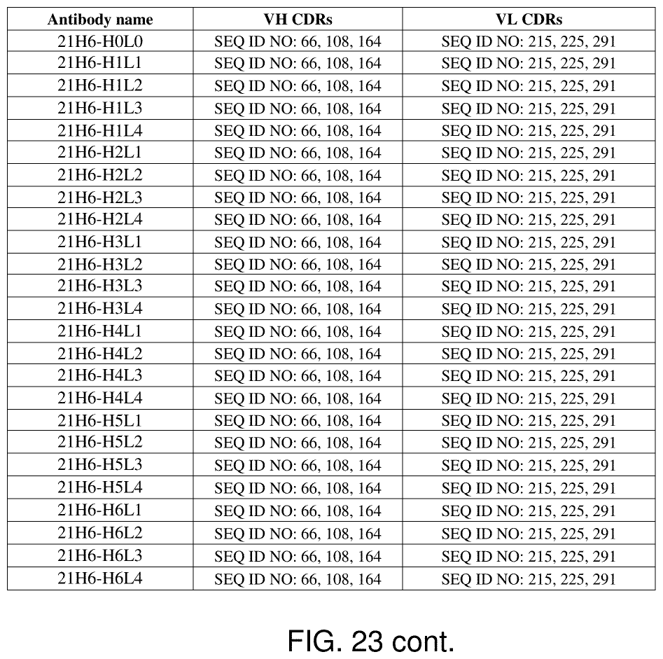

In some embodiments, the antibody or binding fragment thereof comprises a combination of a V_{L}-CDR1, a V_{L}-CDR2, a V_{L}-CDR3, a V_{H}-CDR1, a V_{H}-CDR2, and a V_{H}-CDR3 as illustrated in FIG. 23.

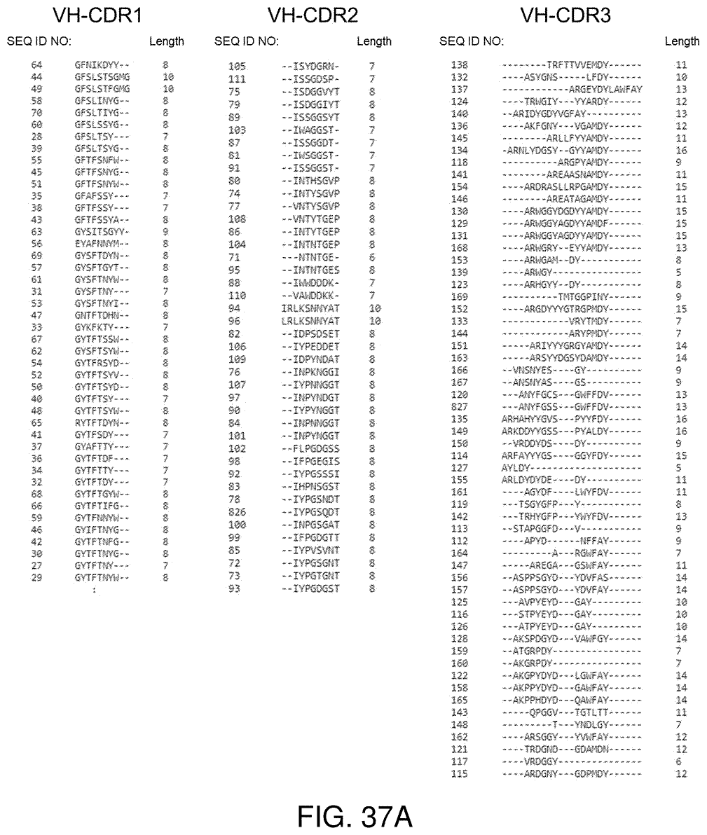

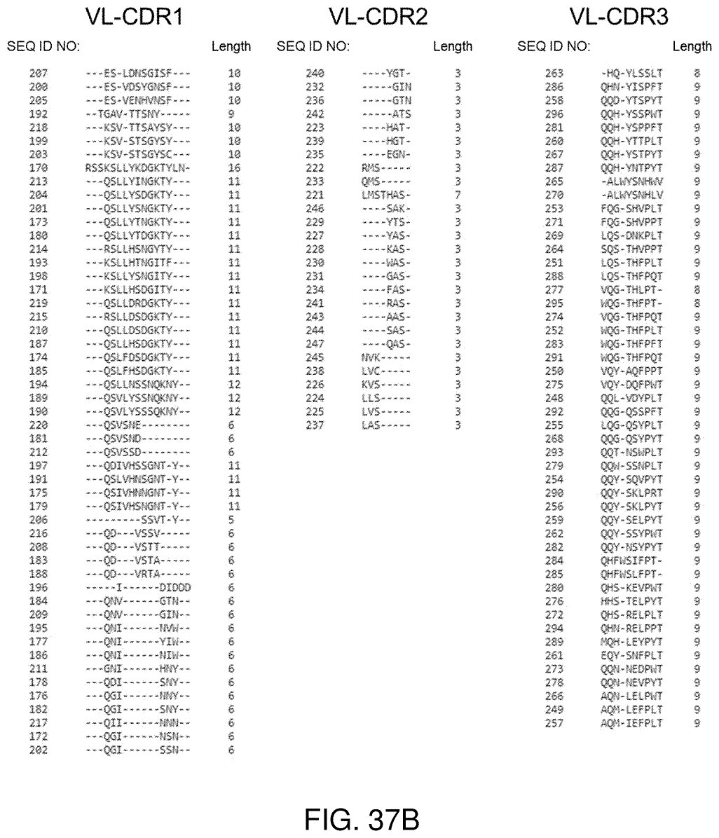

In some embodiments, the antibody or binding fragment thereof comprises a combination of a V_{H}-CDR1, a V_{H}-CDR2, a V_{H}-CDR3, V_{L}-CDR1, a V_{L}-CDR2, and a V_{L}-CDR3, where one or more of these CDRs is defined by a consensus sequence. The consensus sequences provided herein have been derived from the alignments of CDRs depicted in FIG. 37A-B. However, it is envisioned that alternative alignments may be done (e.g. using global or local alignment, or with different algorithms, such as Hidden Markov Models, seeded guide trees, Needleman-Wunsch algorithm, or Smith-Waterman algorithm) and as such, alternative consensus sequences can be derived.

In some embodiments, the V_{H}-CDR1 is defined by the formula X₁X₂X₃X₄X₅X₆X₇X₈X₉X₁₀, where X₁ is E, G, or R; X₂ is F, N, or Y; X₃ is A, I, K, N, S, or T; X₄ is F, I, or L; X₅ is I, K, N, R, S, or T; X₆ is D, G, I, N, S, or T; X₇ is F, G, H, S, or Y; X₈ is no amino acid, A, D, G, I, M, N, T, V, W, or Y; X₉ is no amino acid, M, or Y; X₁₀ is no amino acid or G; In some embodiments, the V_{H}-CDR1 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR1 comprises a sequence having 0, 1, 2, 3, 4, 5, or 6 substitutions from this consensus sequence.

In some embodiments, the V_{H}-CDR2 is defined by the formula X₁X₂X₃X₄X₅X₆X₇X₈X₉X₁₀, where X₁ is no amino acid, I, or L; X₂ is no amino acid or R; X₃ is no amino acid, F, I, L, or V; X₄ is A, D, F, H, K, L, N, S, W, or Y; X₅ is A, D, P, S, T, W, or Y; X₆ is D, E, G, H, K, N, S, V, or Y; X₇ is D, E, G, N, S, or T; X₈ is D, G, I, K, N, Q, R, S, V, or Y; X₉ is A, D, E, G, I, K, N, P, S, T, V, or Y; X₁₀ is no amino acid, I, P, S, or T. In some embodiments, the V_{H}-CDR2 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR2 comprises a sequence having 0, 1, 2, 3, 4, 5, or 6 substitutions from this consensus sequence.

In some embodiments, the V_{H}-CDR3 is defined by the formula X₁X₂X₃X₄X₅X₆X₇X₈X₉X₁₀X₁X₁₂X₁₃X₁₄X₁₅X₁₆X₁₇X₁₈X₁₉X₂₀X₂₁X₂₂X₂₃X₂₄X₂₅, where X₁ is no amino acid or A; X₂ is no amino acid, A, R, or Y; X₃ is no amino acid, A, F, H, K, L, R, S, or V; X₄ is no amino acid, A, D, K, N, R, S, or T; X₅ is no amino acid, A, D, G, H, I, L, N, P, R, S, T, V, or Y; X₆ is no amino acid, A, D, G, H, K, N, P, Q, R, S, or Y; X₇ is no amino acid, D, F, G, H, P, R, S, W, or Y; X₈ is no amino acid, A, D, E, G, I, R, or S; X₉ is no amino acid, A, C, D, E, F, G, I, N, R, S, T, V, or Y; X₁₀ is no amino acid, A, D, M, P, R, S, T, V, or Y; X₁₁ is no amino acid, A, D, E, F, L, T, V, or Y; X₁₂ is no amino acid, A, G, L, M, R, or T; X₁₃ is no amino acid, A, D, E, F, G, R, S, T, or V; X₁₄ is no amino acid, A, D, G, L, P, Q, R, S, T, V, or Y; X₁₅ is no amino acid, A, D, G, N, S, V, W, or Y; X₁₆ is no amino acid, A, D, E, F, L, P, T, V, W, or Y; X₁₇ is no amino acid, F, I, L, M, R, or Y; X₁₈ is no amino acid, A, D, G, N, or T; X₁₉ is no amino acid, F, N, S, T, V, or Y; X₂₀ is no amino acid or L; X₂₁ is no amino acid or A; X₂₂ is no amino acid or W; X₂₃ is no amino acid or F; X₂₄ is no amino acid or A; X₂₅ is no amino acid or Y. In some embodiments, the V_{H}-CDR3 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR3 comprises a sequence having 0, 1, 2, 3, 4, 5, or 6 substitutions from this consensus sequence.

In some embodiments, the V_{L}-CDR1 is defined by the formula X₁X₂X₃X₄X₅X₆X₇X₈X₉X₁₀X₁₁X₁₂X₁₃X₁₄X₁₅X₁₆X₁₇, where X₁ is no amino acid or R; X₂ is no amino acid or S; X₃ is no amino acid, S, or T; X₄ is no amino acid, E, G, K, Q, or R; X₅ is no amino acid, A, D, G, I, N, or S; X₆ is no amino acid, I, L, or V; X₇ is no amino acid, F, L, S, or V; X₈ is no amino acid, D, E, H, N, S, T, or Y; X₉ is no amino acid, D, E, I, K, N, R, S, T, or V; X₁₀ is no amino acid, D, H, N, R, S, or Y; X₁₁ is no amino acid, A, G, N, S, T, or V; X₁₂ is no amino acid, A, I, K, N, Q, T, V, or Y; X₁₃ is no amino acid, D, G, H, K, N, S, T, or Y; X₁₄ is no amino acid, C, F, I, N, S, T, V, or Y; X₁₅ is no amino acid, D, L, N, W, or Y; X₁₆ is no amino acid, N, or D; X₁₇ is no amino acid or D. In some embodiments, the V_{L}-CDR1 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR1 comprises a sequence having 0, 1, 2, 3, 4, 5, or 6 substitutions from this consensus sequence.

In some embodiments, the V_{L}-CDR2 is defined by the formula X₁X₂X₃X₄X₅X₆X₇X₈, where X₁ is no amino acid, K, L, N, Q, or R; X₂ is no amino acid, A, L, M, or V; X₃ is no amino acid, C, K, or S; X₄ is no amino acid or T; X₅ is no amino acid, A, E, F, G, H, K, Q, R, S, W, or Y; X₆ is no amino acid, A, G, or T; X₇ is no amino acid, I, K, N, S, or T; X₈ is no amino acid, N, or S. In some embodiments, the V_{L}-CDR2 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR2 comprises a sequence having 0, 1, 2, 3, 4, 5, or 6 substitutions from this consensus sequence.

In some embodiments, the V_{L}-CDR3 is defined by the formula X₁X₂X₃X₄X₅X₆X₇X₈X₉X₁₀, where X₁ is no amino acid, A, E, F, H, L, M, Q, S, V, or W; X₂ is A, H, or Q; X₃ is D, F, G, H, L, M, N, Q, S, T, W, or Y; X₄ is no amino acid or W; X₅ is A, D, I, K, L, N, Q, R, S, T, V, or Y; X₆ is D, E, H, I, K, L, N, Q, S, or T; X₇ is D, F, K, L, N, P, S, T, V, W, or Y; X₈ is H, P, or S; X₉ is F, L, P, Q, R, T, W, or Y; X₁₀ is no amino acid, T, or V. In some embodiments, the V_{L}-CDR3 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR3 comprises a sequence having 0, 1, 2, 3, 4, 5, or 6 substitutions from this consensus sequence.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID NOs: 297-373, 803, 806-820, 940, 955-968, 1067-1109, 1415-1439. In some embodiments, the light chain variable region of the antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID NOs: 374-447, 821-835, 941-943, 969-982, 1110-1152, 1440-1464. In some embodiments, the antibodies or binding fragments thereof are anti-Gal3 antibodies or binding fragments thereof.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID NOs: 297-373, 803, 806-820, 940, 955-968, 1067-1109, 1415-1439. In some embodiments, the light chain variable region of the antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID NOs: 374-447, 821-835, 941-943, 969-982, 1110-1152, 1440-1464. In some embodiments, the antibodies or binding fragments thereof are anti-Gal3 antibodies or binding fragments thereof.

In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a VL sequence, a V_{H} sequence, a VL/VH pairing, and/or V_{L}-CDR1, V_{L}-CDR2, V_{L}-CDR3, V_{H}-CDR1, V_{H}-CDR2, V_{H}-CDR3 (including 1, 2, 3, 4, or 5 amino acid substitutions of any one or more of these CDRs) set from the heavy chain and light chain sequences as depicted in FIG. 25.

In some embodiments, antibodies or binding fragments thereof are provided. In some embodiments, the antibodies are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises one of the amino acid sequences of SEQ ID NOs: 27-70, the V_{H}-CDR2 comprises one of the amino acid sequences of SEQ ID NOs: 71-111, 801, 951, 952, the V_{H}-CDR3 comprises one of the amino acid sequences of SEQ ID NO: 112-169, 802, 953, 954, the V_{L}-CDR1 comprises one of the amino acid sequences of SEQ ID NOs: 170-220, the V_{L}-CDR2 comprises one of the amino acid sequences of SEQ ID NOs: 211-247, the V_{L}-CDR3 comprises one of the amino acid sequences of SEQ ID NOs: 248-296, the heavy chain variable region has a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one of the amino acid sequences of SEQ ID NOs: 297-373, 803, 806-820, 940, 955-968, 1067-1109, 1415-1439, and the light chain variable region has a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one of the amino acid sequences of SEQ ID NOs: 374-447, 821-835, 941-943, 969-982,1110-1152, 1440-1464.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID NOs: 448-494, 804, 836-850, 983-996, 1153-1195, 1411, 1465-1489. In some embodiments, the antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514. In some embodiments, the antibodies or binding fragments thereof are anti-Gal3 antibodies or binding fragments thereof.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID NOs: 448-494, 804, 836-850, 983-996, 1153-1195, 1411, 1465-1489. In some embodiments, the antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% similarity to the sequence selected from SEQ ID NOs: 495-538, 805, 851-865, 997-1010, 1196-1238, 1412, 1490-1514. In some embodiments, the antibodies or binding fragments thereof are anti-Gal3 antibodies or binding fragments thereof.

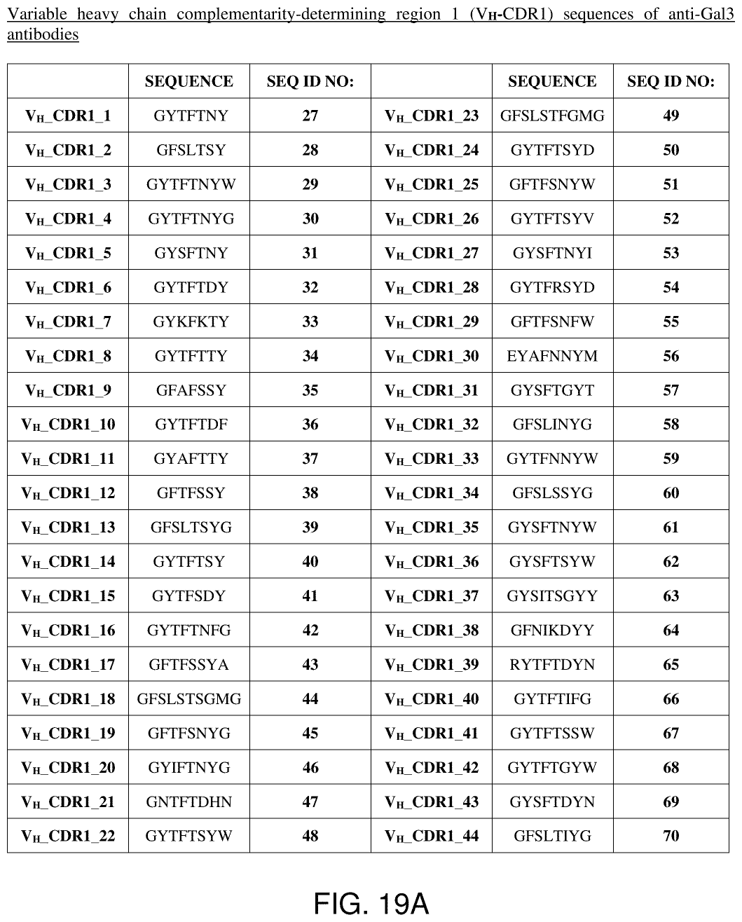

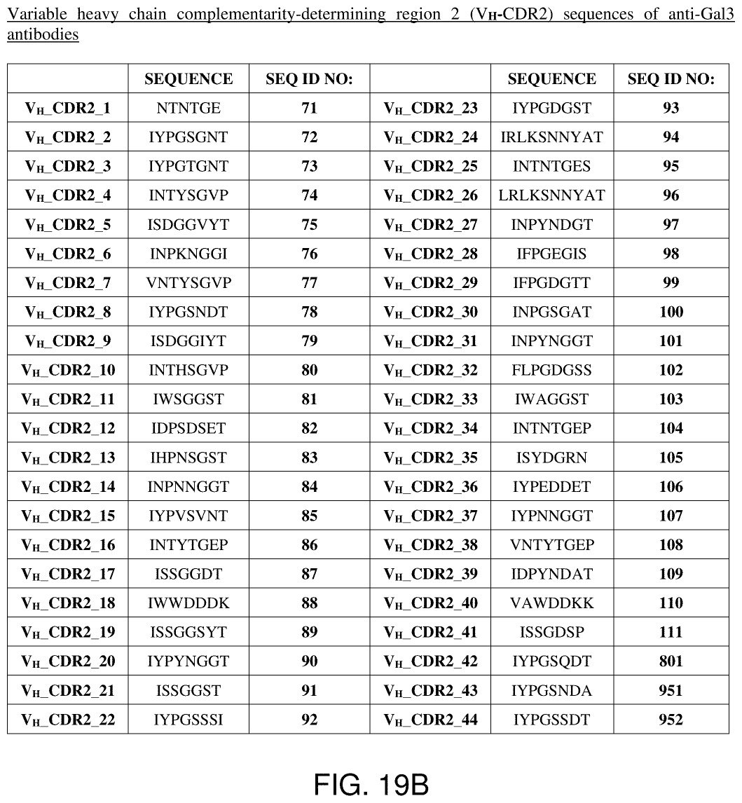

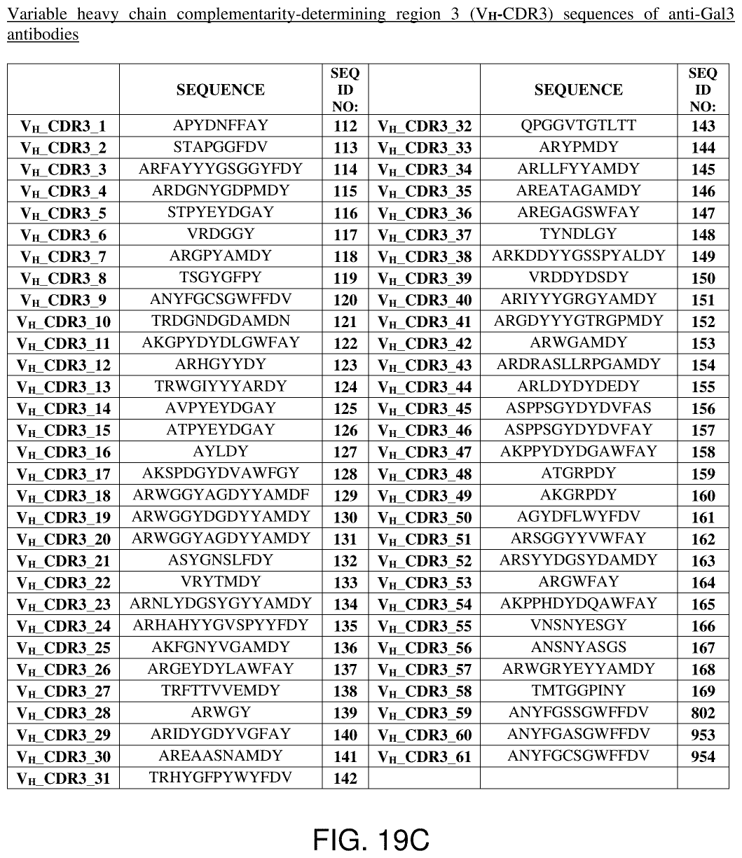

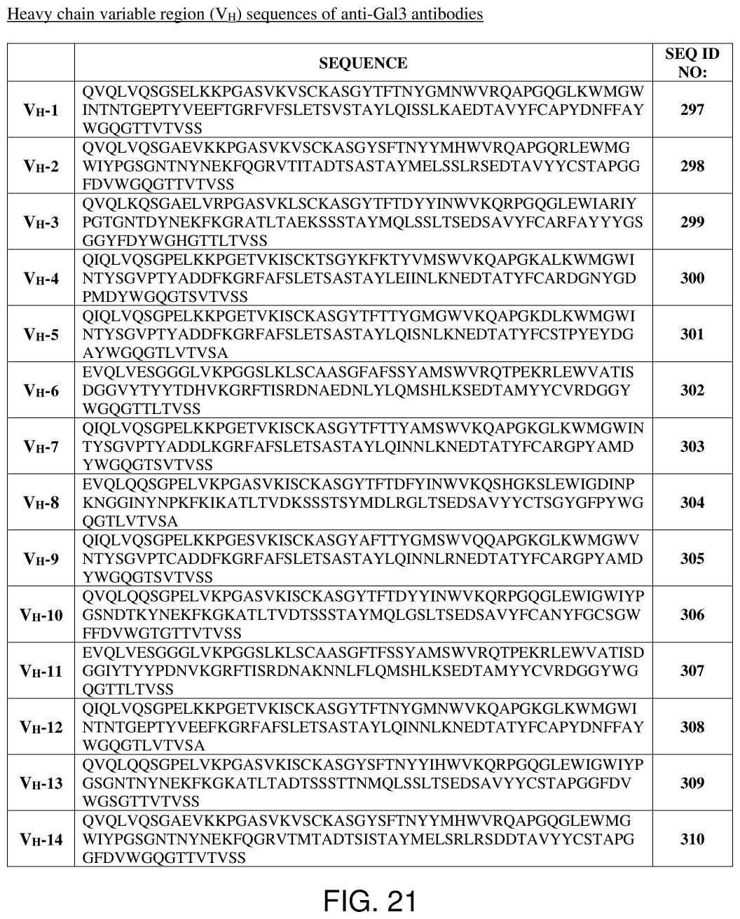

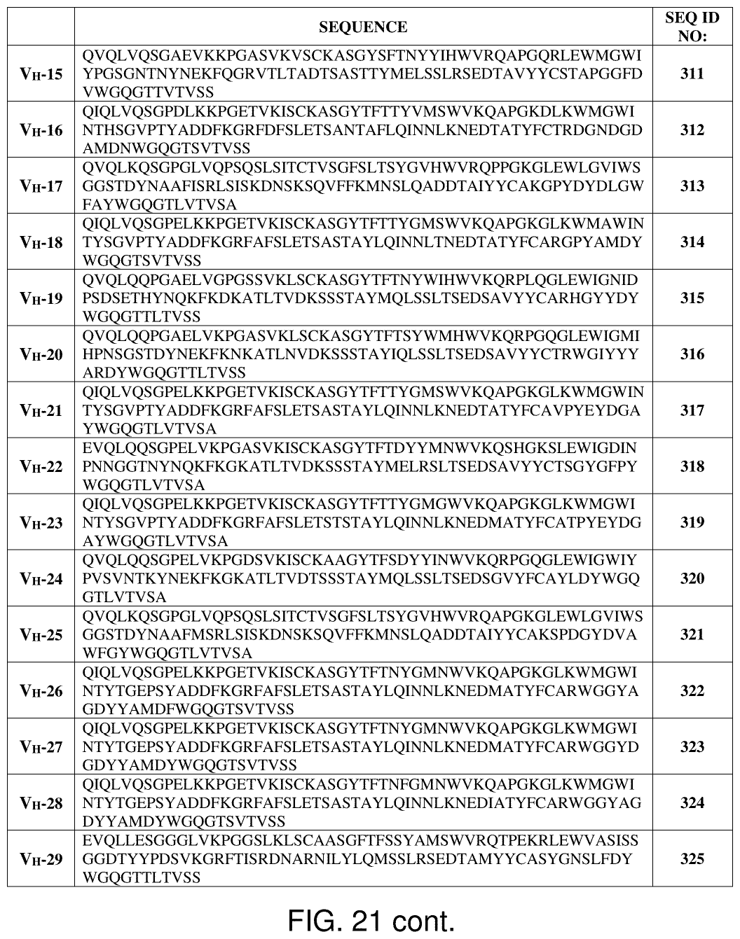

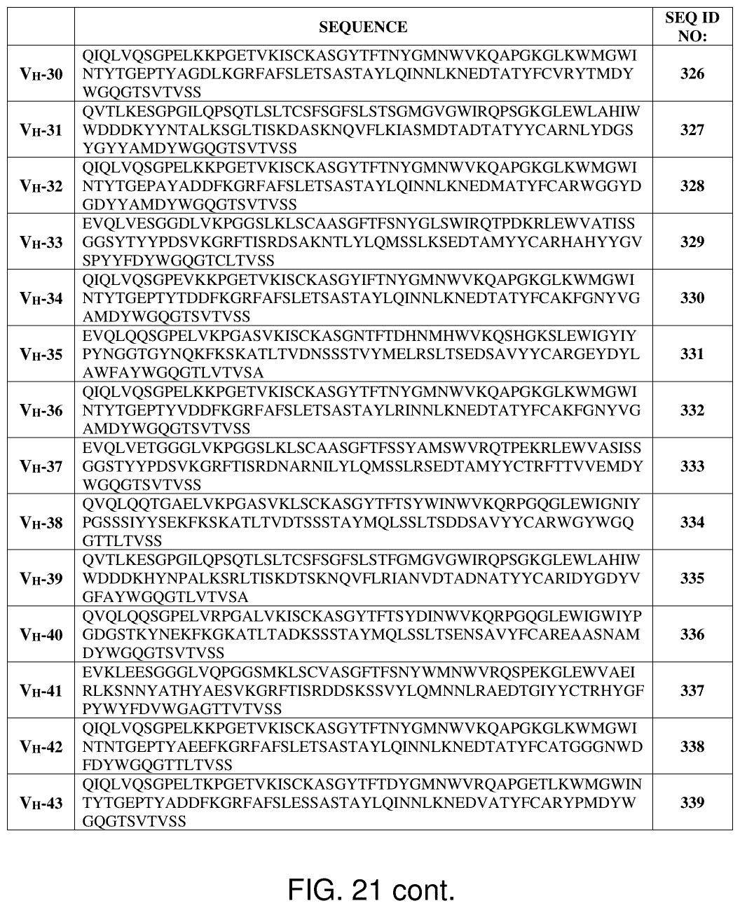

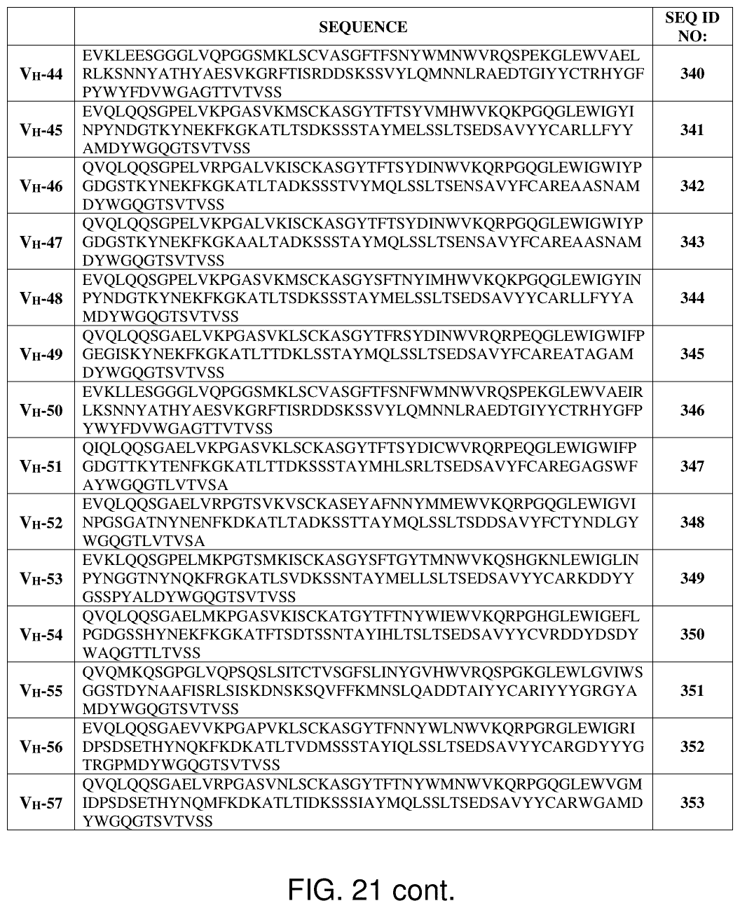

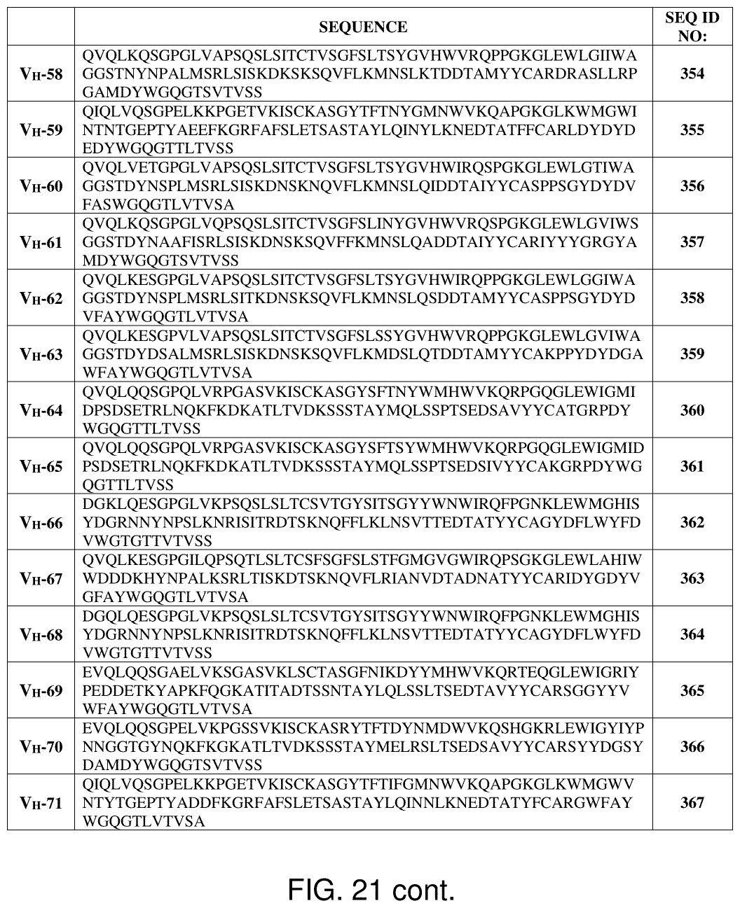

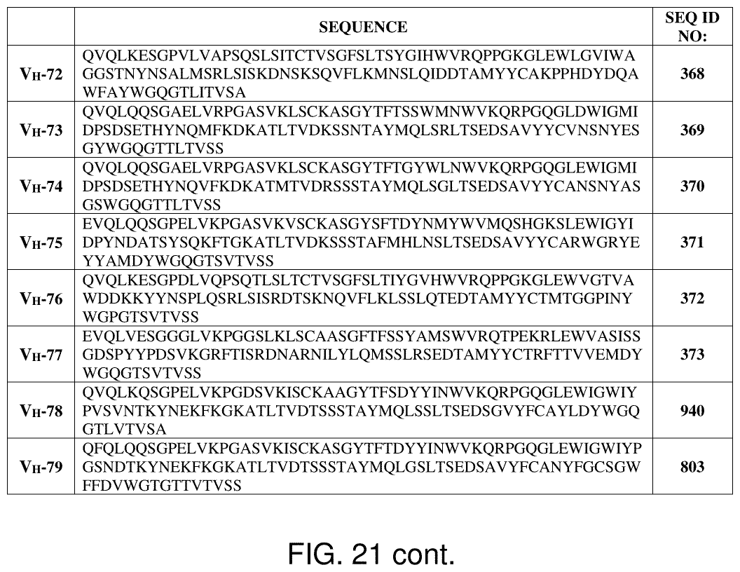

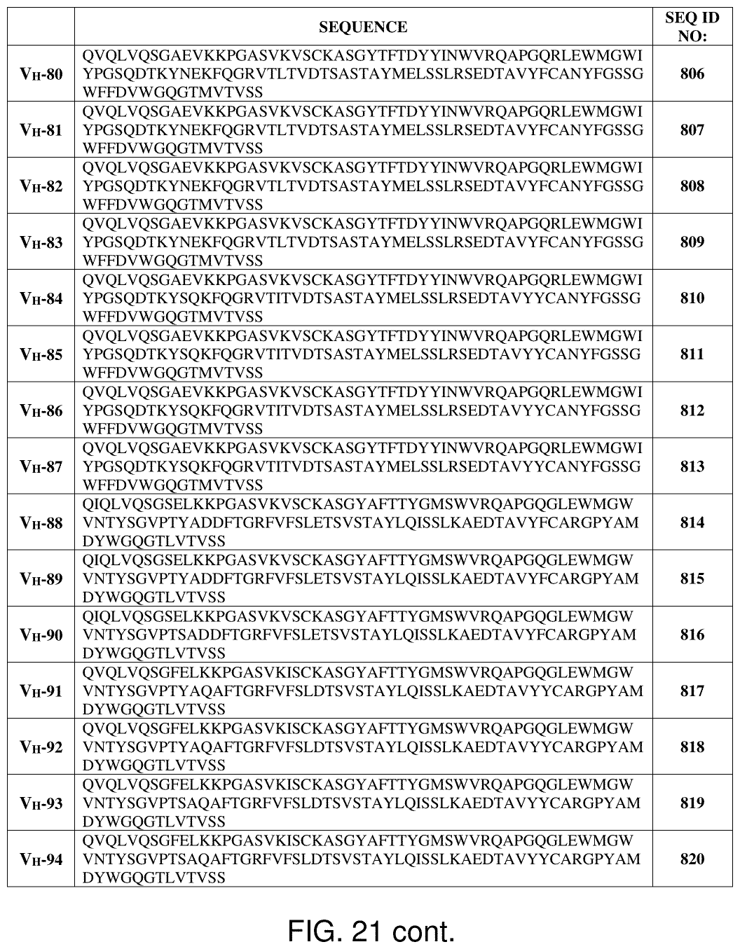

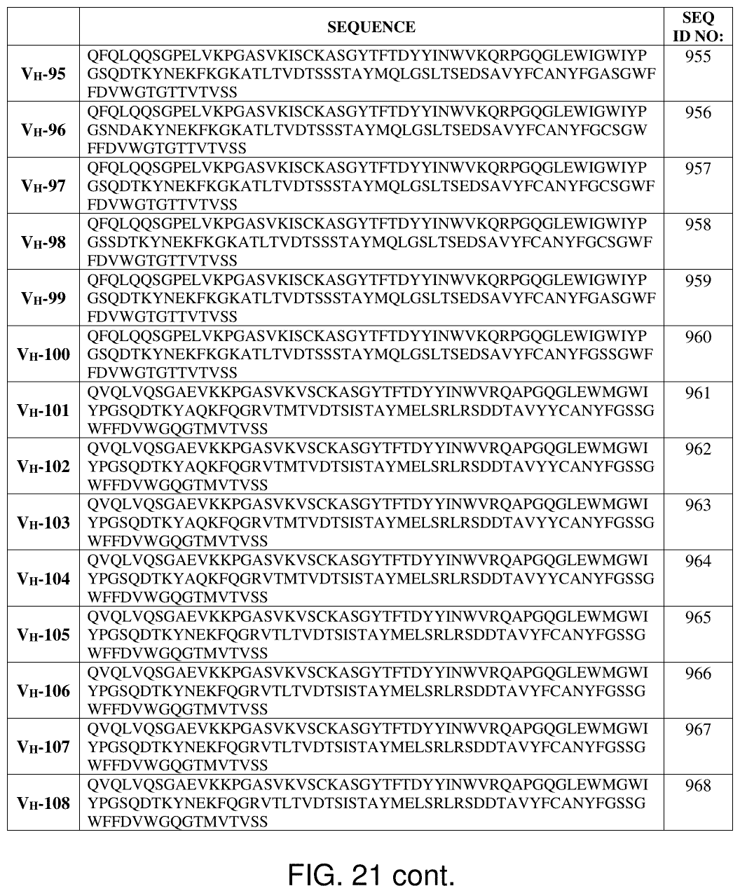

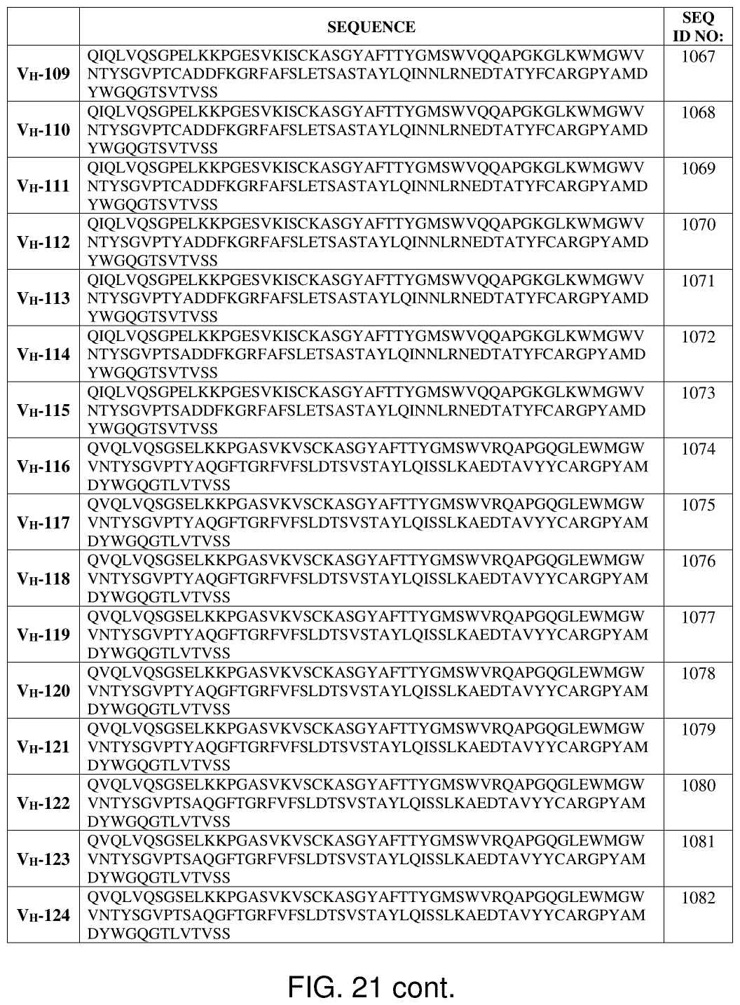

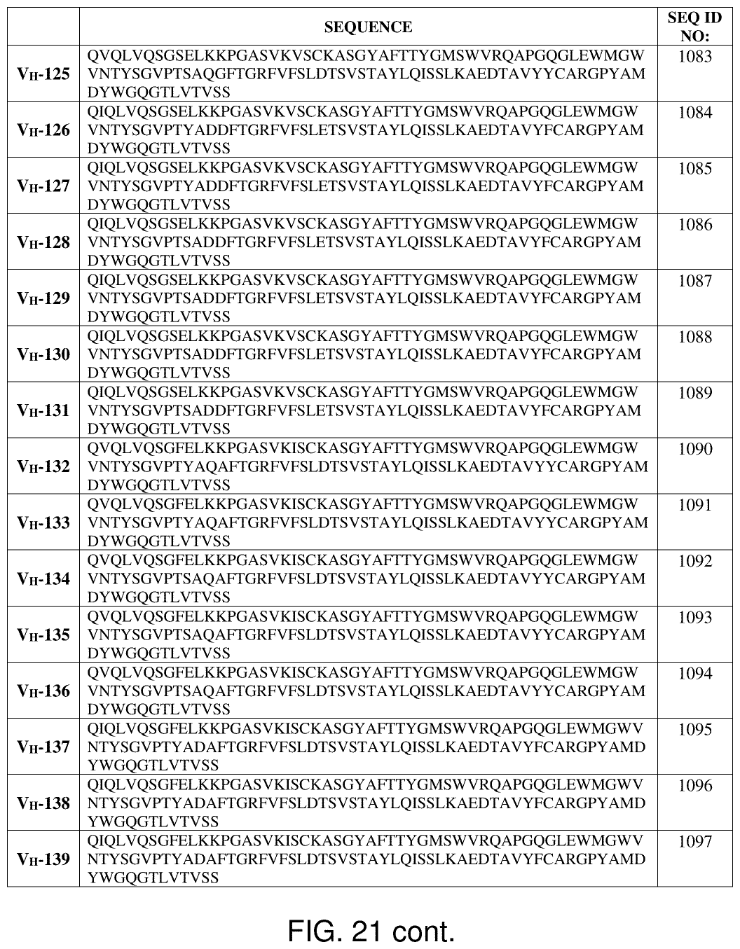

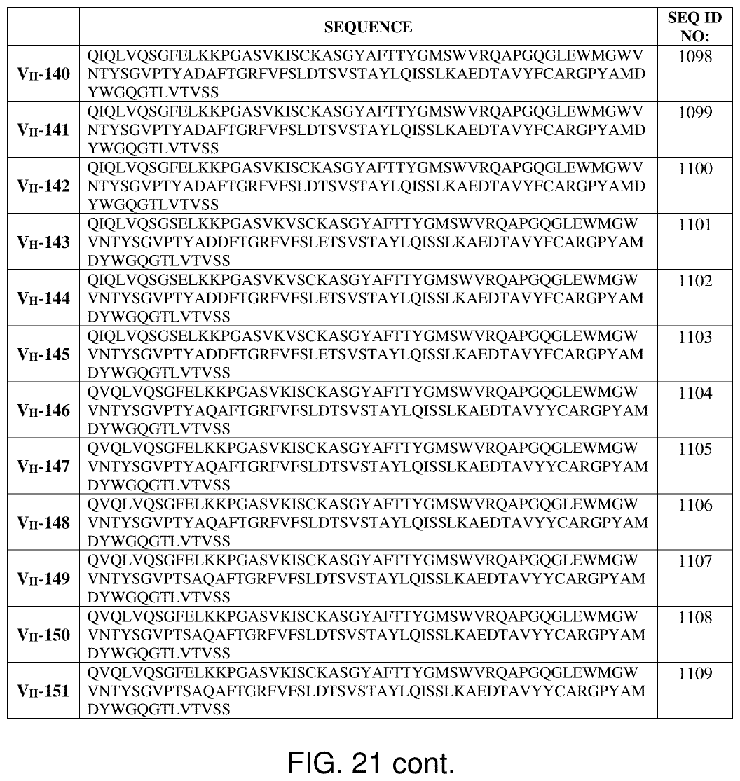

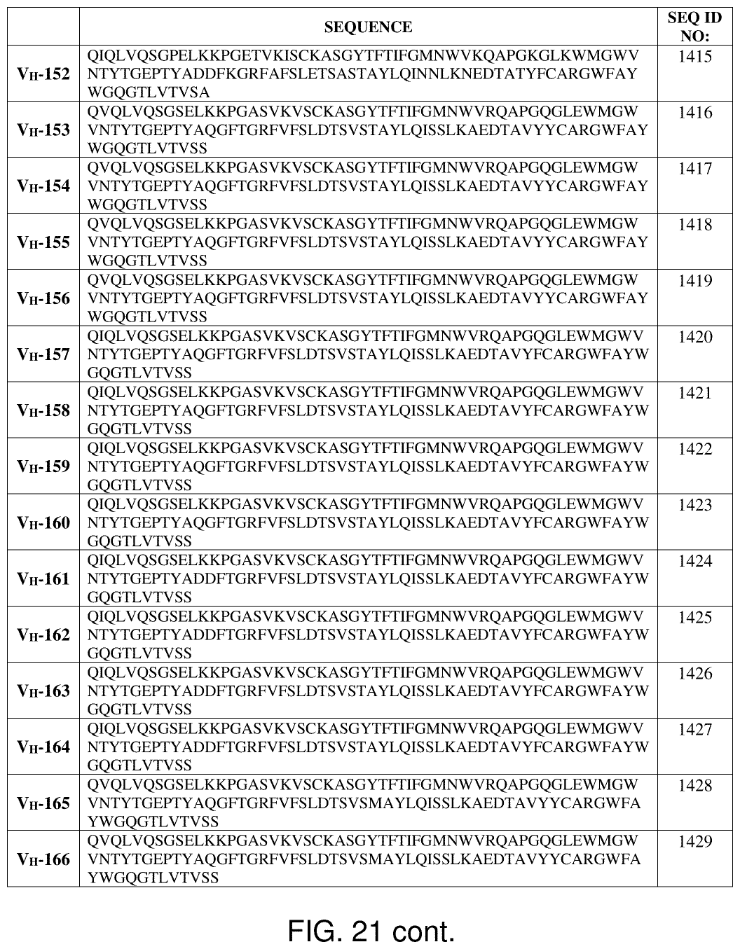

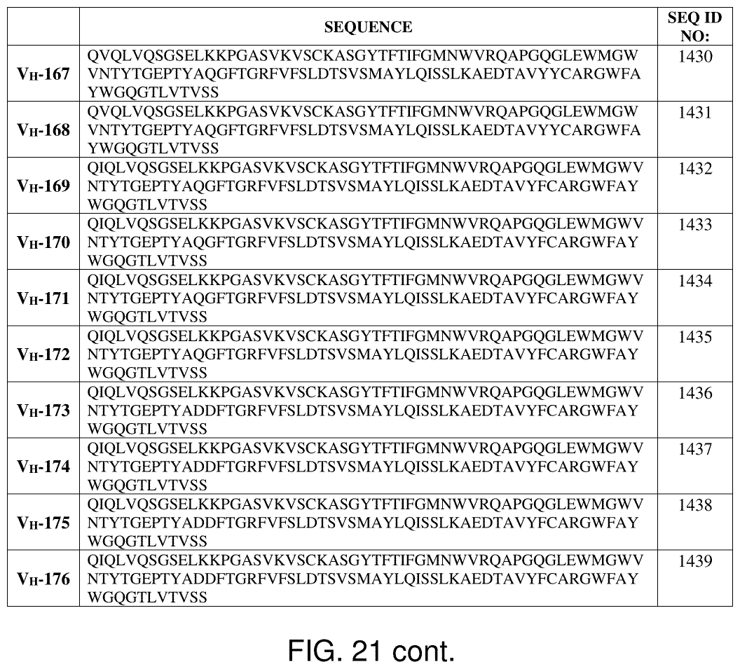

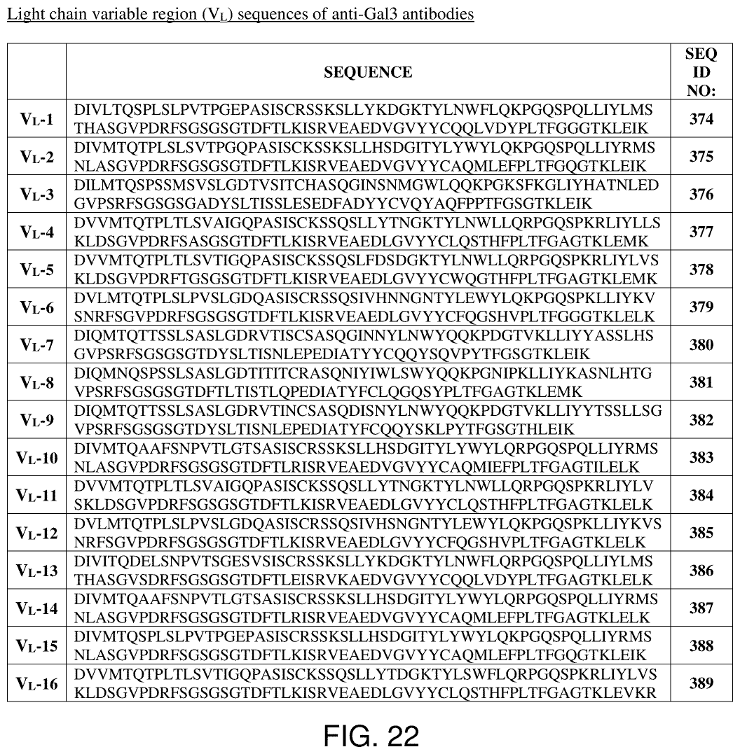

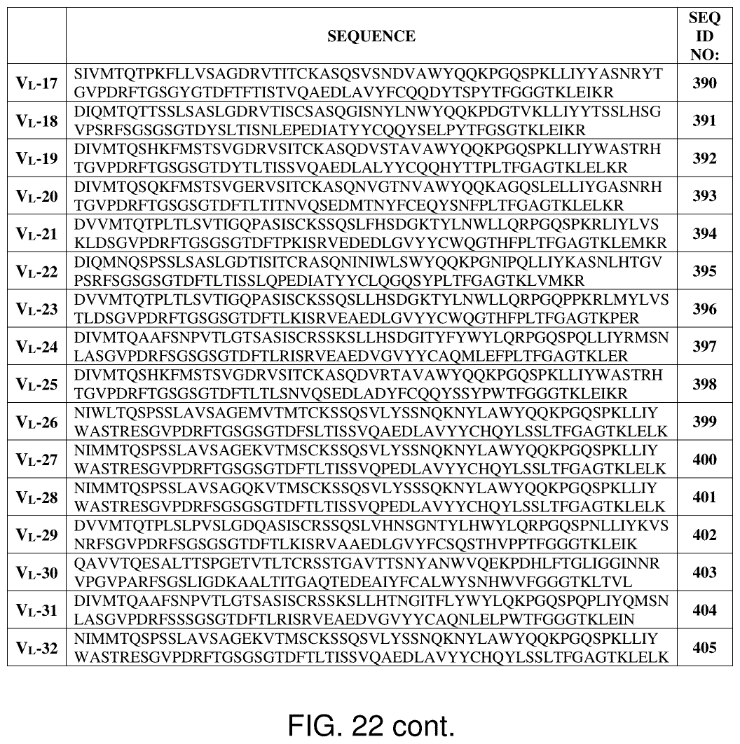

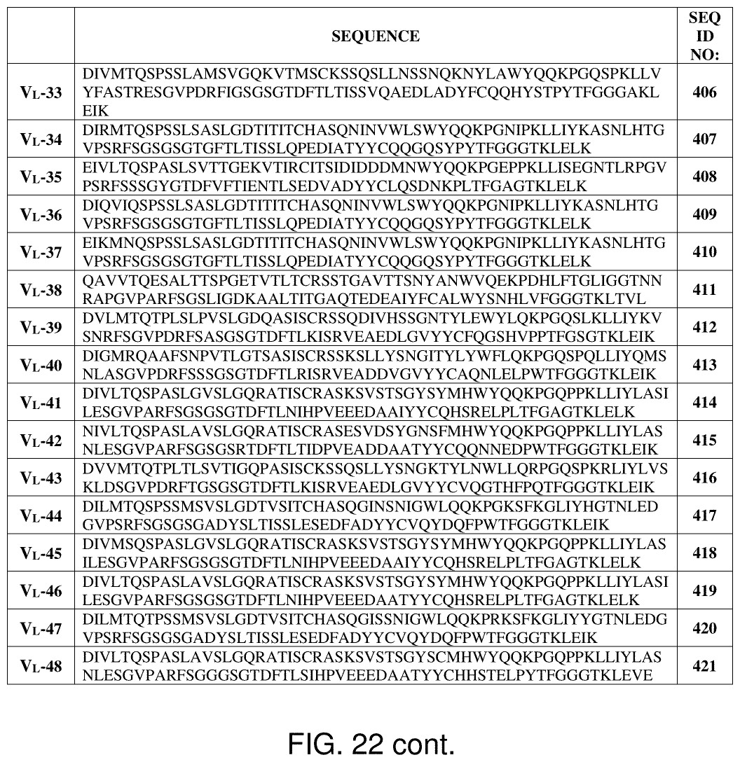

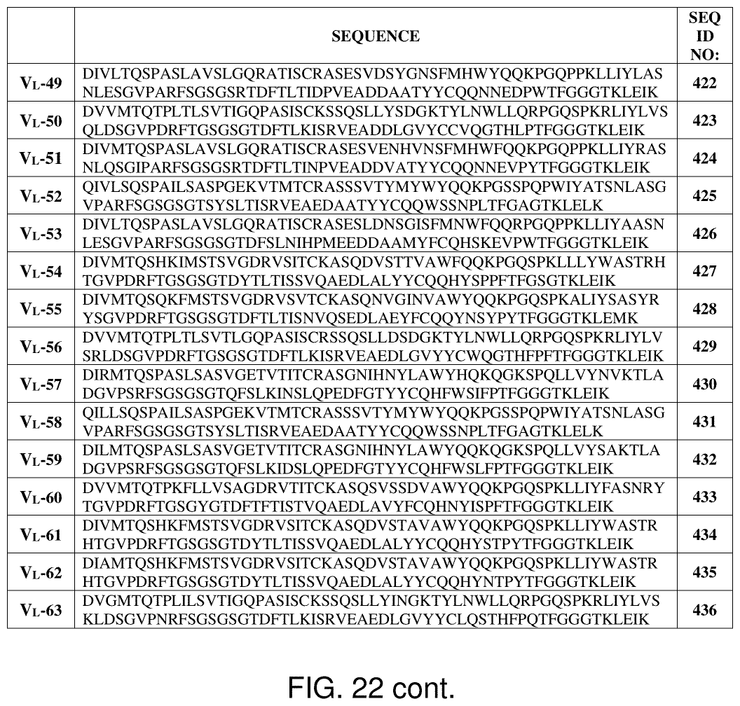

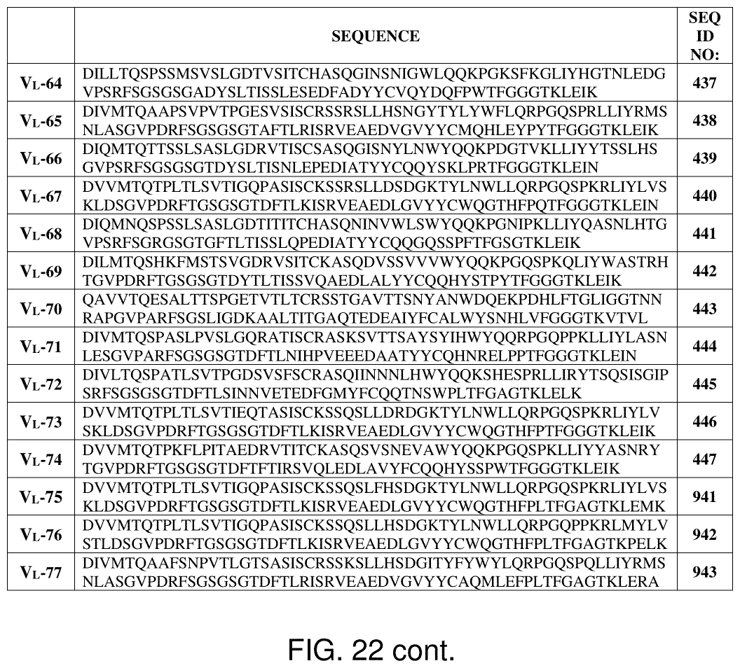

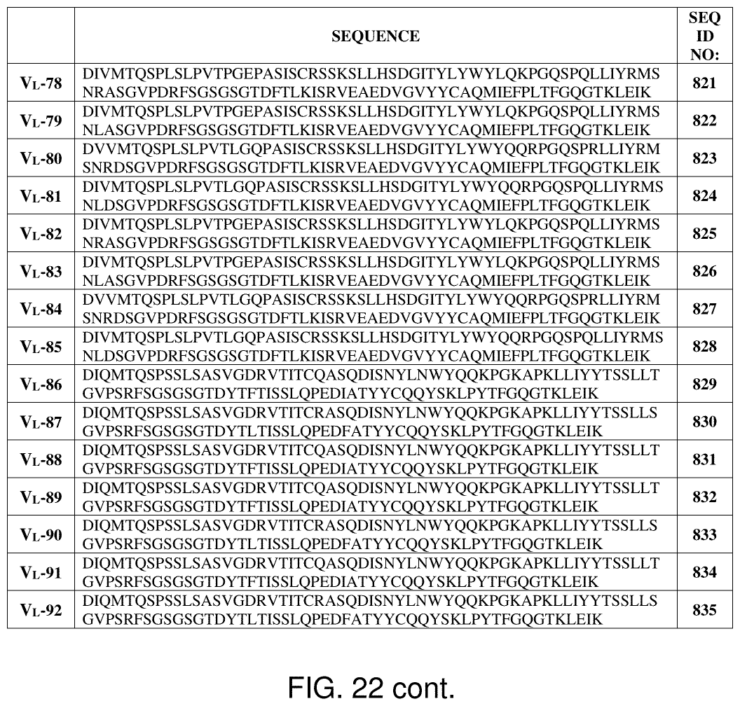

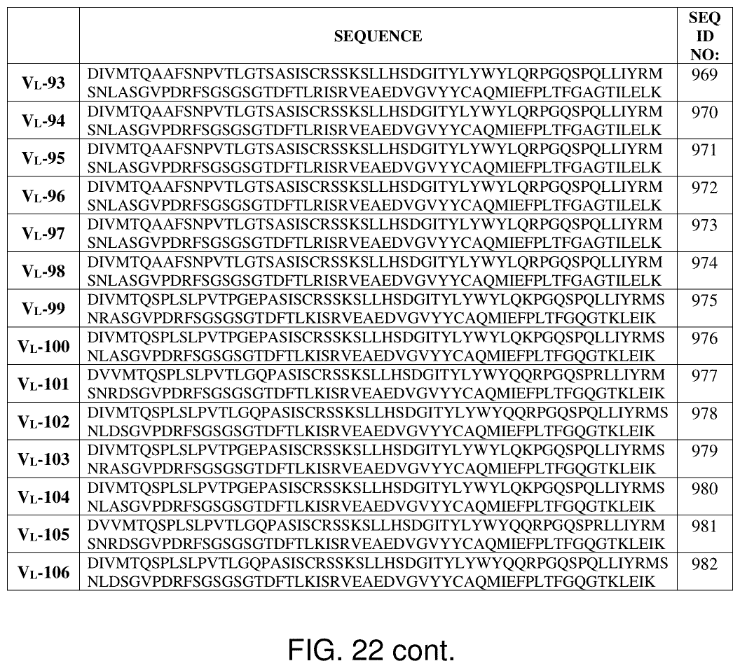

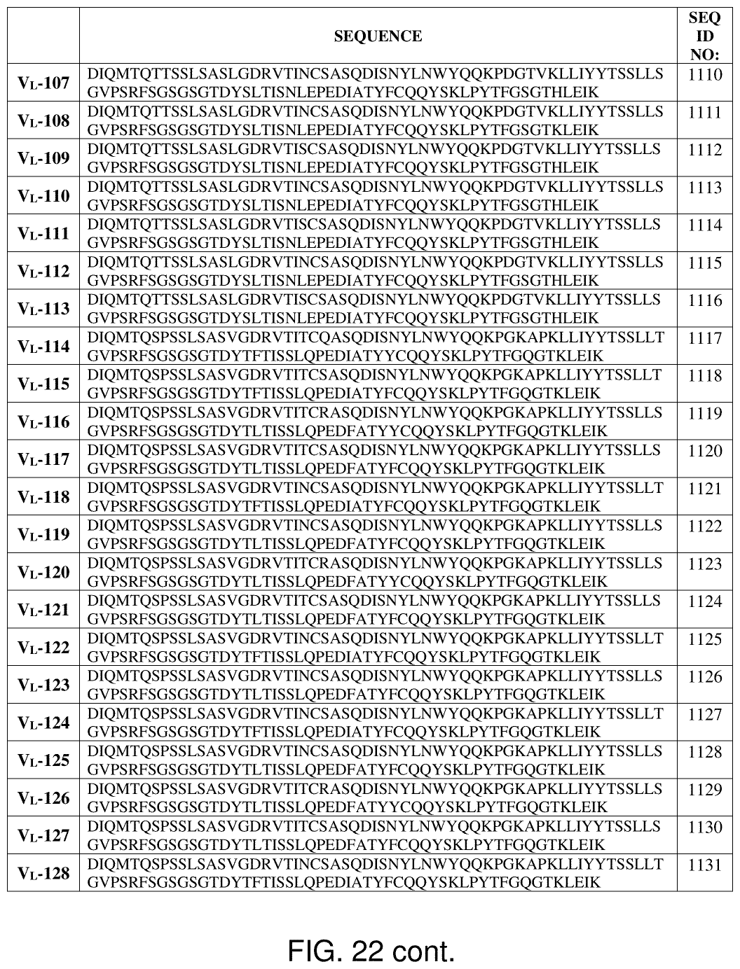

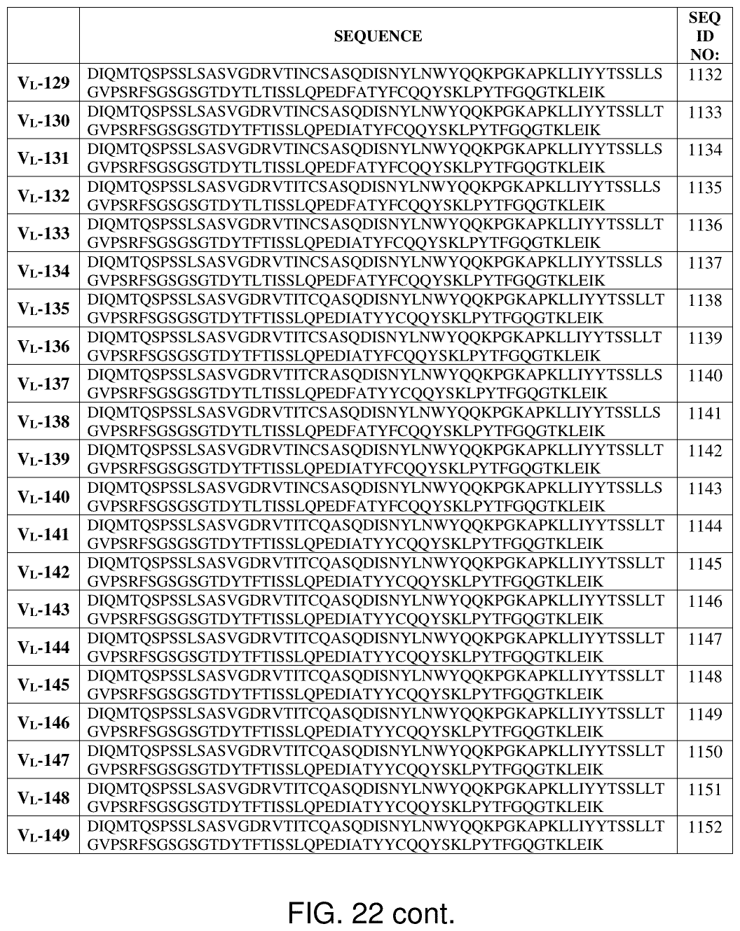

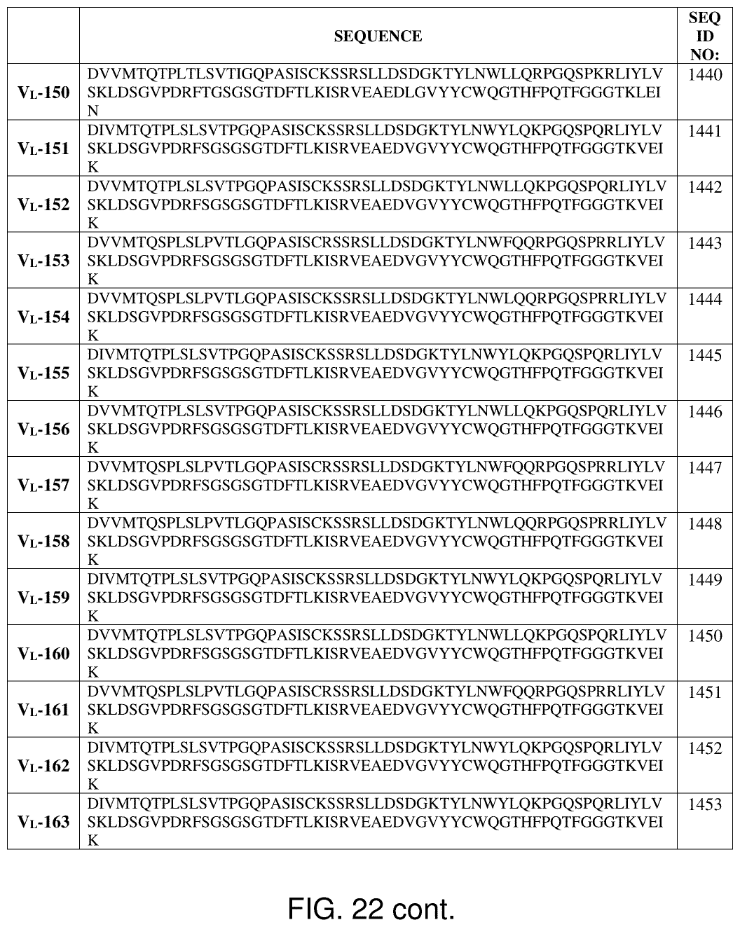

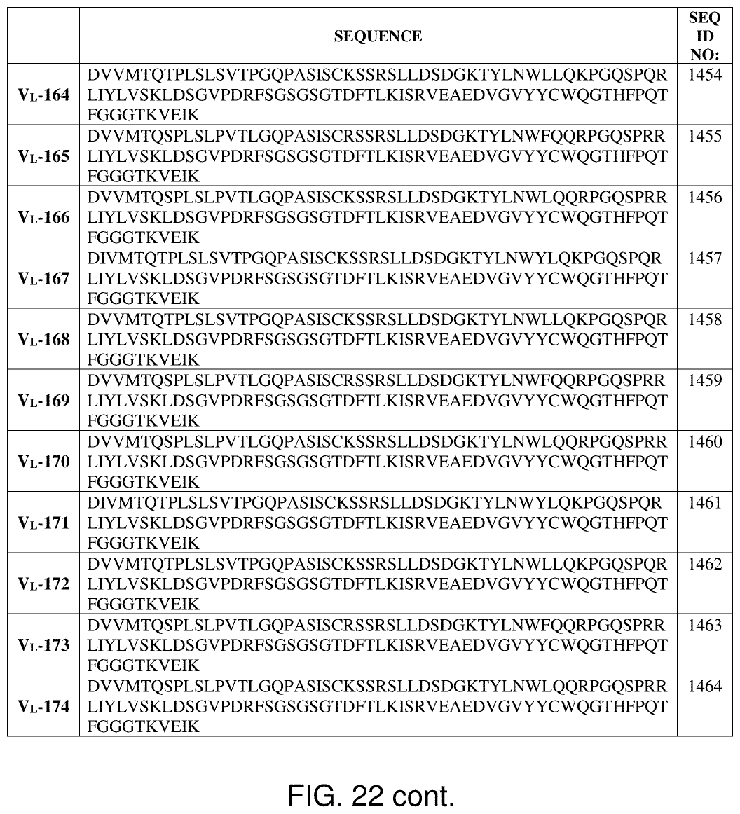

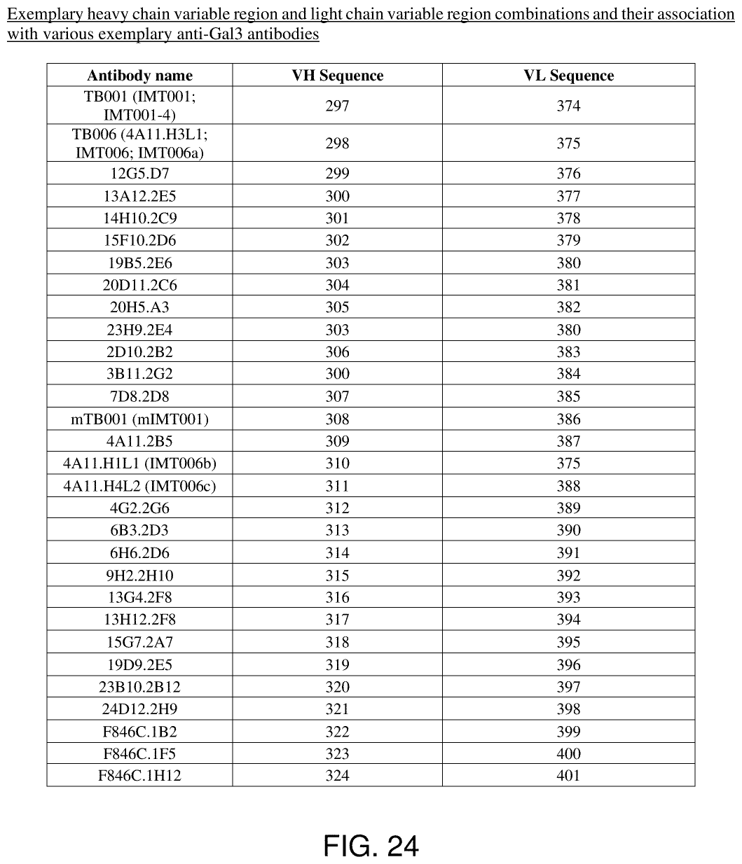

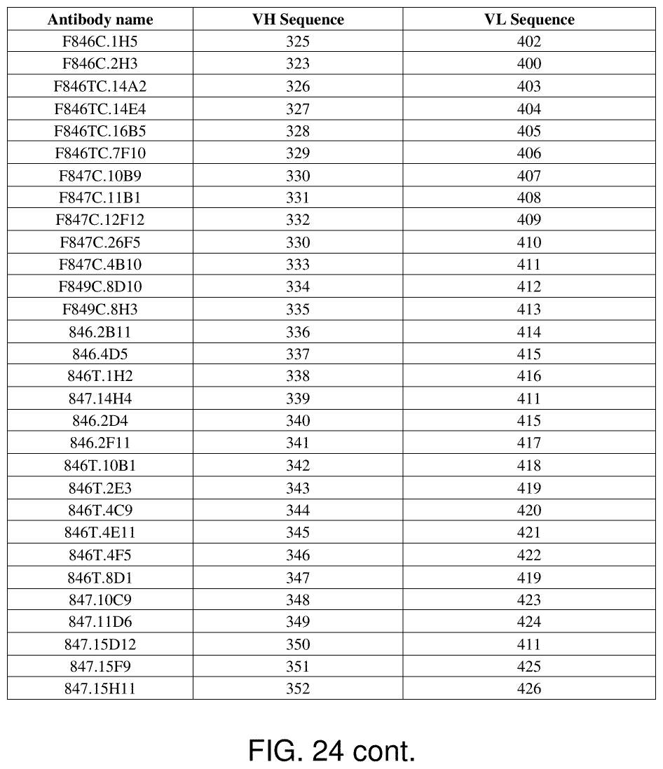

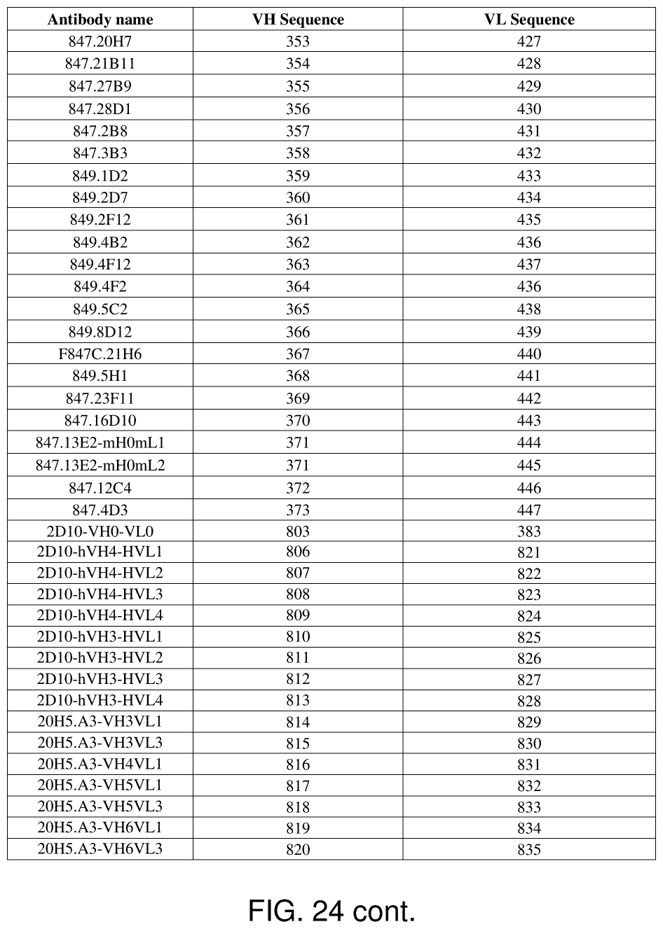

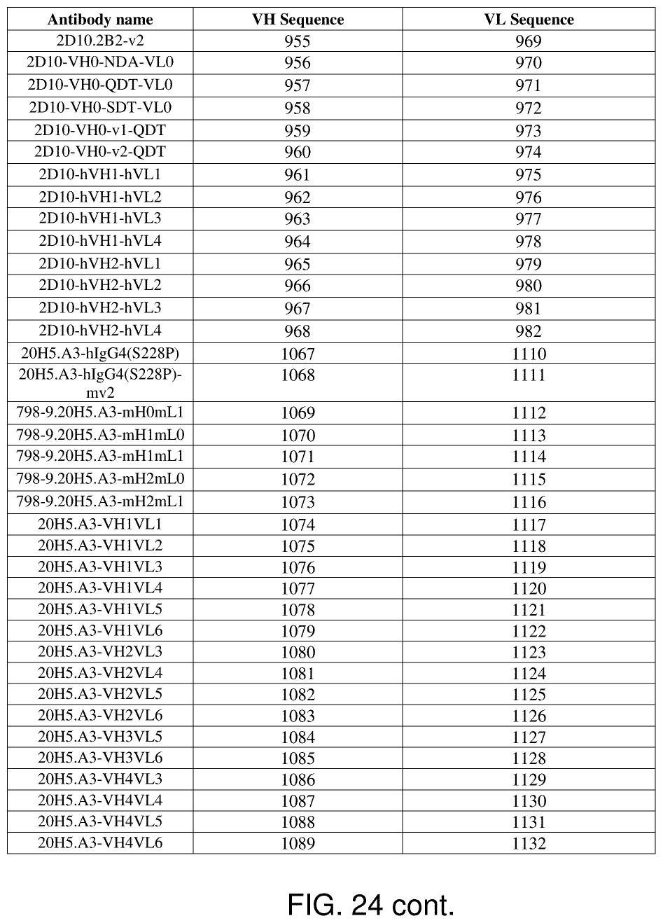

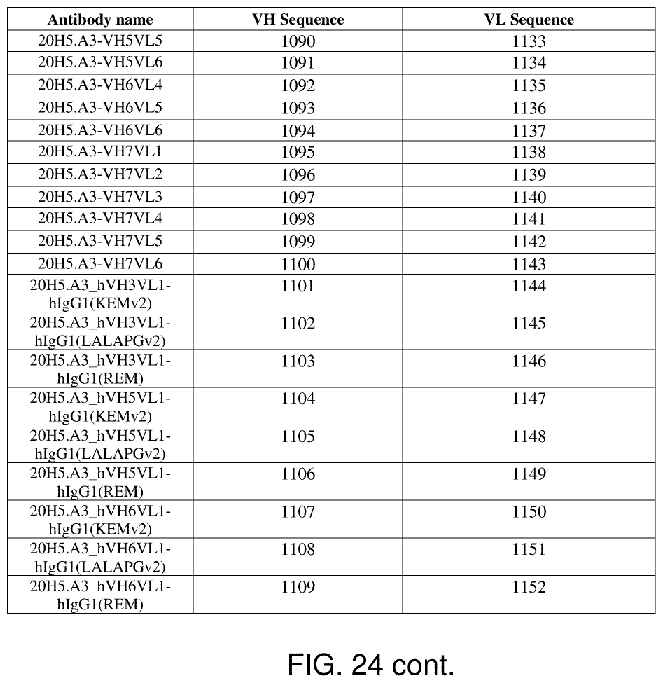

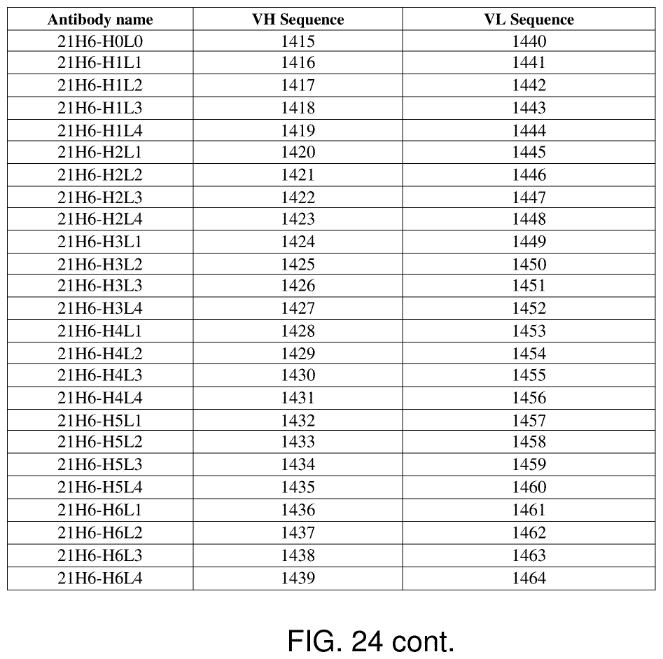

In some embodiments, antibodies or binding fragments thereof are provided. In some embodiments, the antibodies are anti-Gal3 antibodies or binding fragments thereof. In some embodiments, the anti-Gal3 antibodies or binding fragments thereof comprise a heavy chain variable region and a light chain variable region. In some embodiments, the heavy chain variable region is paired with an IgG4 heavy chain constant domain, an IgG2 heavy chain constant domain, or an IgG1 heavy chain constant domain. In some embodiments, the IgG4 heavy chain constant domain, IgG2 heavy chain constant domain, or the IgG1 heavy chain constant domain are human or murine. In some embodiments, the IgG4 heavy chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 945. In some embodiments, the IgG4 heavy chain constant domain is an S228P mutant. In some embodiments, the IgG2 heavy chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 947 or SEQ ID NO: 948. In some embodiments, the IgG2 heavy chain constant domain is a LALAPG or a LALA mutant. In some embodiments, the IgG1 heavy chain constant domain is a KEM, REM, or LALAPGv2 mutant. In some embodiments, the IgG1 heavy chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 1615, 1616, or 1617. In some embodiments, the light chain variable region is paired with an IgG4 kappa chain constant domain. In some embodiments, the IgG4 kappa chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 946. In some embodiments, the light chain variable region and/or heavy chain variable region may be selected from those depicted in FIGS. 21 and 22 and/or the combinations of light chain variable region and heavy chain variable region as depicted in FIG. 24. In some embodiments, the light chain variable region and/or heavy chain variable regions comprise one or more CDRs depicted in FIGS. 19A-C, 20A-C and/or the combinations of CDRs depicted in FIG. 23.

In some embodiments, the antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

In some embodiments, the antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

In some embodiments, the antibody or binding fragment thereof comprises a sequence (e.g. CDR, VL, VH, LC, HC) having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

In some embodiments, the antibody or binding fragment thereof comprises a sequence (e.g. CDR, VL, VH, LC, HC) having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to a sequence of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, 20H5.A3-VH6VL3, or binding fragment thereof.

In some embodiments, antibodies or binding fragments thereof are provided. In some embodiments, the antibodies or binding fragments thereof are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 32. In some embodiments, the V_{H}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 801, 951, 952. In some embodiments, the V_{H}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 802, 953, 954. In some embodiments, the V_{L}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 171. In some embodiments, the V_{L}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 222. In some embodiments, the V_{L}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 257.

In some embodiments, antibodies or binding fragments thereof are provided. In some embodiments, the antibodies or binding fragments thereof are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 32. In some embodiments, the V_{H}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 801, 951, 952. In some embodiments, the V_{H}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 802, 953, 954. In some embodiments, the V_{L}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 171. In some embodiments, the V_{L}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 222. In some embodiments, the V_{L}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 257.

In some embodiments, antibodies or binding fragments thereof are provided. In some embodiments, the antibodies or binding fragments thereof are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 0, 1, 2, 3, 4, 5, or 6 substitutions relative to SEQ ID NO: 32. In some embodiments, the V_{H}-CDR2 comprises an amino acid sequence having at least 0, 1, 2, 3, 4, 5, or 6 substitutions relative to SEQ ID NO: 801, 951, 952. In some embodiments, the V_{H}-CDR3 comprises an amino acid sequence having at least 0, 1, 2, 3, 4, 5, or 6 substitutions relative to SEQ ID NO: 802, 953, 954. In some embodiments, the V_{L}-CDR1 comprises an amino acid sequence having at least 0, 1, 2, 3, 4, 5, or 6 substitutions relative to SEQ ID NO: 171. In some embodiments, the V_{L}-CDR2 comprises an amino acid sequence having at least 0, 1, 2, 3, 4, 5, or 6 substitutions relative to SEQ ID NO: 222. In some embodiments, the V_{L}-CDR3 comprises an amino acid sequence having at least 0, 1, 2, 3, 4, 5, or 6 substitutions relative to SEQ ID NO: 257.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 806-813, 955-968. In some embodiments, the light chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 821-828, 969-982.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 806-813, 955-968. In some embodiments, the light chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 821-828, 969-982.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 836-843, 983-996. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 851-858, 997-1010.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 836-843, 983-996. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 851-858, 997-1010.

In some embodiments, antibodies or binding fragments thereof are provided. In some embodiments, the antibodies or binding fragments thereof are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a VL-CDR3. In some embodiments, the V_{H}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 37. In some embodiments, the V_{H}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 77. In some embodiments, the V_{H}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 118. In some embodiments, the V_{L}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 178. In some embodiments, the V_{L}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 229. In some embodiments, the V_{L}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 256.

In some embodiments, antibodies or binding fragments thereof are provided. In some embodiments, the antibodies or binding fragments thereof are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 37. In some embodiments, the V_{H}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 77. In some embodiments, the V_{H}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 118. In some embodiments, the V_{L}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 178. In some embodiments, the V_{L}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 229. In some embodiments, the V_{L}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to SEQ ID NO: 256.

In some embodiments, antibodies or binding fragments thereof are provided. In some embodiments, the antibodies or binding fragments thereof are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 0, 1, 2, 3, 4, 5, or 6 substitutions relative to SEQ ID NO: 37. In some embodiments, the V_{H}-CDR2 comprises an amino acid sequence having at least 0, 1, 2, 3, 4, 5, or 6 substitutions relative to SEQ ID NO: 77. In some embodiments, the V_{H}-CDR3 comprises an amino acid sequence having at least 0, 1, 2, 3, 4, 5, or 6 substitutions relative to SEQ ID NO: 118. In some embodiments, the V_{L}-CDR1 comprises an amino acid sequence having at least 0, 1, 2, 3, 4, 5, or 6 substitutions relative to SEQ ID NO: 178. In some embodiments, the V_{L}-CDR2 comprises an amino acid sequence having at least 0, 1, 2, 3, 4, 5, or 6 substitutions relative to SEQ ID NO: 229. In some embodiments, the V_{L}-CDR3 comprises an amino acid sequence having at least 0, 1, 2, 3, 4, 5, or 6 substitutions relative to SEQ ID NO: 256.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 814-820, 1067-1109. In some embodiments, the light chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 829-835, 1110-1152.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 814-820, 1067-1109. In some embodiments, the light chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 829-835, 1110-1152.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 844-850, 1153-1195. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 859-865, 1196-1238.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 844-850, 1153-1195. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 859-865, 1196-1238.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 1415-1439. In some embodiments, the light chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 1440-1464.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 1415-1439. In some embodiments, the light chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 1440-1464.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 1465-1489. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the sequences of SEQ ID NOs: 1490-1514.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 1465-1489. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to any one of the sequences of SEQ ID NOs: 1490-1514.

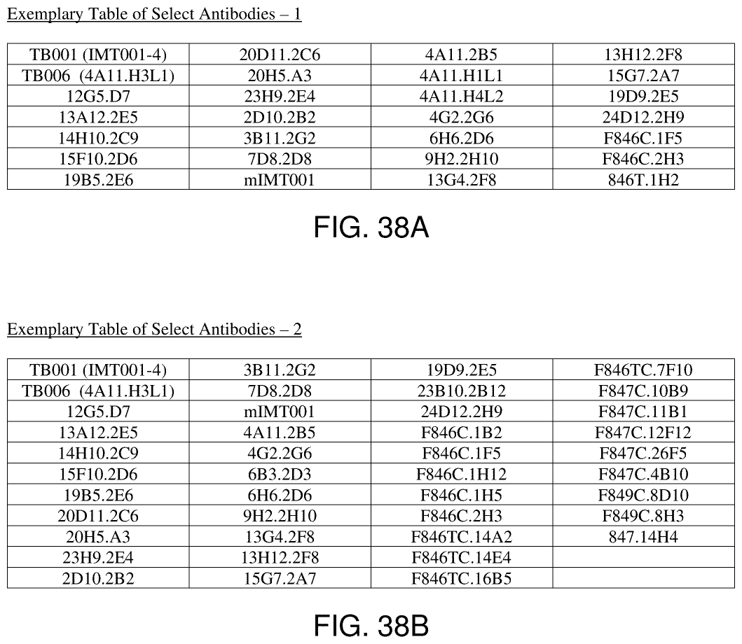

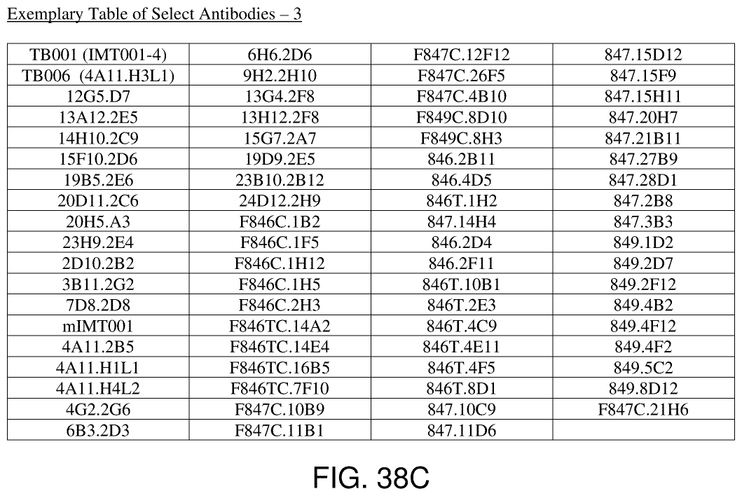

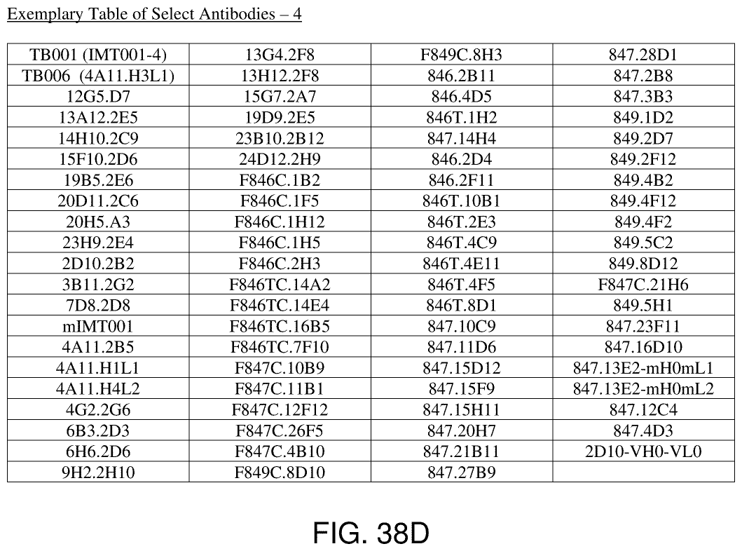

In some embodiments, the anti-Gal3 antibody or binding fragment thereof excludes the subset of named anti-Gal3 antibodies or binding fragments thereof depicted in any one of FIGS. 38A-D. In some embodiments, CDRs (including heavy chain and light chain CDRs), heavy chain variable regions, light chain variable regions, heavy chains, and/or light chains are excluded from the sequences associated with the subset of named anti-Gal3 antibodies or binding fragments thereof depicted in any one of FIGS. 38A-D. In some embodiments, combinations of CDRs (including heavy chain and light chain CDRs), heavy chain variable regions, light chain variable regions, heavy chains, and/or light chains are excluded from the sequences associated with the subset of named anti-Gal3 antibodies or binding fragments thereof depicted in any one of FIGS. 38A-D. In some embodiments, the anti-Gal3 antibody or binding fragment thereof excludes those selected from the group of named anti-Gal3 antibody or binding fragment thereof depicted in FIG. 38A, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 38B, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 38C, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 38D, or excludes any one or more of the CDRs, VH, VL, HC, and/or LC thereof. In some embodiments, the anti-Gal3 antibody or binding fragment thereof blocks the interaction between Gal3 and GLUT (e.g., GLUT1 and/or GLUT4) and excludes those selected from the group of named anti-Gal3 antibody or binding fragment thereof depicted in FIG. 38A, or excludes any one or more of the CDRs, VH, VL, HC, and/or LC thereof. In some embodiments, the anti-Gal3 antibody or binding fragment thereof blocks the interaction between Gal3 and GLUT (e.g., GLUT1 and/or GLUT4) and excludes those selected from the group of named anti-Gal3 antibody or binding fragment thereof depicted in FIG. 38B, or excludes any one or more of the CDRs, VH, VL, HC, and/or LC thereof. In some embodiments, the anti-Gal3 antibody or binding fragment thereof blocks the interaction between Gal3 and GLUT (e.g., GLUT1 and/or GLUT4) and excludes those selected from the group of named anti-Gal3 antibody or binding fragment thereof depicted in FIG. 38C, or excludes any one or more of the CDRs, VH, VL, HC, and/or LC thereof. In some embodiments, the anti-Gal3 antibody or binding fragment thereof blocks the interaction between Gal3 and GLUT (e.g., GLUT1 and/or GLUT4) and excludes those selected from the group of named anti-Gal3 antibody or binding fragment thereof depicted in FIG. 38D, or excludes any one or more of the CDRs, VH, VL, HC, and/or LC thereof. In other embodiments, any of these constructs are used for any of the methods provided herein.

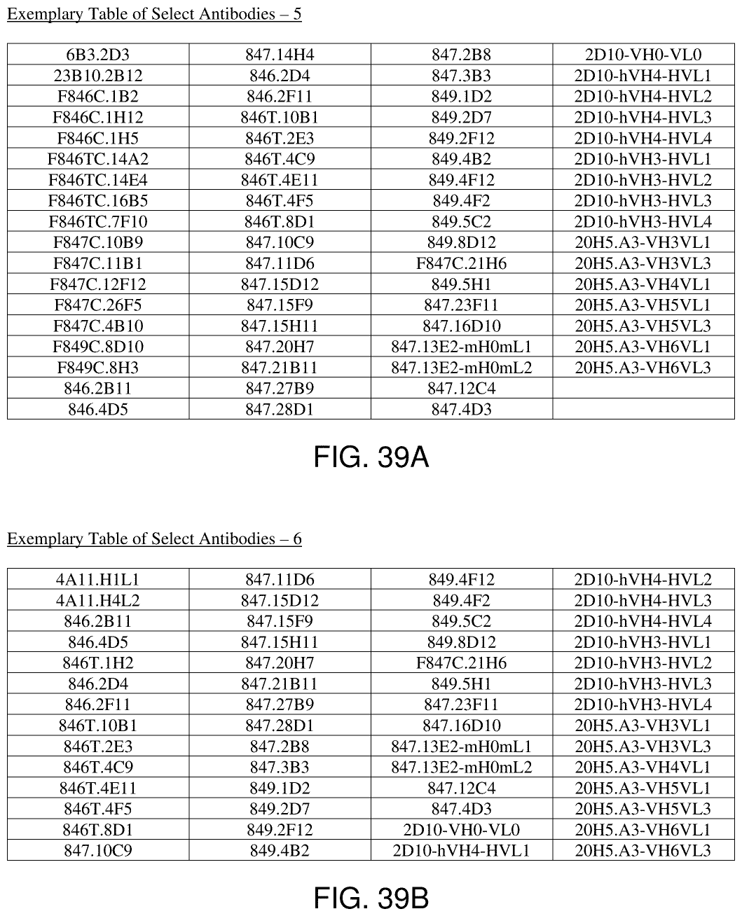

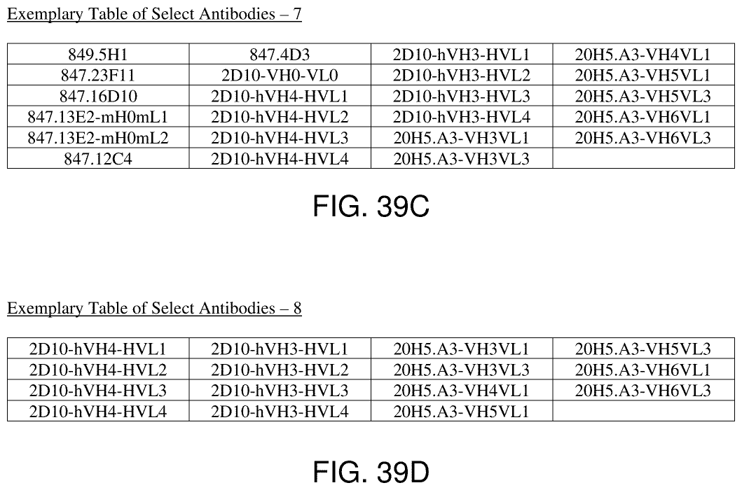

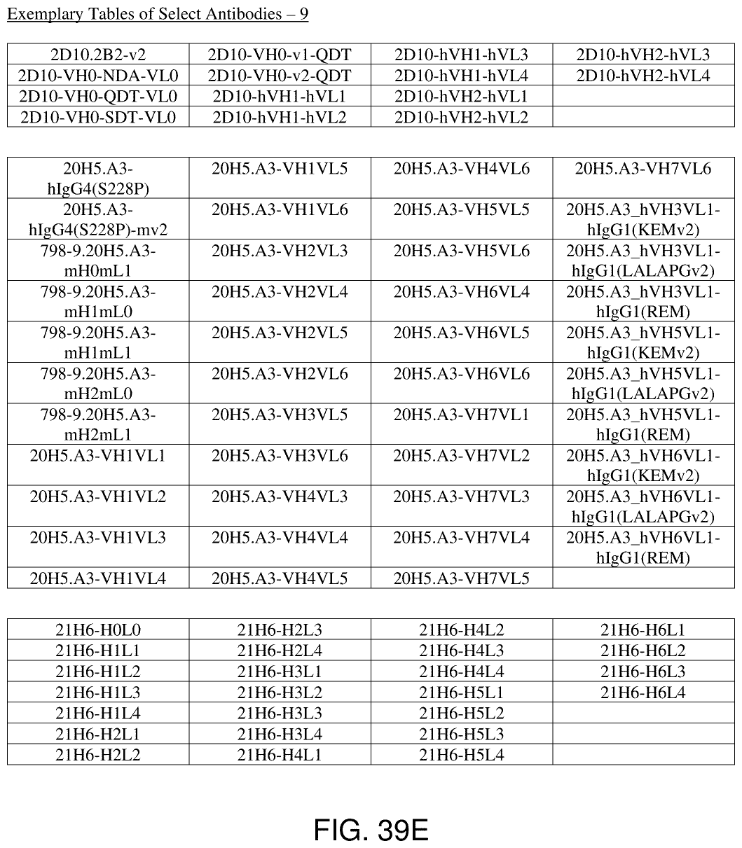

In some embodiments, the anti-Gal3 antibody or binding fragment thereof is selected from the subset of named anti-Gal3 antibodies or binding fragments thereof depicted in any one of FIGS. 39A-E. In some embodiments, CDRs (including heavy chain and light chain CDRs), heavy chain variable regions, light chain variable regions, heavy chains, and/or light chains are selected from the sequences associated with the subset of named anti-Gal3 antibodies or binding fragments thereof depicted in any one of FIGS. 39A-E. In some embodiments, combinations of CDRs (including heavy chain and light chain CDRs), heavy chain variable regions, light chain variable regions, heavy chains, and/or light chains are selected from the sequences associated with the subset of named anti-Gal3 antibodies or binding fragments thereof depicted in any one of FIGS. 39A-E. In some embodiments, the anti-Gal3 antibody or binding fragment thereof is selected from the group of named anti-Gal3 antibody or binding fragment thereof depicted in FIG. 39A, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 39B, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 39C, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 39D, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 39E, or comprises any one or more of the CDRs, VH, VL, HC, and/or LC thereof.

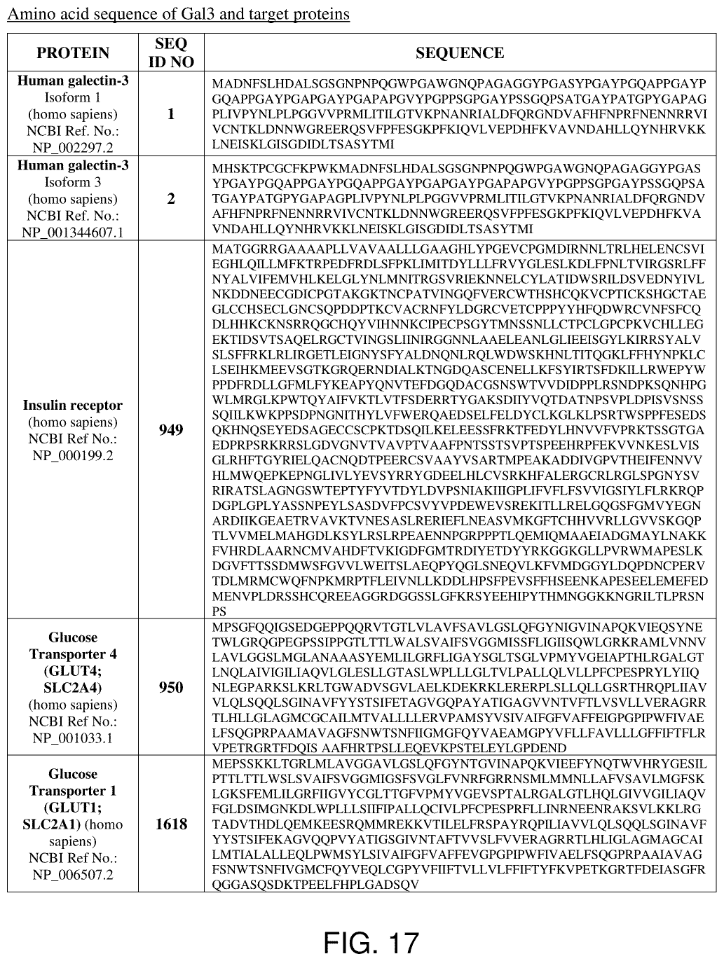

In some embodiments, the anti-Gal3 antibody or binding fragment thereof binds to specific epitopes within a Gal3 protein. In some embodiments, the anti-Gal3 antibody or binding fragment thereof binds to a specific epitope within a Gal3 protein having an amino acid sequence according to SEQ ID NO: 1-2, provided in FIG. 17.

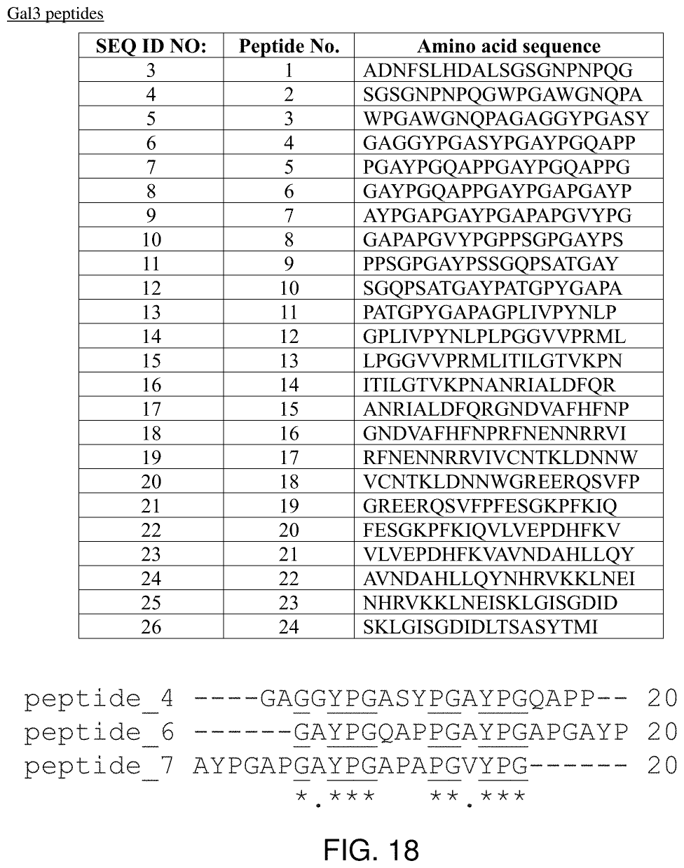

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within a peptide illustrated in FIG. 18 (SEQ ID NOs: 3-26).

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 1-20 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 31-50 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 51-70 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 61-80 of SEQ ID NO: 1-2.

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 1 (SEQ ID NO: 3), Peptide 4 (SEQ ID NO: 6), Peptide 6 (SEQ ID NO: 8), or Peptide 7 (SEQ ID NO: 9). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 1 (SEQ ID NO: 3). In some embodiments, the anti-Gal3 antibody or binding fragment thereof may bind to at least 11, 2, 3, 4, 5, 6, 7, 8, 9, 10, 11, 12, 13, 14, 15, 16, 17, 18, 19, or 20 amino acid residues within Peptide 4 (SEQ ID NO: 6). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 6 (SEQ ID NO: 8). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 7 (SEQ ID NO: 9). In some embodiments, the anti-Gal3 antibody or binding fragment thereof binds to an epitope present within a region of Gal3 defined by Peptide 1 (SEQ ID NO: 3). In some embodiments, the anti-Gal3 antibody or binding fragment thereof binds to an epitope present within a region of Gal3 defined by Peptide 4 (SEQ ID NO: 6). In some embodiments, the anti-Gal3 antibody or binding fragment thereof binds to an epitope present within a region of Gal3 defined by Peptide 6 (SEQ ID NO: 8). In some embodiments, the anti-Gal3 antibody or binding fragment thereof binds to an epitope present within a region of Gal3 defined by Peptide 7 (SEQ ID NO: 9). In some embodiments, the antibody is one that binds to 1, 2, or all 3 of peptides 1, 6, and/or 7.

In some embodiments, an anti-Gal3 antibody or binding fragment thereof as described herein may bind to the N-terminal domain of Gal3 or a portion thereof. In some embodiments, an anti-Gal3 antibody or binding fragment thereof as described herein may bind to an epitope of Gal3 that includes a motif of GxYPG, where x is the amino acids alanine (A), glycine (G), or valine (V). In some embodiments, an anti-Gal3 antibody or binding fragment thereof as described herein may bind to an epitope of Gal3 that includes two GxYPG motifs separated by three amino acids, where x is A, G, or V.

In some embodiments, the anti-Gal3 antibody or binding fragment thereof binds to Gal3. In some embodiments, the anti-Gal3 antibody or binding fragment thereof binds to the N-terminus of Gal3, the N-terminal domain of Gal3, or the TRD of Gal3. In some embodiments, the anti-Gal3 antibody or binding fragment thereof does not bind to the N-terminus of Gal3, the N-terminal domain of Gal3, or the TRD of Gal3. In some embodiments, the anti-Gal3 antibody or binding fragment thereof binds to the C-terminus of Gal3, the C-terminal domain of Gal3, or the CRD of Gal3. In some embodiments, the anti-Gal3 antibody or binding fragment thereof does not bind to the C-terminus of Gal3, the C-terminal domain of Gal3, or the CRD of Gal3. In some embodiments, the anti-Gal3 antibody or binding fragment thereof binds to Gal3 isoform 1. In some embodiments, the anti-Gal3 antibody or binding fragment thereof binds to the N-terminus of Gal3 isoform 1, the N-terminal domain of Gal3 isoform 1, amino acids 1-111 of Gal3 isoform 1, the TRD of Gal3 isoform 1, or amino acids 36-109 of Gal3 isoform 1. In some embodiments, the anti-Gal3 antibody or binding fragment thereof does not bind to the N-terminus of Gal3 isoform 1, the N-terminal domain of Gal3 isoform 1, amino acids 1-111 of Gal3, the TRD of Gal3 isoform 1, or amino acids 36-109 of Gal3 isoform 1. In some embodiments, the anti-Gal3 antibody or binding fragment thereof binds to the C-terminus of Gal3 isoform 1, the C-terminal domain of Gal3 isoform 1, amino acids 112-250 of Gal3, or the CRD of Gal3. In some embodiments, the anti-Gal3 antibody or binding fragment thereof does not bind to the C-terminus of Gal3 isoform 1, the C-terminal domain of Gal3 isoform 1, amino acids 112-250 of Gal3 isoform 1, or the CRD of Gal3. In some embodiments, the anti-Gal3 antibody or binding fragment thereof binds to the N-terminus of Gal3 isoform 3, the N-terminal domain of Gal3 isoform 3, amino acids 1-125 of Gal3, the TRD of Gal3 isoform 3, or amino acids 50-123 of Gal3 isoform 3. In some embodiments, the anti-Gal3 antibody or binding fragment thereof does not bind to the N-terminus of Gal3 isoform 3, the N-terminal domain of Gal3 isoform 3, amino acids 1-125 of Gal3 isoform 3, the TRD of Gal3, or amino acids 50-123 of Gal3 isoform 3. In some embodiments, the anti-Gal3 antibody or binding fragment thereof binds to the C-terminus of Gal3 isoform 3, the C-terminal domain of Gal3 isoform 3, amino acids 126-264 of Gal3 isoform 3, or the CRD of Gal3. In some embodiments, the anti-Gal3 antibody or binding fragment thereof does not bind to the C-terminus of Gal3 isoform 3, the C-terminal domain of Gal3 isoform 3, amino acids 126-264 of Gal3 isoform 3, or the CRD of Gal3 isoform 3.

In some embodiments, the interaction between Gal3 and a cell surface marker can be reduced to less than 80%, less than 75%, less than 70%, less than 60%, less than 59%, less than 50%, less than 40%, less than 34%, less than 30%, less than 20%, less than 14%, less than 10%, less than 7%, less than 5%, less than 4%, or less than 1%.

In some embodiments, the anti-Gal3 antibody or binding fragment thereof binds to Gal3 with a dissociation constant (K_{D}) of less than 1 nM, less than 1.2 nM, less than 2 nM, less than 5 nM, less than 10 nM, less than 13.5 nM, less than 15 nM, less than 20 nM, less than 25 nM, or less than 30 nM. In some embodiments, the anti-Gal3 antibody or binding fragment thereof binds to Gal3 with a K_{D} of less than 1 nM. In some embodiments, the anti-Gal3 antibody or binding fragment thereof binds to Gal3 with a K_{D} of less than 1.2 nM. In some embodiments, the anti-Gal3 antibody or binding fragment thereof binds to Gal3 with a K_{D} of less than 2 nM. In some embodiments, the anti-Gal3 antibody or binding fragment thereof binds to Gal3 with a K_{D} of less than 5 nM. In some embodiments, the anti-Gal3 antibody or binding fragment thereof binds to Gal3 with a K_{D} of less than 10 nM. In some embodiments, the anti-Gal3 antibody or binding fragment thereof binds to Gal3 with a K_{D} of less than 13.5 nM. In some embodiments, the anti-Gal3 antibody or binding fragment thereof binds to Gal3 with a K_{D} of less than 15 nM. In some embodiments, the anti-Gal3 antibody or binding fragment thereof binds to Gal3 with a K_{D} of less than 20 nM. In some embodiments, the anti-Gal3 antibody or binding fragment thereof binds to Gal3 with a K_{D} of less than 25 nM. In some embodiments, the anti-Gal3 antibody or binding fragment thereof binds to Gal3 with a K_{D} of less than 30 nM.

In some embodiments, the anti-Gal3 antibody or binding fragment thereof comprises any one of the variable heavy chain complementarity-determining region 1 (V_{H}-CDR1) sequences illustrated in FIG. 19A (SEQ ID NOs: 27-70). In some embodiments, the anti-Gal3 antibody comprises a V_{H}-CDR1 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 27-70. In some embodiments, the anti-Gal3 antibody comprises a V_{H}-CDR1 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 27-70.

In some embodiments, the anti-Gal3 antibody or binding fragment thereof comprises any one of the variable heavy chain complementarity-determining region 2 (V_{H}-CDR2) sequences illustrated in FIG. 19B (SEQ ID NOs: 71-111, 801, 951, 952). In some embodiments, the anti-Gal3 antibody or binding fragment thereof comprises a V_{H}-CDR2 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 71-111, 801, 951, 952. In some embodiments, the anti-Gal3 antibody or binding fragment thereof comprises a V_{H}-CDR2 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 71-111, 801, 951, 952.

In some embodiments, the anti-Gal3 antibody or binding fragment thereof comprises any one of the variable heavy chain complementarity-determining region 3 (V_{H}-CDR3) sequences illustrated in FIG. 19C (SEQ ID NOs: 112-169, 802, 953, 954). In some embodiments, the anti-Gal3 antibody or binding fragment thereof comprises a V_{H}-CDR3 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 112-169, 802, 953, 954. In some embodiments, the anti-Gal3 antibody or binding fragment thereof comprises a V_{H}-CDR3 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 112-169, 802, 953, 954.

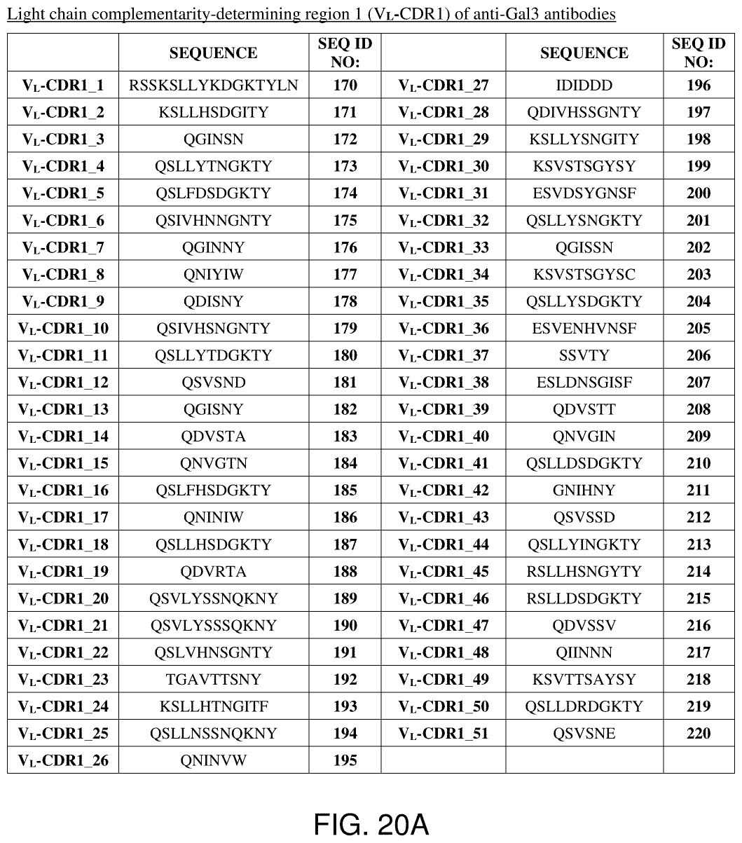

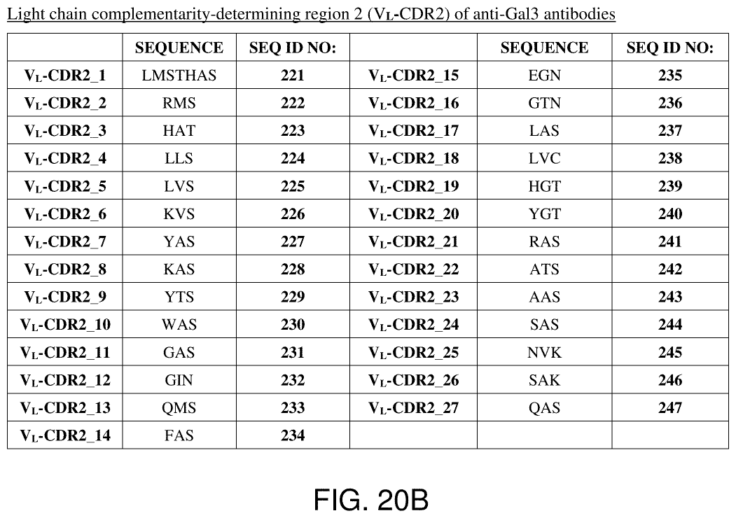

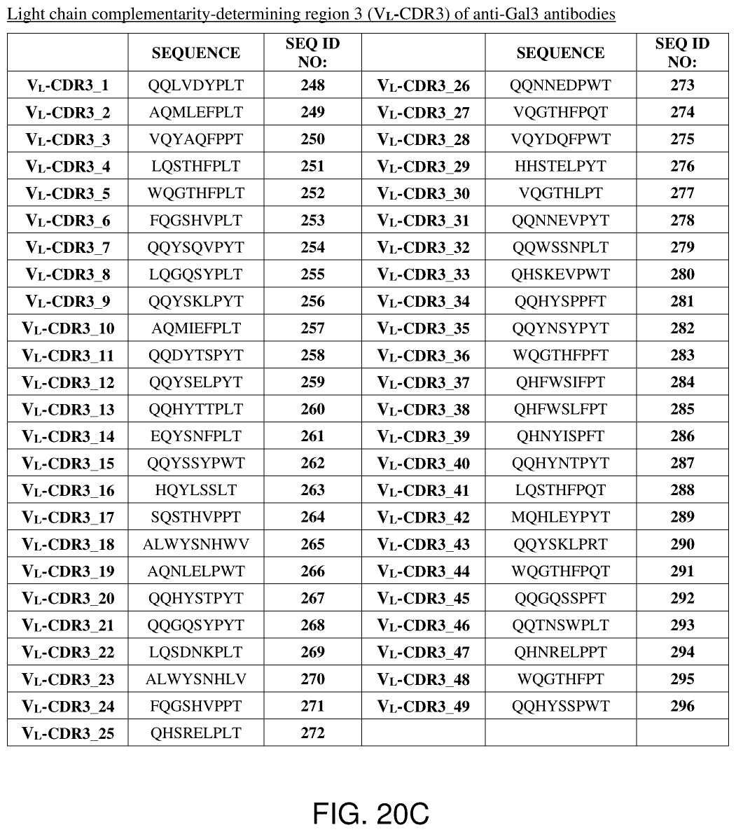

In some embodiments, the anti-Gal3 antibody or binding fragment thereof comprises any one of the variable light chain complementarity-determining region 1 (V_{L}—CDR1) sequences illustrated in FIG. 20A (SEQ ID NOs: 170-220). In some embodiments, the anti-Gal3 antibody or binding fragment thereof comprises a V_{L}-CDR1 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 170-220. In some embodiments, the anti-Gal3 antibody or binding fragment thereof comprises a V_{L}-CDR1 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 170-220.

In some embodiments, the anti-Gal3 antibody or binding fragment thereof comprises any one of the variable light chain complementarity-determining region 2 (V_{L}—CDR2) sequences illustrated in FIG. 20B (SEQ ID NOs: 221-247). In some embodiments, the anti-Gal3 antibody or binding fragment thereof comprises a V_{L}-CDR2 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 221-247. In some embodiments, the anti-Gal3 antibody or binding fragment thereof comprises a V_{L}-CDR2 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 221-247.

In some embodiments, the anti-Gal3 antibody or binding fragment thereof comprises any one of the variable light chain complementarity-determining region 3 (VL-CDR3) sequences illustrated in FIG. 20C (SEQ ID NOs: 248-296). In some embodiments, the anti-Gal3 antibody or binding fragment thereof comprises a V_{L}-CDR3 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 248-296. In some embodiments, the anti-Gal3 antibody or binding fragment thereof comprises a V_{L}-CDR3 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 248-296.

In some embodiments, the anti-Gal3 antibody or binding fragment thereof comprises a heavy chain variable region (V_{H}) and a light chain variable region (V_{L}). In some embodiments, the V_{H} may comprise a V_{H}-CDR1, a V_{H}-CDR2, and/or a V_{H}-CDR3 selected from any of FIG. 19A-C. In some embodiments, the V_{L} may comprise a V_{L}-CDR1, a V_{L}-CDR2, and/or a V_{L}-CDR3 selected from any of FIG. 20A-C. In some embodiments, the anti-Gal3 antibody or binding fragment thereof comprises CDRs within the V_{H} and V_{L} sequences as illustrated in FIGS. 21 and 22.

In some embodiments, the anti-Gal3 antibody or binding fragment thereof comprises a heavy chain variable region (V_{H}) sequence selected from FIG. 21 (SEQ ID NOs: 297-373, 803, 806-820, 940, 955-968, 1067-1109, 1415-1439). In some embodiments, the anti-Gal3 antibody or binding fragment thereof comprises a V_{H}- sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 297-373, 803, 806-820, 940, 955-968, 1067-1109, 1415-1439. In some embodiments, the anti-Gal3 antibody or binding fragment thereof comprises a V_{H}- sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 297-373, 803, 806-820, 940, 955-968, 1067-1109, 1415-1439.

In some embodiments, the anti-Gal3 antibody or binding fragment thereof comprises a light chain variable region (V_{L}) sequence selected from FIG. 22 (SEQ ID NOs: 374-447, 821-835, 941-943, 969-982, 1110-1152, 1440-1464). In some embodiments, the anti-Gal3 antibody or binding fragment thereof comprises a V_{L} sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 374-447, 821-835, 941-943, 969-982, 1110-1152, 1440-1464. In some embodiments, the anti-Gal3 antibody or binding fragment thereof comprises a VL sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 374-447, 821-835, 941-943, 969-982, 1110-1152, 1440-1464.

In some embodiments, the anti-Gal3 antibody or binding fragment thereof comprises a combination of heavy chain variable region and light chain variable region as illustrated in FIG. 24.

In some embodiments, the anti-Gal3 antibody or binding fragment thereof comprises heavy chain and light chain sequences as illustrated in FIG. 25 (SEQ ID NOs: 448-538, 804-805, 836-865, 983-1010,1153-1238, 1465-1514.

In some embodiments, the anti-Gal3 antibody or binding fragment thereof is selected from the group of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or a binding fragment thereof.

In some embodiments, the anti-Gal3 antibody or binding fragment thereof comprises, consists essentially of, or consists of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or a binding fragment thereof.

In some embodiments, the anti-Gal3 antibody or binding fragment thereof is selected from the group of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or a binding fragment thereof.

In some embodiments, the anti-Gal3 antibody or binding fragment thereof comprises, consists essentially of, or consists of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or a binding fragment thereof.

In some embodiments, the anti-Gal3 antibody or binding fragment thereof comprises one or more heavy chain variable region CDRs depicted in FIG. 19A-C. In some embodiments, the anti-Gal3 antibody or binding fragment thereof comprises one or more light chain variable region CDRs depicted in FIG. 20A-C. In some embodiments, the anti-Gal3 antibody or binding fragment thereof comprises a heavy chain variable region depicted in FIG. 21. In some embodiments, the anti-Gal3 antibody or binding fragment thereof comprises a light chain variable region depicted in FIG. 22. In some embodiments, the anti-Gal3 antibody or binding fragment thereof comprises a combination of heavy chain variable region and light chain variable region depicted in FIG. 24. In some embodiments, the anti-Gal3 antibody or binding fragment thereof comprises a heavy chain and/or light chain depicted in FIG. 25. In some embodiments, the anti-Gal3 antibody or binding fragment thereof can comprise or include any one or more of the sequences provided in any one or more of FIG. 19A-C, 20A-C, 21, 22, 23, 24, 25, or any one or more of a sequence that is at least 80%, 81%, 82%, 83%, 84%, 85%, 86%, 87%, 88%, 89%, 90%, 91%, 92%, 93%, 94%, 95%, 96%, 97%, 98%, 99% or greater identical thereto. In some embodiments, the anti-Gal3 antibody or binding fragment thereof can comprise or include any one or more of the sequences provided in any one or more of FIG. 19A-C, 20A-C, 21, 22, 23, 24, 25, or any one or more of a sequence that is at least 80%, 81%, 82%, 83%, 84%, 85%, 86%, 87%, 88%, 89%, 90%, 91%, 92%, 93%, 94%, 95%, 96%, 97%, 98%, 99% or greater similar thereto.

In some embodiments, the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a VL-CDR3. In some embodiments, the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 32, 37, or 66. In some embodiments, the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 801, 951, 952, 77, or 108. In some embodiments, the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 953, 954, 802, 118, or 164. In some embodiments, the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 171, 178, or 215. In some embodiments, the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 222, 229, or 225. In some embodiments, the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 257, 256, or 291.

In some embodiments, the heavy chain variable region comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 806-820, 955-968, 1067-1109, 1415-1439. In some embodiments, the light chain variable region comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 821-835, 969-982, 1110-1152, 1440-1464.

In some embodiments, the anti-Gal3 antibody or binding fragment thereof comprises a heavy chain, wherein the heavy chain comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 836-850,983-996, 1153-1195, 1411, 1465-1489. In some embodiments, the anti-Gal3 antibody or binding fragment thereof comprises a light chain, wherein the light chain comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 851-865, 997-1010, 1196-1238, 1412, 1490-1514.

In some embodiments, the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

In some embodiments, the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), or binding fragment thereof.

In some embodiments, the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof.

In some embodiments, the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, or binding fragment thereof.

In some embodiments, the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

In some embodiments, the anti-Gal3 antibody or binding fragment thereof comprises a humanized antibody or binding fragment thereof. In other instances, the anti-Gal3 antibody or binding fragment thereof comprises a chimeric antibody or binding fragment thereof. In some embodiments, the anti-Gal3 antibody comprises a full-length antibody or a binding fragment thereof. In some embodiments, the anti-Gal3 antibody or binding fragment thereof comprises a bispecific antibody or a binding fragment thereof. In some embodiments, the anti-Gal3 antibody or binding fragment thereof comprises a monovalent Fab′, a divalent Fab2, a single-chain variable fragment (scFv), a diabody, a minibody, a nanobody, a single-domain antibody (sdAb), or a camelid antibody or binding fragment thereof.

In some embodiments, the anti-Gal3 antibody or binding fragment thereof is a bispecific antibody or binding fragment thereof. Exemplary bispecific antibody formats include, but are not limited to, Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), Azymetric, Biclonics, Fab-scFv-Fc, Two-in-one/Dual Action Fab (DAF), FinomAb, scFv-Fc-(Fab)-fusion, Dock-aNd-Lock (DNL), Tandem diAbody (TandAb), Dual-affinity-ReTargeting (DART), nanobody, triplebody, tandems scFv (taFv), triple heads, tandem dAb/VHH, triple dAb/VHH, or tetravalent dAb/VHH. In some embodiments, the anti-Gal3 antibody or binding fragment thereof is a bispecific antibody or binding fragment thereof comprising a bispecific antibody format illustrated in Brinkmann and Kontermann, “The making of bispecific antibodies,” MABS 9(2): 182-212 (2017).

In some embodiments, the anti-Gal3 antibody or binding fragment thereof can comprise an IgM, IgG (e.g., IgG1, IgG2, IgG3, or IgG4), IgA, or IgE framework. The IgG framework can be IgG1, IgG2, IgG3 or IgG4. In some embodiments, the anti-Gal3 antibody or binding fragment thereof comprises an IgG1 framework. In some embodiments, the anti-Gal3 antibody or binding fragment thereof comprises an IgG2 framework. In some embodiments, the anti-Gal3 antibody or binding fragment thereof comprises an IgG4 framework. The anti-Gal3 antibody or binding fragment thereof can further comprise a Fc mutation.

In some embodiments, the Fc region comprises one or more mutations that modulate Fc receptor interactions, e.g., to enhance effector functions such as ADCC and/or CDC. In such instances, exemplary residues when mutated modulate effector functions include S239, K326, A330, 1332, or E333, in which the residue position correspond to IgG1 and the residue numbering is in accordance to Kabat numbering (EU index of Kabat et al 1991 Sequences of Proteins of Immunological Interest). In some embodiments, the one or more mutations comprise S239D, K326W, A330L, 1332E, E333A, E333S, or a combination thereof. In some embodiments, the one or more mutations comprise S239D, 1332E, or a combination thereof. In some embodiments, the one or more mutations comprise S239D, A330L, 1332E, or a combination thereof. In some embodiments, the one or more mutations comprise K326W, E333S, or a combination thereof. In some embodiments, the mutation comprises E333A.

In some embodiments, an anti-Gal3 antibody or binding fragment thereof comprises a humanization score of above 70, above 80, above 81, above 82, above 83, above 84, above 85, above 86, above 87, above 88, above 89, above 90, or above 95. In some embodiments, the anti-Gal3 antibody or binding fragment thereof comprises a humanization score of above 80. In some embodiments, the anti-Gal3 antibody or binding fragment thereof comprises a humanization score of above 83. In some embodiments, the anti-Gal3 antibody or binding fragment thereof comprises a humanization score of above 85. In some embodiments, the anti-Gal3 antibody or binding fragment thereof comprises a humanization score of above 87. In some embodiments, the anti-Gal3 antibody or binding fragment thereof comprises a humanization score of above 90. In some embodiments, the anti-Gal3 antibody or binding fragment thereof comprises a humanization score of the heavy chain of above 70, above 80, above 81, above 82, above 83, above 84, above 85, above 86, above 87, above 88, above 89, above 90, or above 95, optionally above 80, above 85, or above 87. In some embodiments, the anti-Gal3 antibody or binding fragment thereof comprises a humanization score of the light chain of above 70, above 80, above 81, above 82, above 83, above 84, above 85, above 86, above 87, above 88, above 89, above 90, or above 95, optionally above 80, above 83, or above 85.

Also disclosed herein are proteins. In some embodiments, the proteins comprise one or more of SEQ ID NOs: 27-538, 801-865, 955-1010, 1067-1238, 1415-1514. In some embodiments, the proteins comprise a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of SEQ ID NOs: 27-538, 801-865, 955-1010, 1067-1238, 1415-1514. In some embodiments, the proteins comprise a sequence having at least 0, 1, 2, 3, 4, 5, or 6 substitutions relative to any one or more sequences of SEQ ID NOs: 27-538, 801-865, 955-1010, 1067-1238, 1415-1514. In some embodiments, the proteins comprise six sequences selected from each of SEQ ID NOs: 27-70; SEQ ID NOs: 71-111, 801, 951, 952; SEQ ID NOs: 112-169, 802, 953, 954; SEQ ID NOs: 170-220; SEQ ID NOs: 221-247; SEQ ID NOs: 248-296. In some embodiments, the proteins comprise two sequences selected from each of SEQ ID NOs: 279-373, 803, 806-820, 955-968, 1067-1190, 1415-1439 and SEQ ID NOs: 374-447, 821-835, 969-982, 1110-1152, 1440-1464. In some embodiments, the proteins comprise two sequences selected from each of SEQ ID NOs: 448-494, 804, 836-850, 983-996, 1153-1195, 1465-1489 and SEQ ID NOs: 495-538, 805, 851-865, 997-1010, 1196-1238, 1490-1514. In some embodiments, the proteins comprise any one or more of the sequences depicted in FIGS. 19A-C, 20A-C, 21, 22, 23, 24, 25.

In some embodiments, the protein comprises one or more sequences defined by a consensus sequence. The consensus sequences provided herein have been derived from the alignments of CDRs depicted in FIG. 37A-B. However, it is envisioned that alternative alignments may be done (e.g. using global or local alignment, or with different algorithms, such as Hidden Markov Models, seeded guide trees, Needleman-Wunsch algorithm, or Smith-Waterman algorithm) and as such, alternative consensus sequences can be derived.

In some embodiments, the protein comprises a sequence defined by the formula X₁X₂X₃X₄X₅X₆X₇X₈X₉X₁₀X₁₁X₁₂X₁₃X₁₄X₁₅X₁₆X₁₇, where X₁ is no amino acid or R; X₂ is no amino acid or S; X₃ is no amino acid, S, or T; X₄ is no amino acid, E, G, K, Q, or R; X₅ is no amino acid, A, D, G, I, N, or S; X₆ is no amino acid, I, L, or V; X₇ is no amino acid, F, L, S, or V; X₈ is no amino acid, D, E, H, N, S, T, or Y; X₉ is no amino acid, D, E, I, K, N, R, S, T, or V; X₁₀ is no amino acid, D, H, N, R, S, or Y; X₁₁ is no amino acid, A, G, N, S, T, or V; X₁₂ is no amino acid, A, I, K, N, Q, T, V, or Y; X₁₃ is no amino acid, D, G, H, K, N, S, T, or Y; X₁₄ is no amino acid, C, F, I, N, S, T, V, or Y; X₁₅ is no amino acid, D, L, N, W, or Y; X₁₆ is no amino acid, N, or D; X₁₇ is no amino acid or D. In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence.

In some embodiments, the protein comprises a sequence defined by the formula X₁X₂X₃X₄X₅X₆X₇X₈, where X₁ is no amino acid, K, L, N, Q, or R; X₂ is no amino acid, A, L, M, or V; X₃ is no amino acid, C, K, or S; X₄ is no amino acid or T; X₅ is no amino acid, A, E, F, G, H, K, Q, R, S, W, or Y; X₆ is no amino acid, A, G, or T; X₇ is no amino acid, I, K, N, S, or T; X₈ is no amino acid, N, or S. In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence.

In some embodiments, the protein comprises a sequence defined by the formula X₁X₂X₃X₄X₅X₆X₇X₈X₉X₁₀, where X₁ is no amino acid, A, E, F, H, L, M, Q, S, V, or W; X₂ is A, H, or Q; X₃ is D, F, G, H, L, M, N, Q, S, T, W, or Y; X₄ is no amino acid or W; X₅ is A, D, I, K, L, N, Q, R, S, T, V, or Y; X₆ is D, E, H, I, K, L, N, Q, S, or T; X₇ is D, F, K, L, N, P, S, T, V, W, or Y; X₈ is H, P, or S; X₉ is F, L, P, Q, R, T, W, or Y; X₁₀ is no amino acid, T, or V. In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence.

In some embodiments, the protein comprises a sequence defined by the formula X₁X₂X₃X₄X₅X₆X₇X₈X₉X₁₀, where X₁ is E, G, or R; X₂ is F, N, or Y; X₃ is A, I, K, N, S, or T; X₄ is F, I, or L; X₅ is I, K, N, R, S, or T; X₆ is D, G, I, N, S, or T; X₇ is F, G, H, S, or Y; X₈ is no amino acid, A, D, G, I, M, N, T, V, W, or Y; X₉ is no amino acid, M, or Y; X₁₀ is no amino acid or G; In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence.

In some embodiments, the protein comprises a sequence defined by the formula X₁X₂X₃X₄X₅X₆X₇X₈X₉X₁₀, where X₁ is no amino acid, I, or L; X₂ is no amino acid or R; X₃ is no amino acid, F, I, L, or V; X₄ is A, D, F, H, K, L, N, S, W, or Y; X₅ is A, D, P, S, T, W, or Y; X₆ is D, E, G, H, K, N, S, V, or Y; X₇ is D, E, G, N, S, or T; X₈ is D, G, I, K, N, Q, R, S, V, or Y; X₉ is A, D, E, G, I, K, N, P, S, T, V, or Y; X₁₀ is no amino acid, I, P, S, or T. In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence.

In some embodiments, the protein comprises a sequence defined by the formula X₁X₂X₃X₄X₅X₆X₇X₈X₉X₁₀X₁₁X₁₂X₁₃X₁₄X₅X₁₆X₁₇X₁₈X₁₉X₂₀X₂₁X₂₂X₂₃X₂₄X₂₅, where X₁ is no amino acid or A; X₂ is no amino acid, A, R, or Y; X₃ is no amino acid, A, F, H, K, L, R, S, or V; X₄ is no amino acid, A, D, K, N, R, S, or T; X₅ is no amino acid, A, D, G, H, I, L, N, P, R, S, T, V, or Y; X₆ is no amino acid, A, D, G, H, K, N, P, Q, R, S, or Y; X₇ is no amino acid, D, F, G, H, P, R, S, W, or Y; X₈ is no amino acid, A, D, E, G, I, R, or S; X₉ is no amino acid, A, C, D, E, F, G, I, N, R, S, T, V, or Y; X₁₀ is no amino acid, A, D, M, P, R, S, T, V, or Y; X₁₁ is no amino acid, A, D, E, F, L, T, V, or Y; X₁₂ is no amino acid, A, G, L, M, R, or T; X₁₃ is no amino acid, A, D, E, F, G, R, S, T, or V; X₁₄ is no amino acid, A, D, G, L, P, Q, R, S, T, V, or Y; X₁₅ is no amino acid, A, D, G, N, S, V, W, or Y; X₁₆ is no amino acid, A, D, E, F, L, P, T, V, W, or Y; X₁₇ is no amino acid, F, I, L, M, R, or Y; X₁₈ is no amino acid, A, D, G, N, or T; X₁₉ is no amino acid, F, N, S, T, V, or Y; X₂₀ is no amino acid or L; X₂₁ is no amino acid or A; X₂₂ is no amino acid or W; X₂₃ is no amino acid or F; X₂₄ is no amino acid or A; X₂₅ is no amino acid or Y. In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence.

### Pharmaceutical Formulations

A pharmaceutical formulation for treating a disease as described herein can comprise an anti-Gal3 antibody or binding fragment thereof described supra. The anti-Gal3 antibody or binding fragment thereof can be formulated for systemic administration. Alternatively, the anti-Gal3 antibody or binding fragment thereof can be formulated for parenteral administration.

In some embodiments, an anti-Gal3 antibody or binding fragment thereof is formulated as a pharmaceutical composition for administration to a subject by, but not limited to, parenteral (e.g., intravenous, subcutaneous, intramuscular, intraarterial, intradermal, intraperitoneal, intravitreal, intracerebral, or intracerebroventricular), oral, intranasal, buccal, rectal, or transdermal administration routes. In some instances, the pharmaceutical composition describe herein is formulated for parenteral (e.g., intravenous, subcutaneous, intramuscular, intraarterial, intradermal, intraperitoneal, intravitreal, intracerebral, or intracerebroventricular) administration. In other instances, the pharmaceutical composition describe herein is formulated for systemic administration. In other instances, the pharmaceutical composition describe herein is formulated for oral administration. In still other instances, the pharmaceutical composition describe herein is formulated for intranasal administration.

In some instances, the pharmaceutical compositions further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical compositions further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation can further comprise an additional therapeutic agent. Non-limiting examples of additional therapeutic agents include alpha-glucosidase inhibitors, including acarbose (Precose®) and miglitol (Glyset®); biguanides, including metformin-alogliptin (Kazano®), metformin-canagliflozin (Invokamet®), metformin-dapagliflozin (Xigduo® XR), metformin-empagliflozin (Synjardy®), metformin-glipizide, metformin-glyburide (Glucovance®), metformin-linagliptin (Jentadueto®), metformin-pioglitazone (Actoplus®), metformin-repaglinide (PrandiMet®), metformin-rosiglitazone (Avandamet®), metformin-saxagliptin (Kombiglyze® XR), and metformin-sitagliptin (Janumet®); dopamine agonists, including Bromocriptine (Cycloset®); Dipeptidyl peptidase-4 (DPP-4) inhibitors, including alogliptin (Nesina®), alogliptin-metformin (Kazano®), alogliptin-pioglitazone (Oseni®), linagliptin (Tradjenta®), linagliptin-empagliflozin (Glyxambi®), linagliptin-metformin (Jentadueto®), saxagliptin (Onglyza®), saxagliptin-metformin (Kombiglyze® XR), sitagliptin (Januvia®), sitagliptin-metformin (Janumet® and Janumet® XR), and sitagliptin and simvastatin (Juvisync®); Glucagon-like peptide-1 receptor agonists (GLP-1 receptor agonists), including albiglutide (Tanzeum®), dulaglutide (Trulicity®), exenatide (Byetta®), exenatide extended-release (Bydureon®), and liraglutide (Victoza®), semaglutide (Ozempic®); Meglitinides, including nateglinide (Starlix®), repaglinide (Prandin®), and repaglinide-metformin (Prandimet®); Sodium-glucose transporter (SGLT) 2 inhibitors, including dapagliflozin (Farxiga®), dapagliflozin-metformin (Xigduo® XR), canagliflozin (Invokana®), canagliflozin-metformin (Invokamet®), empagliflozin (Jardiance®), empagliflozin-linagliptin (Glyxambi®), empagliflozin-metformin (Synjardy®), and ertugliflozin (Steglatro®); Sulfonylureas, including glimepiride (Amaryl®), glimepiride-pioglitazone (Duetact®), glimepiride-rosiglitazone (Avandaryl®), gliclazide, glipizide (Glucotrol®), glipizide-metformin (Metaglip®), glyburide (DiaBeta®, Glynase®, Micronase®), glyburide-metformin (Glucovance®), chlorpropamide (Diabinese®), tolazamide (Tolinase®), and tolbutamide (Orinase®, Tol-Tab®); Thiazolidinediones, including rosiglitazone (Avandia®), rosiglitazone-glimepiride (Avandaryl®), rosiglitazone-metformin (Amaryl M®), pioglitazone (Actos®), pioglitazone-alogliptin (Oseni®), pioglitazone-glimepiride (Duetact®), pioglitazone-metformin (Actoplus Met®, Actoplus Met® XR).

### Exemplary Methods of Use

Any of the anti-Gal3 antibodies or binding fragments thereof, or proteins, may be used in methods, as provided herein.

In some embodiments are disclosed methods of enhancing glucose transporter (GLUT) translocation in a cell. The methods comprise contacting the cell with an anti-Gal3 antibody or binding fragment thereof. In some embodiments, binding of the anti-Gal3 antibody or binding fragment thereof to Gal3 in the cell inhibits Gal3-mediated blocking of GLUT translocation. In some embodiments, the glucose transporter is glucose transporter 1 (GLUT1) and/or glucose transporter 4 (GLUT4). In some embodiments, the method is performed in vitro or in vivo. In some embodiments, GLUT translocation in the cell is enhanced by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% after contacting with the anti-Gal3 antibody or binding fragment thereof relative to a cell that is not contacted with the anti-Gal3 antibody or binding fragment thereof. In some embodiments, the anti-Gal3 antibody or binding fragment thereof selectively binds to an N-terminal domain of Gal3. In some embodiments, the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof. In some embodiments, the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3. In some embodiments, the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70. In some embodiments, the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952. In some embodiments, the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954. In some embodiments, the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220. In some embodiments, the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 221-247. In some embodiments, the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296. In some embodiments, the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 23. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 955-968, 1067-1109, 1415-1439. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943, 969-982, 1110-1152, 1440-1464. In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514. In some embodiments, the anti-Gal3 antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

In some embodiments are disclosed methods of enhancing glucose transporter (GLUT) translocation in a cell. The methods comprise contacting the cell with a preincubated complex of Gal3 and an anti-Gal3 antibody or binding fragment thereof. In some embodiments, the preincubated complex of Gal3 and the anti-Gal3 antibody or binding fragment thereof is prepared with Gal3 and the anti-Gal3 antibody or binding fragment thereof at a mass ratio of or of about 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, or 1:2.5, or any mass ratio within a range defined by any two of the aforementioned mass ratios. In some embodiments, the preincubated complex of Gal3 and the anti-Gal3 antibody or binding fragment is prepared with Gal3 at a concentration of or of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 μg/mL, or any concentration within a range defined by any two of the aforementioned concentrations, and the anti-Gal3 antibody or binding fragment thereof at a concentration of or of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL, or any concentration within a range defined by any two of the aforementioned concentrations. In some embodiments, the glucose transporter is glucose transporter 1 (GLUT1) and/or glucose transporter 4 (GLUT4). In some embodiments, the method is performed in vitro or in vivo. In some embodiments, GLUT translocation in the cell is enhanced by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% after contacting with the preincubated complex of Gal3 and the anti-Gal3 antibody or binding fragment thereof relative to a cell that is not contacted with the preincubated complex of Gal3 and the anti-Gal3 antibody or binding fragment thereof. In some embodiments, the anti-Gal3 antibody or binding fragment thereof selectively binds to an N-terminal domain of Gal3. In some embodiments, the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof. In some embodiments, the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3. In some embodiments, the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70. In some embodiments, the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952. In some embodiments, the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954. In some embodiments, the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220. In some embodiments, the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 221-247. In some embodiments, the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296. In some embodiments, the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 23. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 955-968, 1067-1109, 1415-1439. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943, 969-982, 1110-1152, 1440-1464. In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514. In some embodiments, the anti-Gal3 antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

In some embodiments, antibodies that bind the N-terminal domain (NTD) of Gal3 (e.g. TB001, TB006, 2D10-VH0-VL0, 20H5.A3) can be used to reduce the ability of Gal3 to block GLUT (e.g., GLUT1 and/or GLUT4) translocation. In some embodiments, any antibody having 1-6 of the CDRs provided herein, that binds to the NTD, can be used in this manner or for this purpose. In some embodiments, reducing the ability of Gal3 to block GLUT (e.g., GLUT1 and/or GLUT4) translocation may be beneficial towards the treatment, amelioration, or prevention of a disease or disorder, such as insulin resistance syndrome, type 2 diabetes, chronic hyperinsulinemia, gestational diabetes, acanthosis nigricans, polycystic ovary syndrome, obesity, muscle wasting, cardiovascular diseases, cardiac hypertrophy, myocardial ischemia, pancreatic cancer associated diabetes, or cancer.

In some embodiments antibodies that bind to the carbohydrate recognition binding domain (CRD) of Gal3 (e.g. 13E2) can be used to enhance the ability of Gal3 to block GLUT (e.g., GLUT1 and/or GLUT4) translocation. In some embodiments, any antibody having 1-6 of the CDRs provided herein, that binds to the CRD, can be used in this manner or for this purpose. In some embodiments, enhancing the ability of Gal3 to block GLUT (e.g., GLUT1 and/or GLUT4) translocation may be beneficial towards the treatment, amelioration, or prevention of a disease or disorder, such as rhabdomyosarcoma.

Also disclosed herein are methods and uses directed to the treatment of a disease or disorder in a subject. In some embodiments, the methods and uses are directed to administering a protein to a subject having, suspected of having, or at risk of developing a disease or disorder. In some embodiments, the protein is an anti-Gal3 antibody or binding fragment thereof. In some embodiments, the disease or disorder is insulin resistance, a disease or disorder associated with insulin resistance, or a disease or disorder comprising a symptom of insulin resistance. In some embodiments, the method involves administering any antibody or variant thereof as provided herein (including any with one or more of the 6 CDRs provided in the present disclosure), in a therapeutically effective amount, sufficient to interfere with the interaction between GAL3 and GLUT (e.g., GLUT1 and/or GLUT4), so as to treat (either in response to a subject having and/or to reduce the risk of) one or more of: diabetes mellitus, insulin resistance, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular diseases, cardiac hypertrophy, myocardial ischemia, hypertension, pancreatic cancer associated diabetes (PCDM), or cancers.

In some embodiments are methods of improving insulin sensitivity in a subject in need thereof. The methods comprise administering to the subject an anti-Gal3 antibody or binding fragment thereof. In some embodiments, binding of the anti-Gal3 antibody or binding fragment thereof to Gal3 in the subject inhibits Gal3-mediated blocking of GLUT translocation in the subject, thereby improving insulin sensitivity in the subject. In some embodiments, the GLUT is GLUT1 and/or GLUT4. In some embodiments, the methods further comprise identifying the subject as needing improvement in insulin sensitivity prior to the administering step. In some embodiments, the methods further comprise detecting an improvement in insulin sensitivity in the subject following the administering step. In some embodiments, detecting the improvement in insulin sensitivity in the subject is done by measuring blood sugar levels, measuring blood insulin levels, glucose tolerance testing, or hyperinsulinemic euglycemic clamp. In some embodiments, the insulin sensitivity in the subject is improved by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200%, or any percentage within a range defined by any two of the aforementioned percentages, relative to the insulin sensitivity of the subject prior to the administering step. In some embodiments, the anti-Gal3 antibody or binding fragment thereof selectively binds to an N-terminal domain of Gal3. In some embodiments, the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof. In some embodiments, the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3. In some embodiments, the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70. In some embodiments, the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952. In some embodiments, the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954. In some embodiments, the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220. In some embodiments, the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 221-247. In some embodiments, the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296. In some embodiments, the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 23. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 955-968, 1067-1109, 1415-1439. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 969-982, 1110-1152, 1440-1464. In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514. In some embodiments, the anti-Gal3 antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

In some embodiments are methods of improving insulin sensitivity in a subject in need thereof. The methods comprise administering to the subject a preincubated complex of Gal3 and an anti-Gal3 antibody or binding fragment thereof, thereby improving insulin sensitivity in the subject. In some embodiments, the preincubated complex of Gal3 and the anti-Gal3 antibody or binding fragment thereof is prepared with Gal3 and the anti-Gal3 antibody or binding fragment thereof at a mass ratio of or of about 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, or 1:2.5, or any mass ratio within a range defined by any two of the aforementioned mass ratios. In some embodiments, the preincubated complex of Gal3 and the anti-Gal3 antibody or binding fragment is prepared with Gal3 at a concentration of or of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 μg/mL, or any concentration within a range defined by any two of the aforementioned concentrations, and the anti-Gal3 antibody or binding fragment thereof at a concentration of or of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL, or any concentration within a range defined by any two of the aforementioned concentrations. In some embodiments, the GLUT is GLUT1 and/or GLUT4. In some embodiments, the methods further comprise identifying the subject as needing improvement in insulin sensitivity prior to the administering step. In some embodiments, the methods further comprise detecting an improvement in insulin sensitivity in the subject following the administering step. In some embodiments, detecting the improvement in insulin sensitivity in the subject is done by measuring blood sugar levels, measuring blood insulin levels, glucose tolerance testing, or hyperinsulinemic euglycemic clamp. In some embodiments, the insulin sensitivity in the subject is improved by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200%, or any percentage within a range defined by any two of the aforementioned percentages, relative to the insulin sensitivity of the subject prior to the administering step. In some embodiments, the anti-Gal3 antibody or binding fragment thereof selectively binds to an N-terminal domain of Gal3. In some embodiments, the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof. In some embodiments, the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3. In some embodiments, the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70. In some embodiments, the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952. In some embodiments, the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954. In some embodiments, the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220. In some embodiments, the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 221-247. In some embodiments, the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296. In some embodiments, the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 23. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 955-968, 1067-1109, 1415-1439. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 969-982, 1110-1152, 1440-1464. In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850,983-996, 1153-1195, 1411, 1465-1489. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514. In some embodiments, the anti-Gal3 antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

In some embodiments are methods of treating a disease associated with insulin resistance in a subject in need thereof. In some embodiments, the methods comprise administering to the subject an anti-Gal3 antibody or binding fragment thereof, thereby treating the disease associated with insulin resistance in the subject. In some embodiments, the disease associated with insulin resistance comprises diabetes mellitus, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular disease, cardiac hypertrophy, myocardial ischemia, hypertension, pancreatic cancer associated diabetes (PCDM) or cancer. In some embodiments, the methods further comprise identifying the subject as needing treatment of the disease associated with insulin resistance prior to the administering step. In some embodiments, the methods further comprise detecting an improvement in the disease associated with insulin resistance following the administering step. In some embodiments, detecting an improvement in the disease associated with insulin resistance comprises detecting an improvement in insulin sensitivity in the subject. In some embodiments, detecting the improvement in insulin sensitivity in the subject is done by measuring blood sugar levels, measuring blood insulin levels, glucose tolerance testing, or hyperinsulinemic euglycemic clamp. In some embodiments, the insulin sensitivity in the subject is improved by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200%, or any percentage within a range defined by any two of the aforementioned percentages, relative to the insulin sensitivity of the subject prior to the administering step. In some embodiments, the anti-Gal3 antibody or binding fragment thereof selectively binds to an N-terminal domain of Gal3. In some embodiments, the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof. In some embodiments, the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3. In some embodiments, the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70. In some embodiments, the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952. In some embodiments, the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954. In some embodiments, the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220. In some embodiments, the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 221-247. In some embodiments, the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296. In some embodiments, the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 23. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 955-968, 1067-1109, 1415-1439. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943,969-982, 1110-1152, 1440-1464. In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514. In some embodiments, the anti-Gal3 antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

In some embodiments are methods of treating a disease associated with insulin resistance in a subject in need thereof. In some embodiments, the methods comprise administering to the subject a preincubated complex of Gal3 and an anti-Gal3 antibody or binding fragment thereof, thereby treating the disease associated with insulin resistance in the subject. In some embodiments, the preincubated complex of Gal3 and the anti-Gal3 antibody or binding fragment thereof is prepared with Gal3 and the anti-Gal3 antibody or binding fragment thereof at a mass ratio of or of about 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, or 1:2.5, or any mass ratio within a range defined by any two of the aforementioned mass ratios. In some embodiments, the preincubated complex of Gal3 and the anti-Gal3 antibody or binding fragment is prepared with Gal3 at a concentration of or of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 μg/mL, or any concentration within a range defined by any two of the aforementioned concentrations, and the anti-Gal3 antibody or binding fragment thereof at a concentration of or of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL, or any concentration within a range defined by any two of the aforementioned concentrations. In some embodiments, the disease associated with insulin resistance comprises diabetes mellitus, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular disease, cardiac hypertrophy, myocardial ischemia, hypertension, pancreatic cancer associated diabetes (PCDM) or cancer. In some embodiments, the methods further comprise identifying the subject as needing treatment of the disease associated with insulin resistance prior to the administering step. In some embodiments, the methods further comprise detecting an improvement in the disease associated with insulin resistance following the administering step. In some embodiments, detecting an improvement in the disease associated with insulin resistance comprises detecting an improvement in insulin sensitivity in the subject. In some embodiments, detecting the improvement in insulin sensitivity in the subject is done by measuring blood sugar levels, measuring blood insulin levels, glucose tolerance testing, or hyperinsulinemic euglycemic clamp. In some embodiments, the insulin sensitivity in the subject is improved by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200%, or any percentage within a range defined by any two of the aforementioned percentages, relative to the insulin sensitivity of the subject prior to the administering step. In some embodiments, the anti-Gal3 antibody or binding fragment thereof selectively binds to an N-terminal domain of Gal3. In some embodiments, the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof. In some embodiments, the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3. In some embodiments, the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70. In some embodiments, the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952. In some embodiments, the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954. In some embodiments, the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220. In some embodiments, the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 221-247. In some embodiments, the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296. In some embodiments, the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 23. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 955-968, 1067-1109, 1415-1439. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943, 969-982, 1110-1152, 1440-1464. In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514. In some embodiments, the anti-Gal3 antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

In some embodiments are methods of diagnosing a disease or disorder, or a symptom thereof, in a subject. In some embodiments, the disease or disorder is associated with insulin resistance. In some embodiments, the disease or disorder is diabetes mellitus, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular disease, cardiac hypertrophy, myocardial ischemia, hypertension, pancreatic cancer associated diabetes (PCDM) or cancer. In some embodiments, the disease or disorder is diabetes mellitus. In some embodiments, the disease or disorder is insulin-dependent diabetes mellitus. In some embodiments, the disease or disorder is insulin-independent diabetes mellitus. In some embodiments, the disease or disorder is Type I diabetes mellitus. In some embodiments, the disease or disorder is Type II diabetes mellitus. In some embodiments, the methods comprise contacting the subject, or a part of the subject (e.g. tissue, blood/serum) with an anti-Gal3 antibody or binding fragment thereof. In some embodiments, the anti-Gal3 antibody or binding fragment thereof is conjugated to a detectable moiety. In some embodiments, any of the methods disclosed herein involving an anti-Gal3 antibody or binding fragment can be performed with an antigen binding molecule that binds to Gal3.

As applied to any of the methods of use disclosed herein, in some embodiments, the anti-Gal3 antibody or binding fragment thereof binds to one or more peptides of SEQ ID NOs: 3-26. In some embodiments, the anti-Gal3 antibody or binding fragment thereof binds to an epitope present within a region of Gal3 defined by Peptide 1 (ADNFSLHDALSGSGNPNPQG; SEQ ID NO: 3), Peptide 4 (GAGGYPGASYPGAYPGQAPP; SEQ ID NO: 6), Peptide 6 (GAYPGQAPPGAYPGAPGAYP; SEQ ID NO: 8), Peptide 7 (AYPGAPGAYPGAPAPGVYPG; SEQ ID NO: 9), or a combination thereof. In some embodiments, any of the methods disclosed herein involving an anti-Gal3 antibody or binding fragment can be performed with an antigen binding molecule that binds to Gal3.

As applied to any of the methods of use disclosed herein, in some embodiments, the anti-Gal3 antibody or binding fragment thereof binds to an epitope of Gal3 that includes a motif of GxYPG, where x is the amino acids alanine (A), glycine (G), or valine (V). In some embodiments, an anti-Gal3 antibody as described herein binds to an epitope of Gal3 that includes two GxYPG motifs separated by three amino acids, where x is A, G, or V. In some embodiments, any of the methods disclosed herein involving an anti-Gal3 antibody or binding fragment can be performed with an antigen binding molecule that binds to Gal3.

As applied to any of the methods of use disclosed herein, in some embodiments, the anti-Gal3 antibody or binding fragment thereof comprises (1) a light chain variable region comprising a V_{L}-CDR1, a V_{L}-CDR2, and a V_{L}-CDR3 and (2) a heavy chain variable region comprising a V_{H}-CDR1, a V_{H}-CDR2, and a V_{H}-CDR3. In some embodiments, the V_{L}-CDR1 comprises an amino acid sequence having at least 60%, at least 70%, at least 80%, at least 90%, or 100% sequence identity to any amino acid sequence according to SEQ ID NOs: 170-220. In some embodiments, the V_{L}-CDR2 comprises an amino acid sequence having at least 60%, at least 70%, at least 80%, at least 90%, or 100% sequence identity to any amino acid sequence according to SEQ ID NOs: 221-247. In some embodiments, the V_{L}-CDR3 comprises an amino acid sequence having at least 60%, at least 70%, at least 80%, at least 90%, or 100% sequence identity to any amino acid sequence according to SEQ ID NOs: 248-296. In some embodiments, the V_{H}-CDR1 comprises an amino acid sequence having at least 60%, at least 70%, at least 80%, at least 90%, or 100% sequence identity to any amino acid sequence according to SEQ ID NOs: 27-70. In some embodiments, the V_{H}-CDR2 comprises an amino acid sequence having at least 60%, at least 70%, at least 80%, at least 90%, or 100% sequence identity to any amino acid sequence according to SEQ ID NOs: 71-111, 801, 951, 952. In some embodiments, the V_{H}-CDR3 comprises an amino acid sequence having at least 60%, at least 70%, at least 80%, at least 90%, or 100% sequence identity to any amino acid sequence according to SEQ ID NOs: 112-169, 802, 953, 954. In some embodiments, any of the methods disclosed herein involving an anti-Gal3 antibody or binding fragment can be performed with an antigen binding molecule that binds to Gal3.

As applied to any of the methods of use disclosed herein, in some embodiments, exemplary V_{H}-CDR1 sequences are depicted in FIG. 19A. In some embodiments, exemplary V_{H}-CDR2 sequences are depicted in FIG. 19B. In some embodiments, exemplary V_{H}-CDR3 sequences are depicted in FIG. 19C. In some embodiments, exemplary V_{L}-CDR1 sequences are depicted in FIG. 20A. In some embodiments, exemplary V_{L}-CDR2 sequences are depicted in FIG. 20B. In some embodiments, exemplary V_{L}-CDR3 sequences are depicted in FIG. 20C.

As applied to any of the methods of use disclosed herein, in some embodiments, the heavy chain variable region of any of the anti-Gal3 antibodies or binding fragments thereof disclosed herein comprises an amino acid sequence having at least 75%, 80%, 85%, 90%, 95%, or 100% sequence identity to any sequence according to SEQ ID NOs: 297-373, 803, 806-820, 955-968, 1067-1109, 1415-1439. In some embodiments, the heavy chain variable region of any of the anti-Gal3 antibodies or binding fragments thereof disclosed herein is selected from the group consisting of SEQ ID NOs: 297-373, 803, 806-820, 955-968, 1067-1109, 1415-1439. In some embodiments, exemplary V_{H} are depicted in FIG. 21. In some embodiments, any of the methods disclosed herein involving an anti-Gal3 antibody or binding fragment can be performed with an antigen binding molecule that binds to Gal3.

As applied to any of the methods of use disclosed herein, in some embodiments, the light chain variable region of any of the anti-Gal3 antibodies or binding fragments thereof disclosed herein comprises an amino acid sequence having at least 75%, 80%, 85%, 90%, 95%, or 100% sequence identity to any sequence according to SEQ ID NOs: 374-447, 821-835, 941-943, 969-982, 1110-1152, 1440-1464. In some embodiments, the light chain variable region of any of the anti-Gal3 antibodies or binding fragments thereof disclosed herein is selected from the group consisting of SEQ ID NOs: 374-447, 821-835, 941-943, 969-982, 1110-1152, 1440-1464. In some embodiments, exemplary V_{L} are depicted in FIG. 22. In some embodiments, any of the methods disclosed herein involving an anti-Gal3 antibody or binding fragment can be performed with an antigen binding molecule that binds to Gal3.

As applied to any of the methods of use disclosed herein, in some embodiments, the anti-Gal3 antibody or binding fragment thereof comprises the heavy chain sequence of any one of SEQ ID NOs: 448-494, 804, 836-850, 983-996, 1153-1195, 1411, 1465-1489. In some embodiments, the anti-Gal3 antibody or binding fragment thereof comprises the light chain sequence of any one of SEQ ID NOs: 495-538, 805, 851-865, 997-1010,1196-1238, 1412, 1490-1514.

As applied to any of the methods of use disclosed herein, in some embodiments, the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

As applied to any of the methods of use disclosed herein, in some embodiments, the anti-Gal3 antibody or binding fragment thereof excludes the subset of named anti-Gal3 antibodies or binding fragments thereof depicted in any one of FIGS. 38A-D. In some embodiments, CDRs (including heavy chain and light chain CDRs), heavy chain variable regions, light chain variable regions, heavy chains, and/or light chains are excluded from the sequences associated with the subset of named anti-Gal3 antibodies or binding fragments thereof depicted in any one of FIGS. 38A-D. In some embodiments, combinations of CDRs (including heavy chain and light chain CDRs), heavy chain variable regions, light chain variable regions, heavy chains, and/or light chains are excluded from the sequences associated with the subset of named anti-Gal3 antibodies or binding fragments thereof depicted in any one of FIGS. 38A-D, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 38A, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 38B, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 38C, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 38D, or excludes any one or more of the CDRs, VH, VL, HC, and/or LC thereof. In some embodiments, the anti-Gal3 antibody or binding fragment thereof blocks the interaction between Gal3 and GLUT (e.g., GLUT1 and/or GLUT4) and excludes those selected from the group of named anti-Gal3 antibody or binding fragment thereof depicted in FIG. 38A, or excludes any one or more of the CDRs, VH, VL, HC, and/or LC thereof. In some embodiments, the anti-Gal3 antibody or binding fragment thereof blocks the interaction between Gal3 and GLUT (e.g., GLUT1 and/or GLUT4) and excludes those selected from the group of named anti-Gal3 antibody or binding fragment thereof depicted in FIG. 38B, or excludes any one or more of the CDRs, VH, VL, HC, and/or LC thereof. In some embodiments, the anti-Gal3 antibody or binding fragment thereof blocks the interaction between Gal3 and GLUT (e.g., GLUT1 and/or GLUT4) and excludes those selected from the group of named anti-Gal3 antibody or binding fragment thereof depicted in FIG. 38C, or excludes any one or more of the CDRs, VH, VL, HC, and/or LC thereof. In some embodiments, the anti-Gal3 antibody or binding fragment thereof blocks the interaction between Gal3 and GLUT (e.g., GLUT1 and/or GLUT4) and excludes those selected from the group of named anti-Gal3 antibody or binding fragment thereof depicted in FIG. 38D, or excludes any one or more of the CDRs, VH, VL, HC, and/or LC thereof. In other embodiments, any of these constructs (e.g., those in FIG. 38A-38D) are used for any of the methods provided herein.

As applied to any of the methods of use disclosed herein, in some embodiments, the anti-Gal3 antibody or binding fragment thereof is selected from the subset of named anti-Gal3 antibodies or binding fragments thereof depicted in any one of FIGS. 39A-E. In some embodiments, CDRs (including heavy chain and light chain CDRs), heavy chain variable regions, light chain variable regions, heavy chains, and/or light chains are selected from the sequences associated with the subset of named anti-Gal3 antibodies or binding fragments thereof depicted in any one of FIGS. 39A-E. In some embodiments, combinations of CDRs (including heavy chain and light chain CDRs), heavy chain variable regions, light chain variable regions, heavy chains, and/or light chains are selected from the sequences associated with the subset of named anti-Gal3 antibodies or binding fragments thereof depicted in any one of FIGS. 39A-E. In some embodiments, the anti-Gal3 antibody or binding fragment thereof is selected from the group of named anti-Gal3 antibody or binding fragment thereof depicted in FIG. 39A, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 39B, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 39C, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 39D, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 39E, or comprises any one or more of the CDRs, VH, VL, HC, and/or LC thereof.

As applied to any of the methods of use disclosed herein, in some embodiments, the anti-Gal3 antibody or binding fragment inhibits, blocks, or disrupts an interaction between Gal3 and a glucose transporter (e.g., GLUT1 and/or GLUT4). In some embodiments, the anti-Gal3 antibody or binding fragment inhibits, blocks, or disrupts the interaction with a quantifiable IC50. In some embodiments, the anti-Gal3 antibody or binding fragments inhibits, blocks, or disrupts the interaction between Gal3 and the glucose transporter (e.g., GLUT1 and/or GLUT4) with an IC50 of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 μg/mL, or no more than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 μg/mL, or any IC50 concentration within a range defined by any two of the aforementioned concentrations.

As applied to any of the methods of use disclosed herein, in some embodiments, the anti-Gal3 antibody or binding fragment thereof comprises a payload. In some embodiments, the payload is conjugated to the anti-Gal3 antibody or binding fragment thereof. In some embodiments, the payload is a cytotoxic payload, microtubule disrupting agent, DNA modifying agent, Akt inhibitor, polymerase inhibitor, detectable moiety, immunomodulatory agent, immune modulator, immunotoxin, nucleic acid polymer, aptamer, peptide, or any combination thereof. In some embodiments, the payload is a detectable moiety. In some embodiments, any of the methods disclosed herein involving an anti-Gal3 antibody or binding fragment can be performed with an antigen binding molecule that binds to Gal3.

As applied to any of the methods of use disclosed herein, in some embodiments, the anti-Gal3 antibody or binding fragment thereof is or comprises a humanized antibody. In some embodiments, the anti-Gal3 antibody or binding fragment thereof is or comprises a full-length antibody or a binding fragment thereof. In some embodiments, the anti-Gal3 antibody or binding fragment thereof is or comprises a bispecific antibody or a binding fragment thereof. In some embodiments, the anti-Gal3-antibody or binding fragment thereof is or comprises a monovalent Fab′, a divalent Fab2, a single-chain variable fragment (scFv), a diabody, a minibody, a nanobody, a single-domain antibody (sdAb), or a camelid antibody, or binding fragment thereof. In some embodiments, the anti-Gal3 antibody or binding fragment thereof is or comprises an IgG framework. In some embodiments, the anti-Gal3 antibody or binding fragment thereof is or comprises an IgG1, IgG2, or IgG4 framework. In some embodiments, any of the methods disclosed herein involving an anti-Gal3 antibody or binding fragment can be performed with an antigen binding molecule that binds to Gal3.

As applied to any of the methods of treatment disclosed herein, in some embodiments, in some embodiments, the anti-Gal3 antibody or binding fragment thereof is administered enterally, orally, intranasally, parenterally, intracranially, subcutaneously, intramuscularly, intradermally, or intravenously, or any combination thereof.

As applied to any of the methods of treatment disclosed herein, in some embodiments, the anti-Gal3 antibody or binding fragment thereof is formulated for systemic administration. In some embodiments, the anti-Gal3 antibody or binding fragment thereof is formulated for parenteral administration. In some embodiments, more than one anti-Gal3 antibody or binding fragment is administered. In some embodiments, when more than one anti-Gal3 antibody or binding fragment is administered, the more than one anti-Gal3 antibodies or binding fragments thereof may be selected from the anti-Gal3 antibodies or binding fragments thereof disclosed herein. In some embodiments, any of the methods disclosed herein involving an anti-Gal3 antibody or binding fragment can be performed with an antigen binding molecule that binds to Gal3.

As applied to any of the methods of use or treatment disclosed herein, the subject is a mammal. In some embodiments, the mammal is a human, cat, dog, mouse, rat, hamster, rodent, pig, cow, horse, sheep, or goat. In some embodiments, the mammal is a human.

### Therapeutic Regimens

In some embodiments, the anti-Gal3 antibodies or binding fragments thereof disclosed herein are administered for therapeutic applications. In some embodiments, the anti-Gal3 antibody or binding fragment thereof is administered once per day, twice per day, three times per day or more. The anti-Gal3 antibody or binding fragment thereof is administered daily, every day, every alternate day, five days a week, once a week, every other week, two weeks per month, three weeks per month, once a month, twice a month, three times per month, or more. In some embodiments, the anti-Gal3 antibody or binding fragment thereof is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the anti-Gal3 antibody or binding fragment thereof is given continuously; alternatively, the dose of the anti-Gal3 antibody or binding fragment thereof being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, can be reduced, as a function of the symptoms, to a level at which the treated disease, disorder, or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Polynucleotides and Vectors

In some embodiments, the present disclosure provides isolated nucleic acids encoding any of the anti-Gal3 antibodies or binding fragments thereof disclosed herein. In another embodiment, the present disclosure provides vectors comprising a nucleic acid sequence encoding any anti-Gal3 antibody or binding fragment thereof disclosed herein. In some embodiments, this disclosure provides isolated nucleic acids that encode heavy chain variable regions, light chain variable regions, heavy chains, or light chains of an anti-Gal3 antibody or binding fragment thereof disclosed herein.

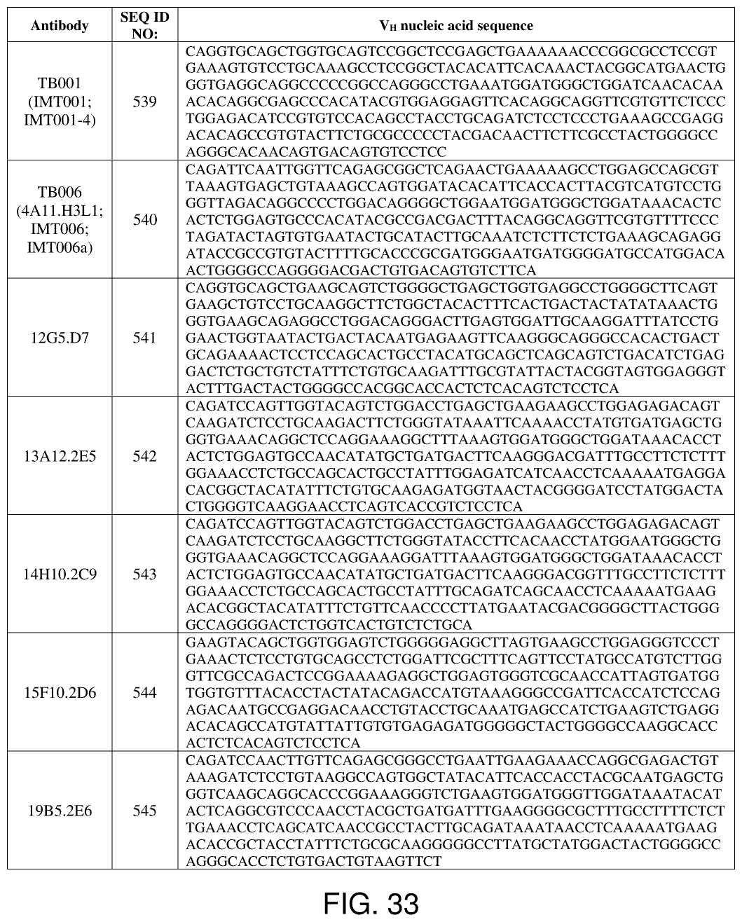

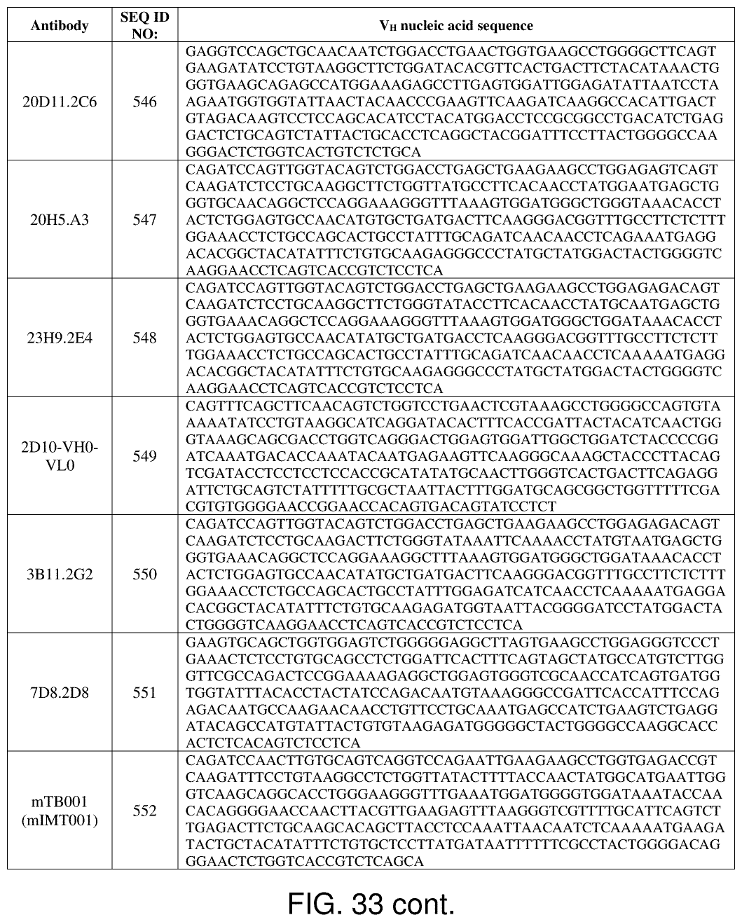

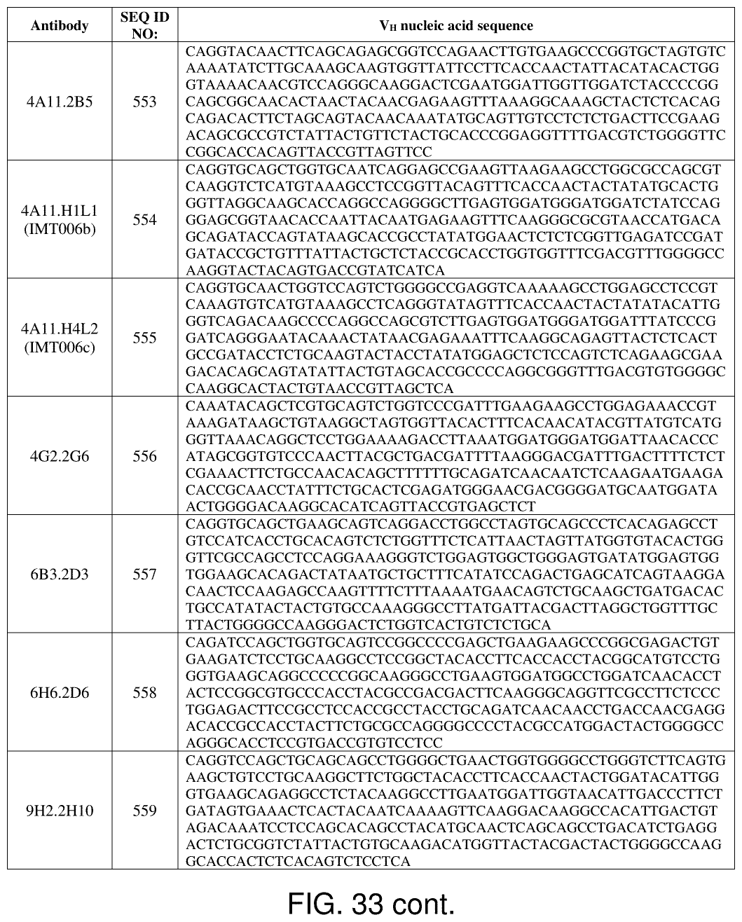

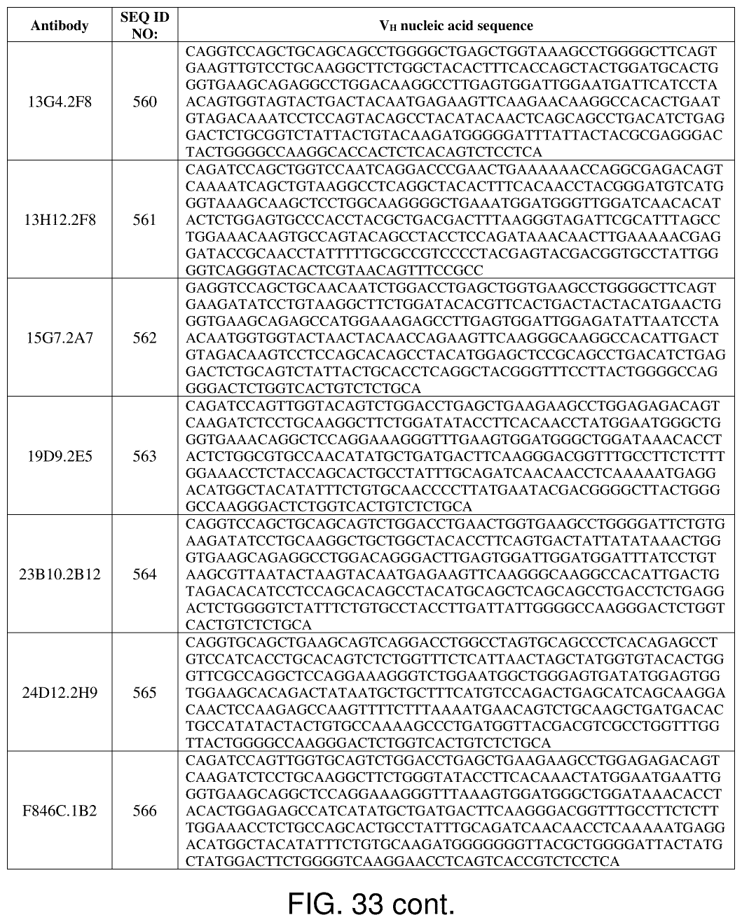

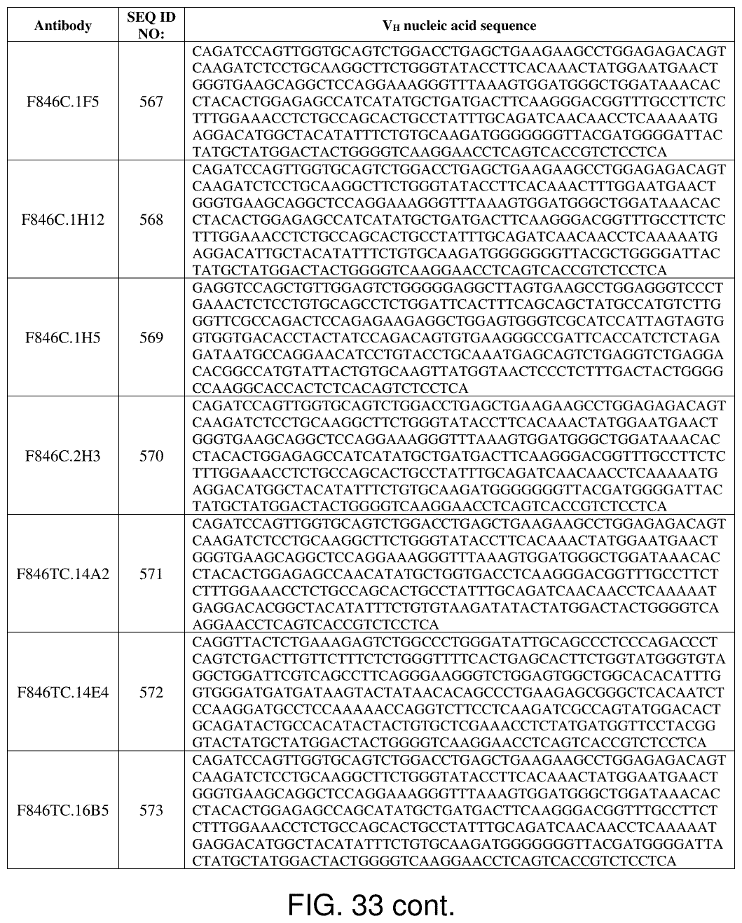

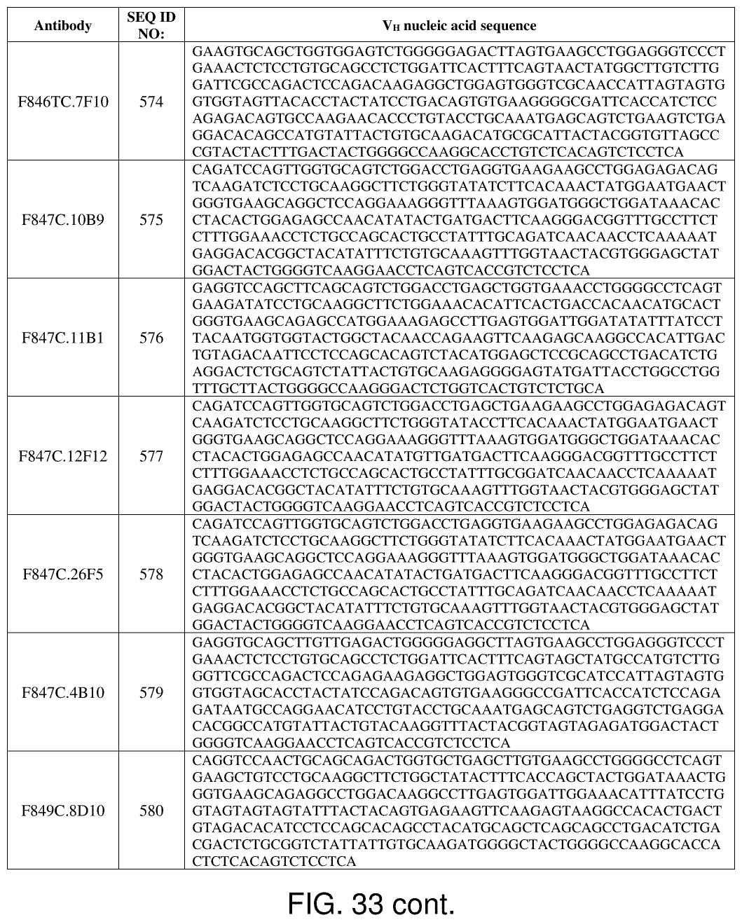

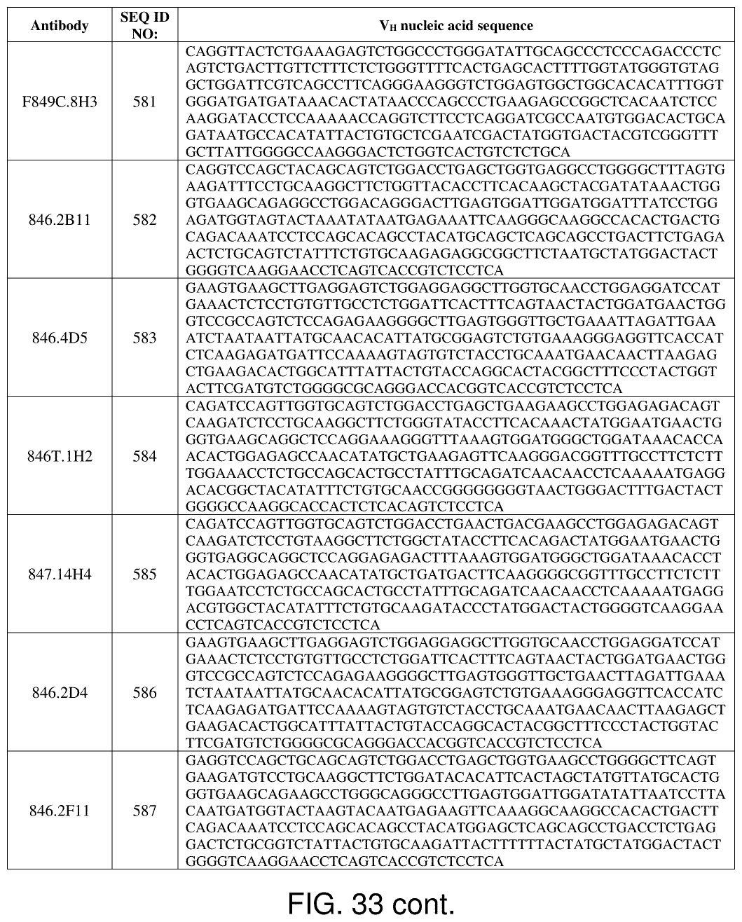

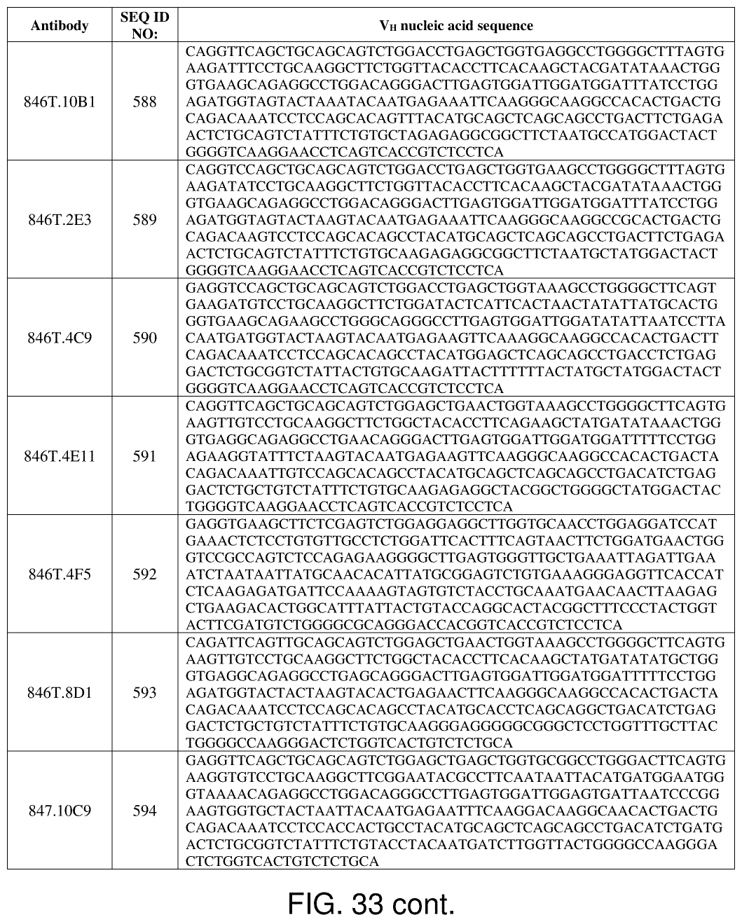

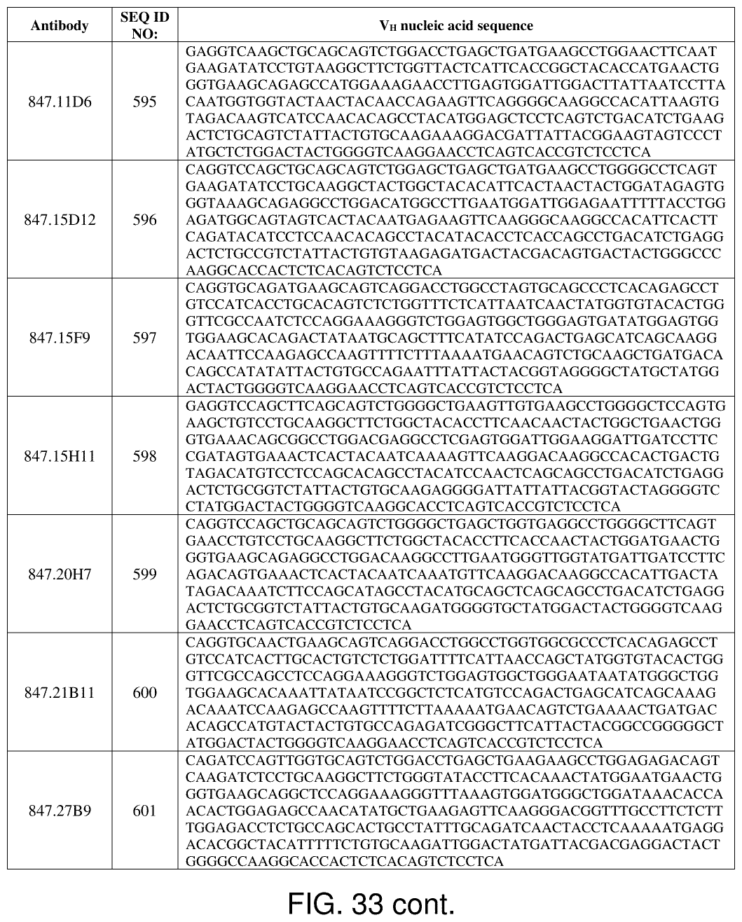

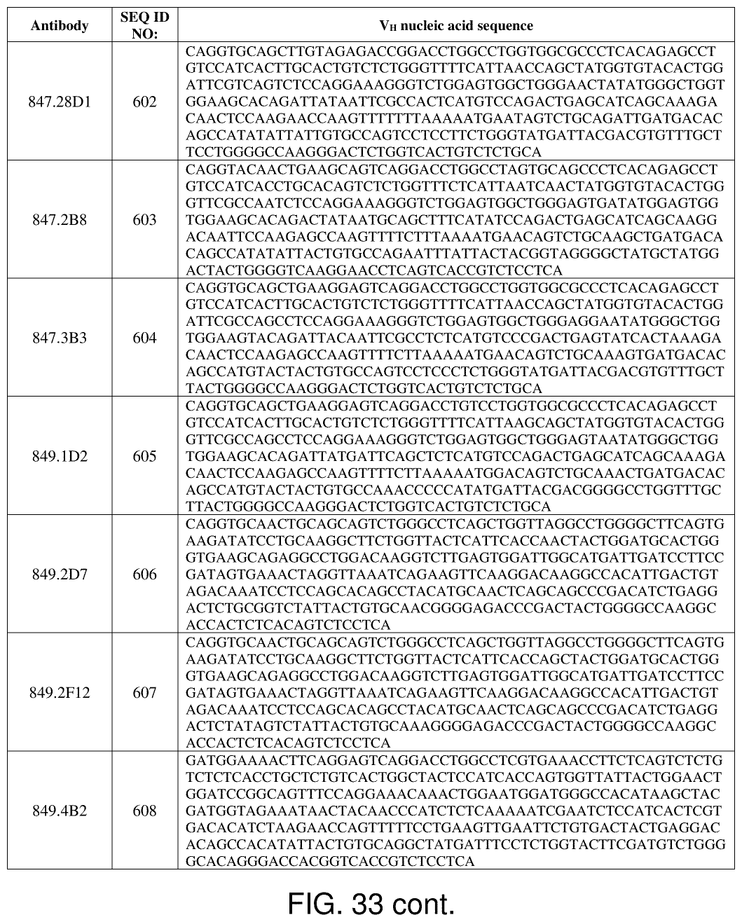

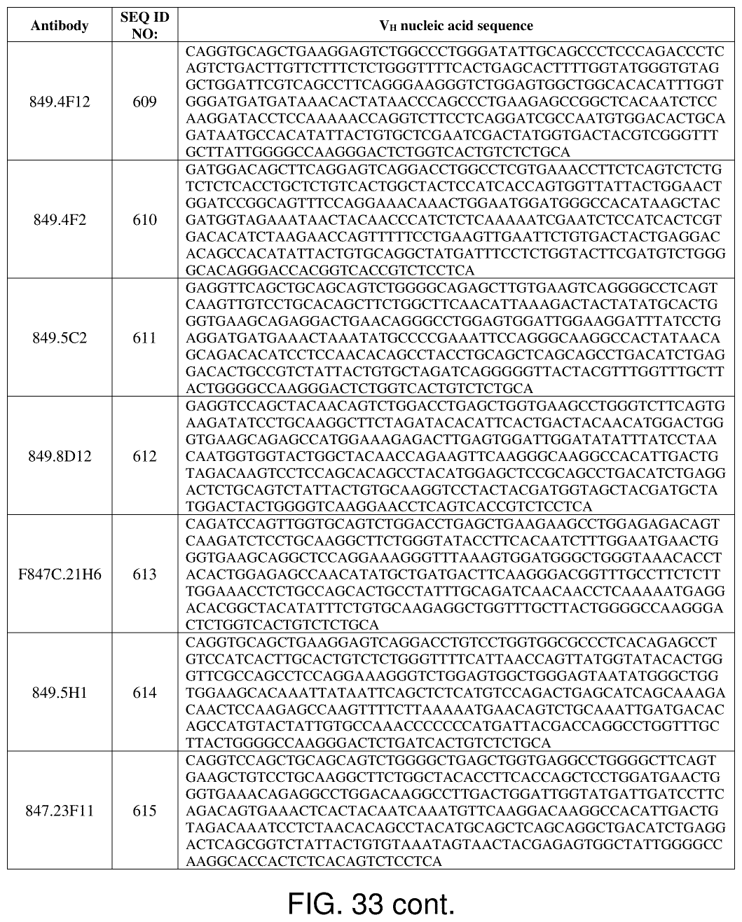

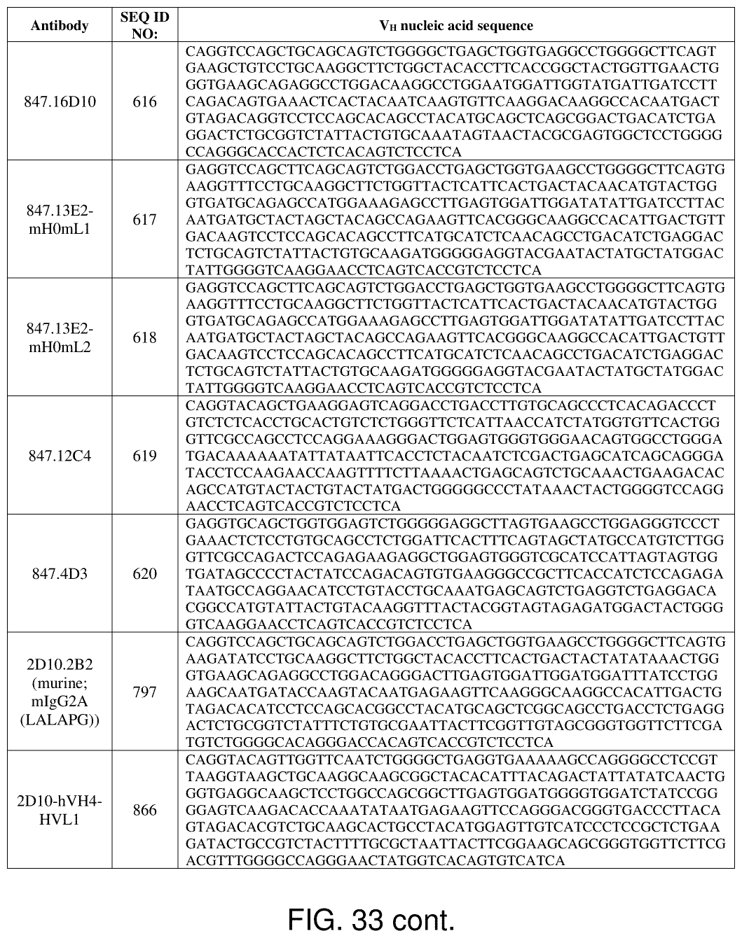

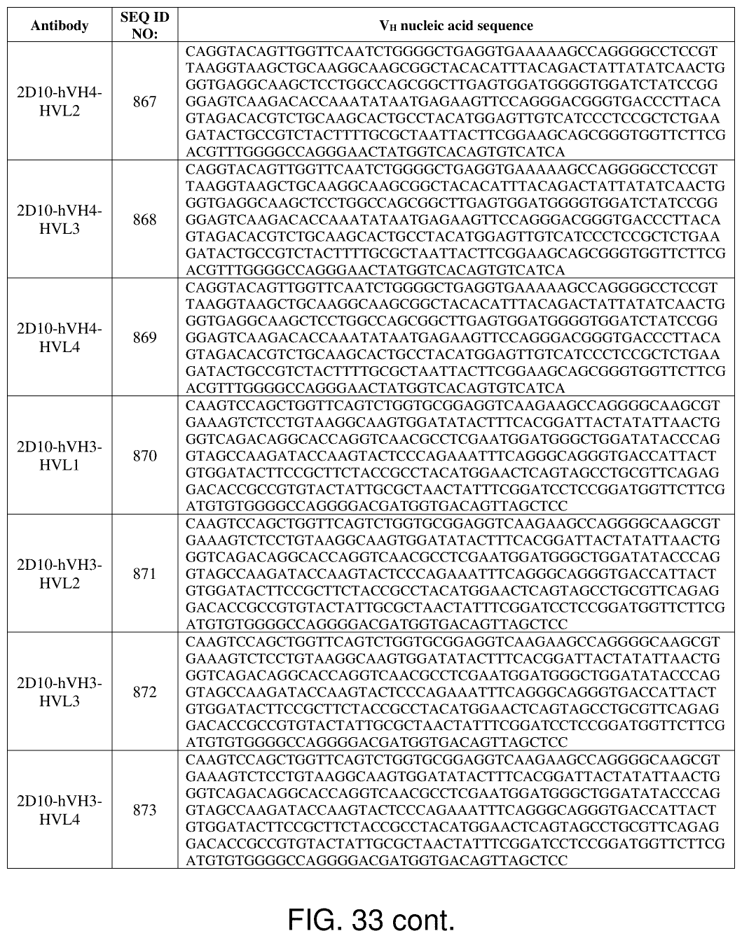

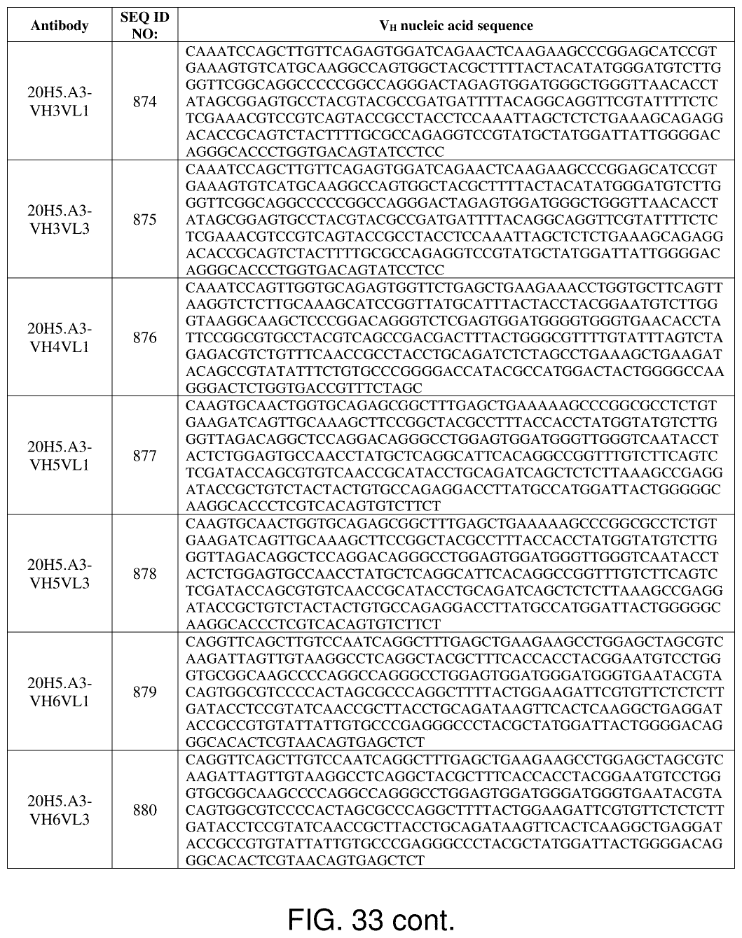

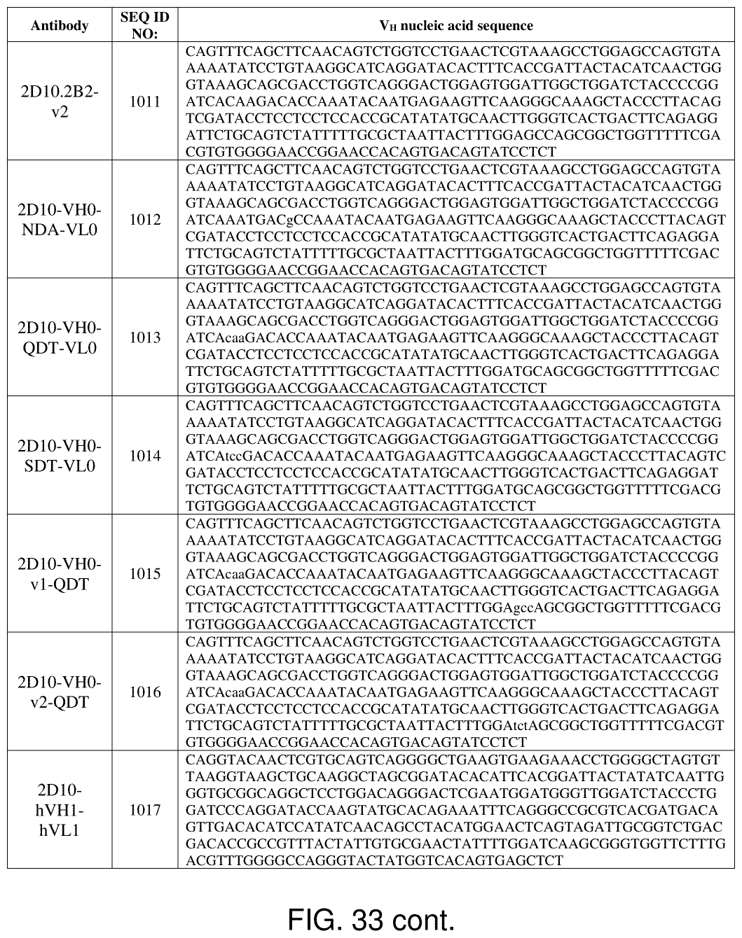

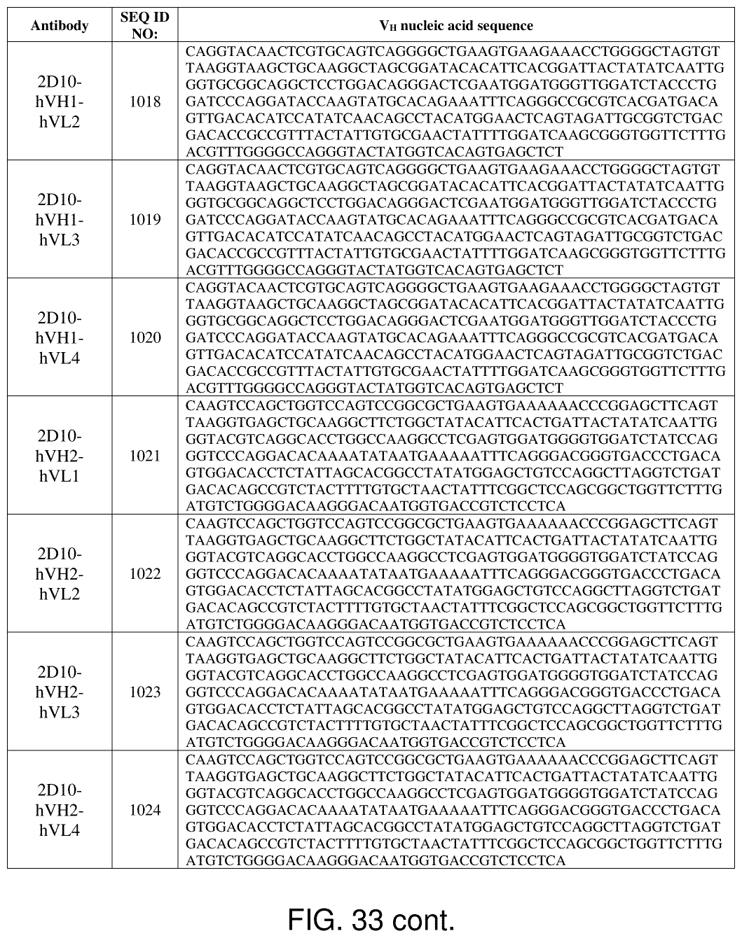

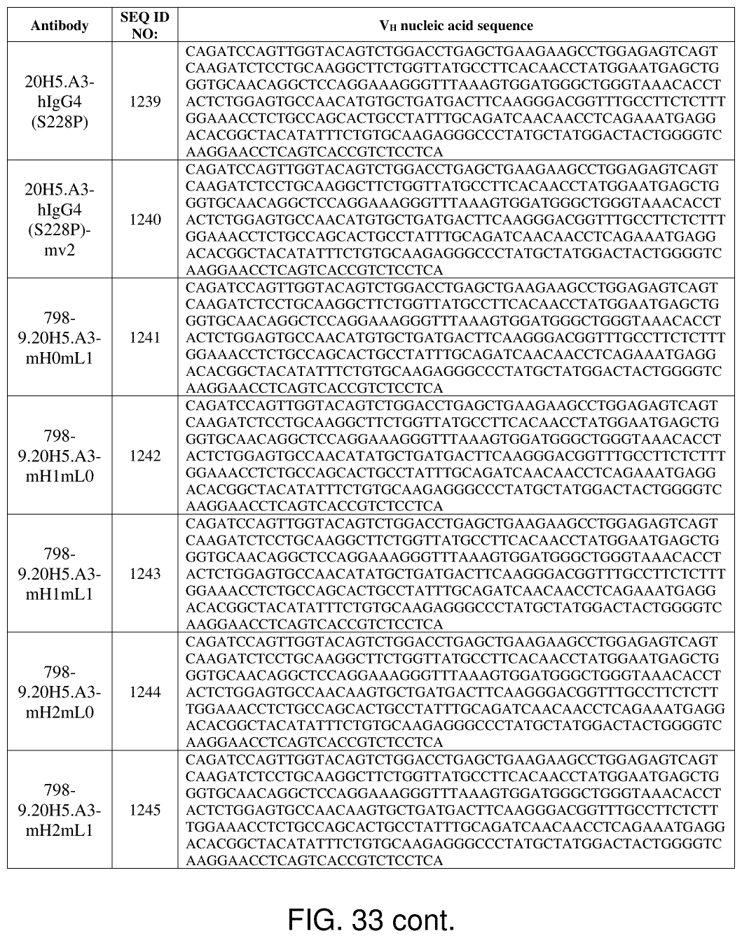

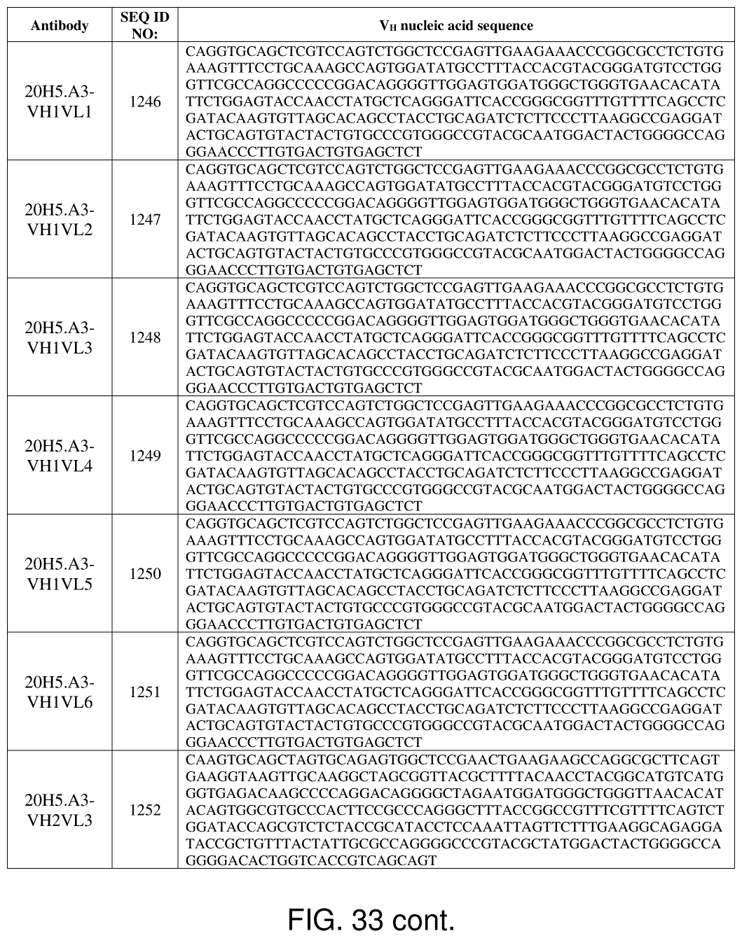

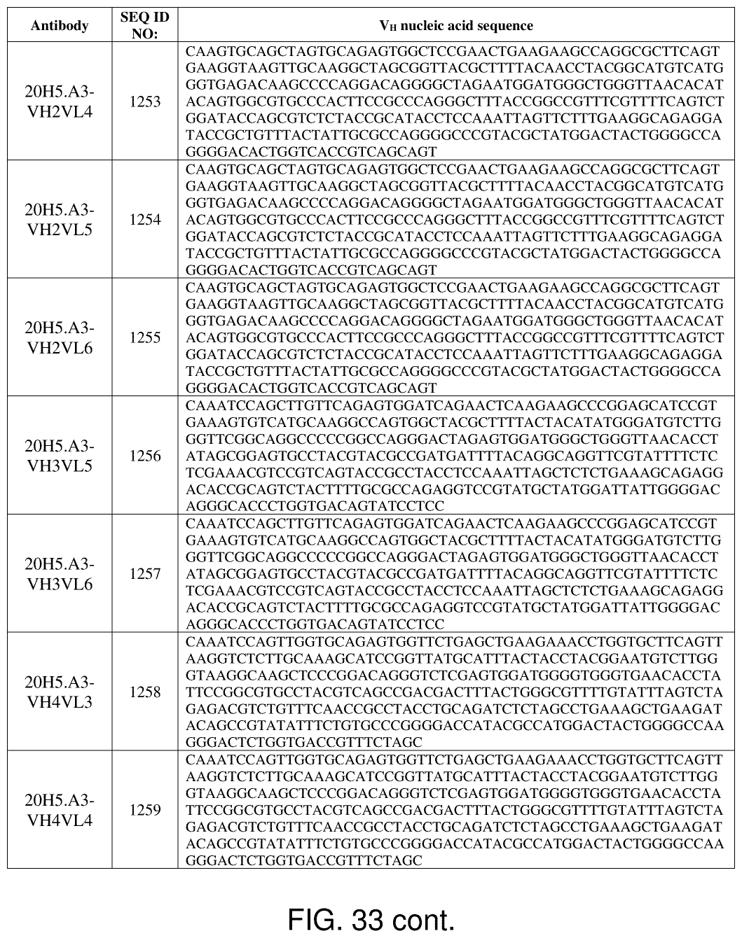

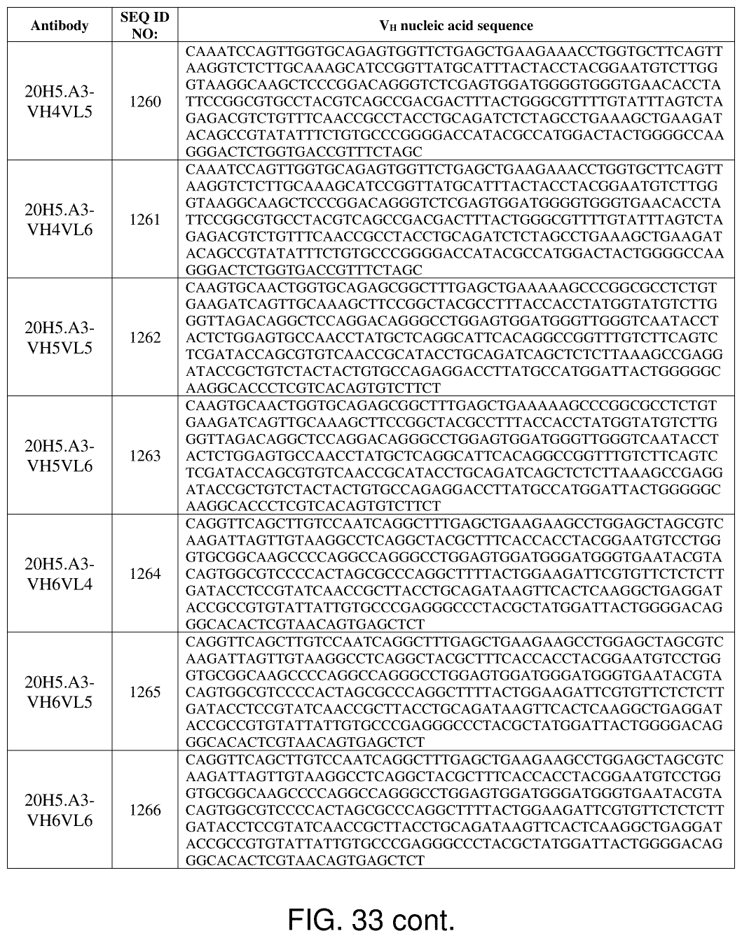

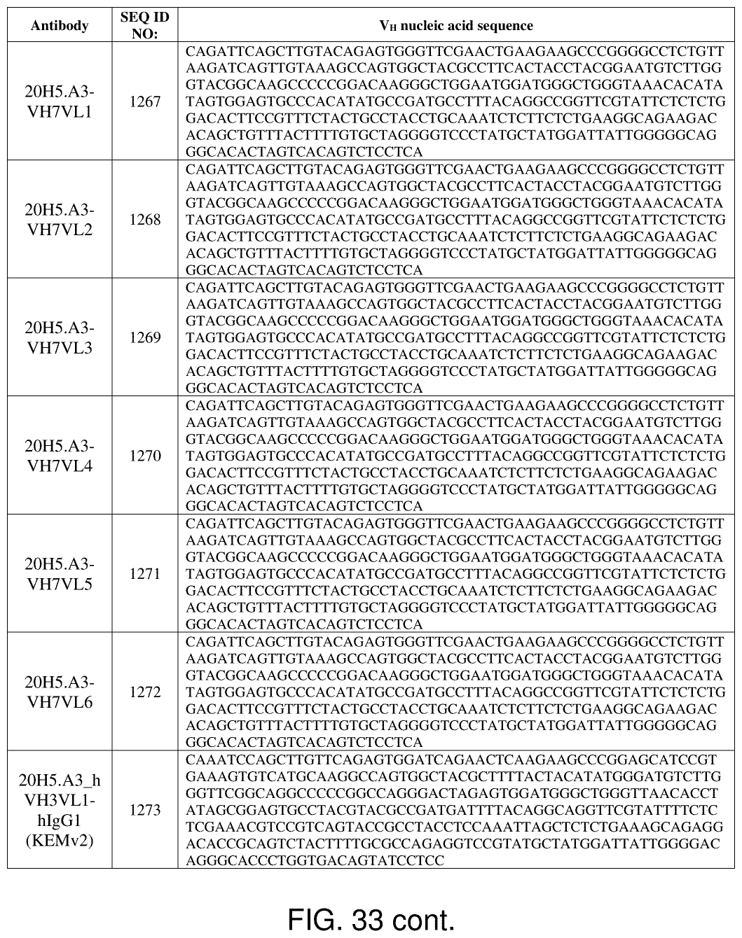

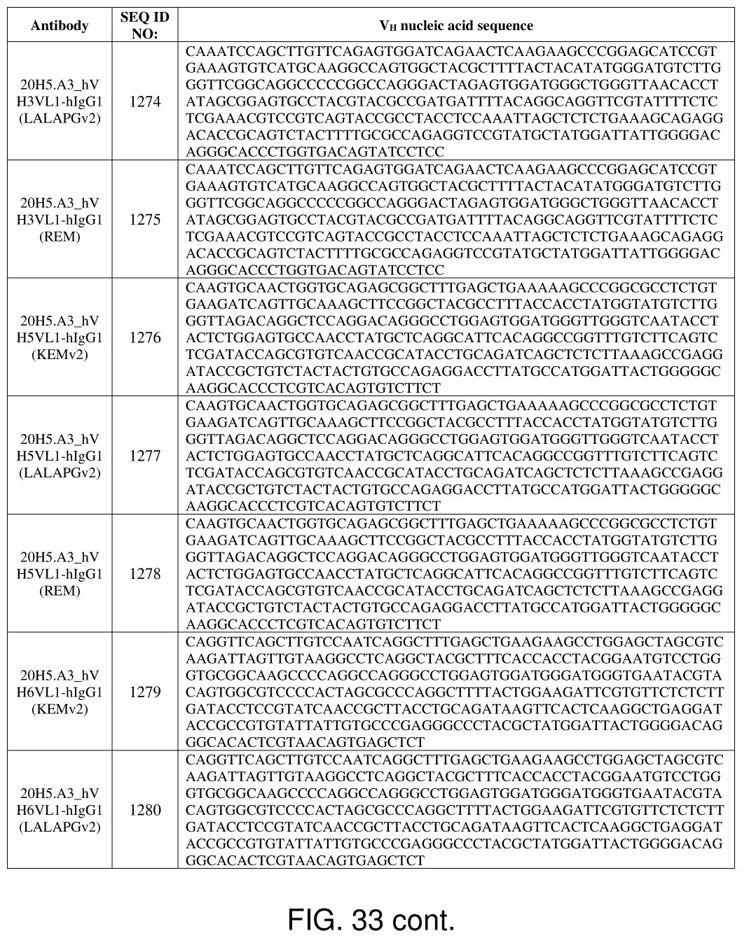

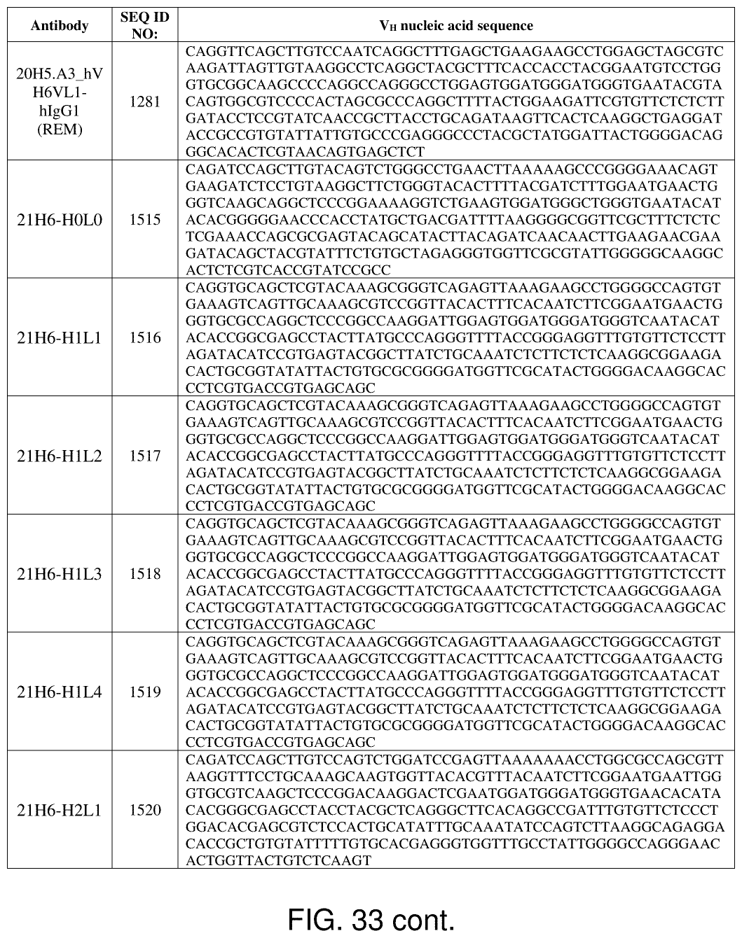

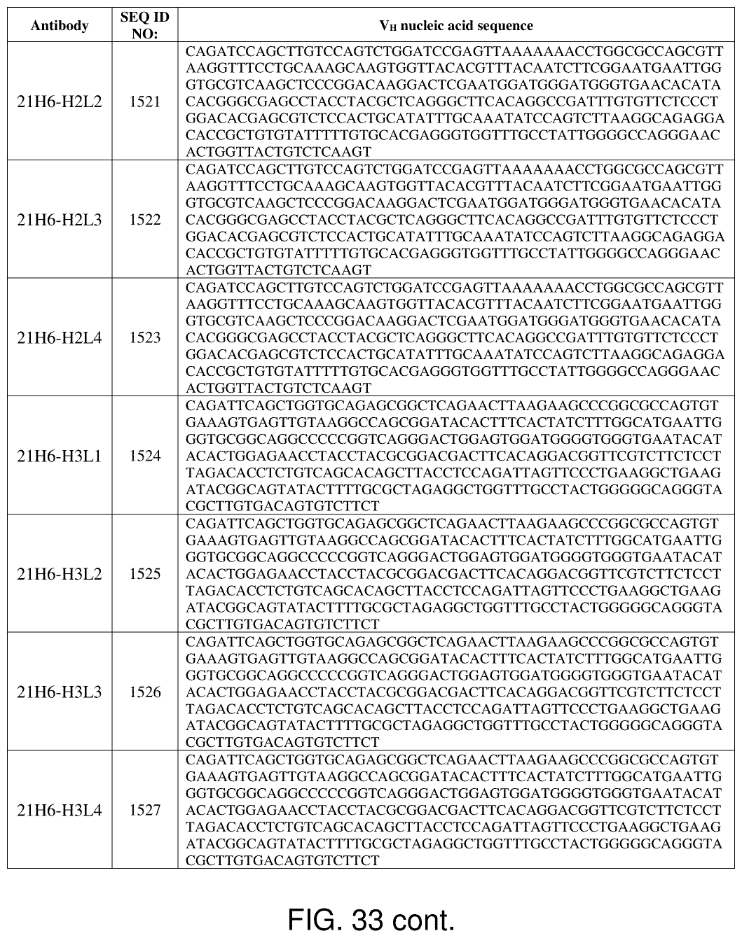

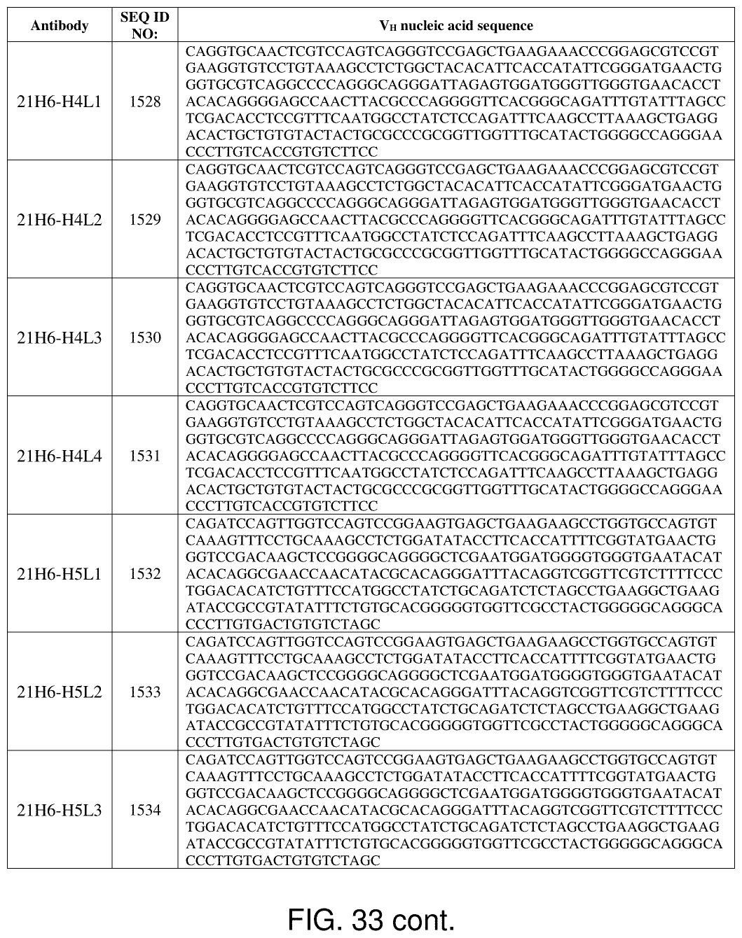

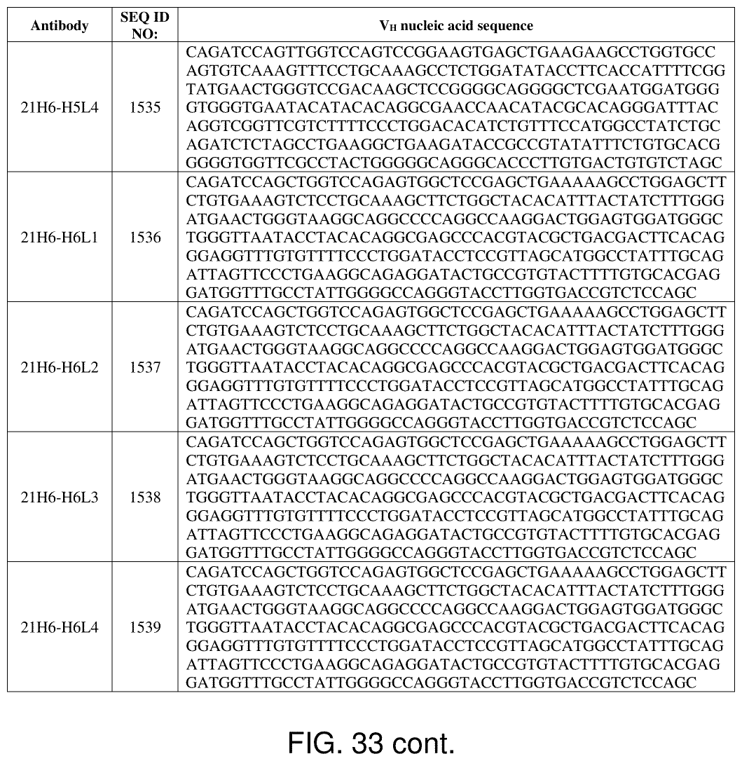

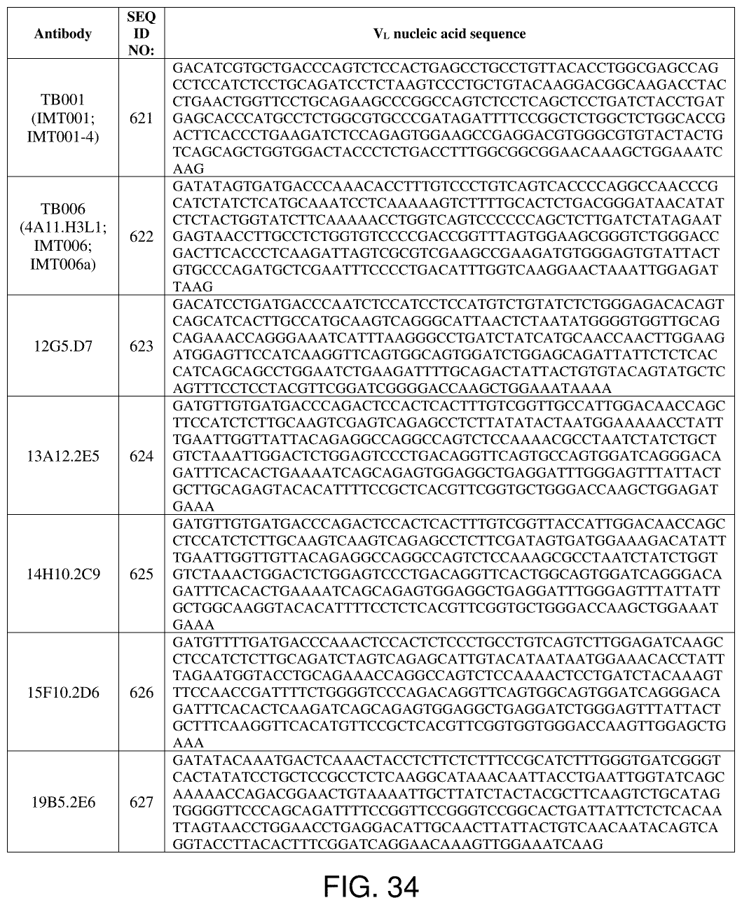

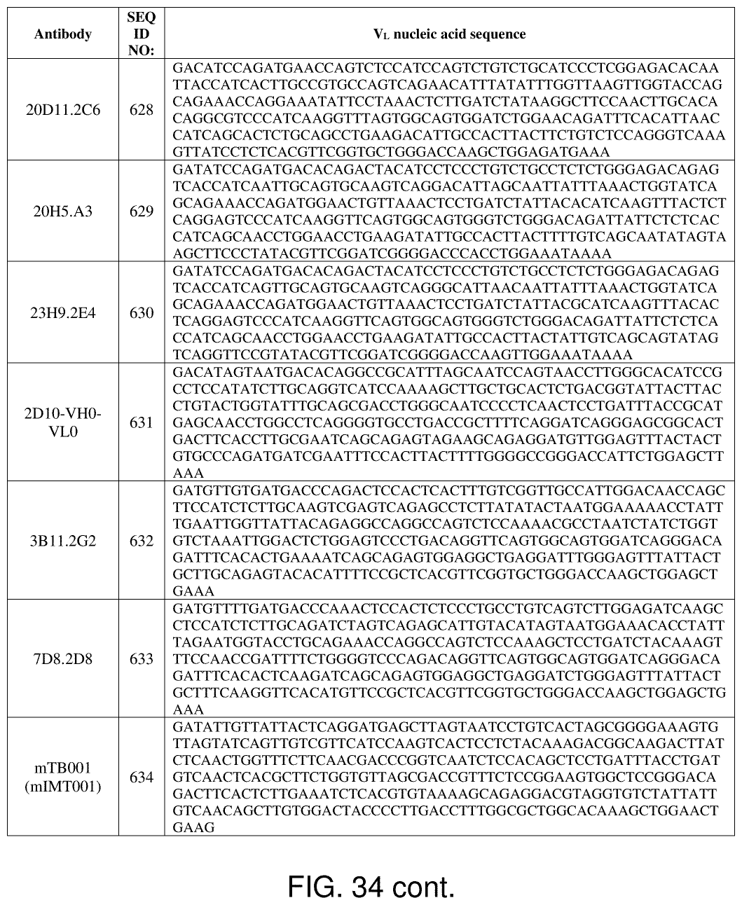

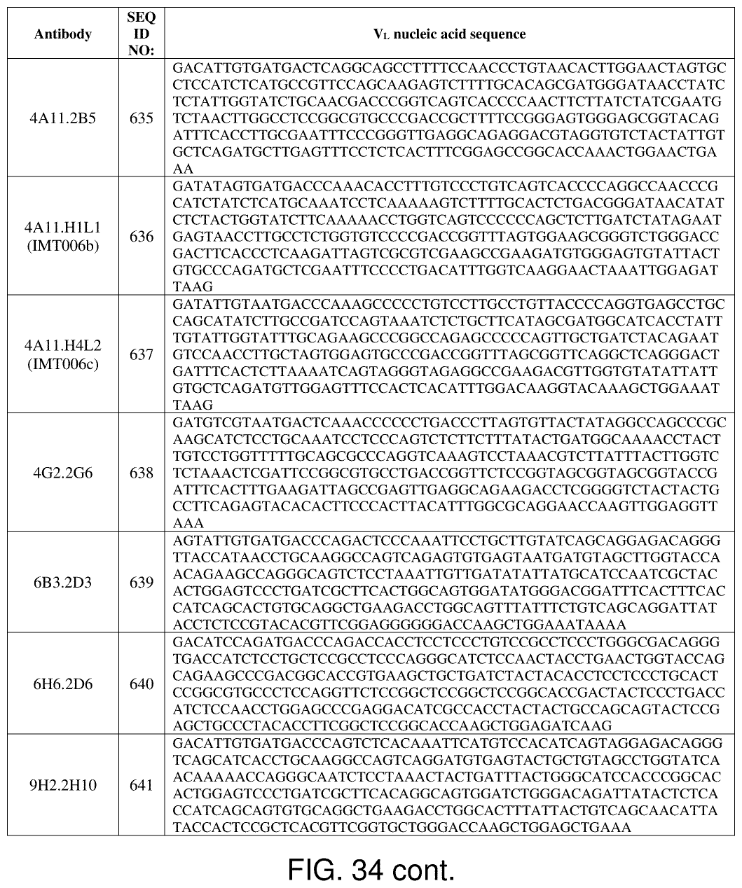

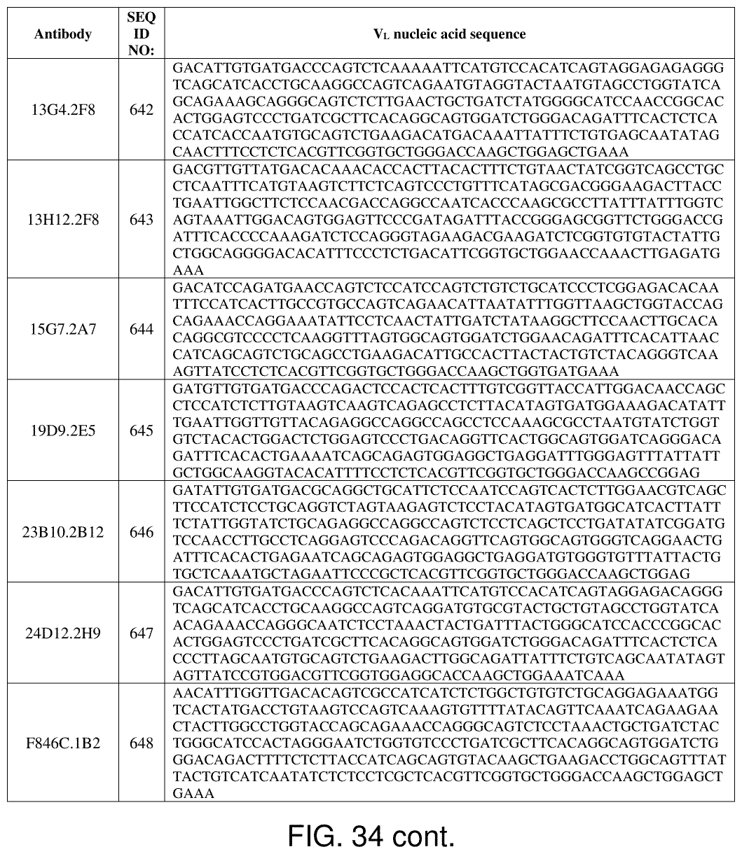

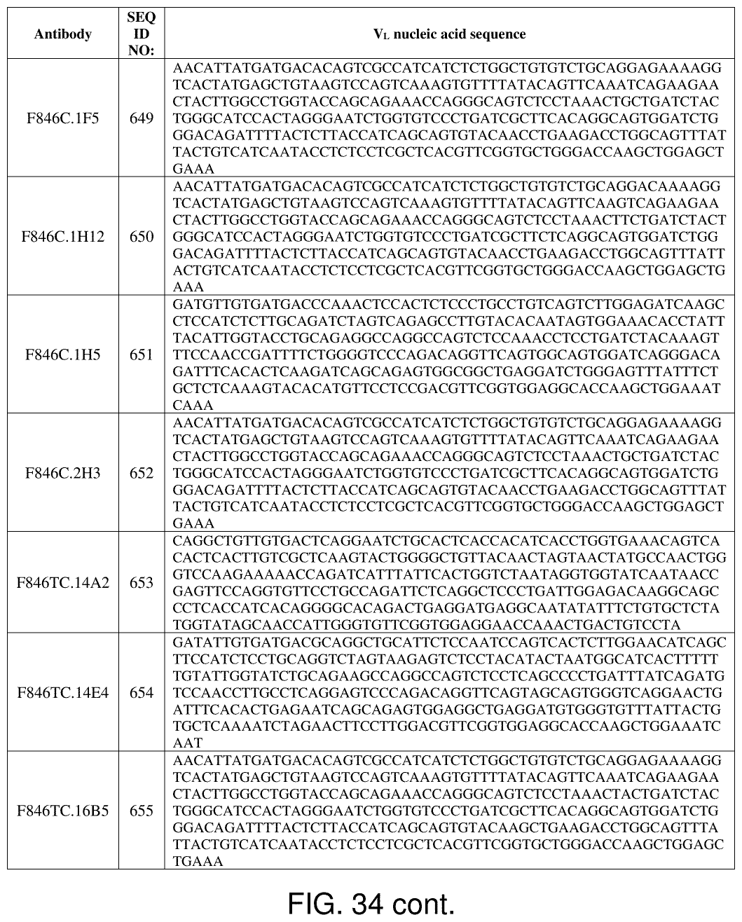

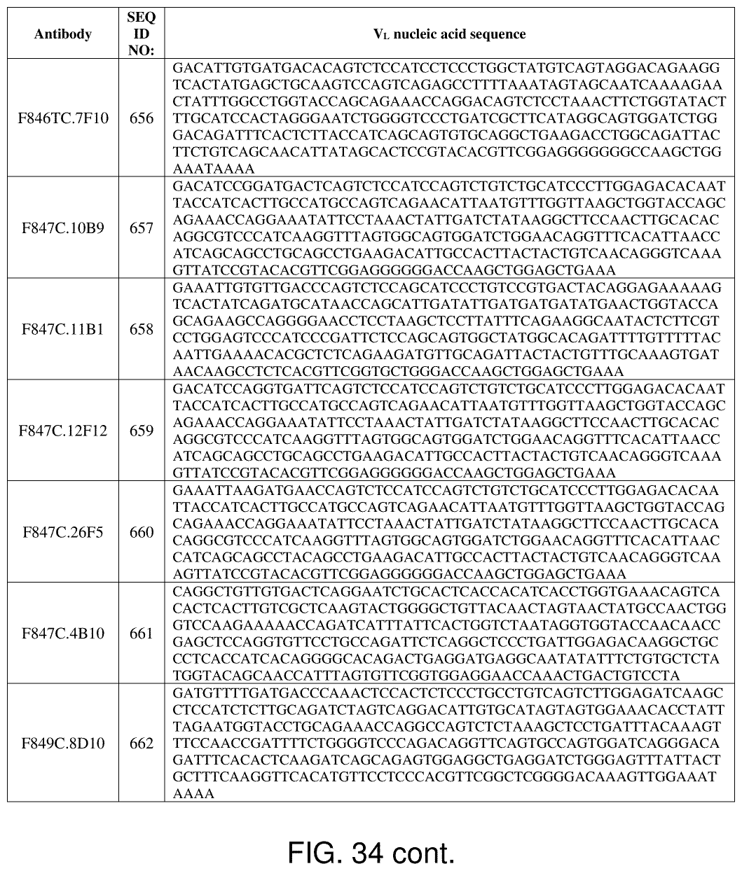

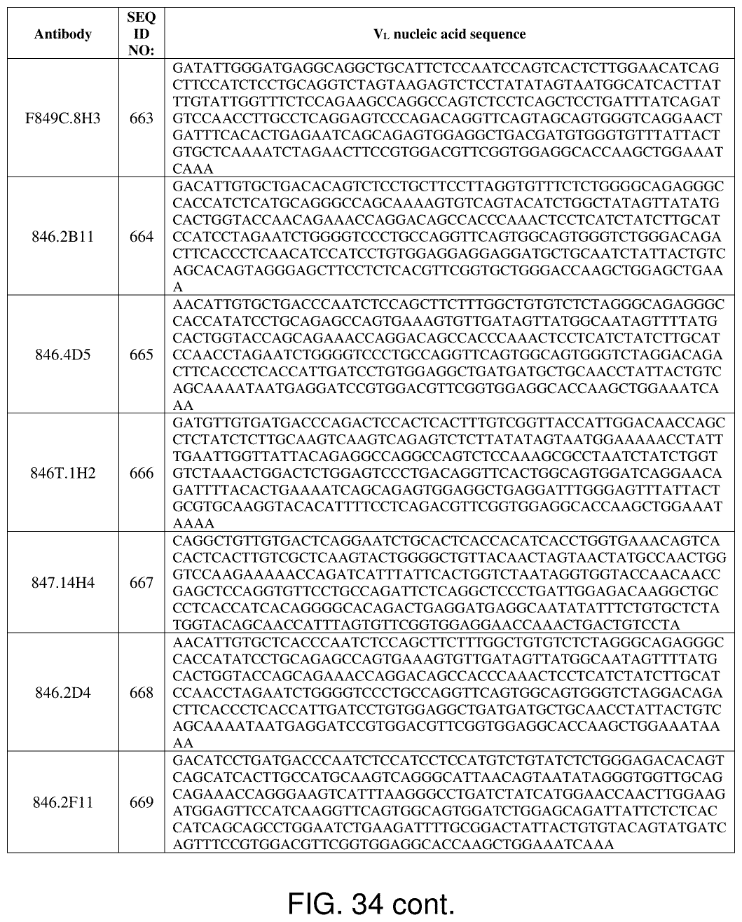

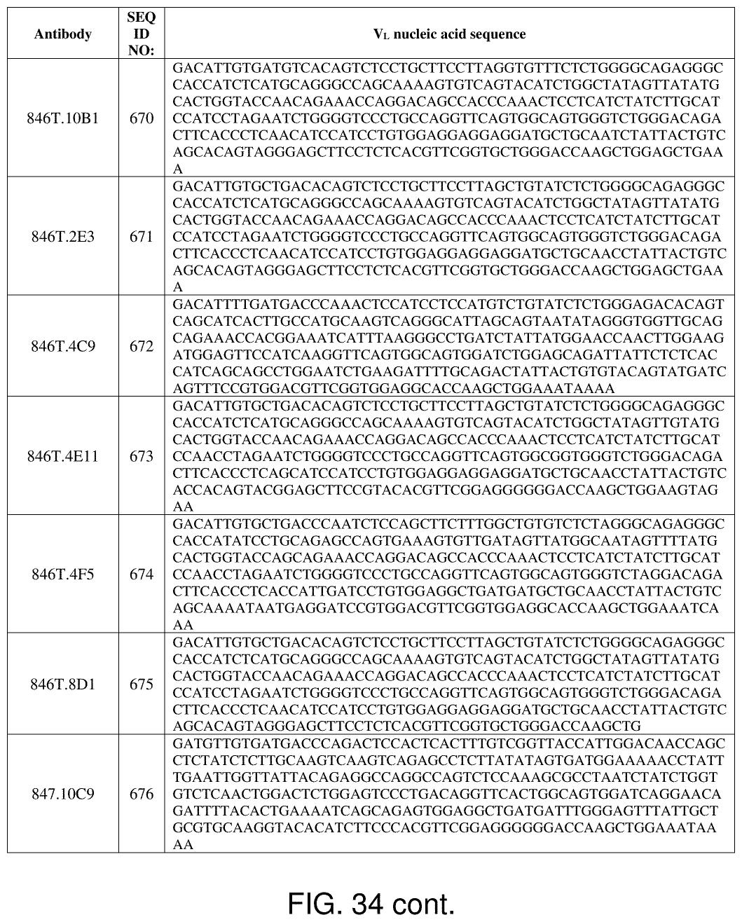

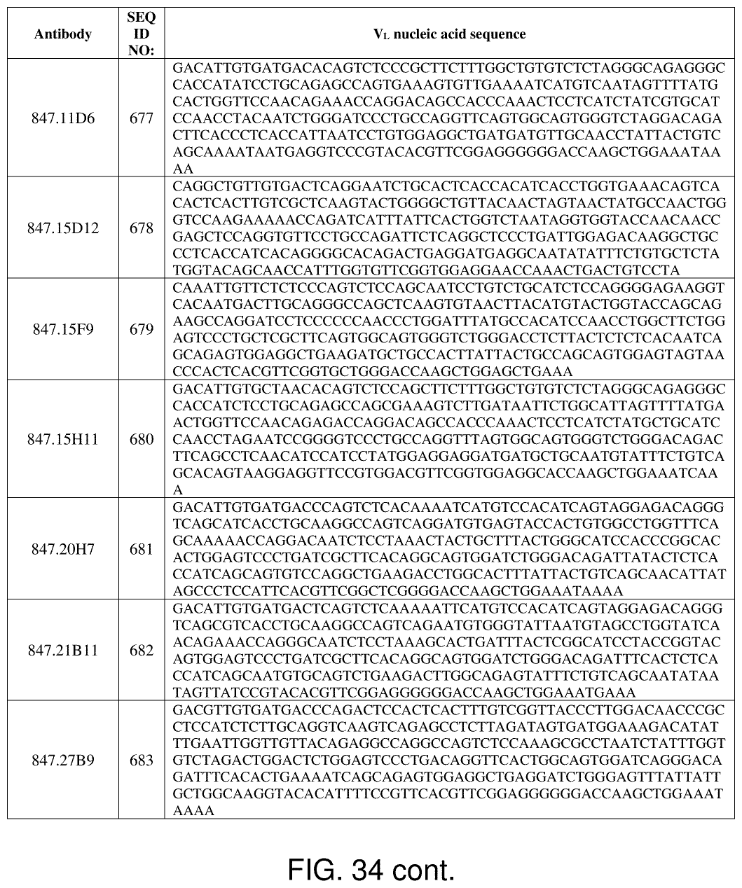

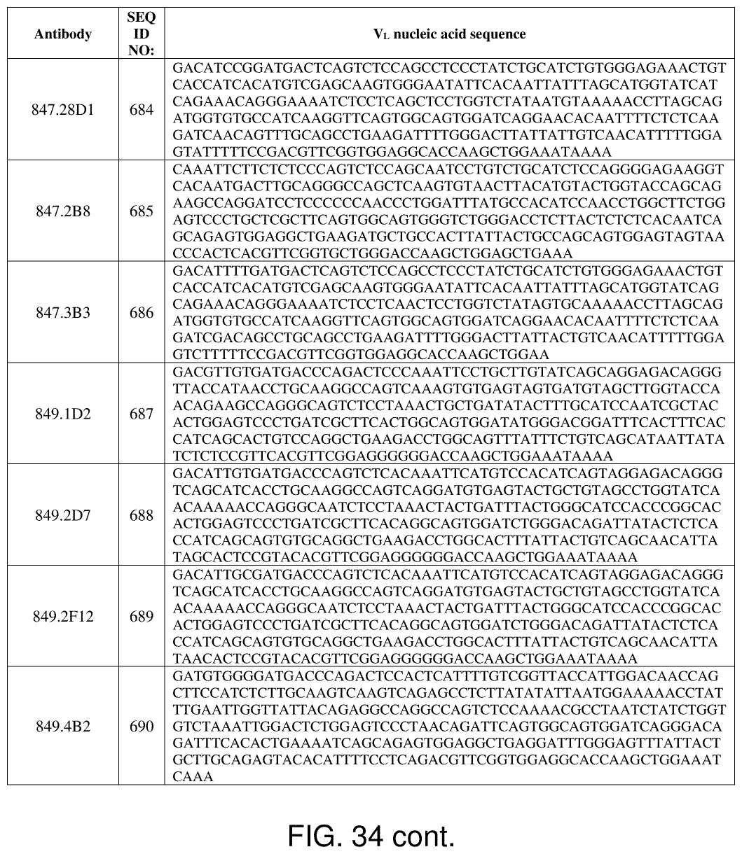

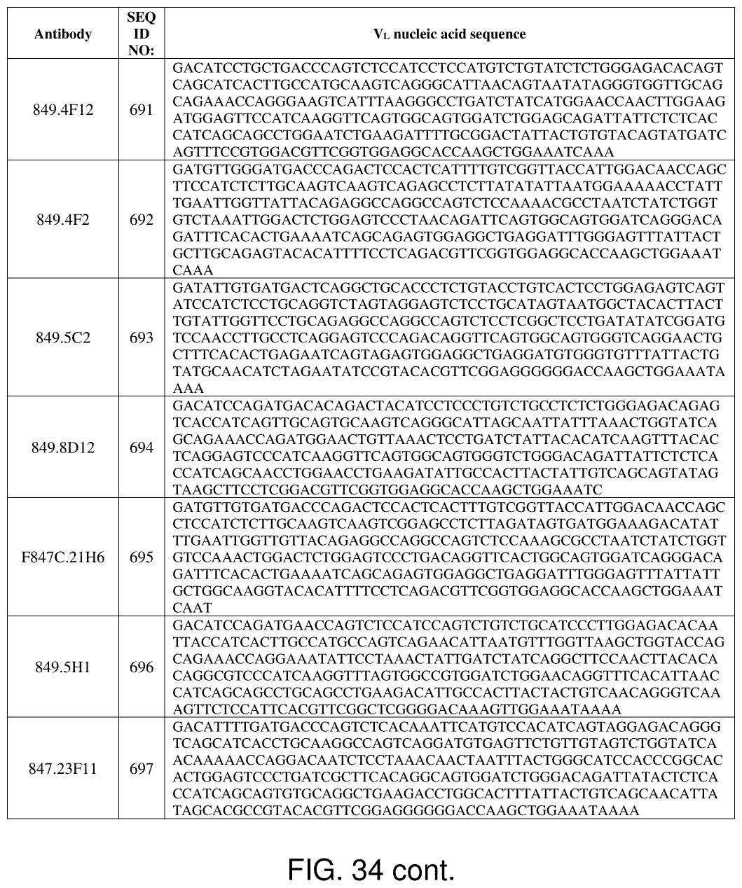

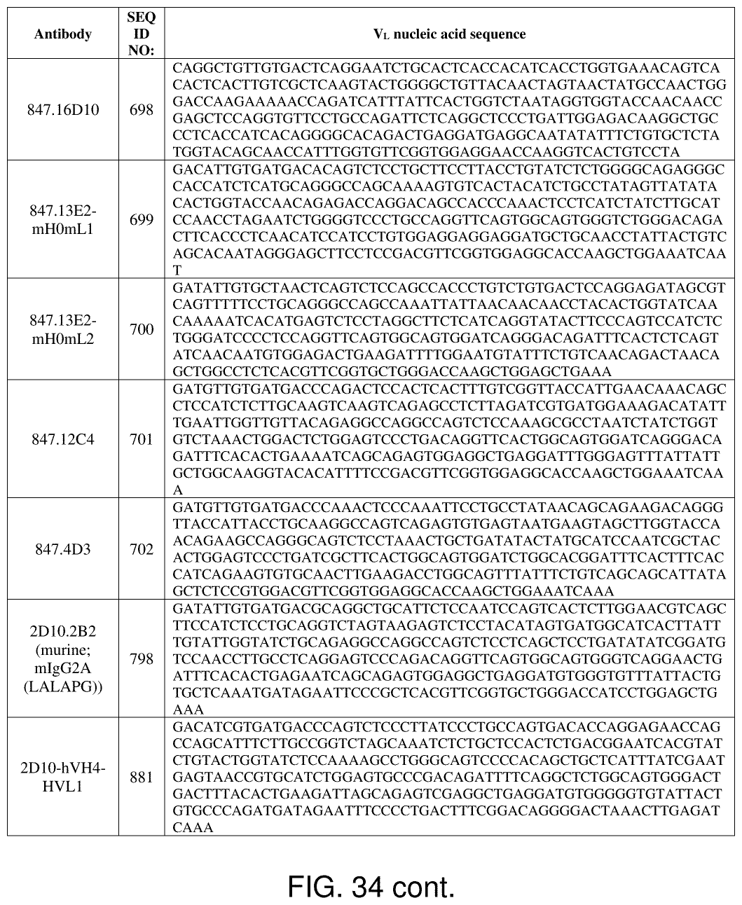

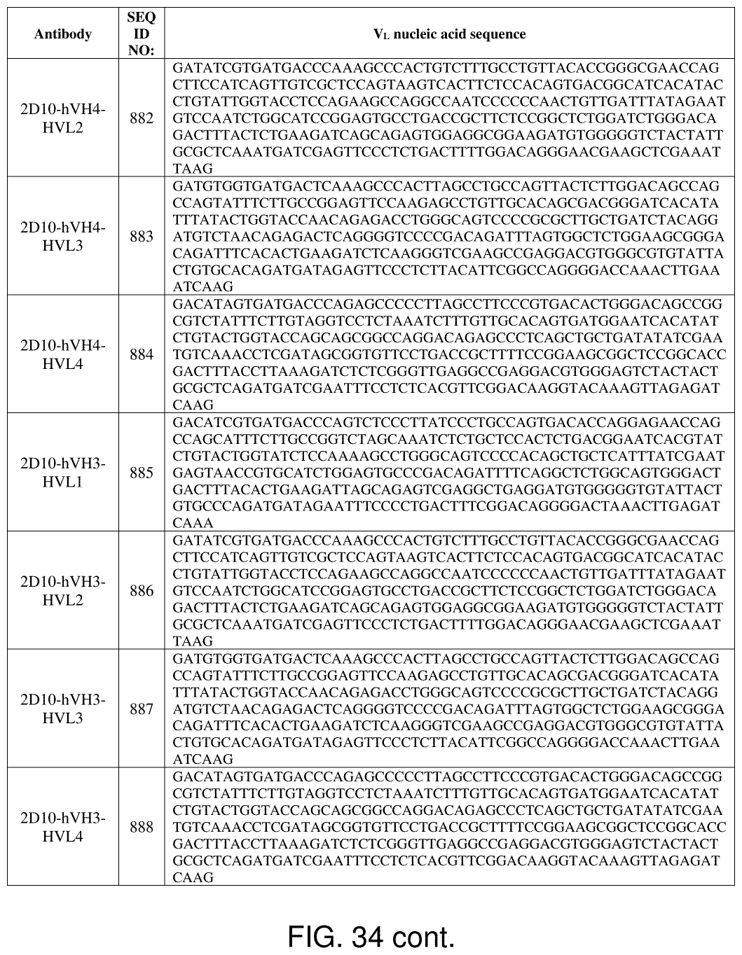

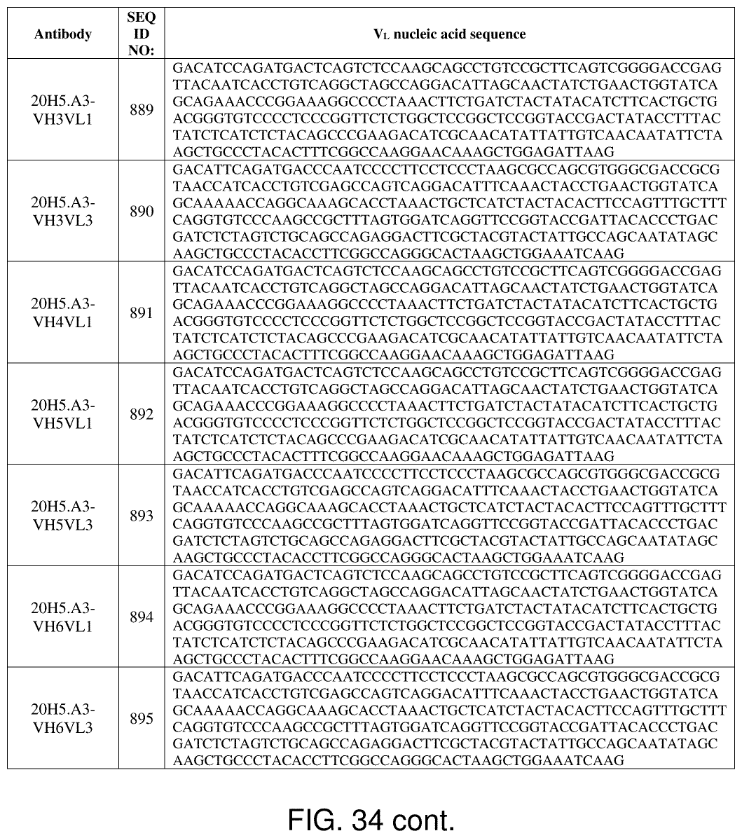

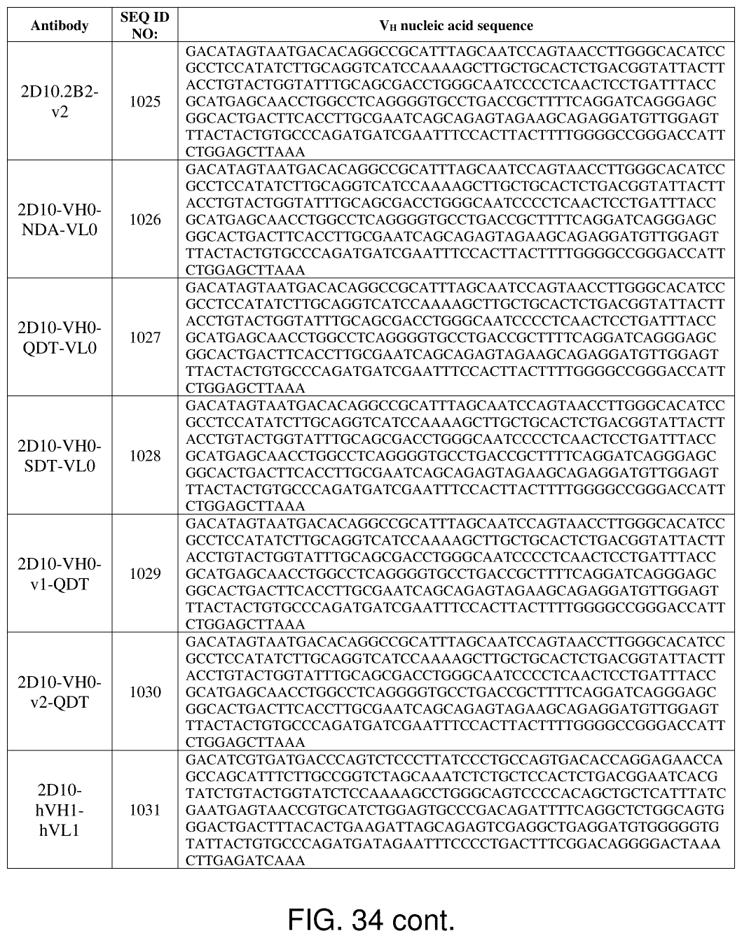

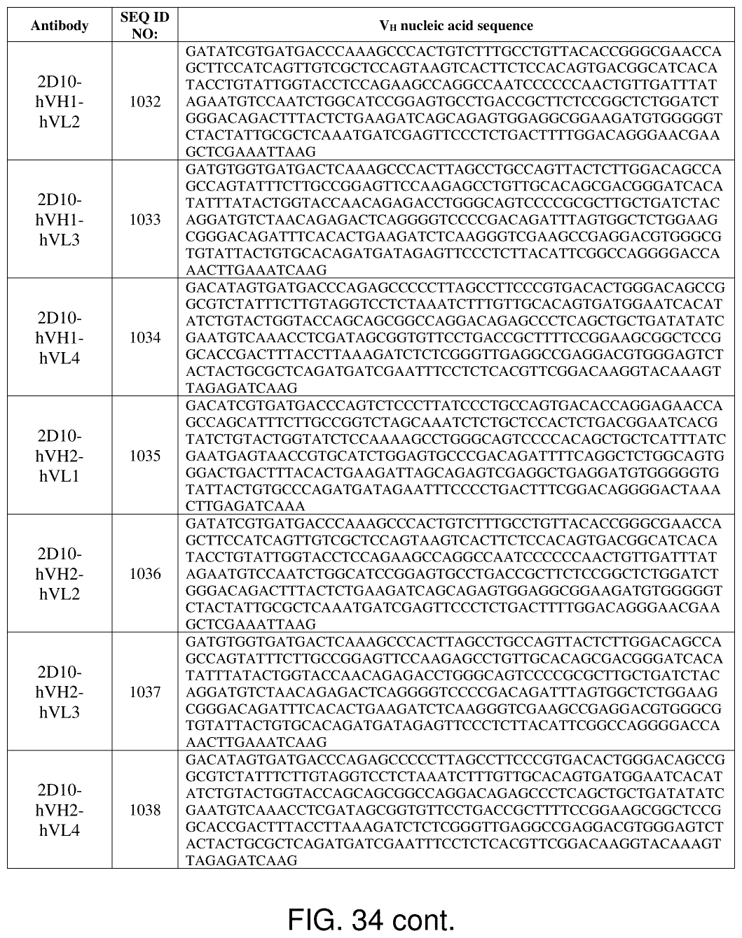

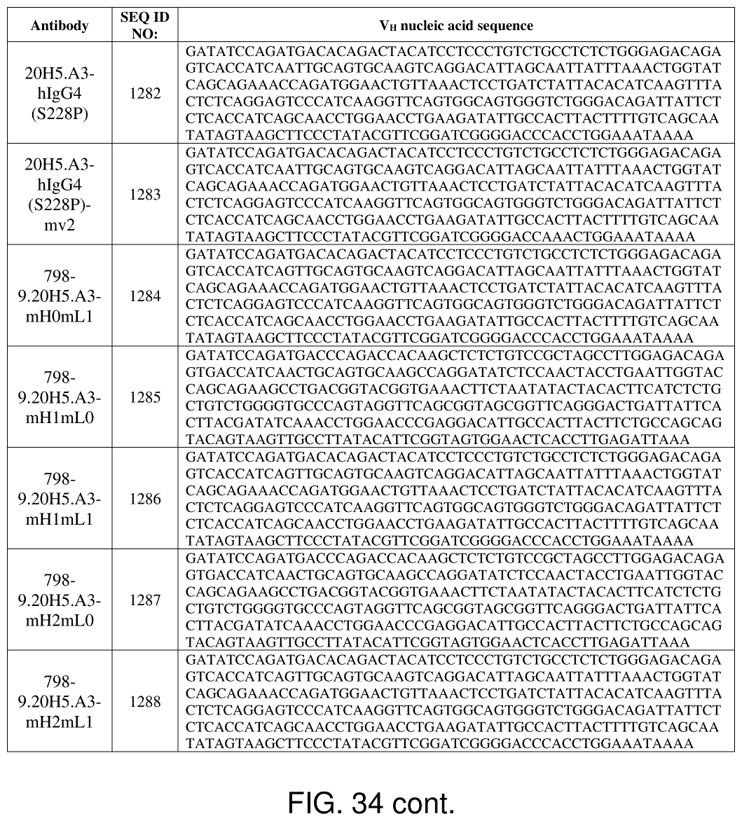

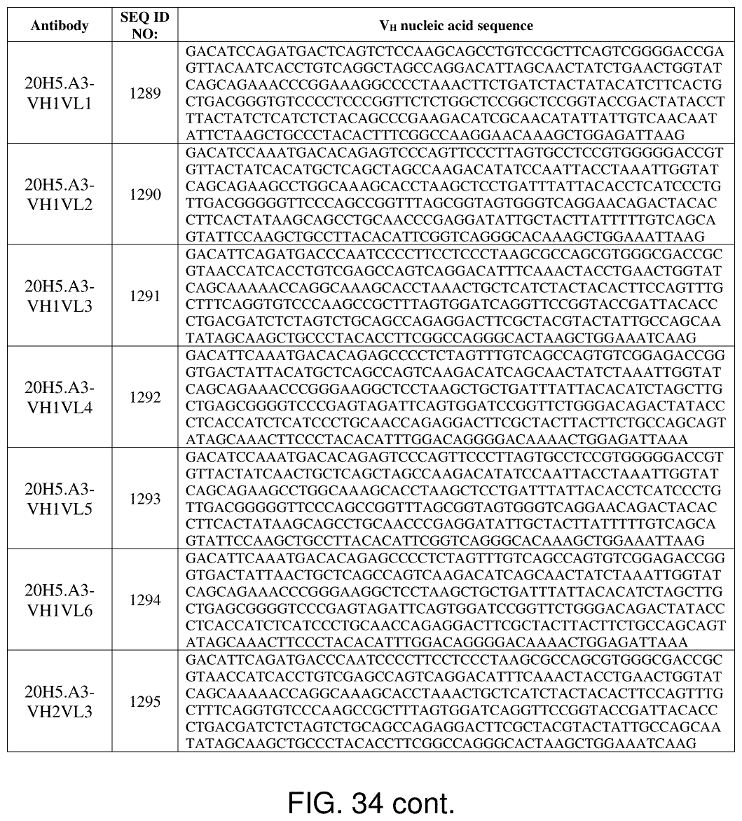

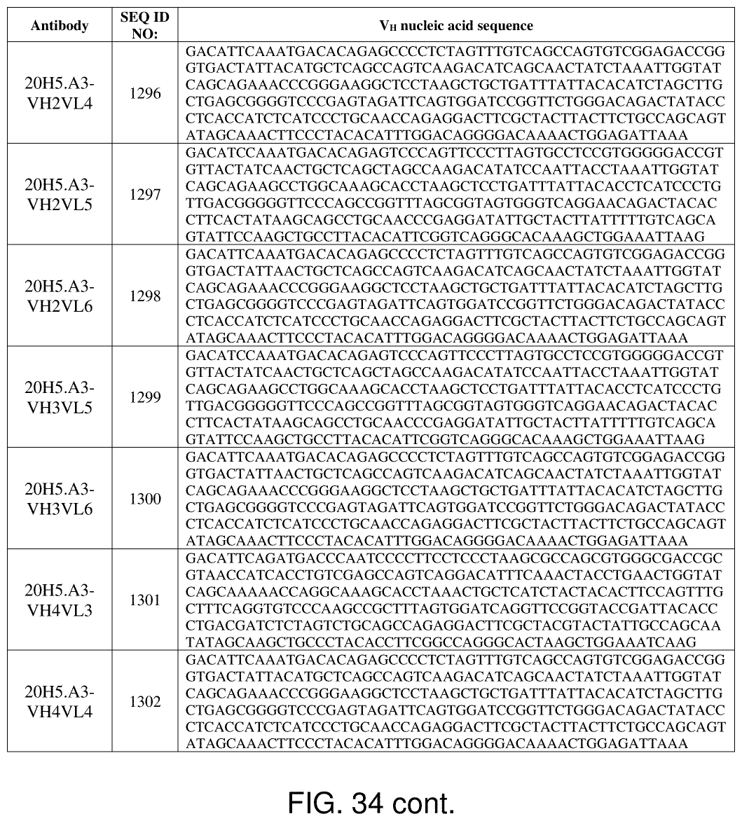

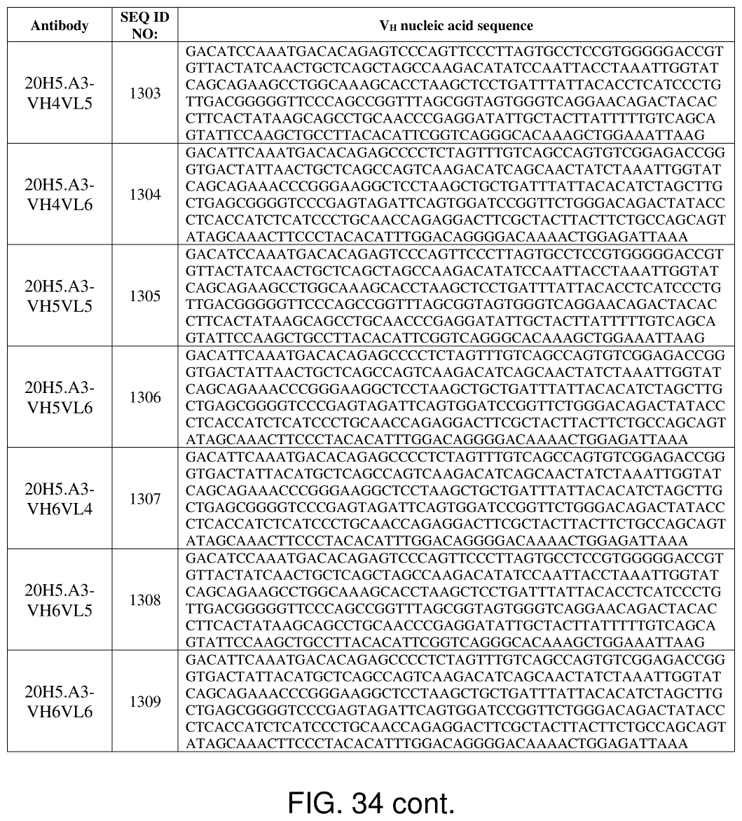

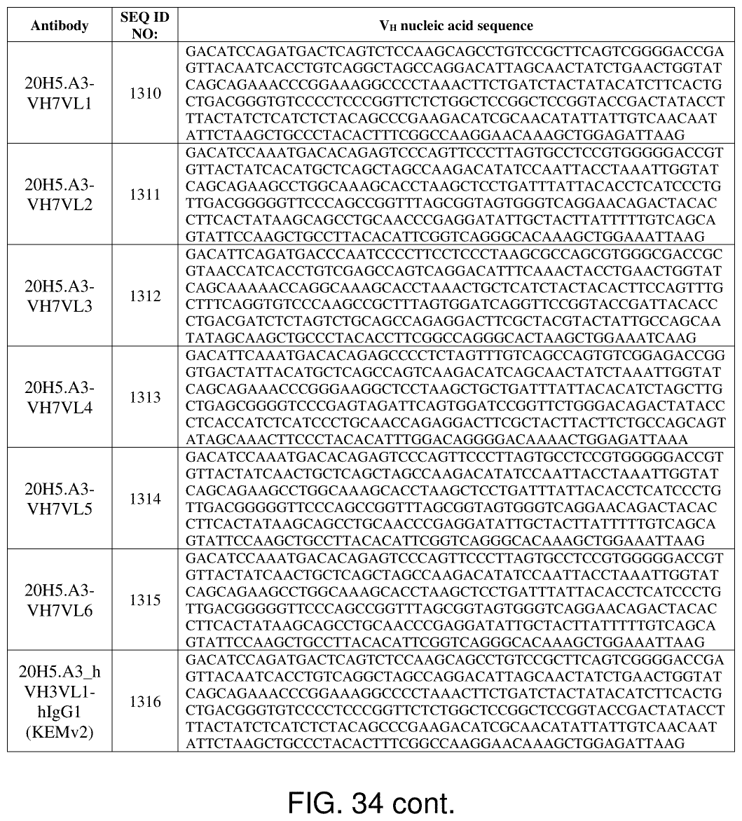

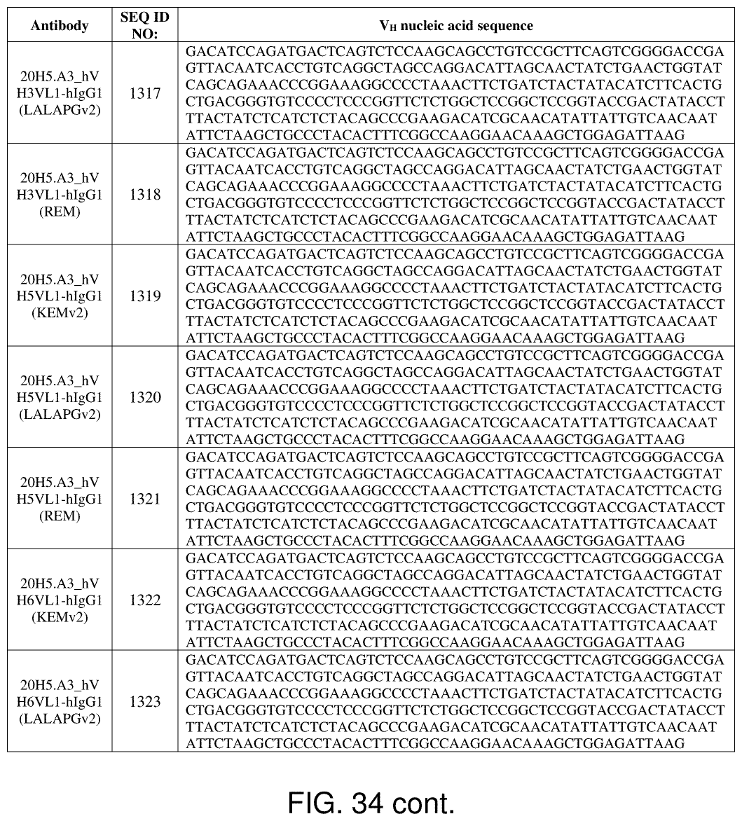

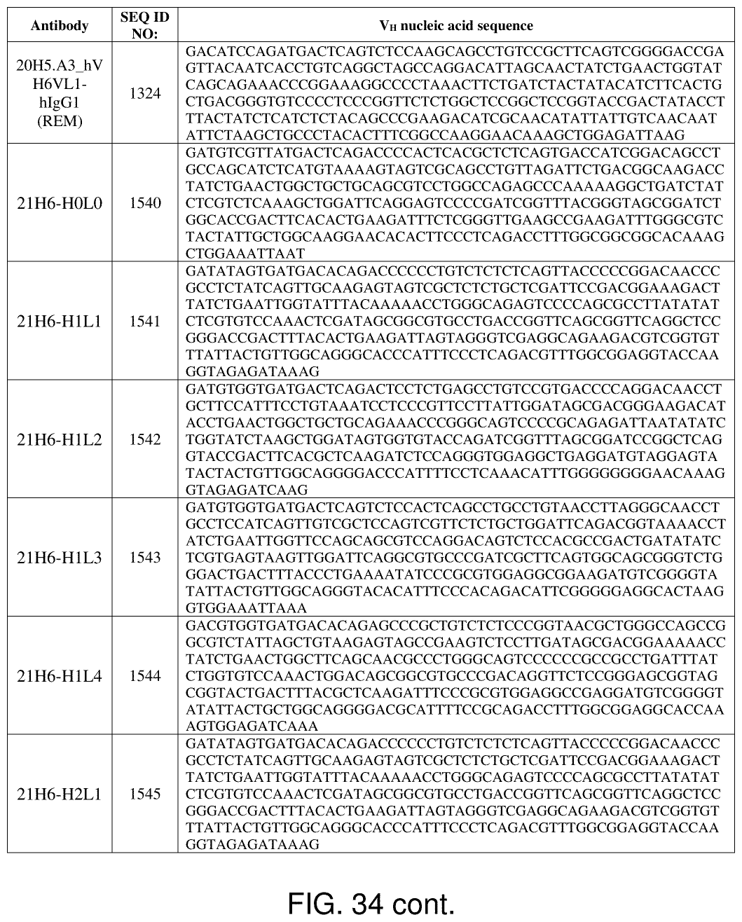

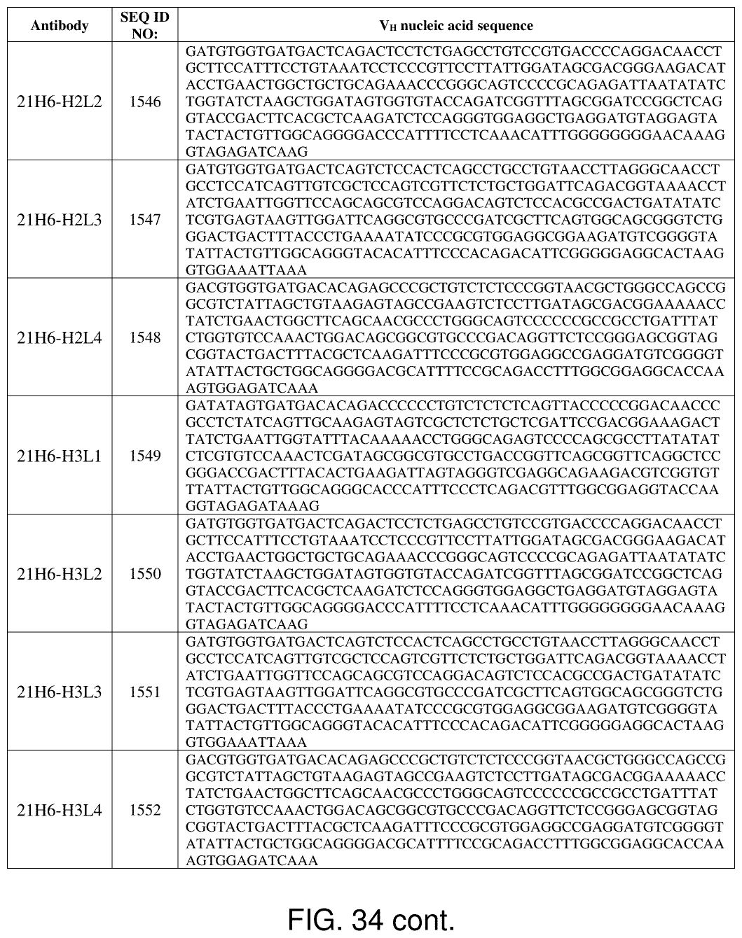

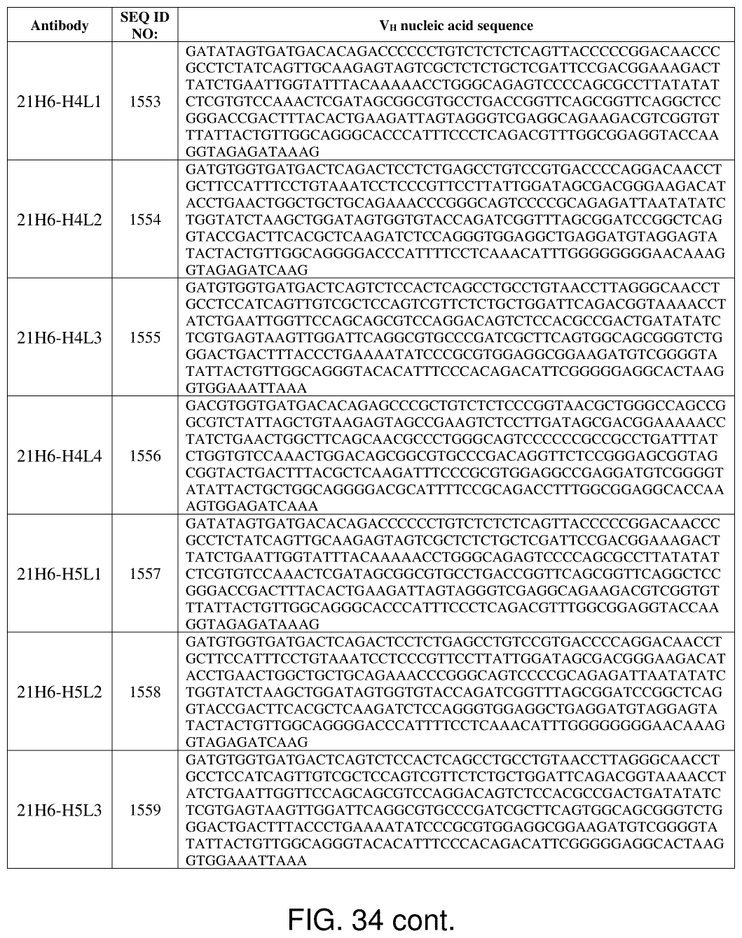

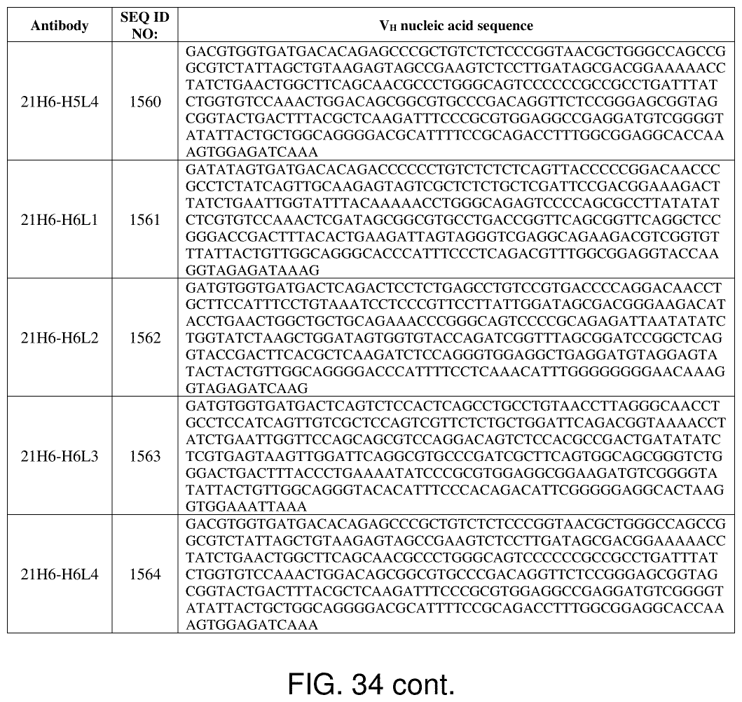

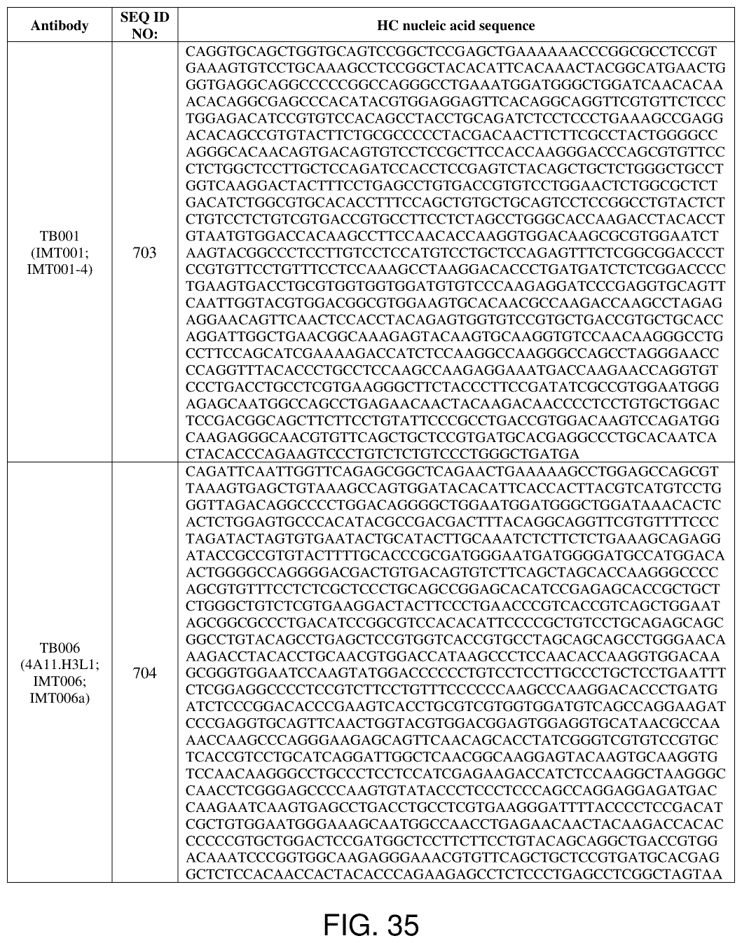

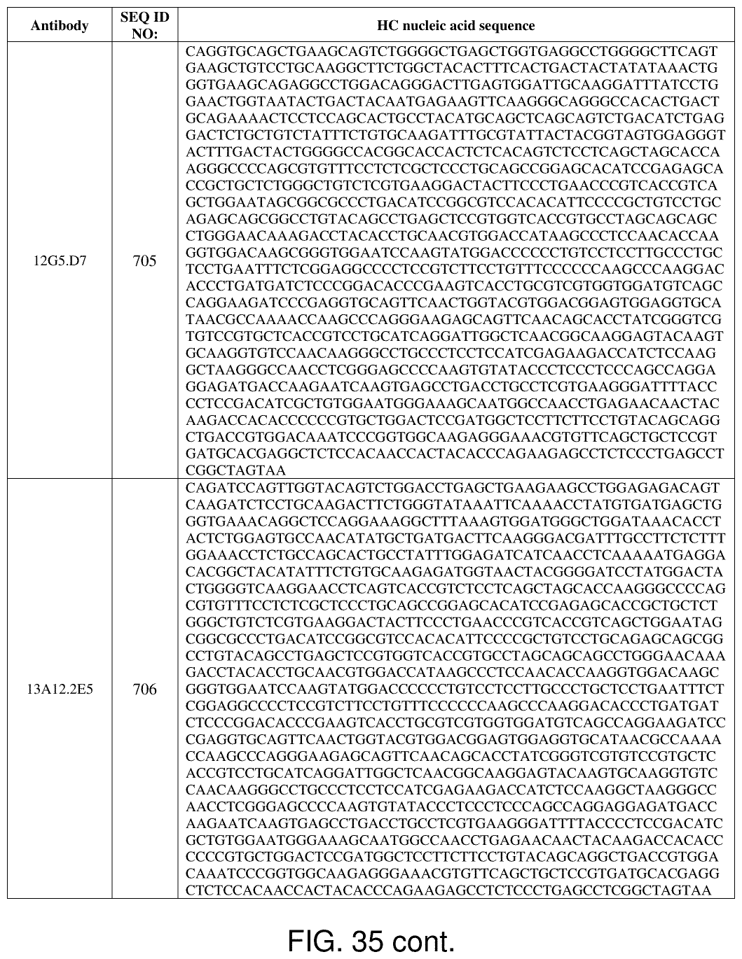

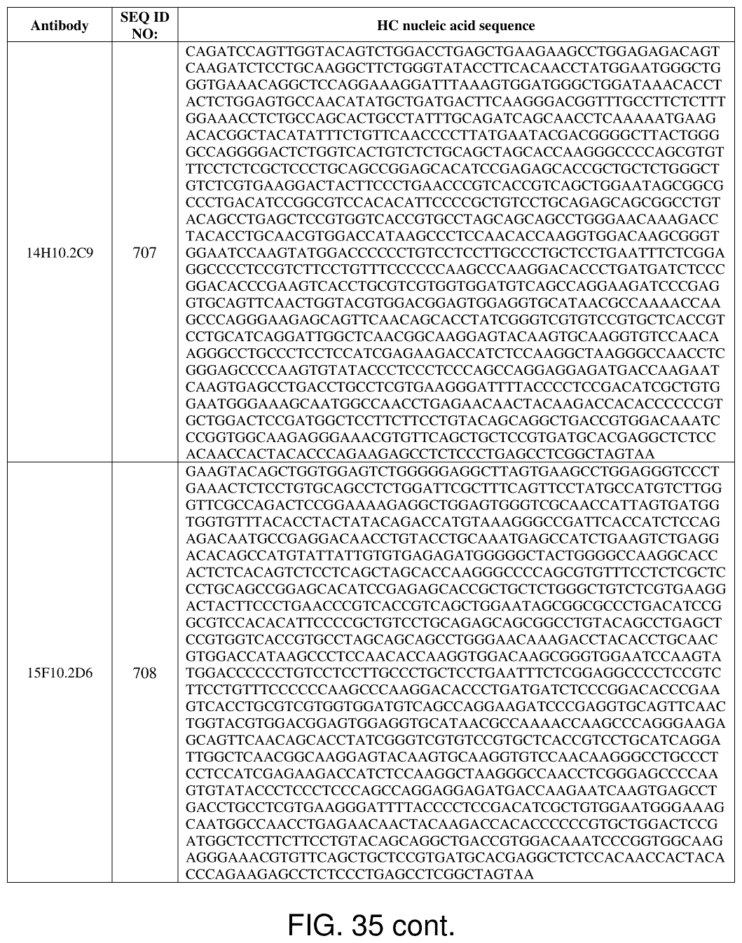

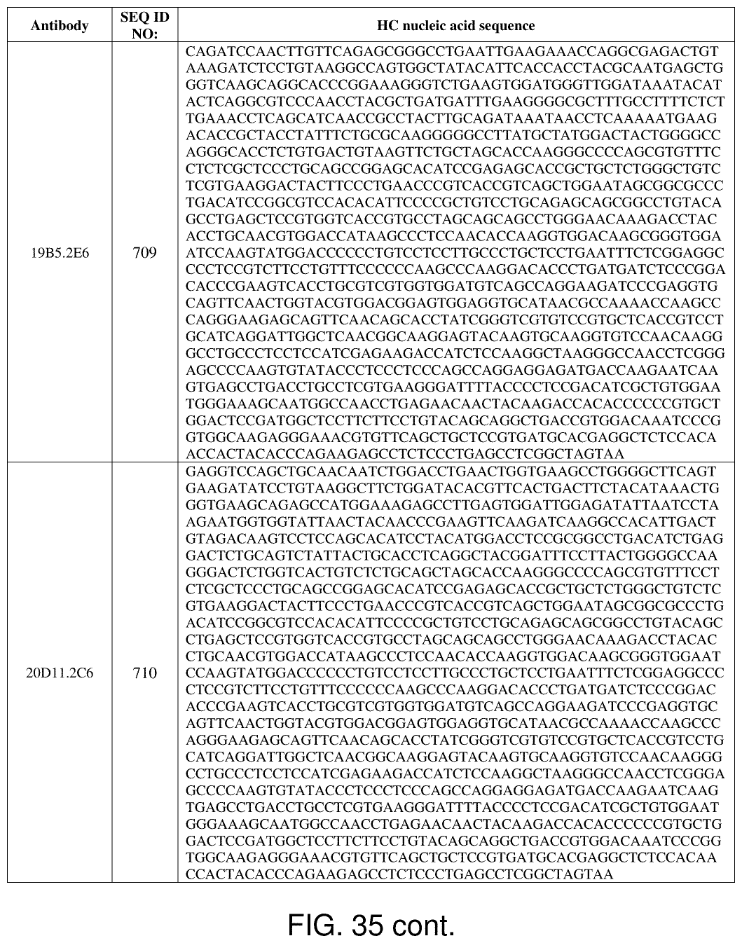

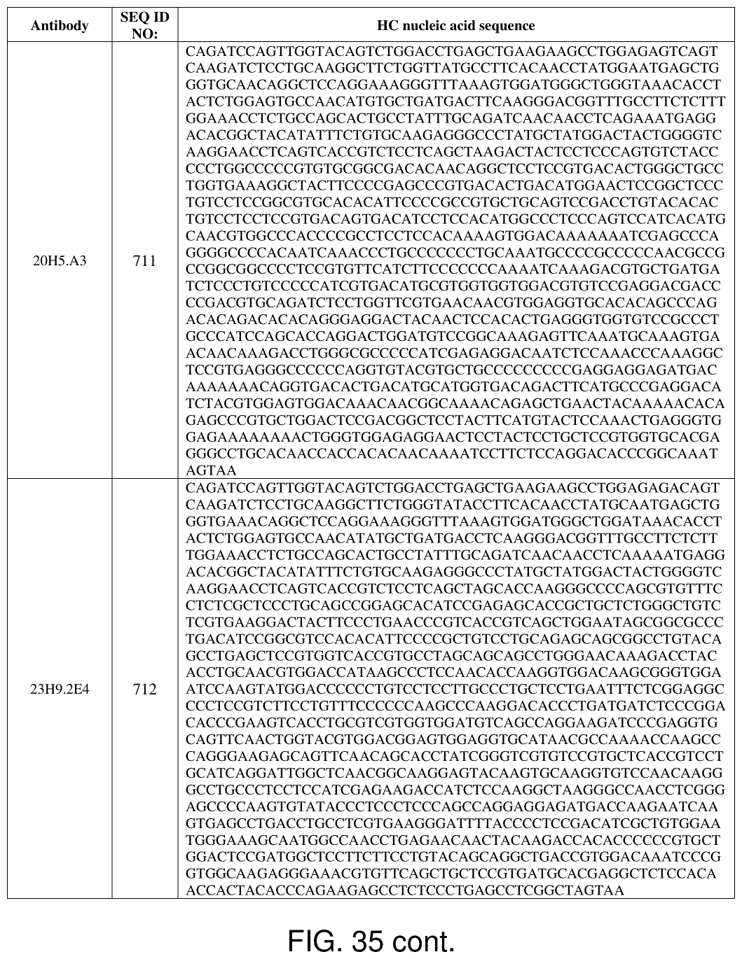

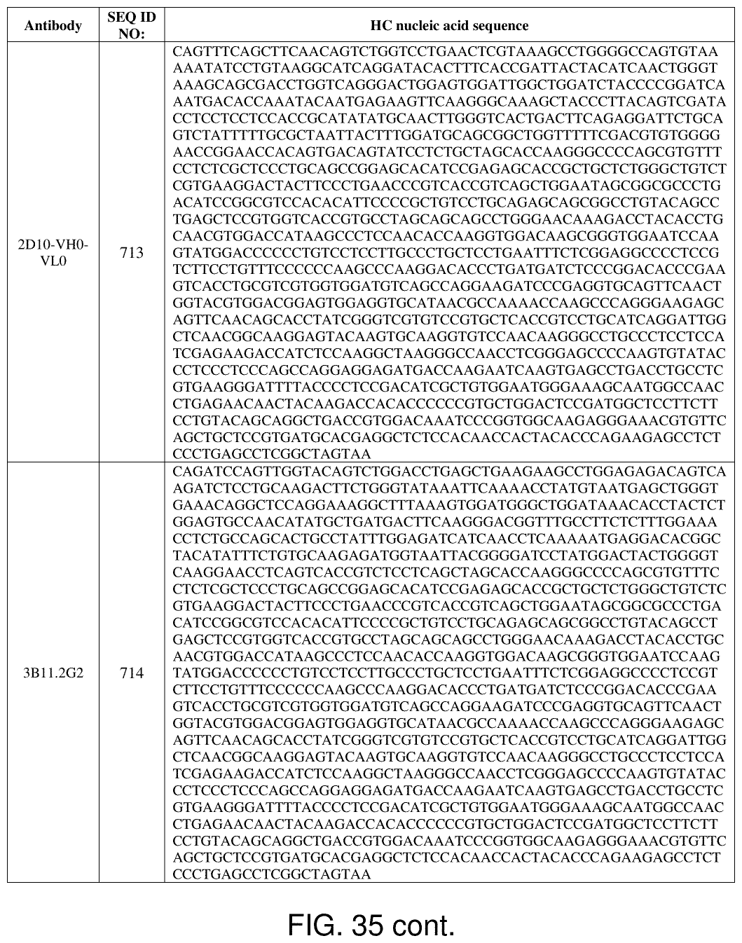

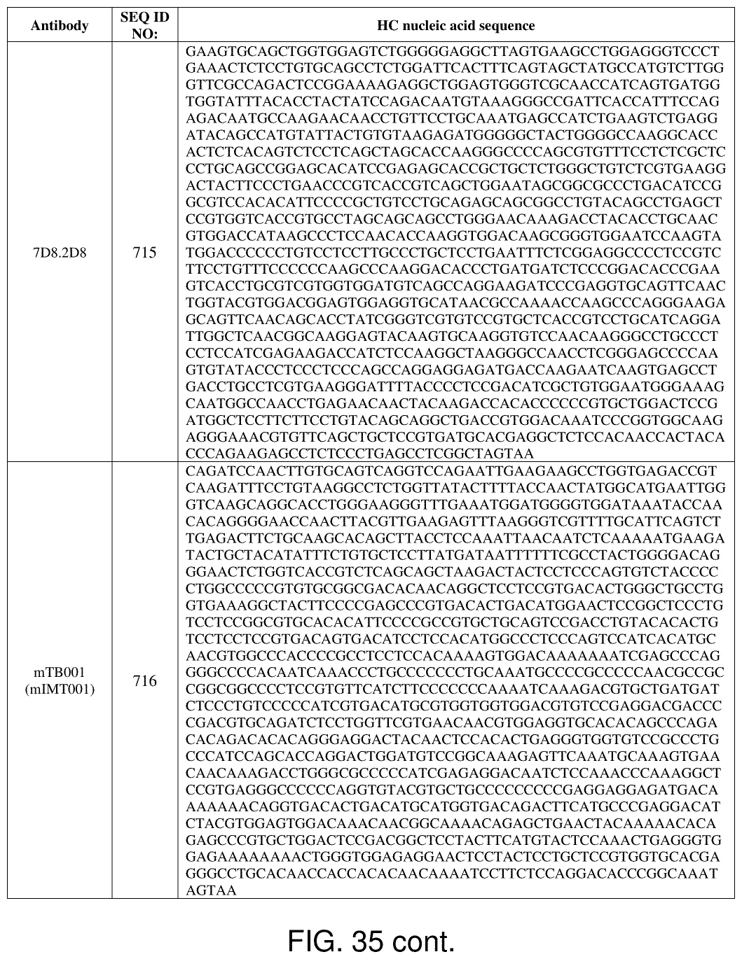

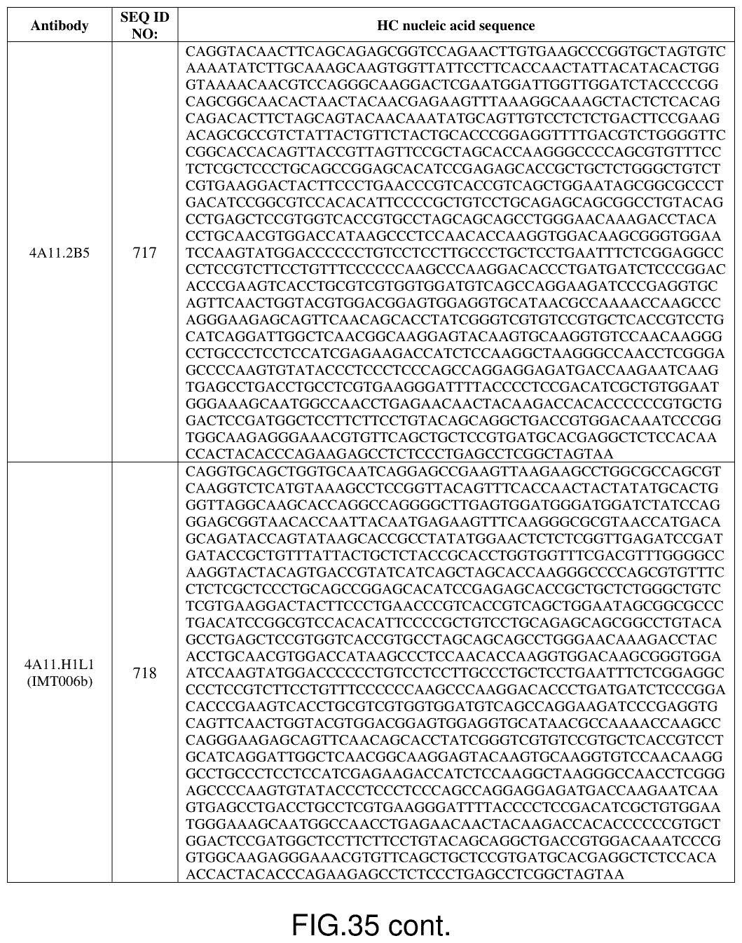

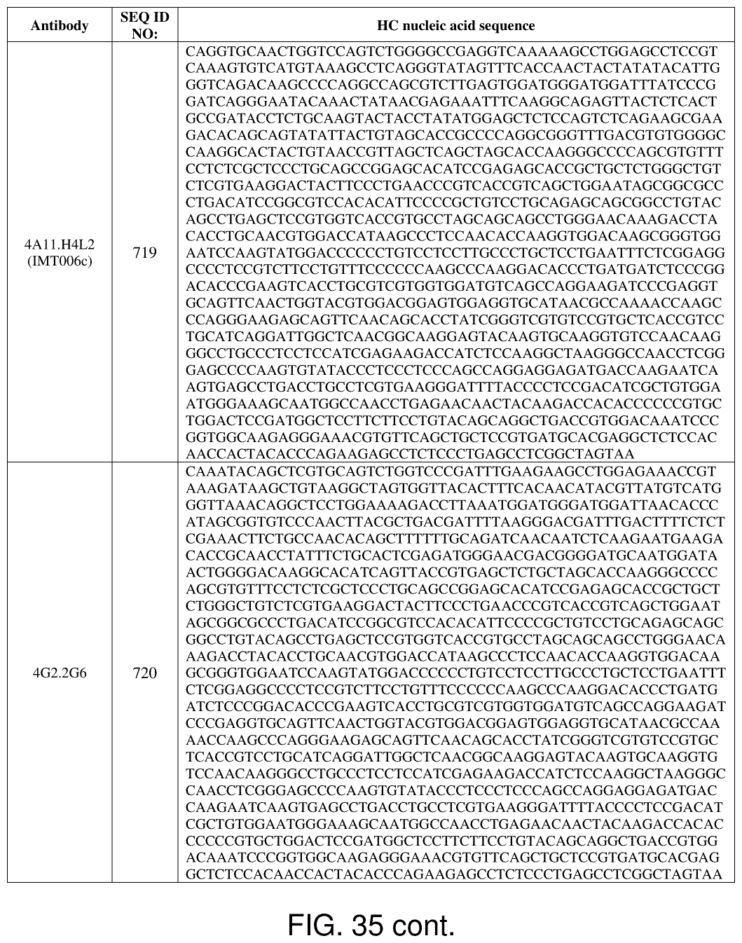

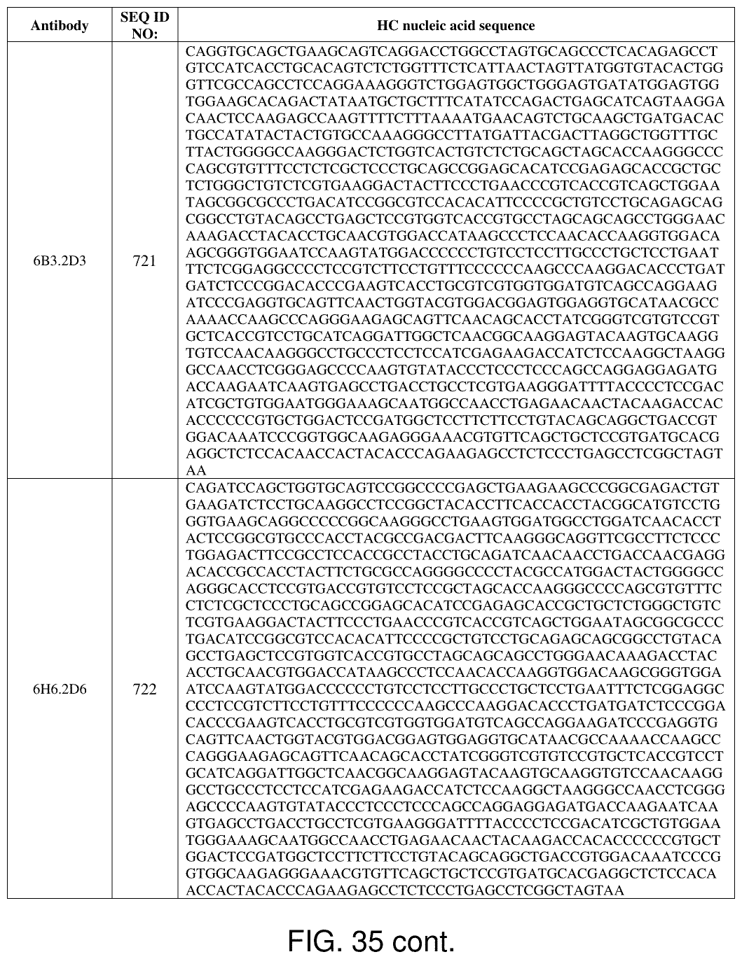

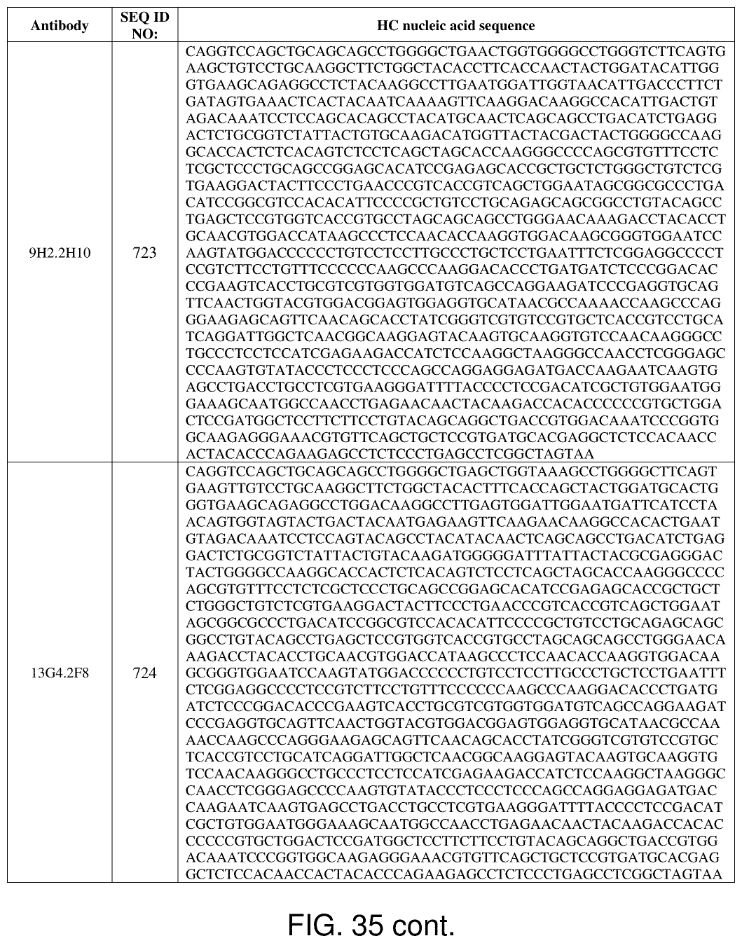

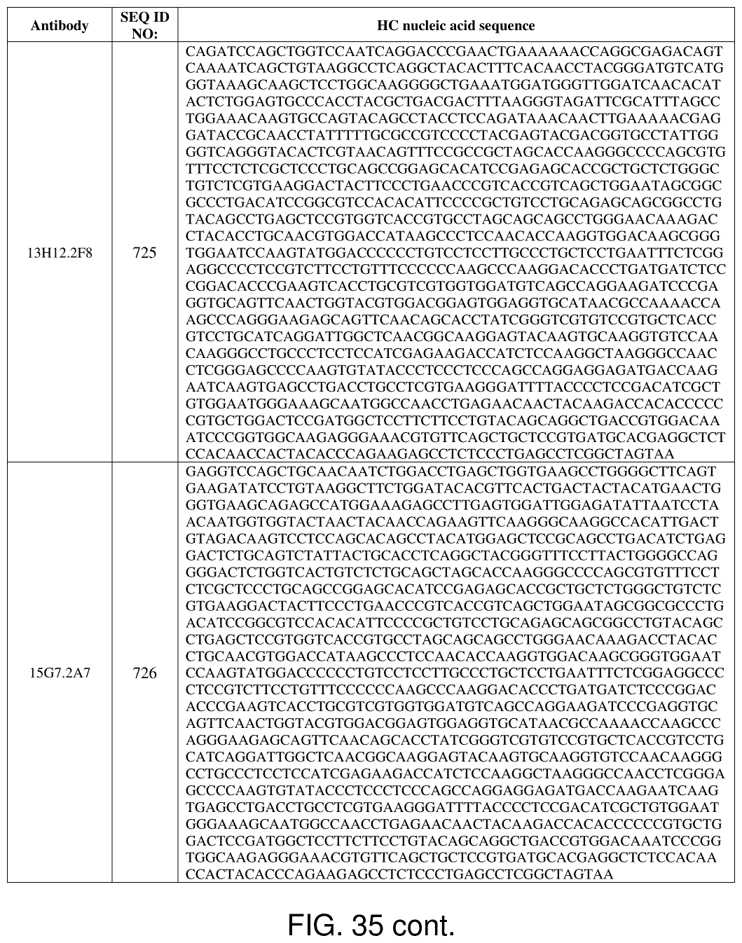

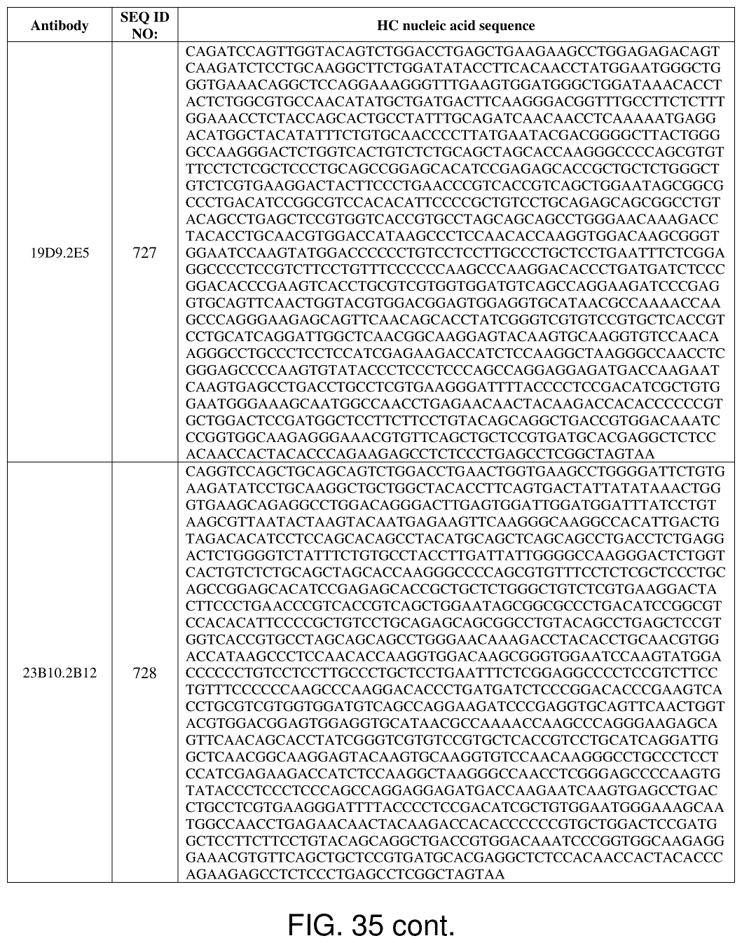

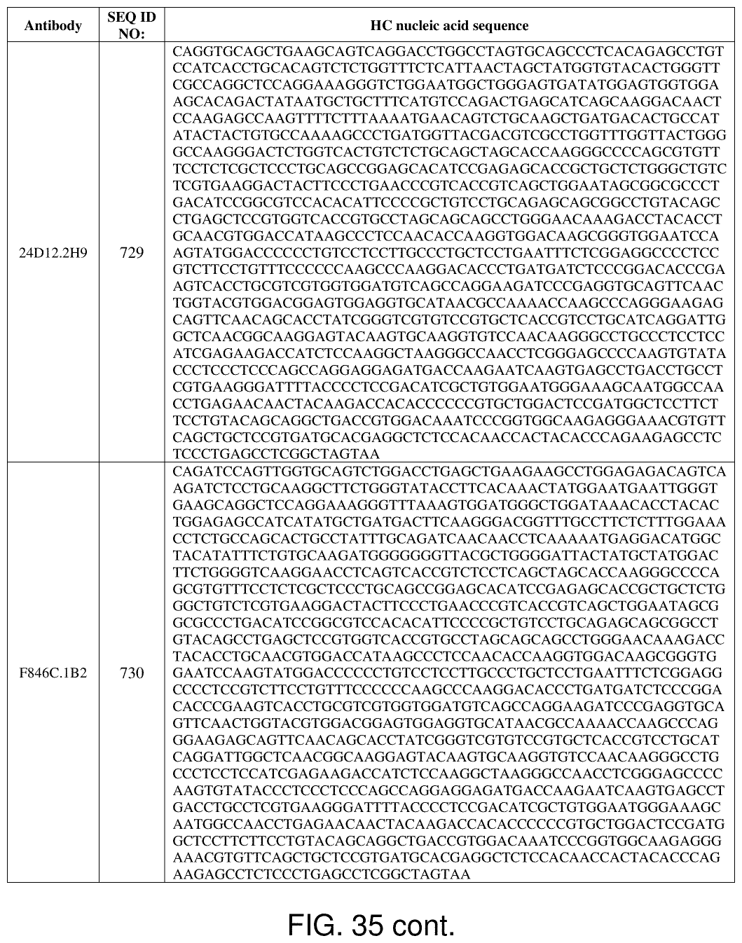

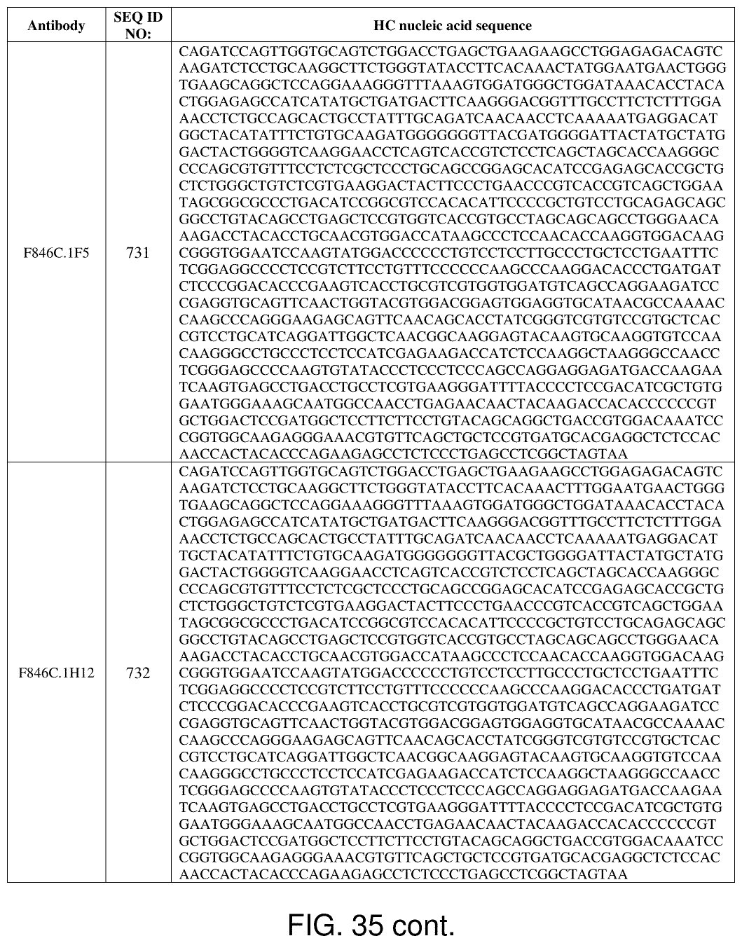

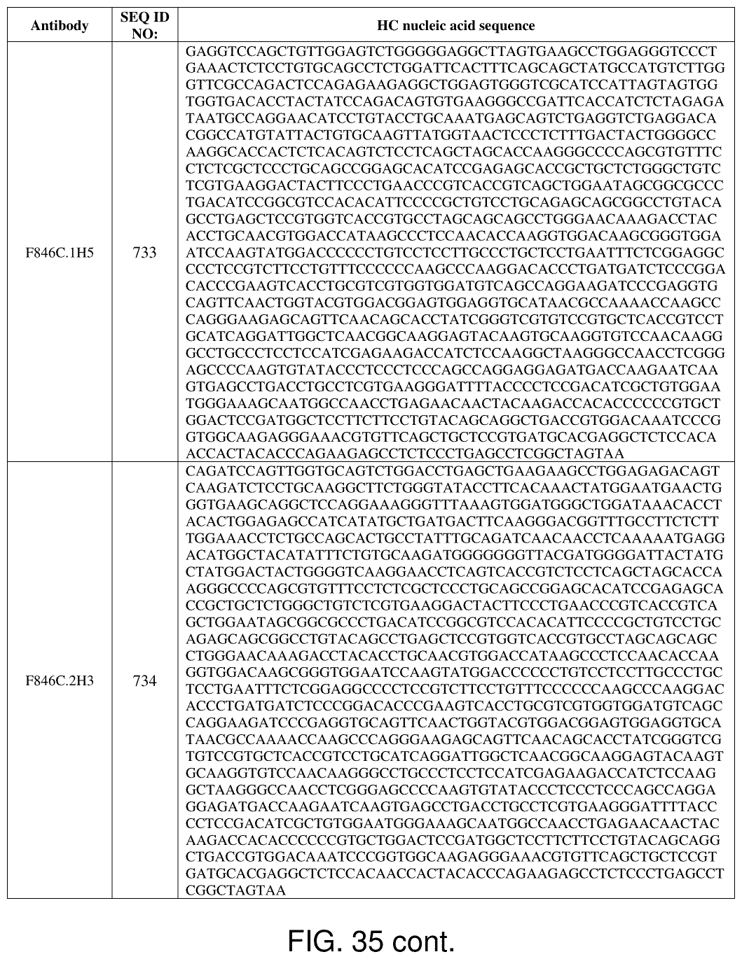

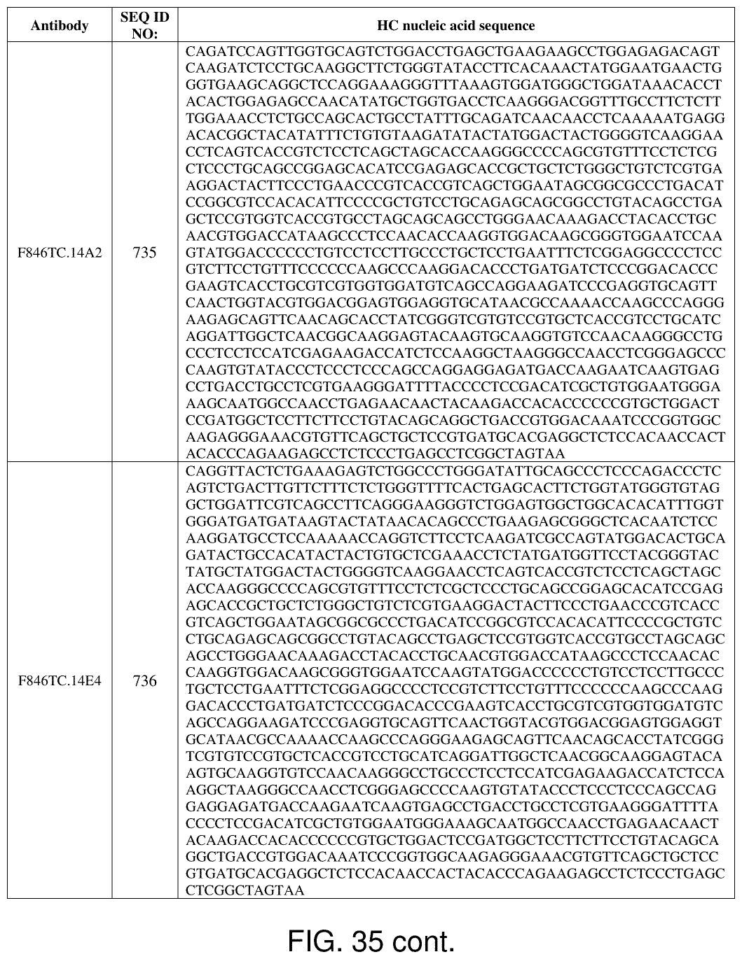

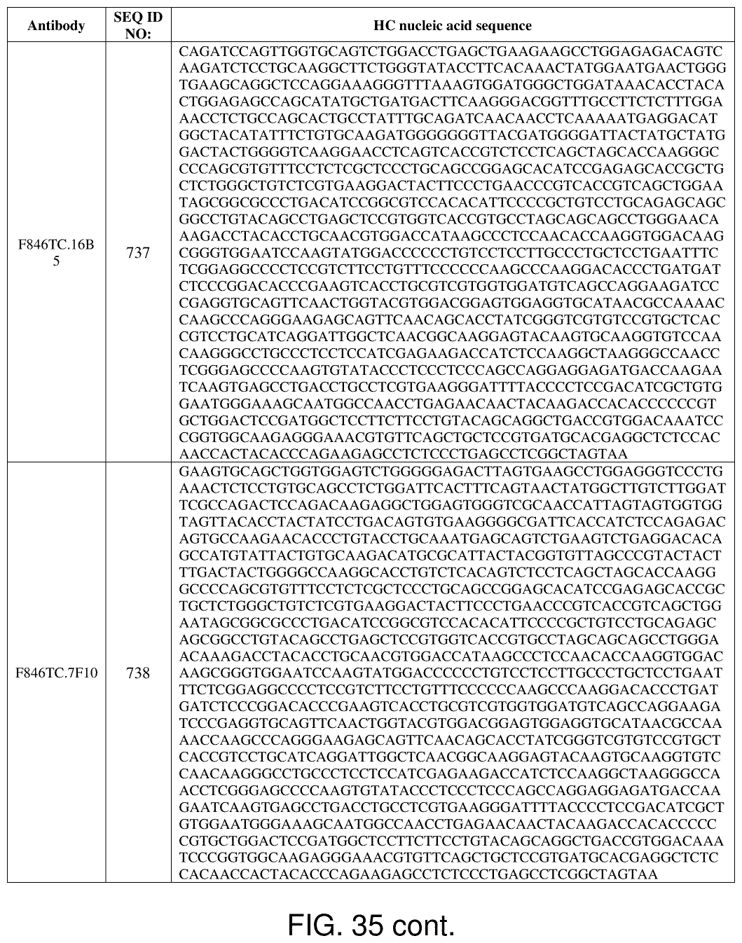

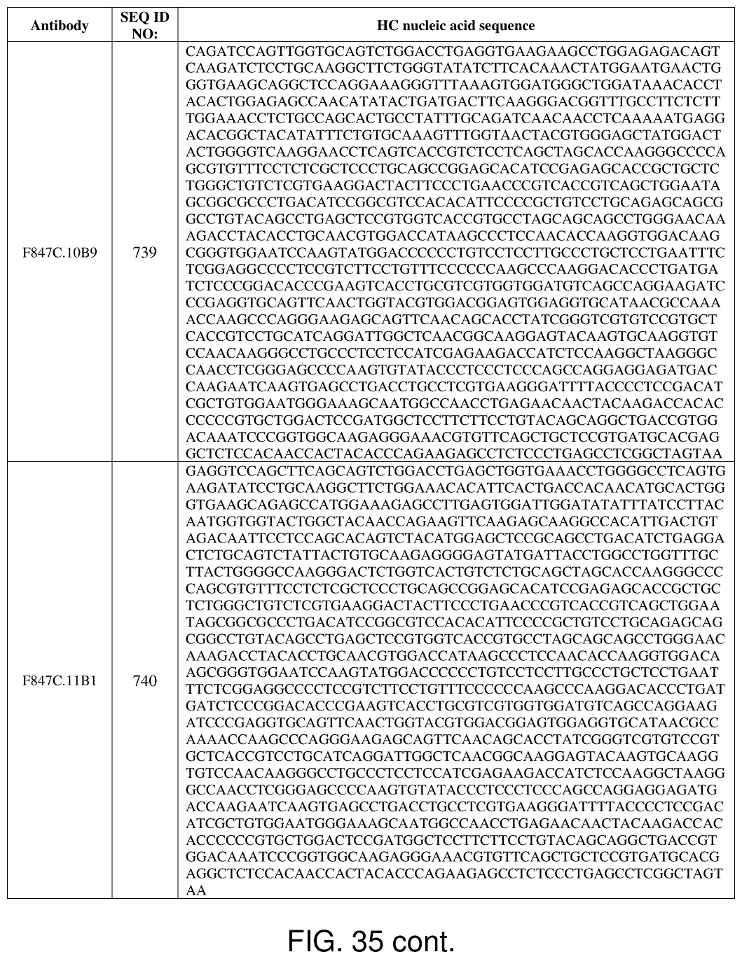

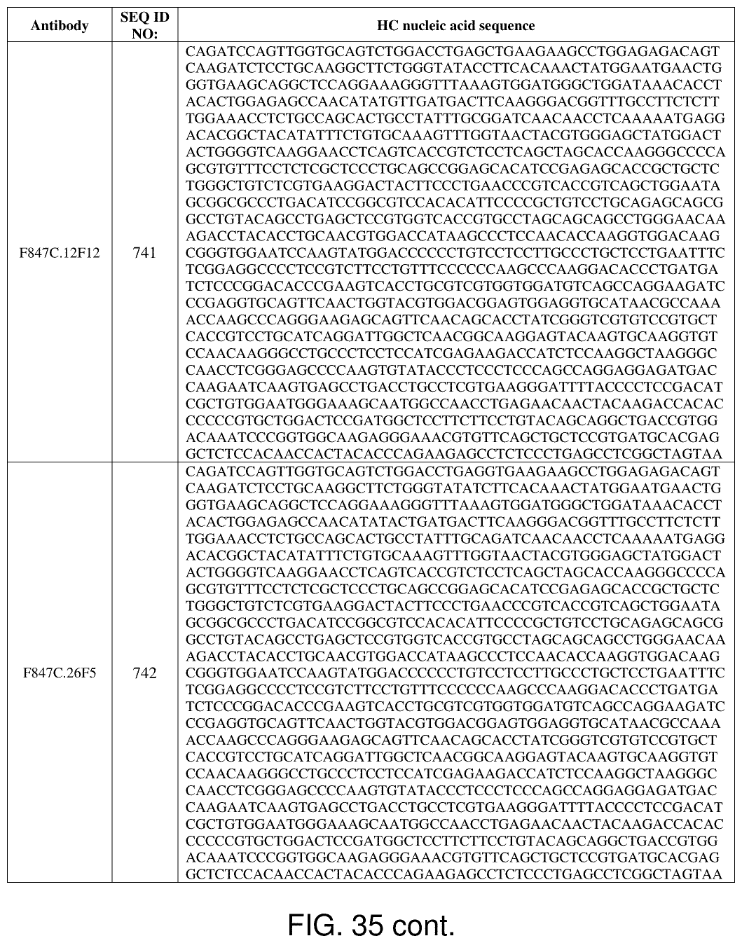

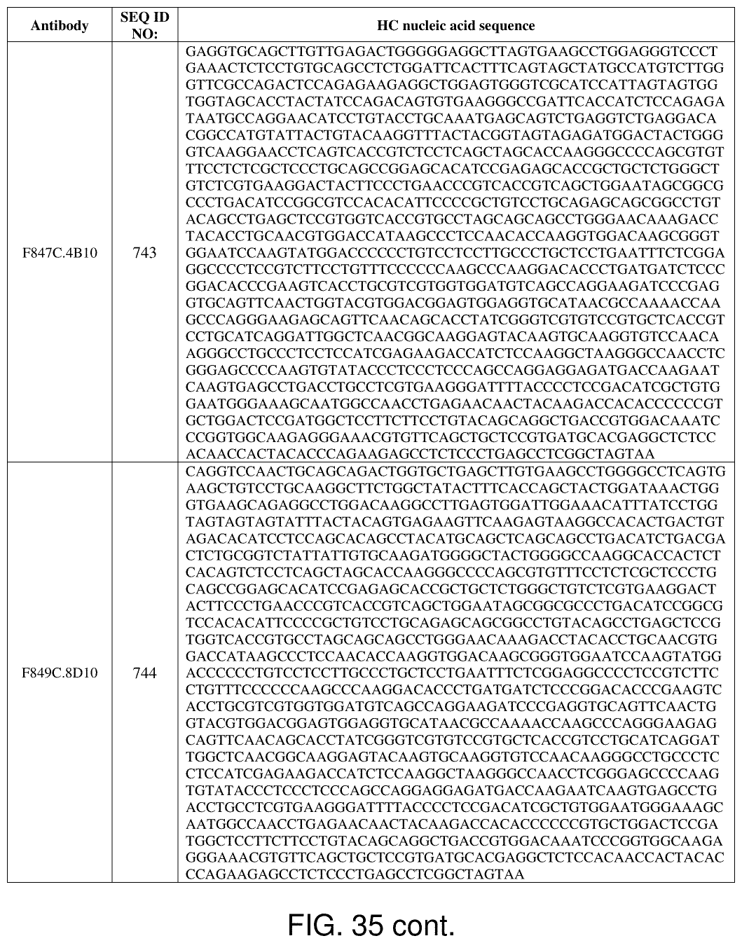

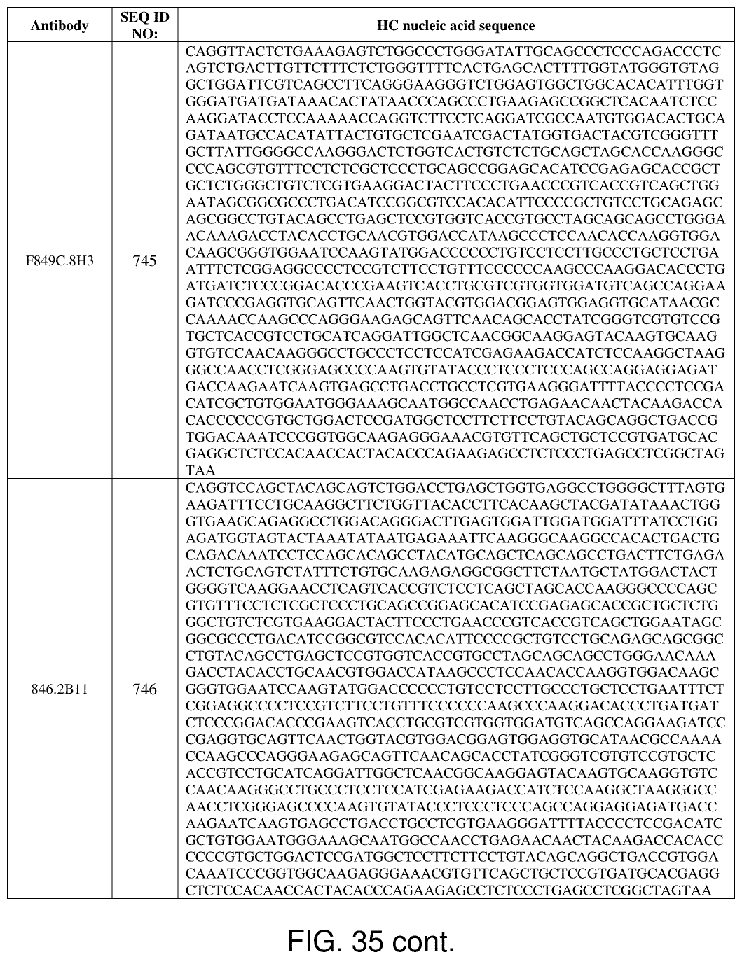

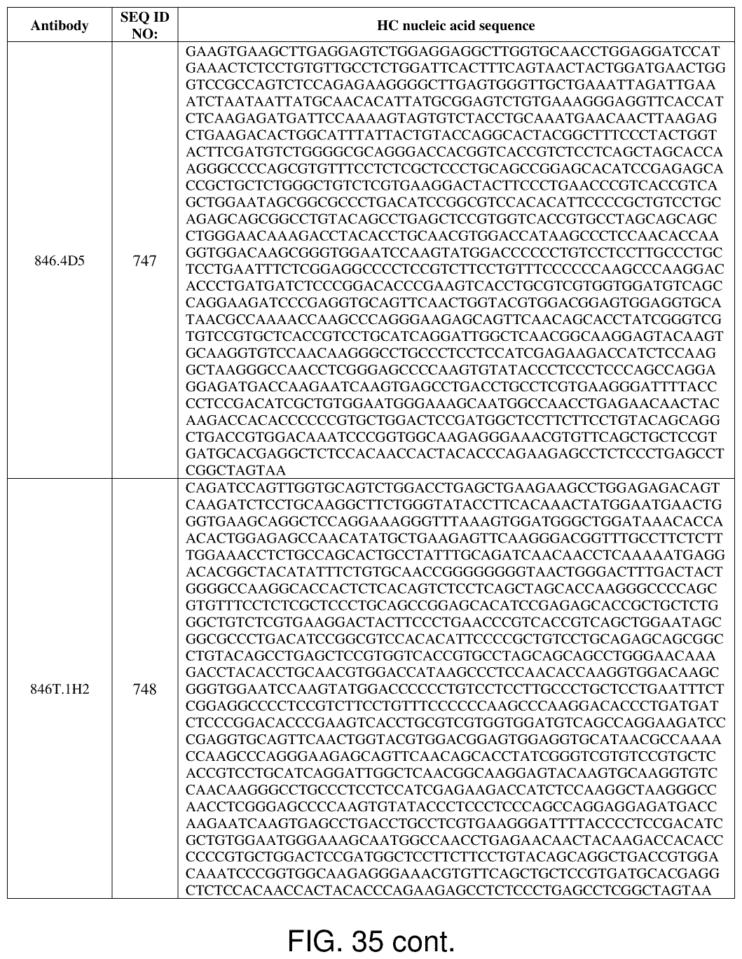

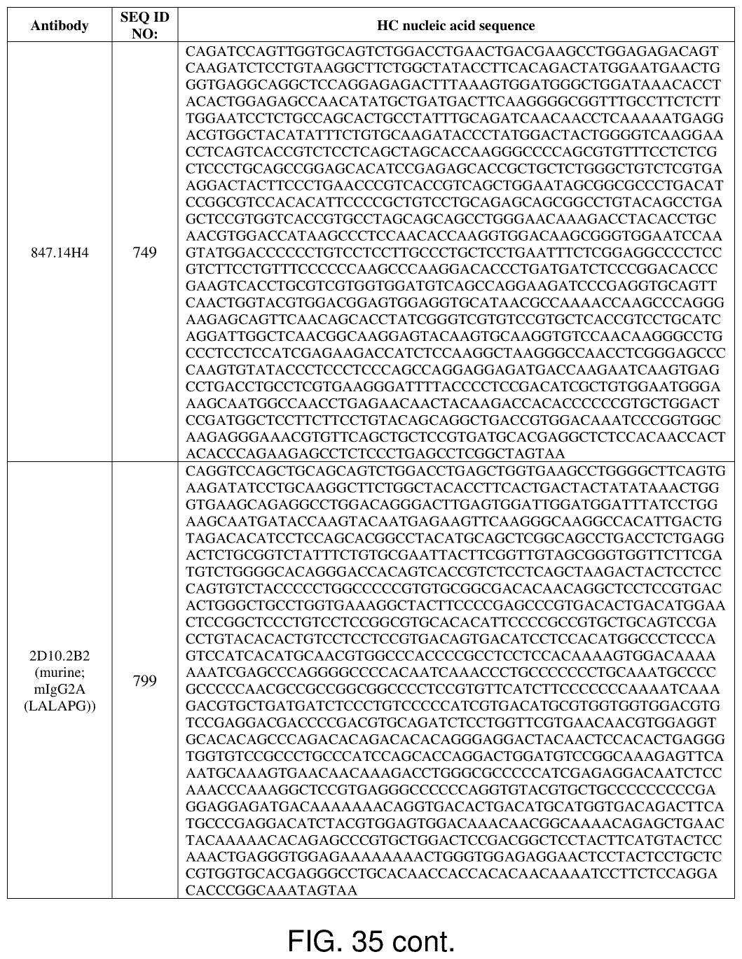

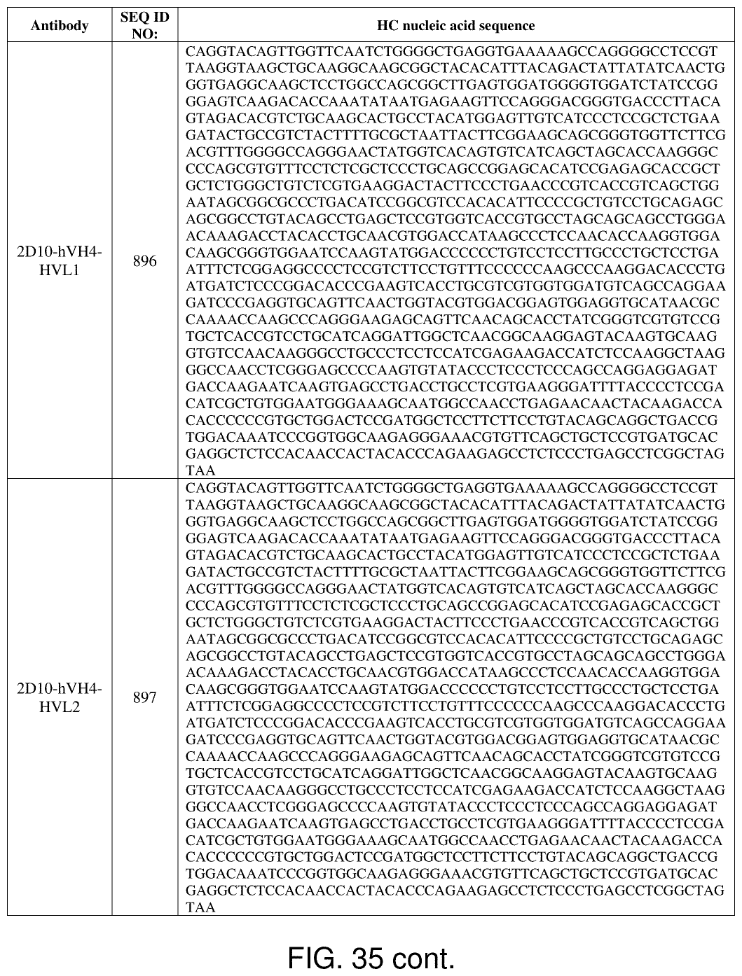

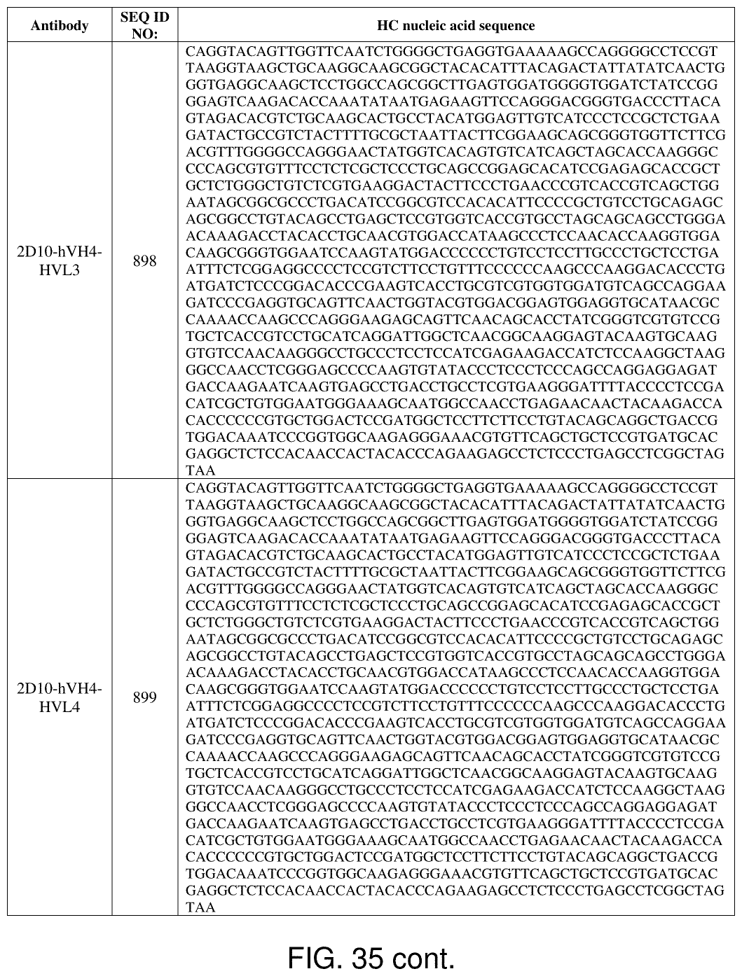

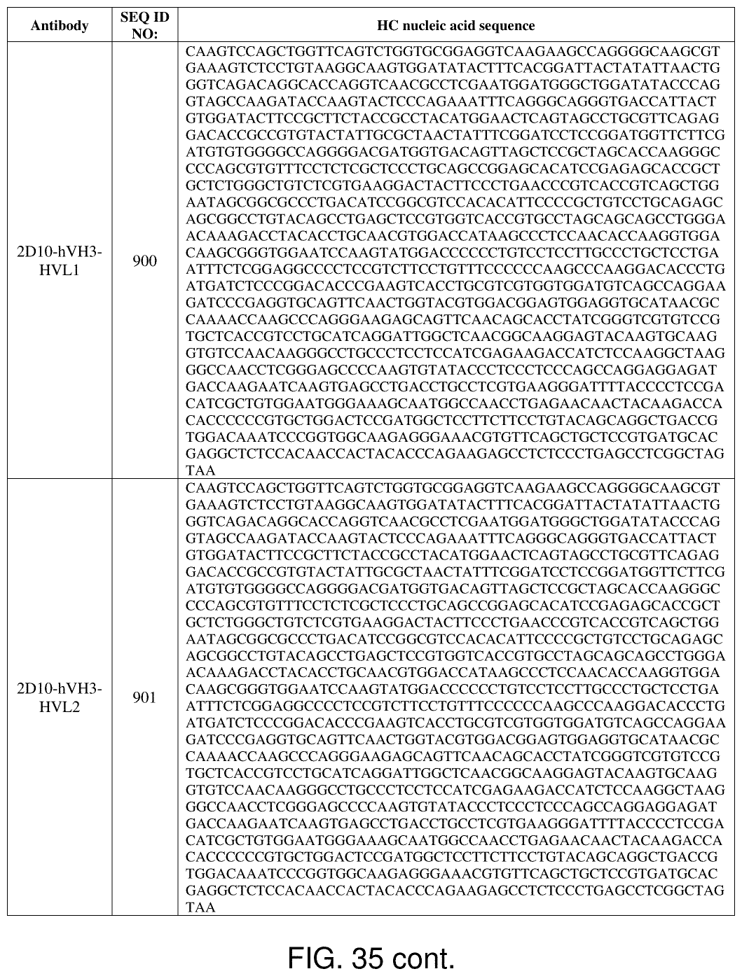

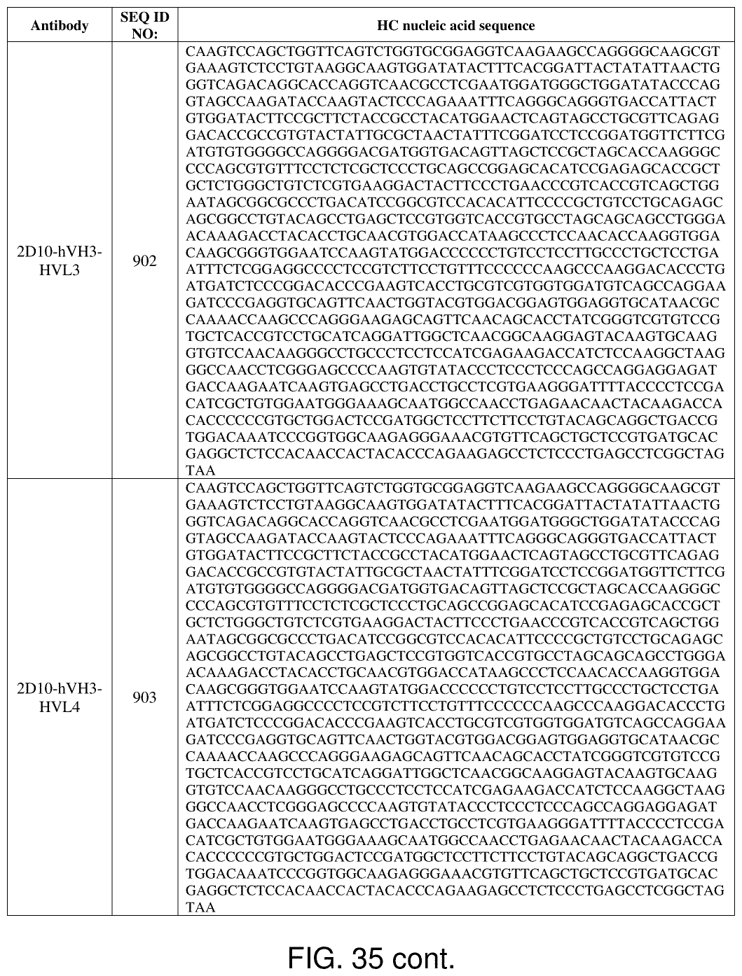

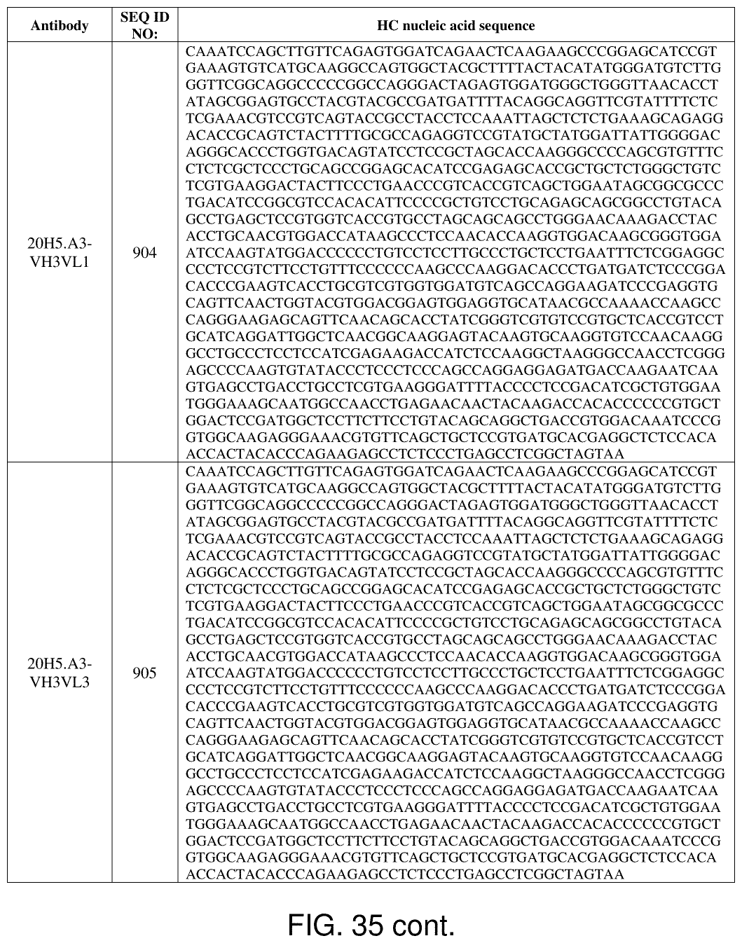

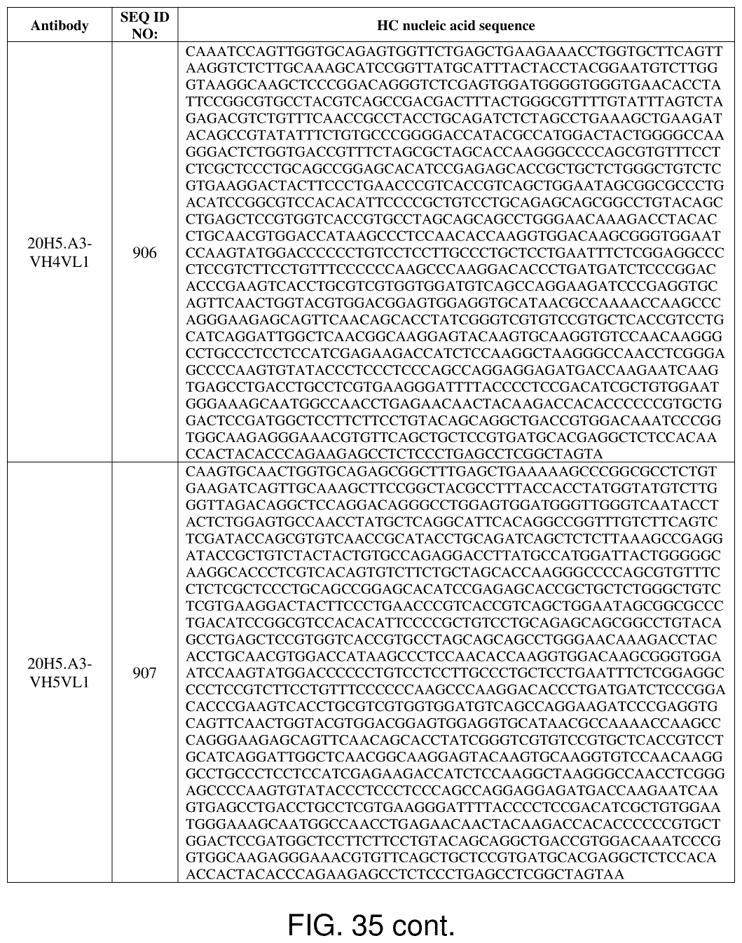

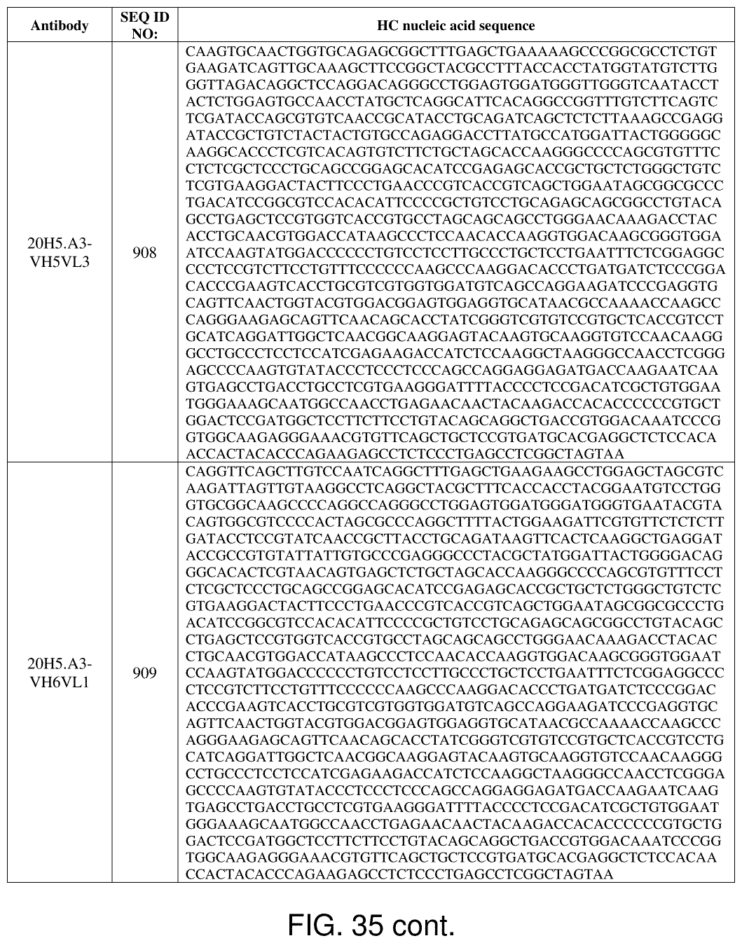

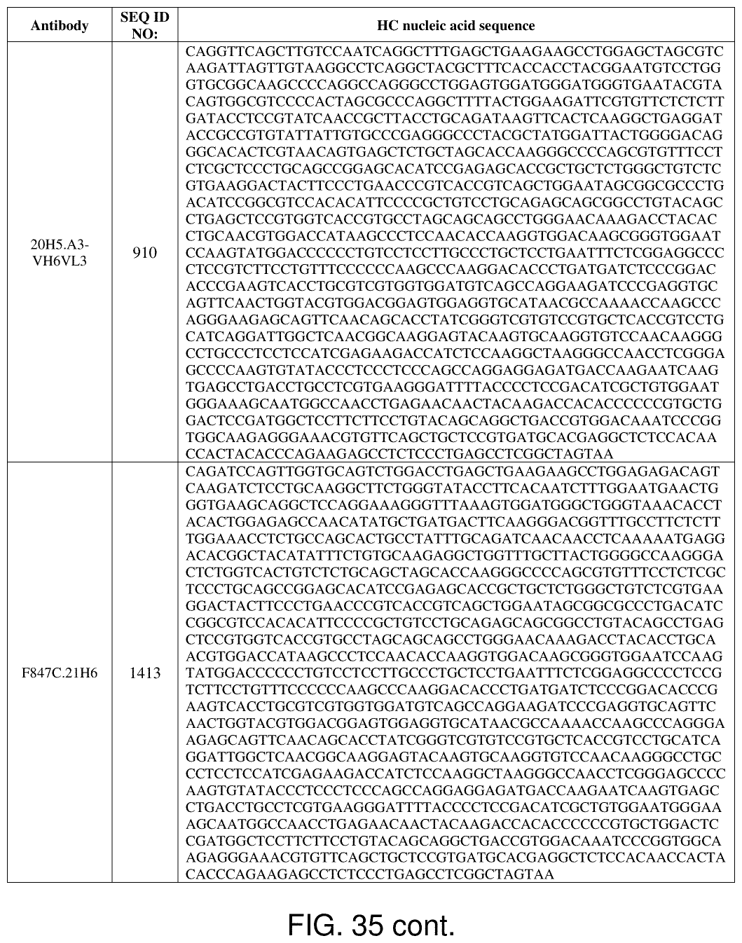

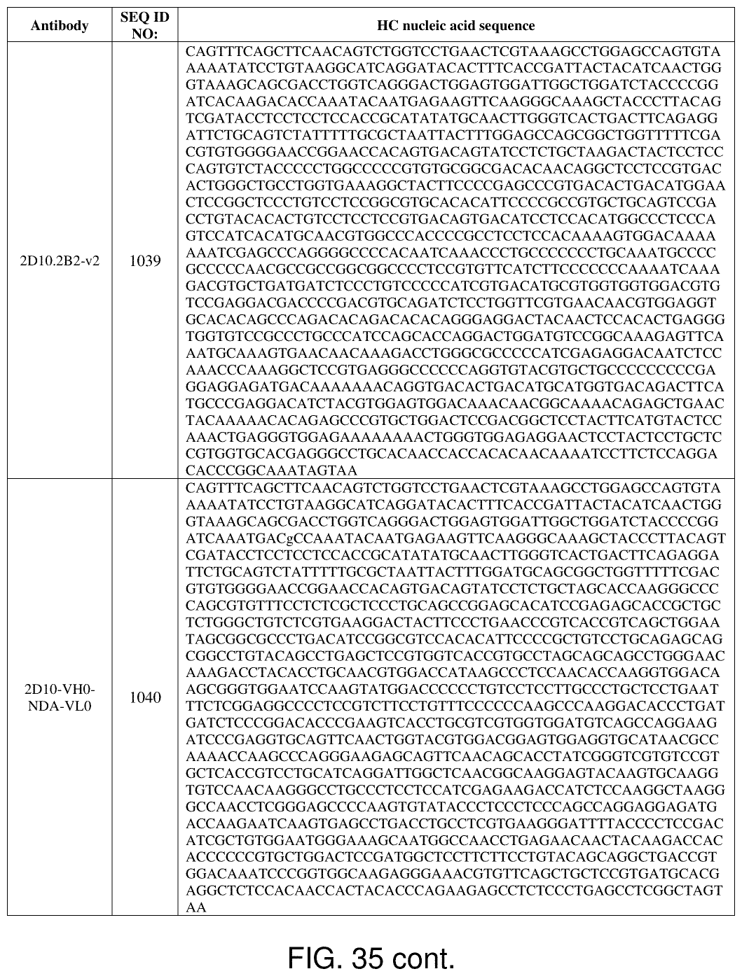

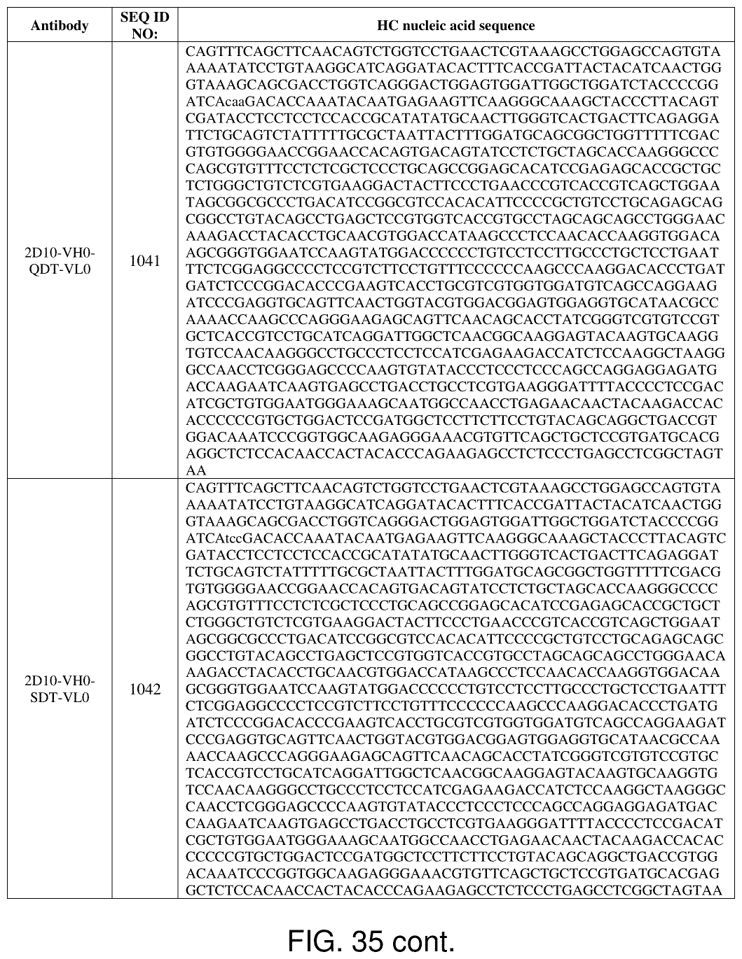

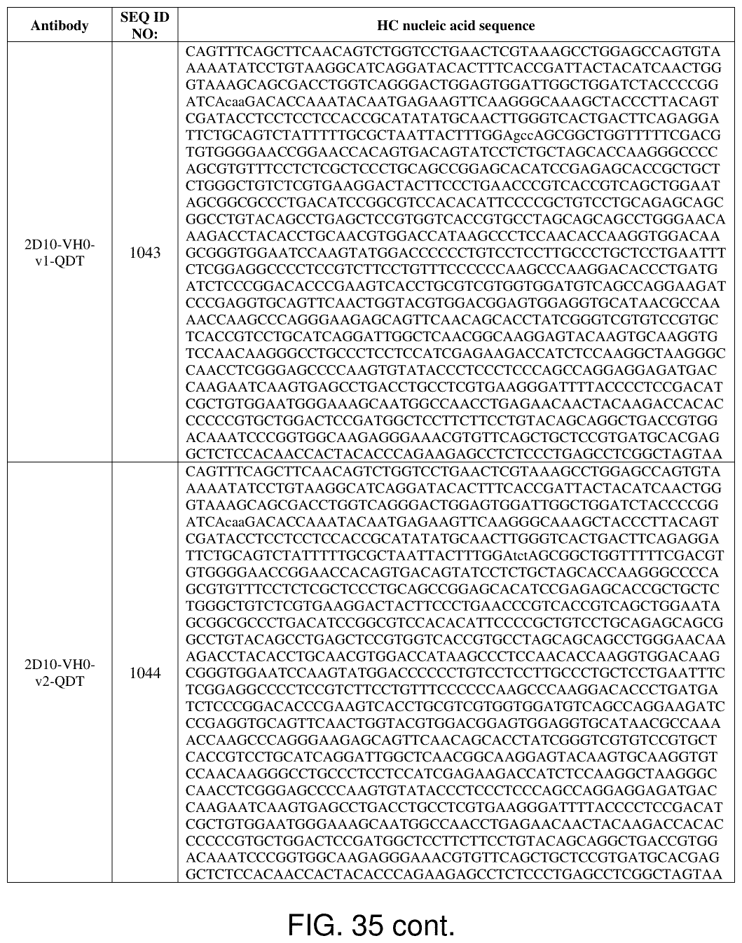

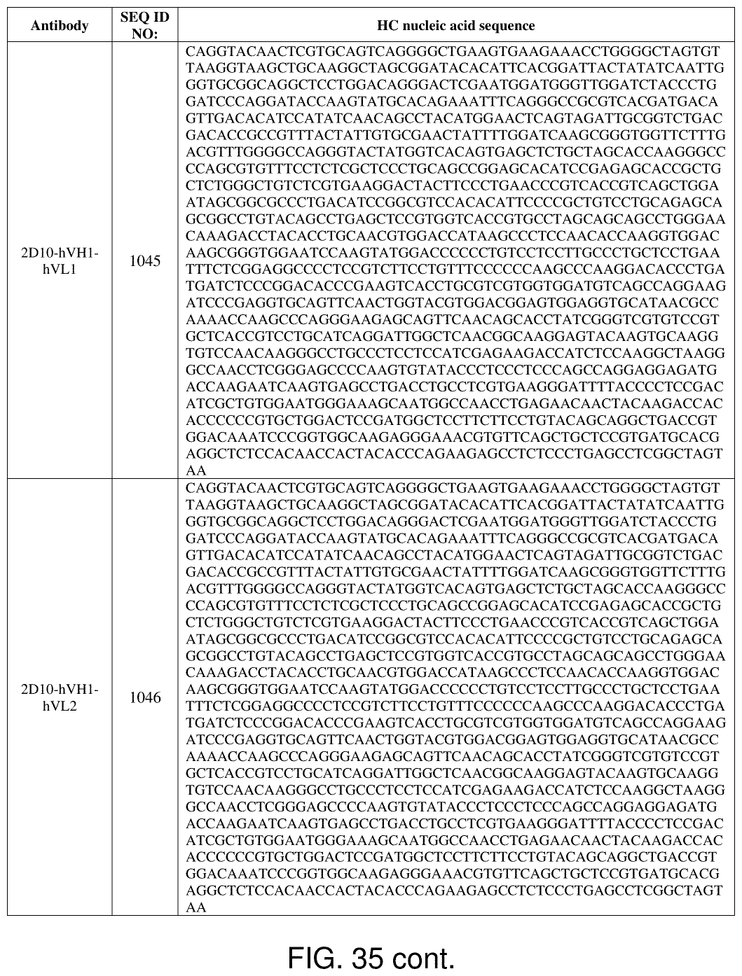

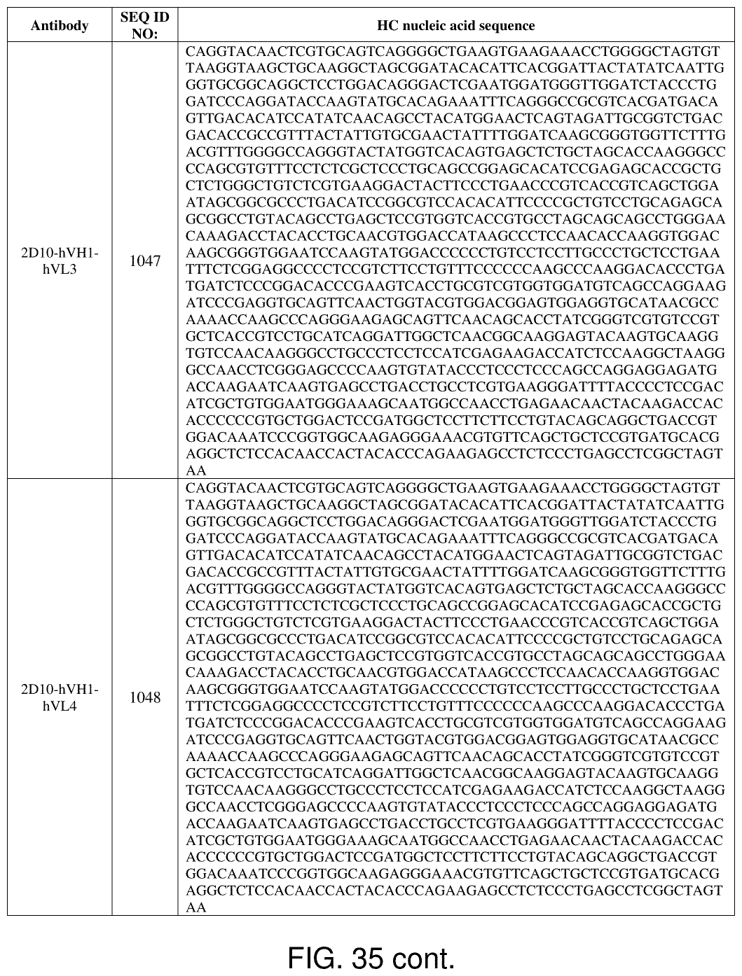

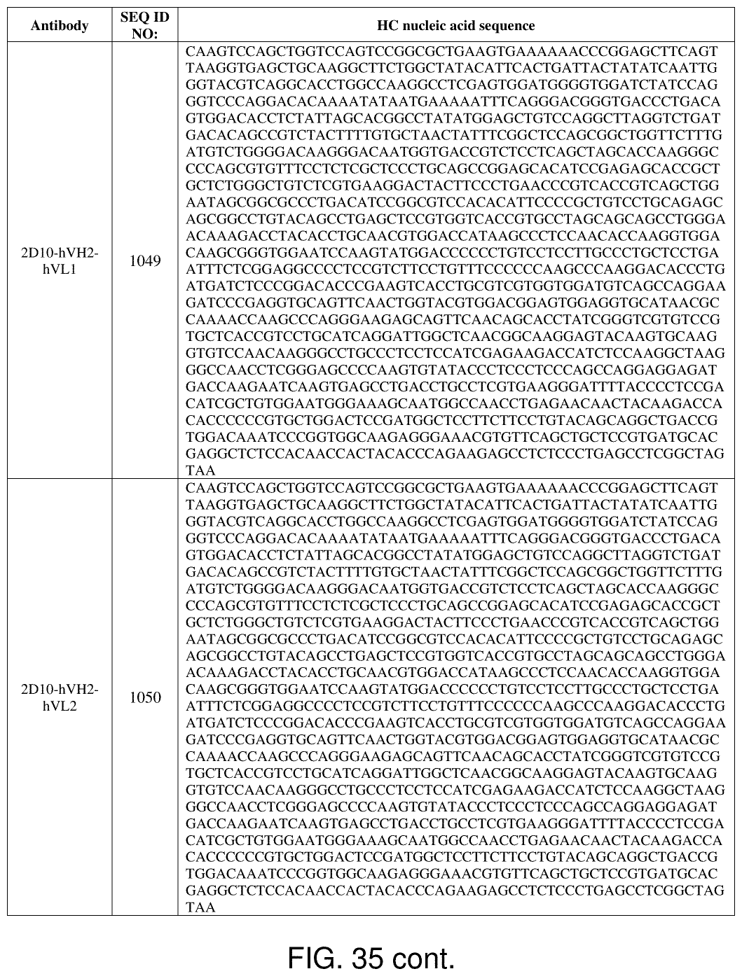

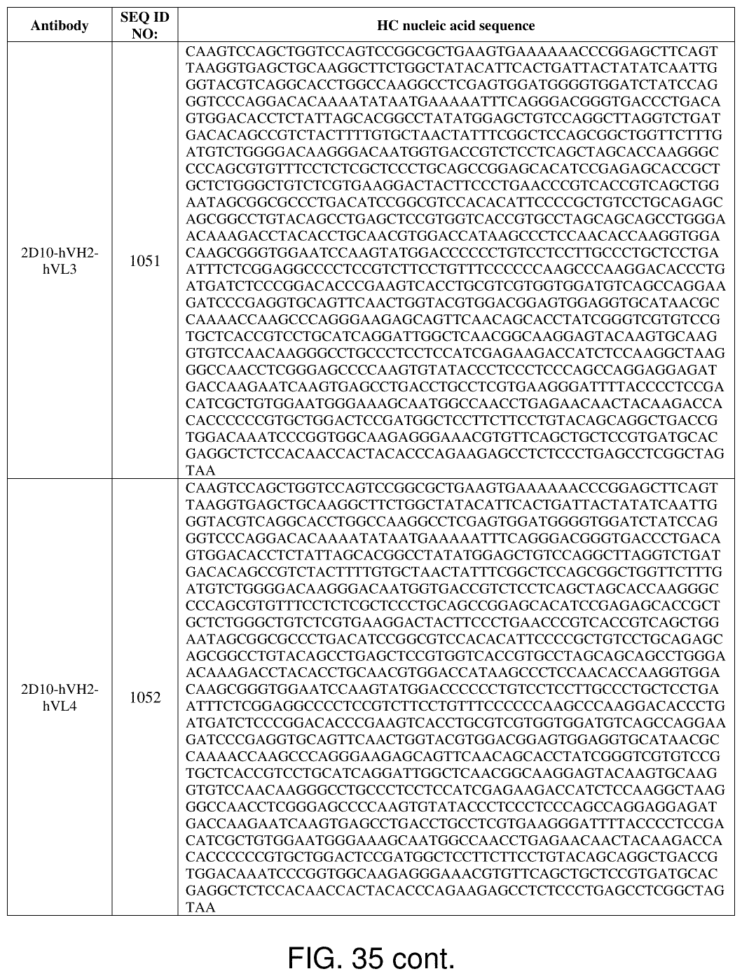

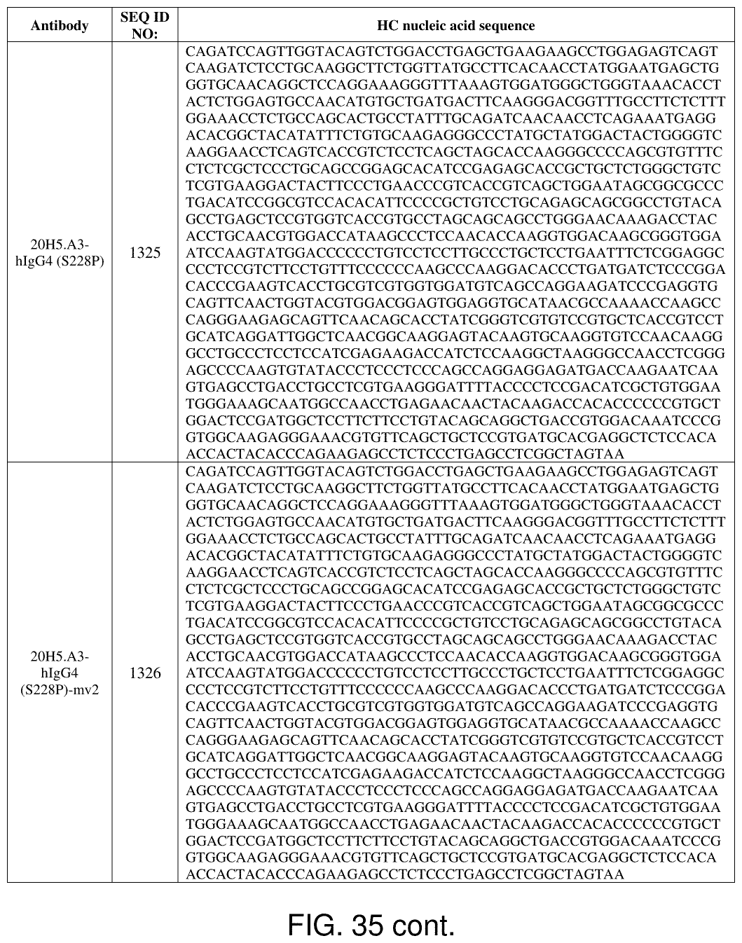

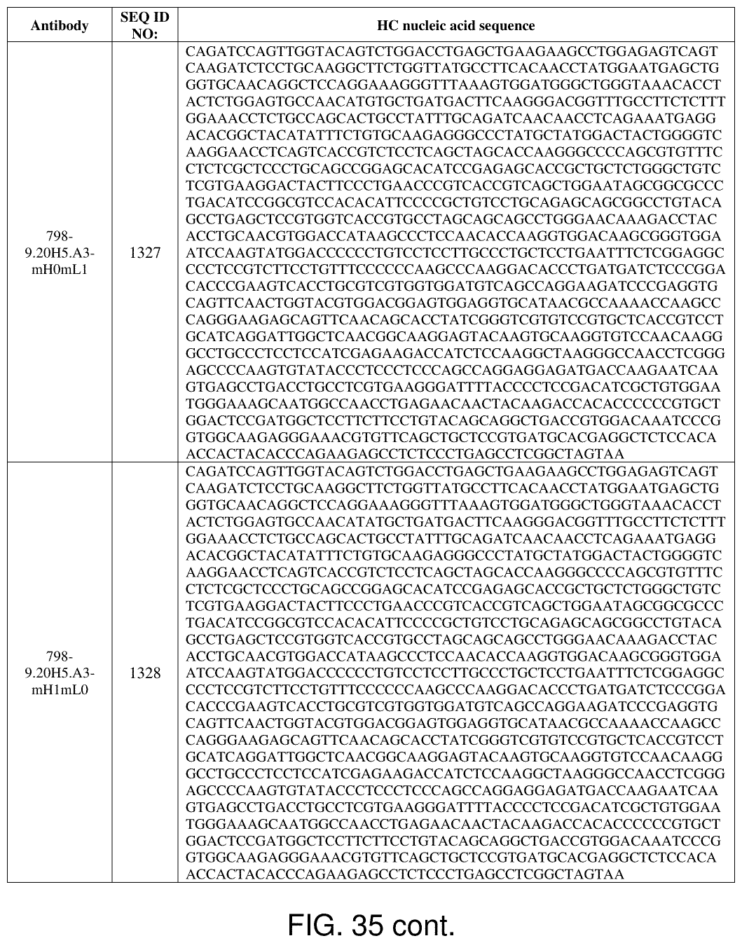

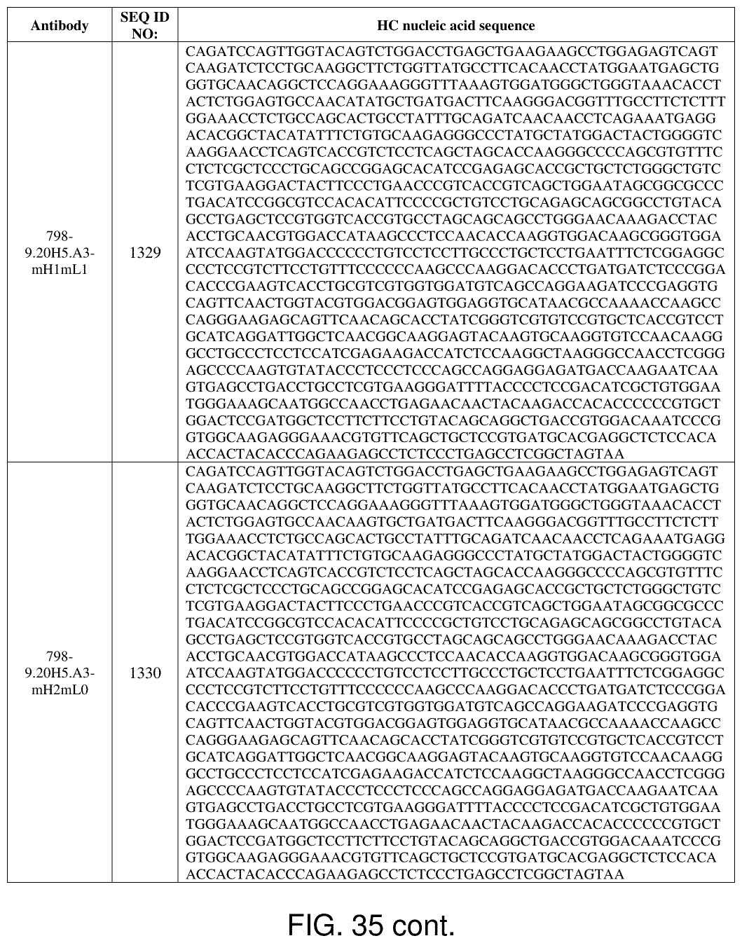

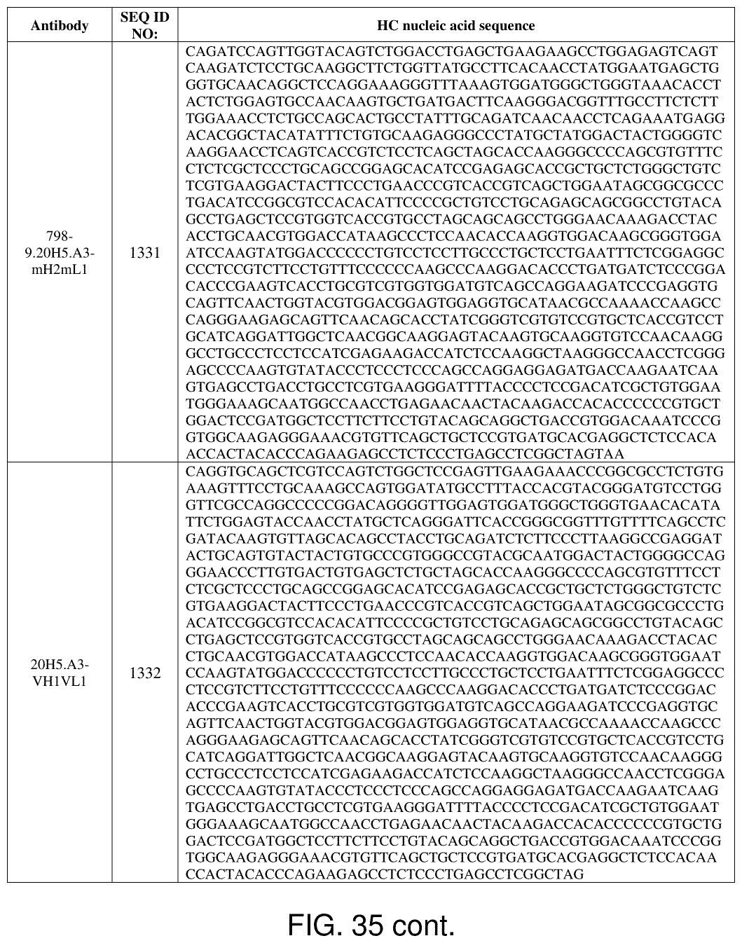

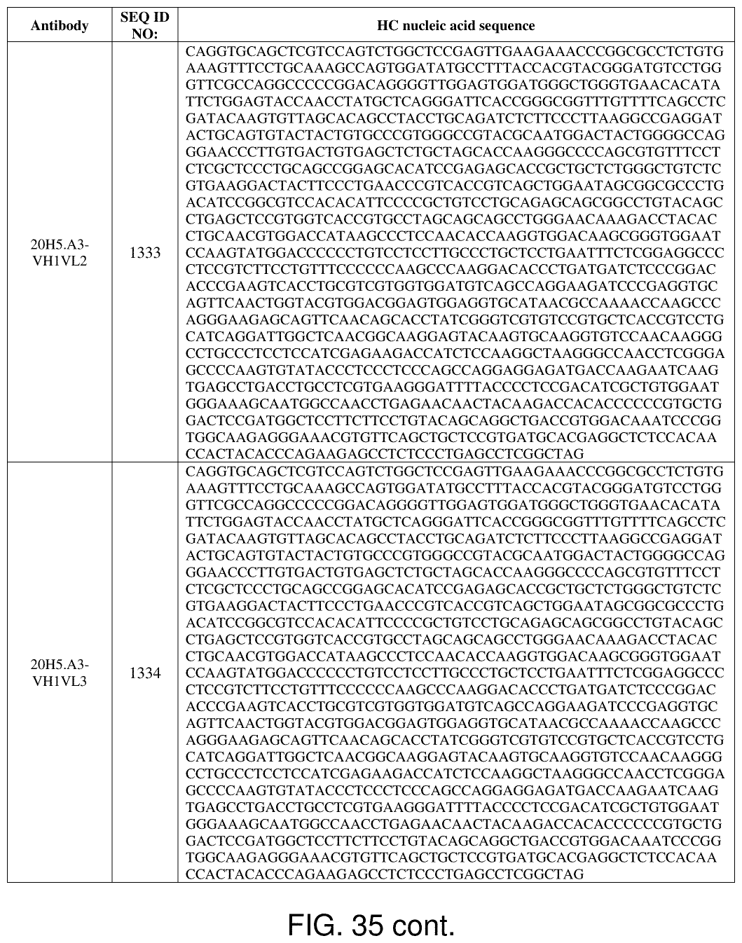

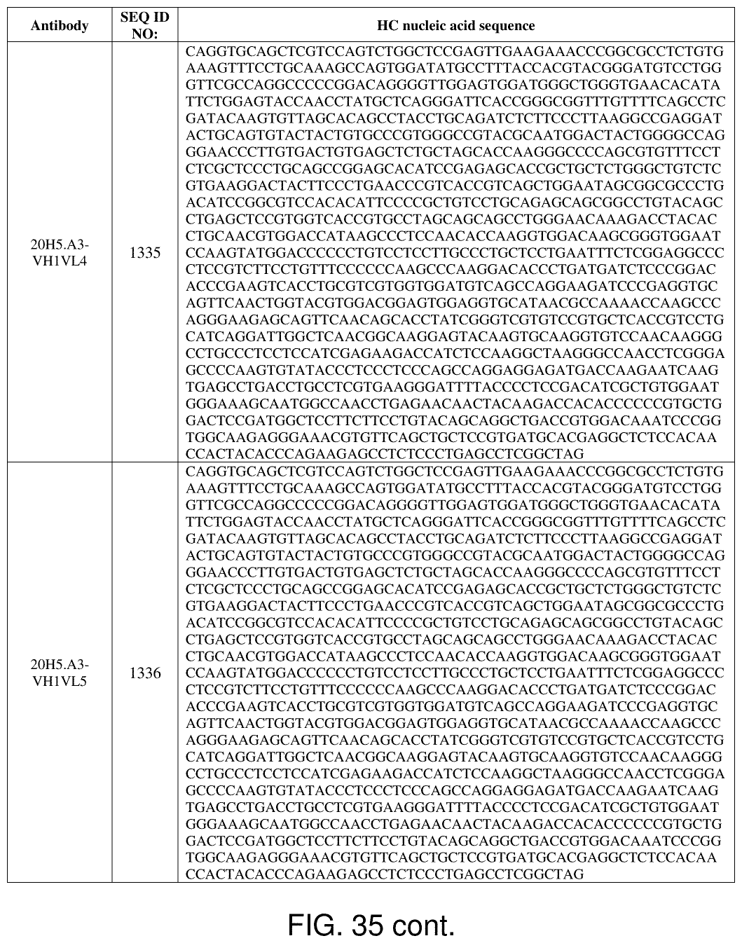

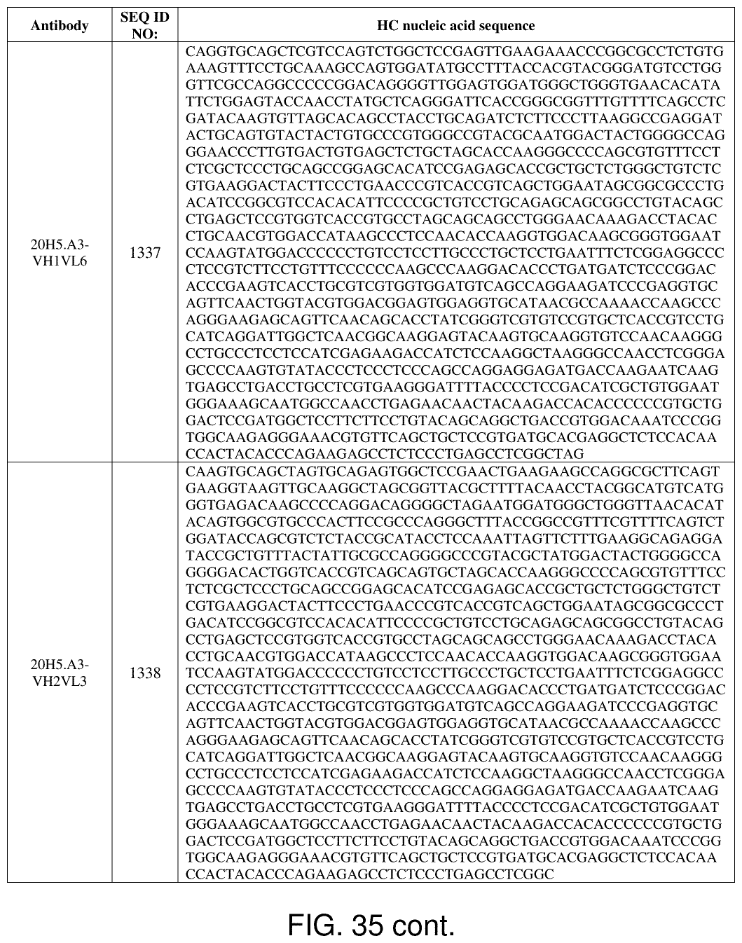

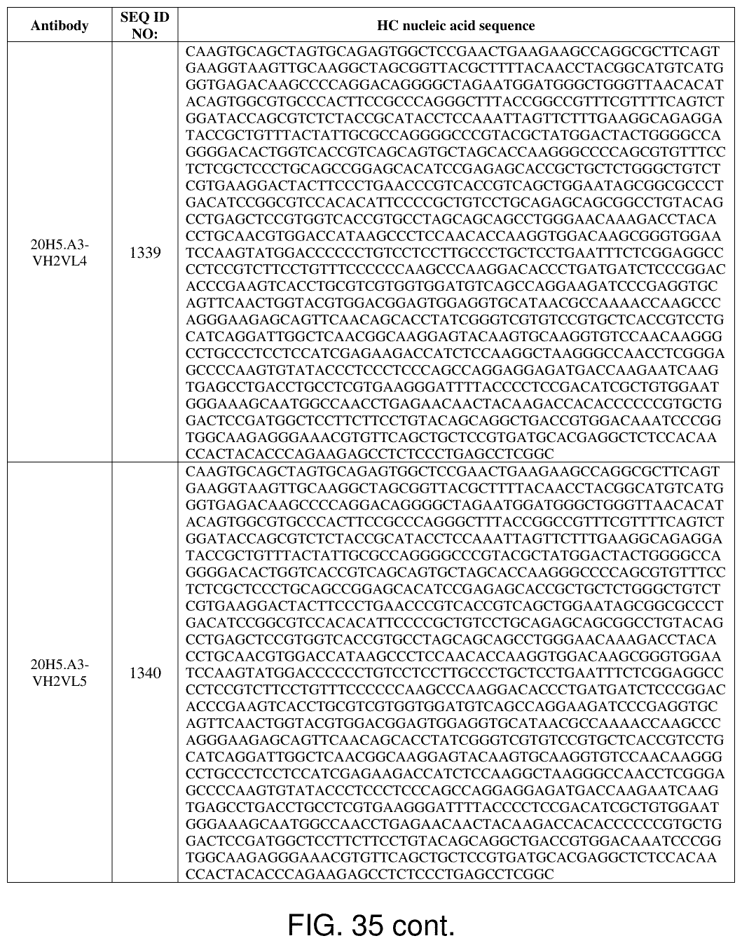

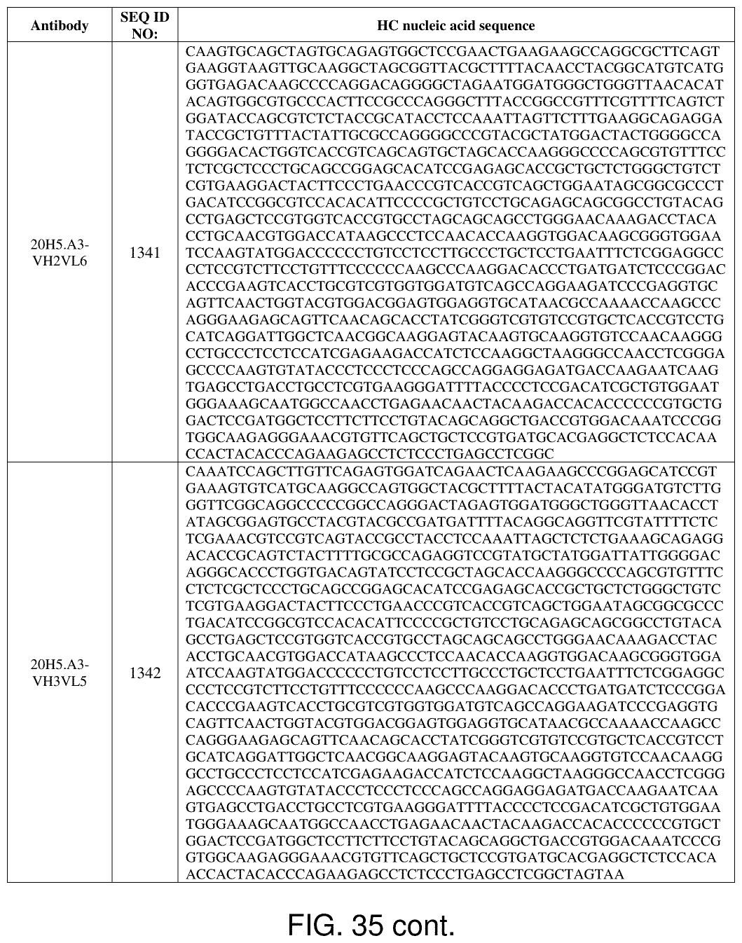

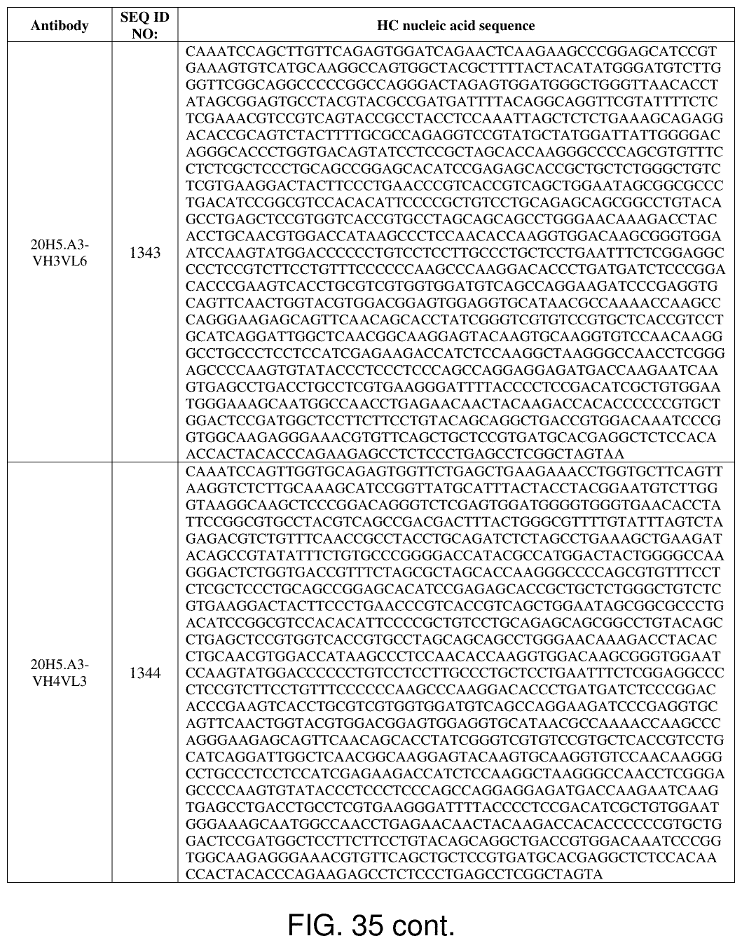

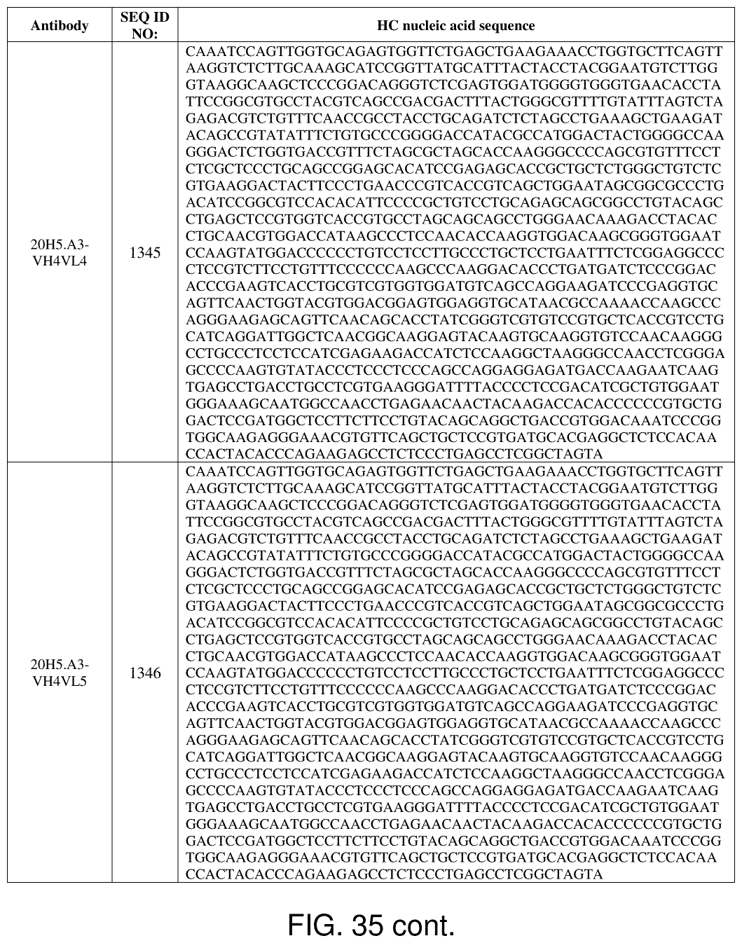

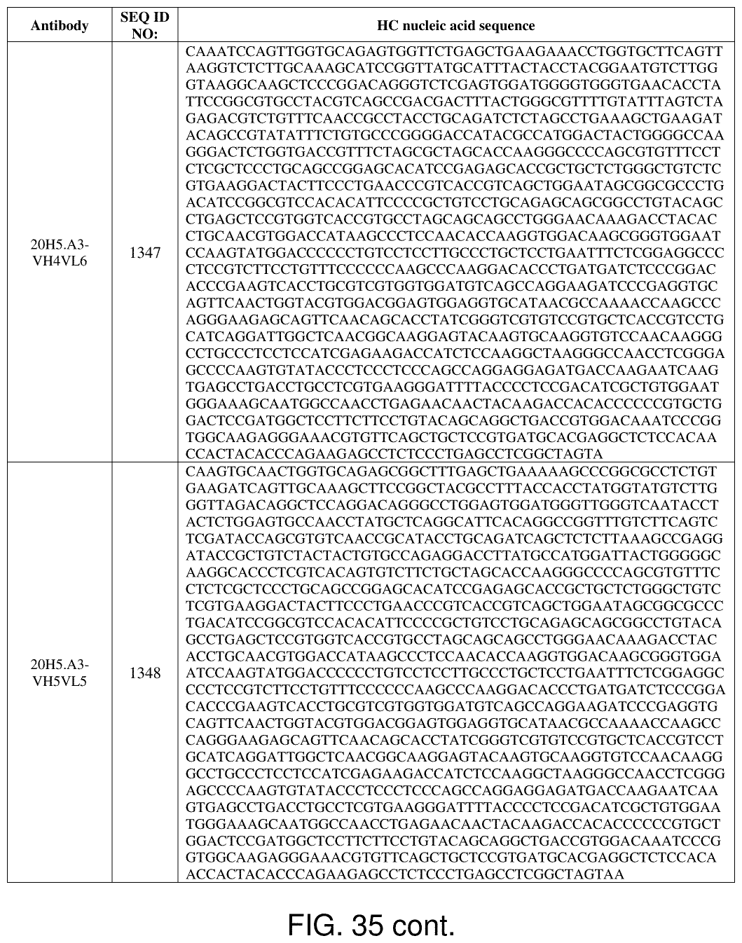

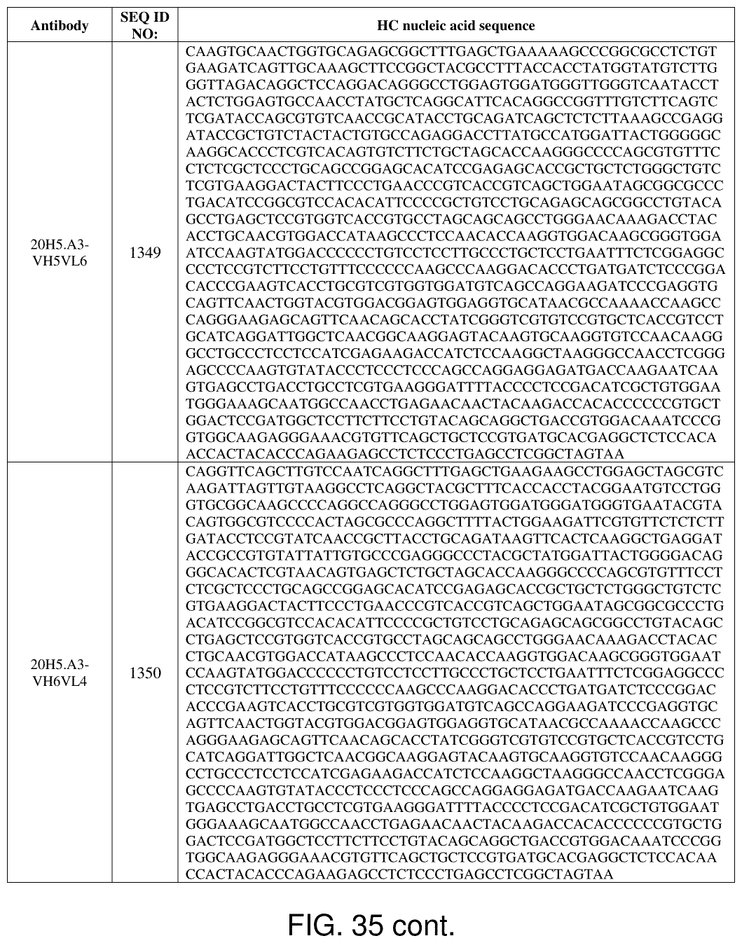

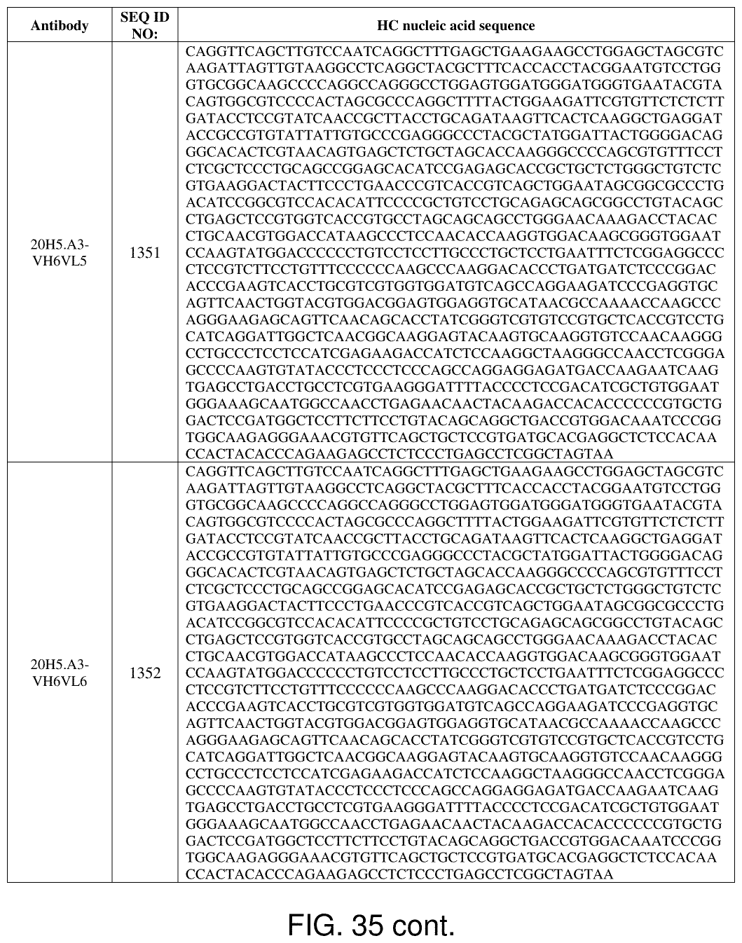

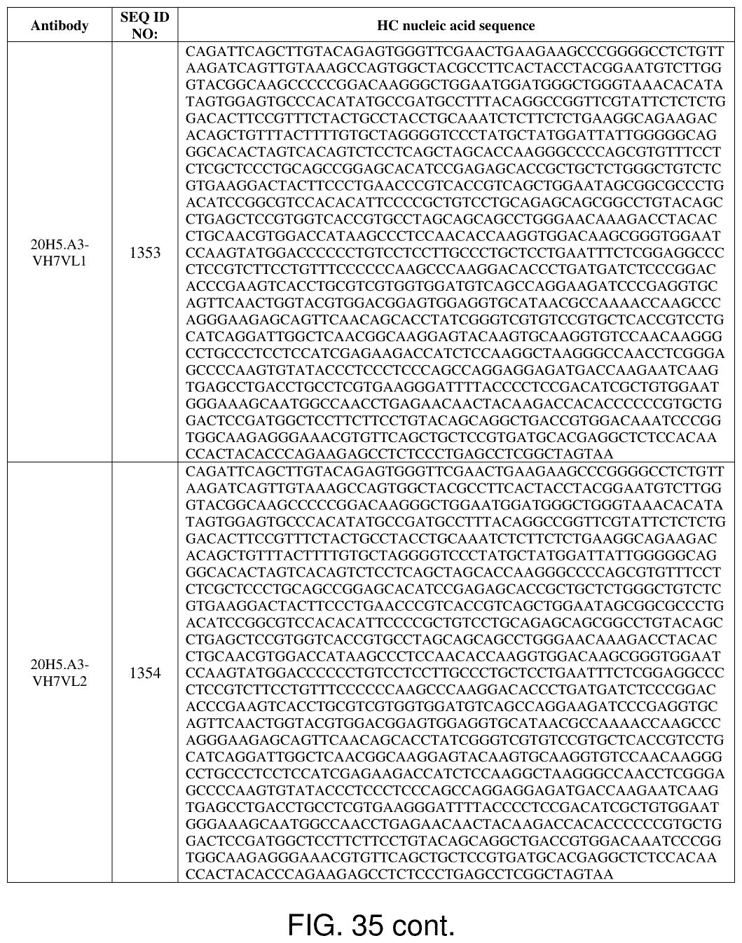

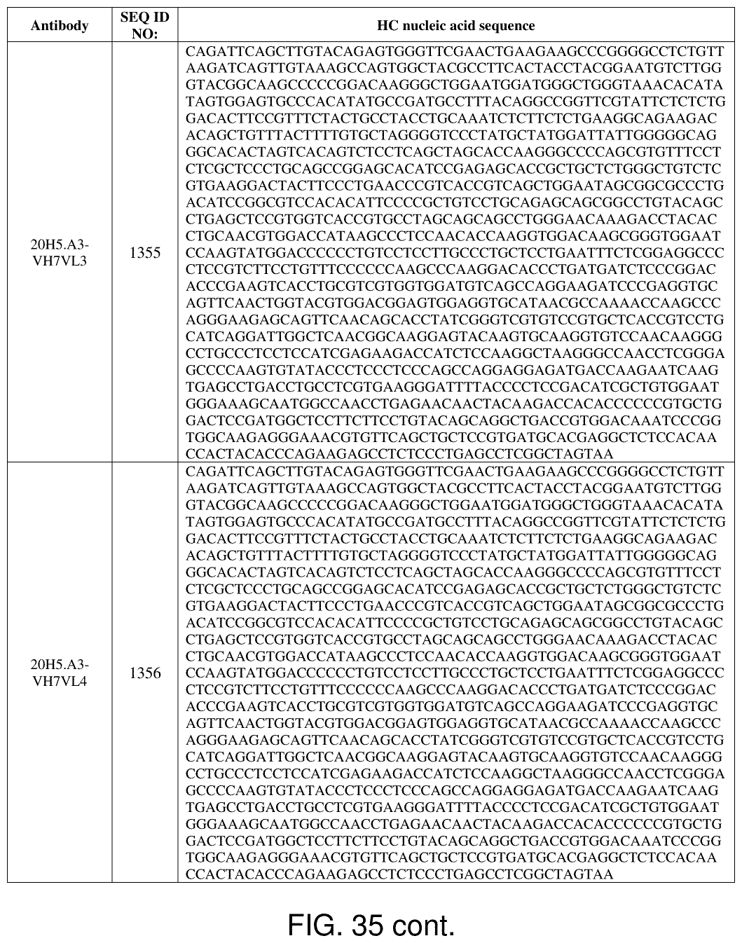

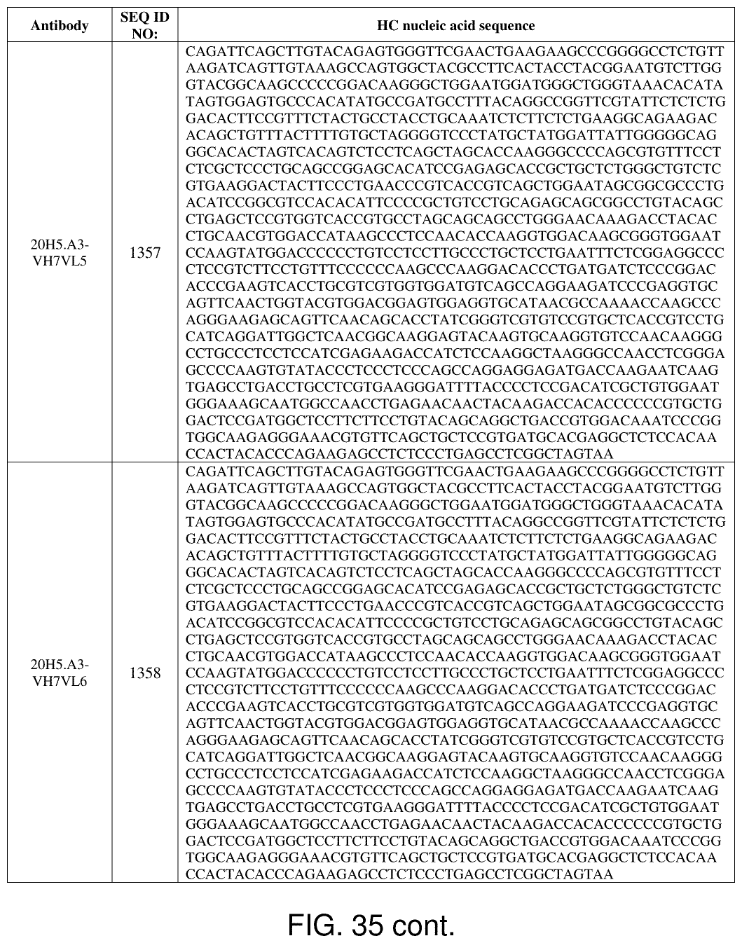

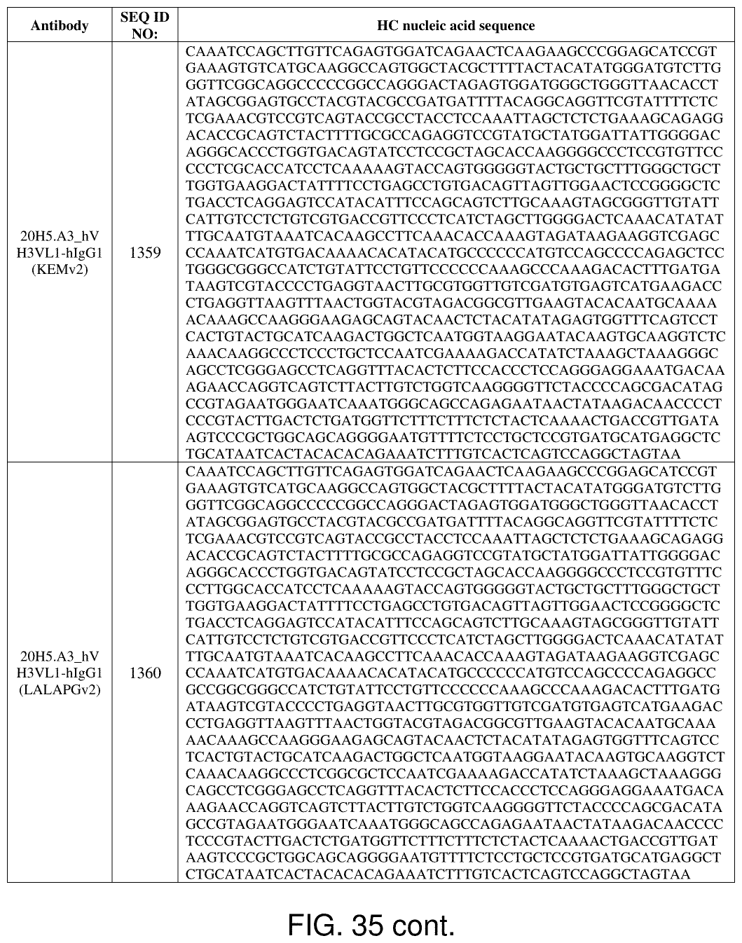

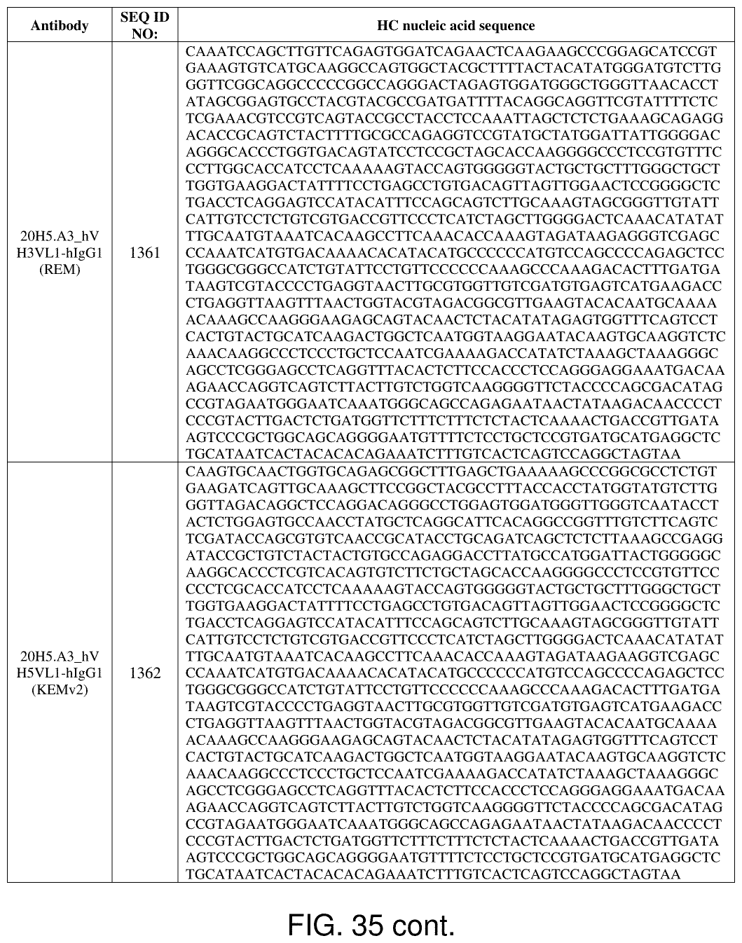

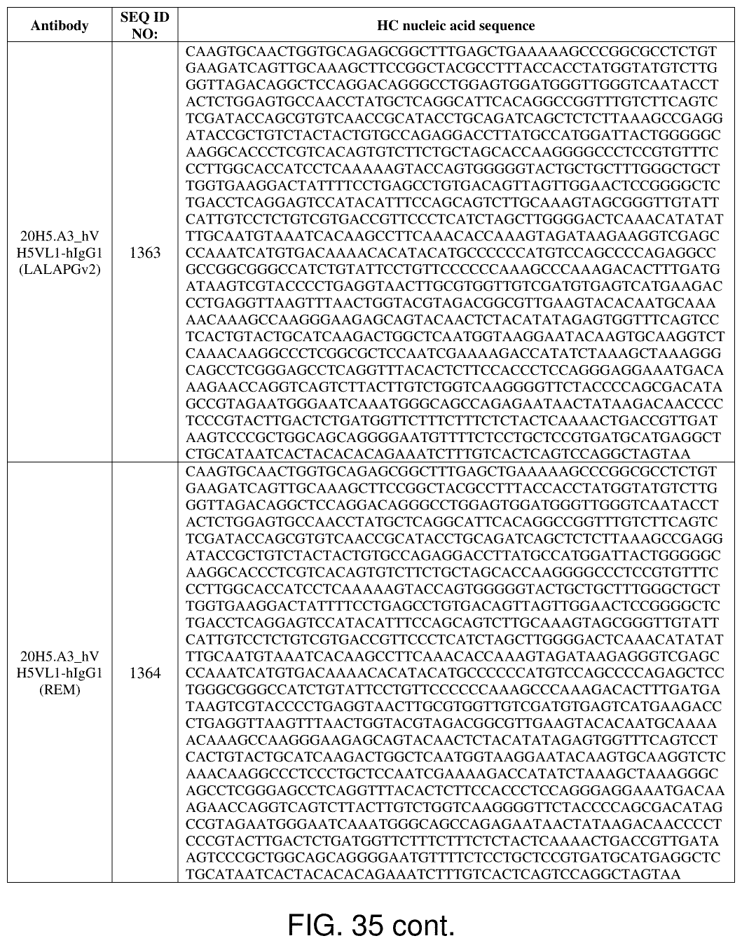

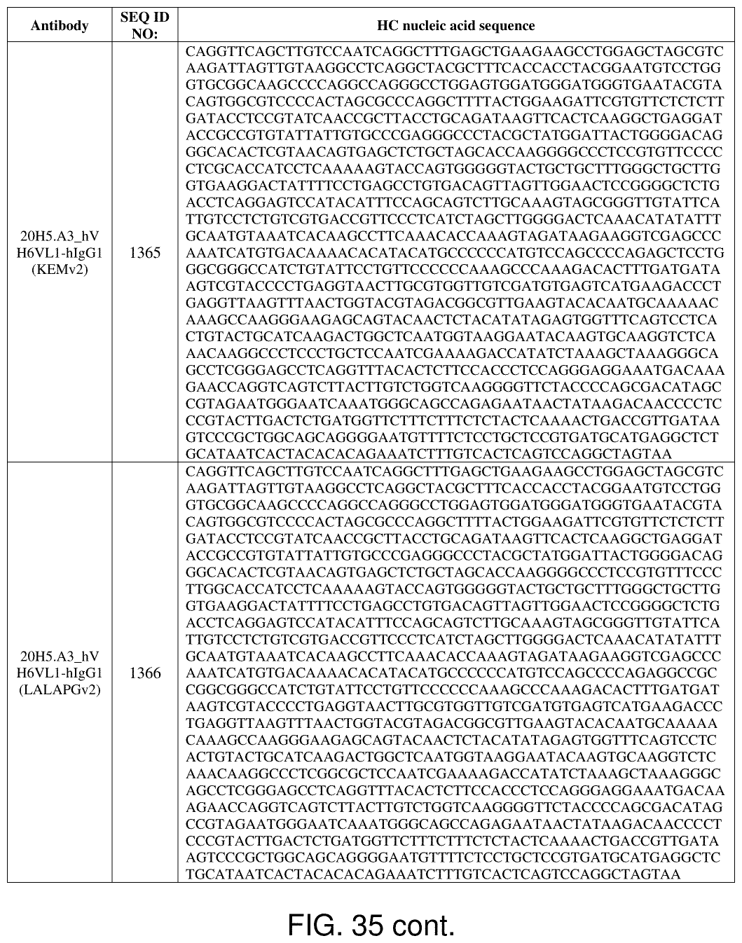

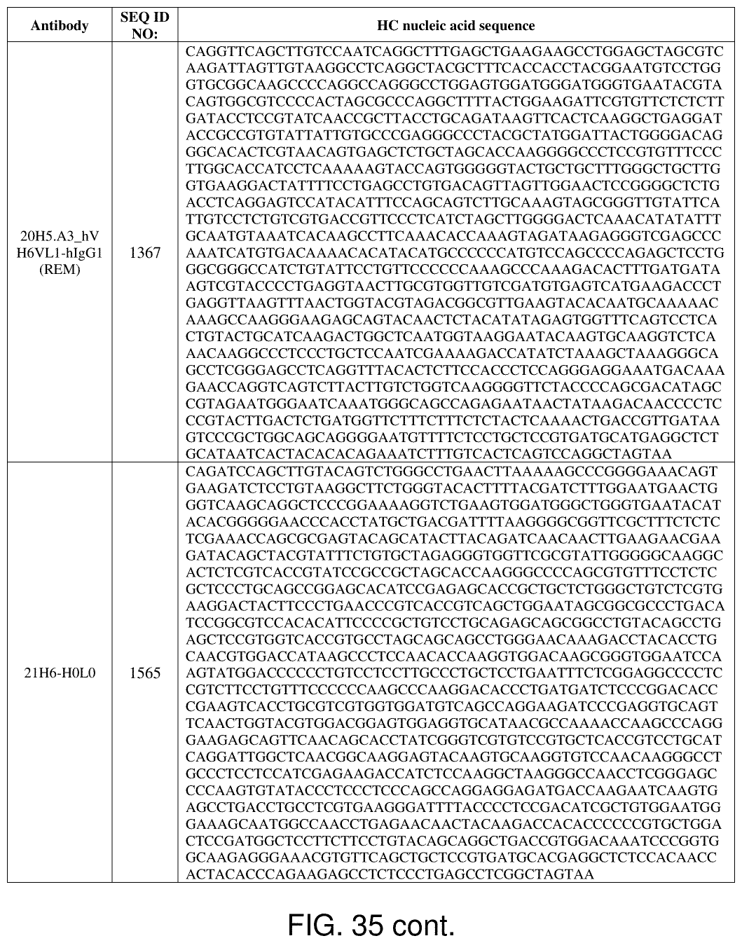

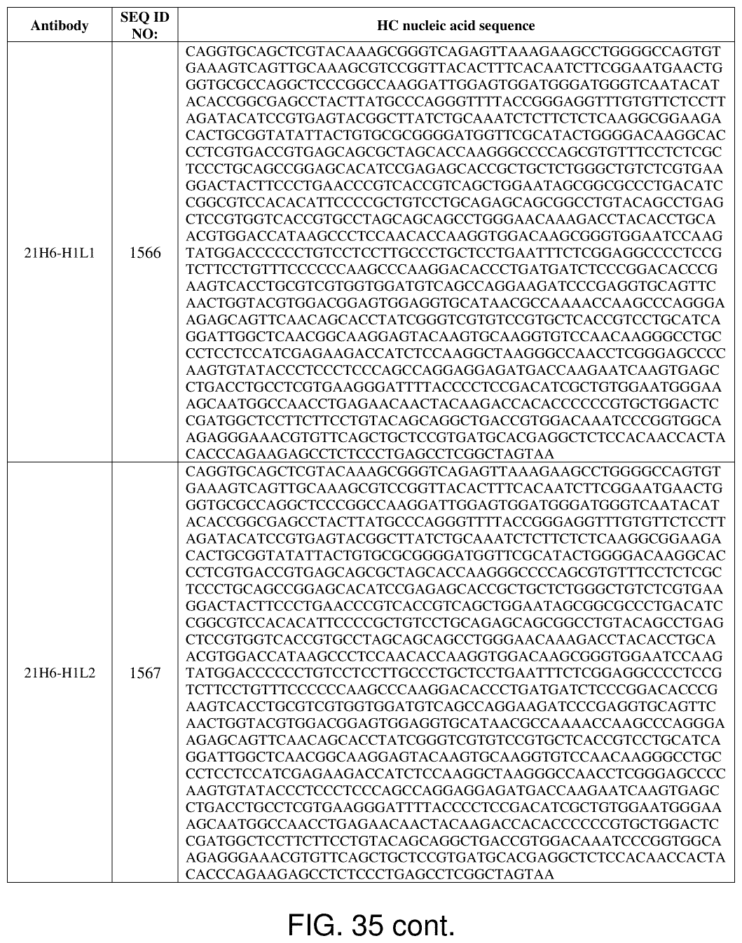

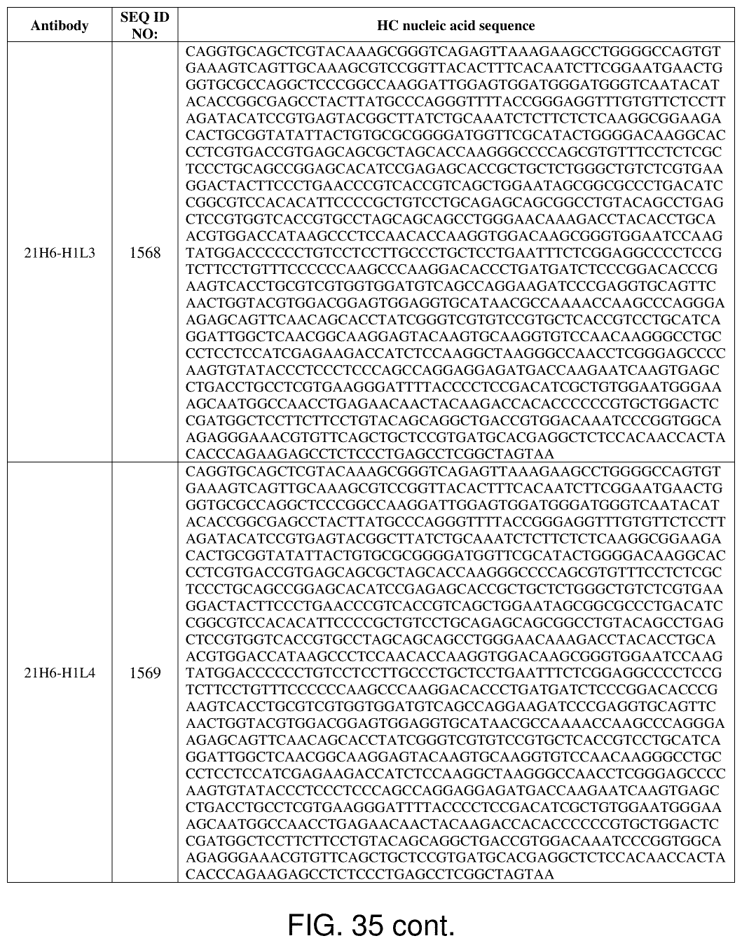

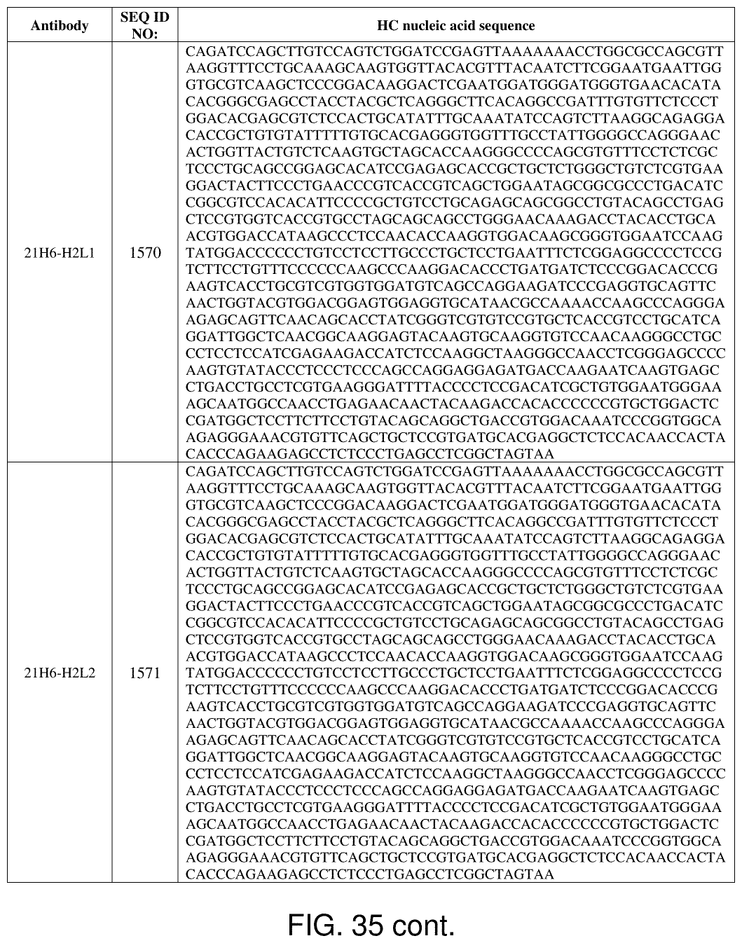

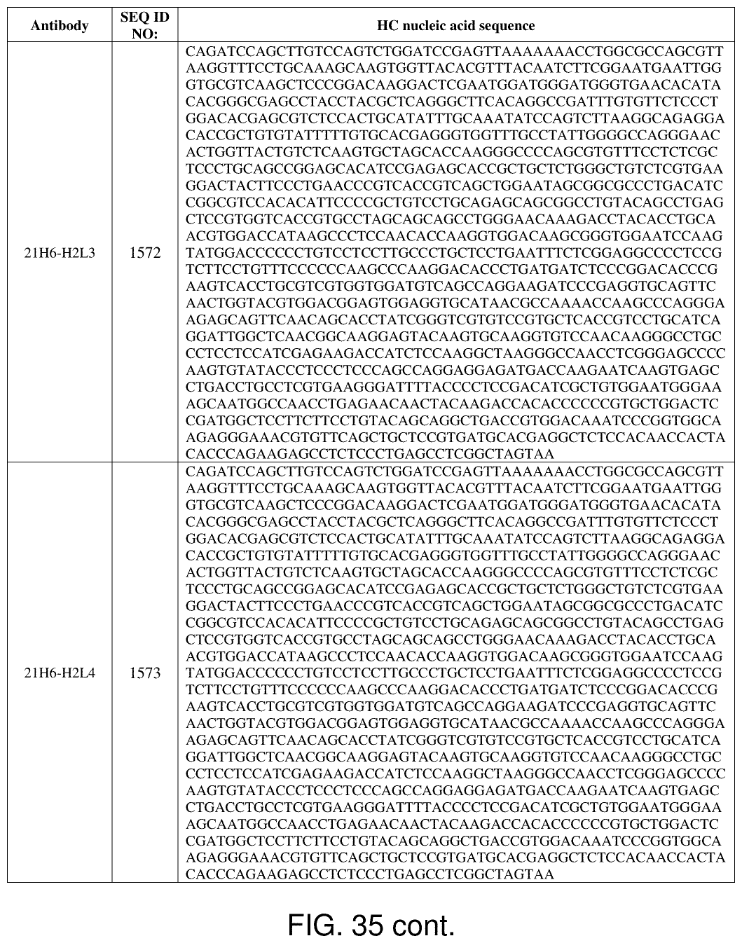

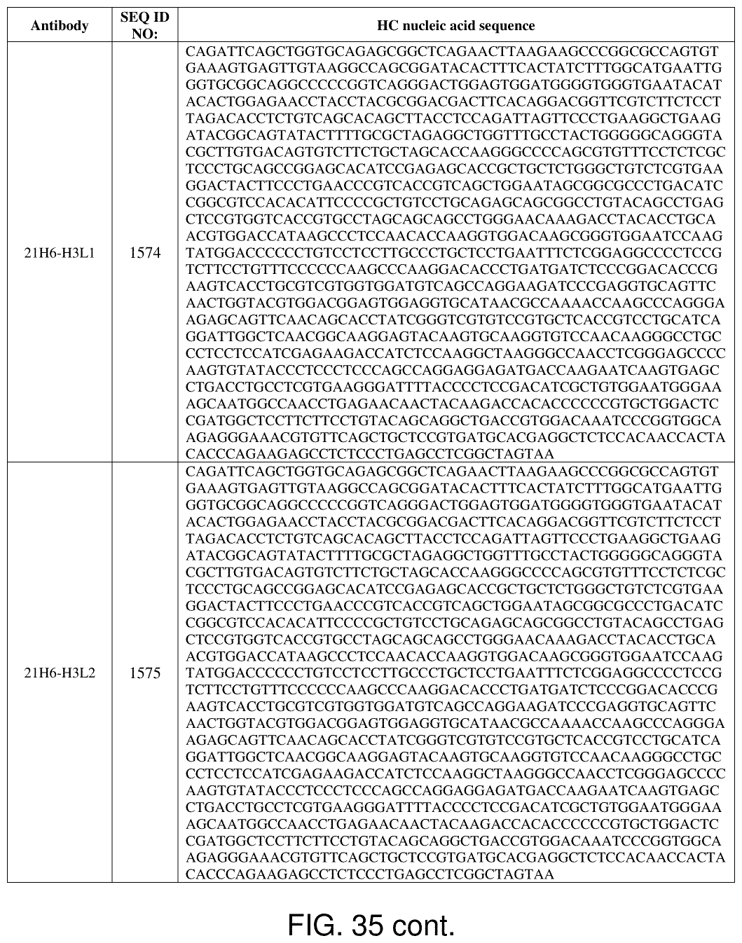

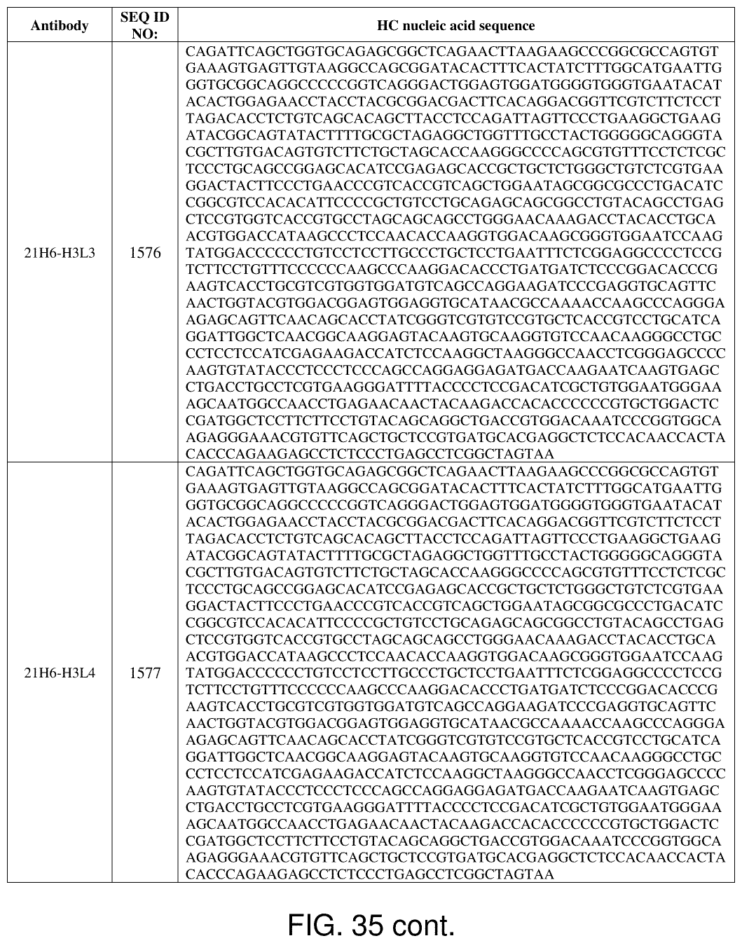

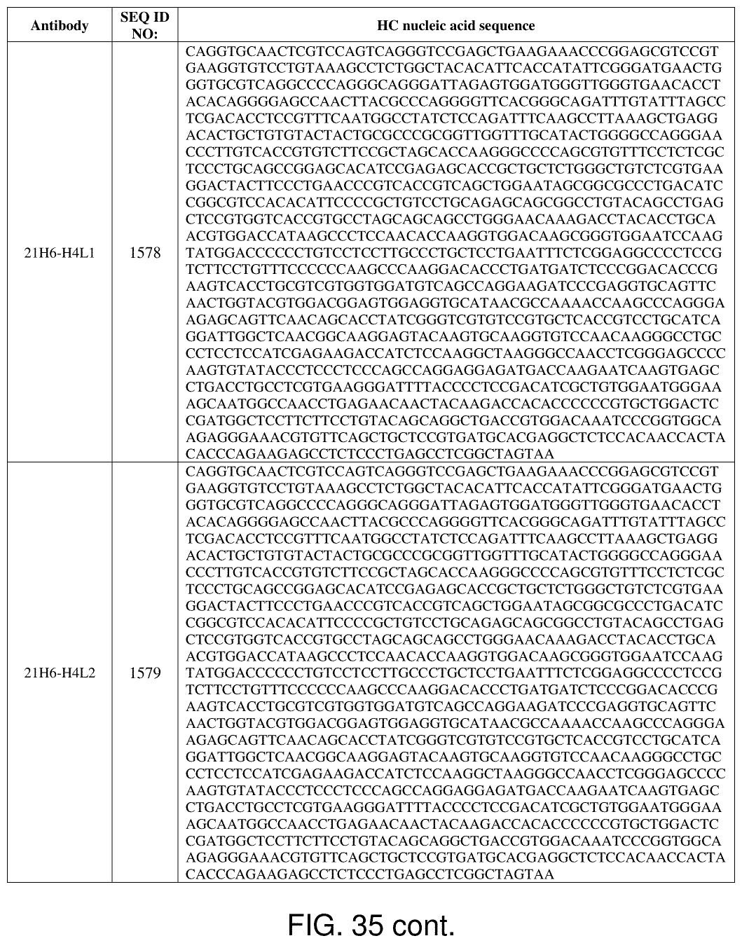

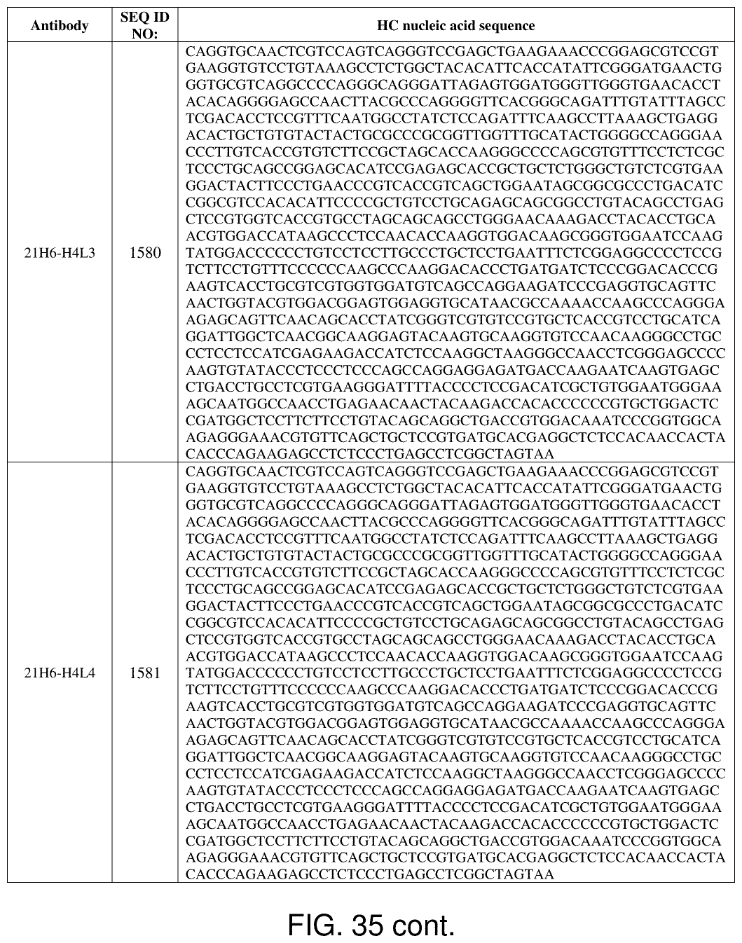

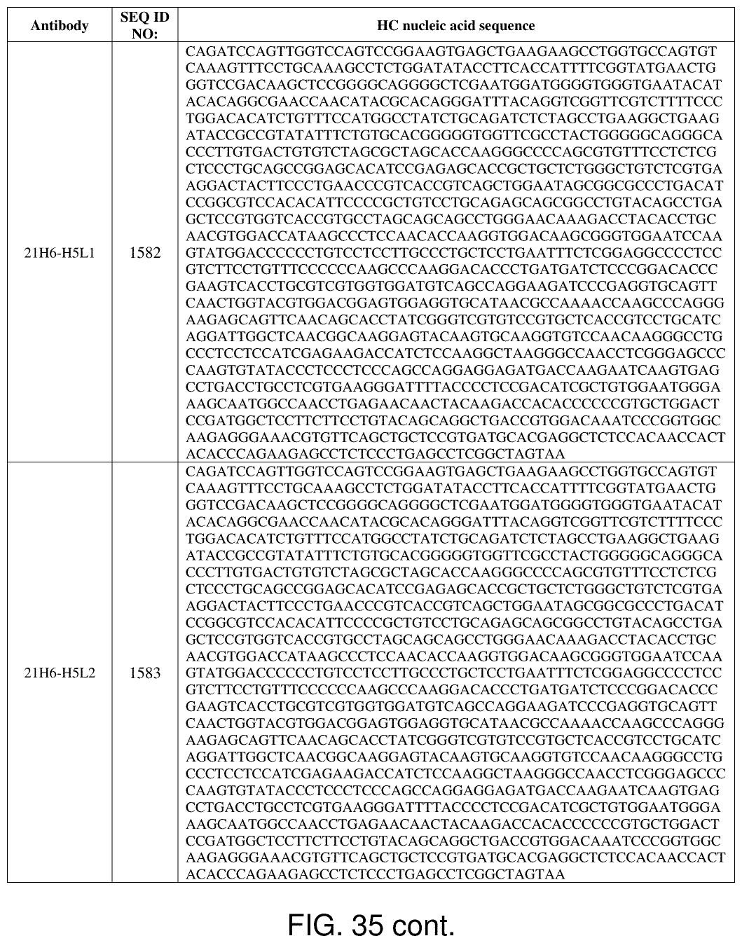

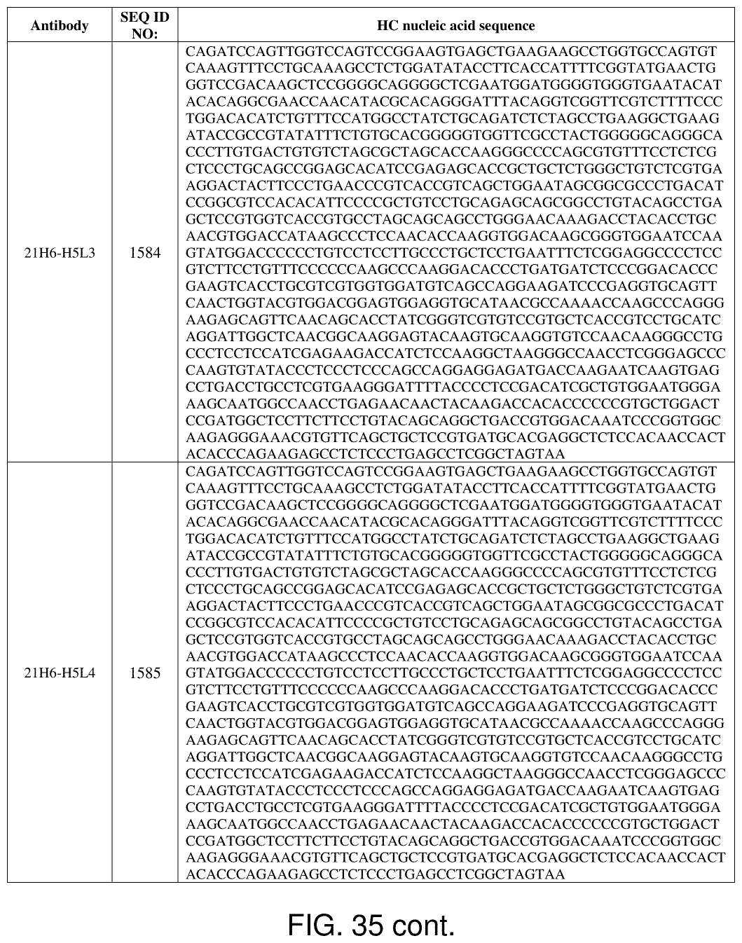

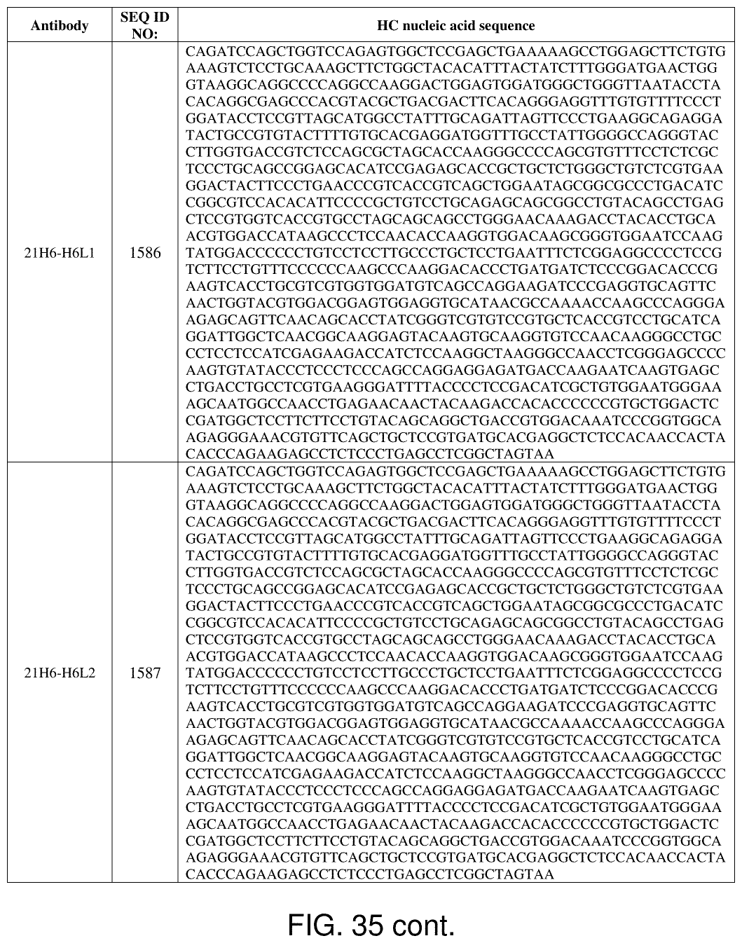

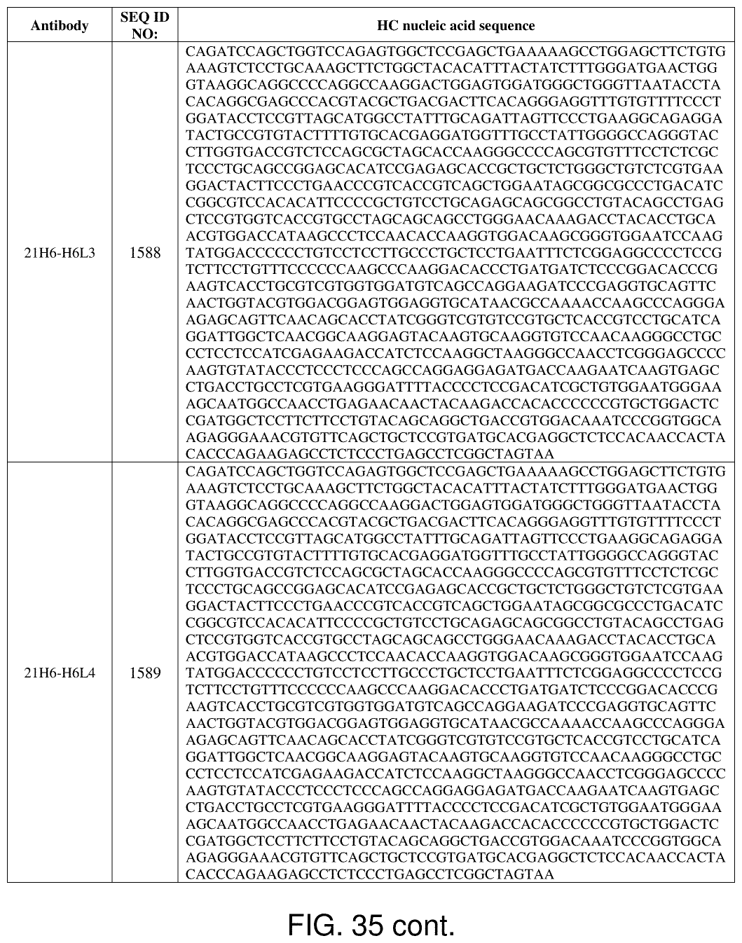

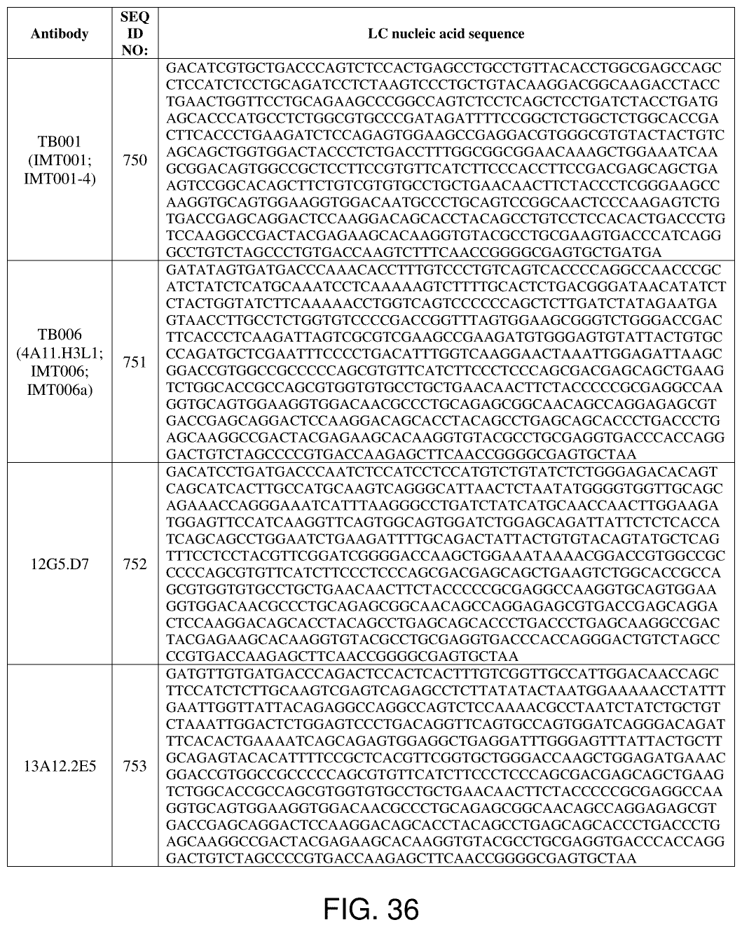

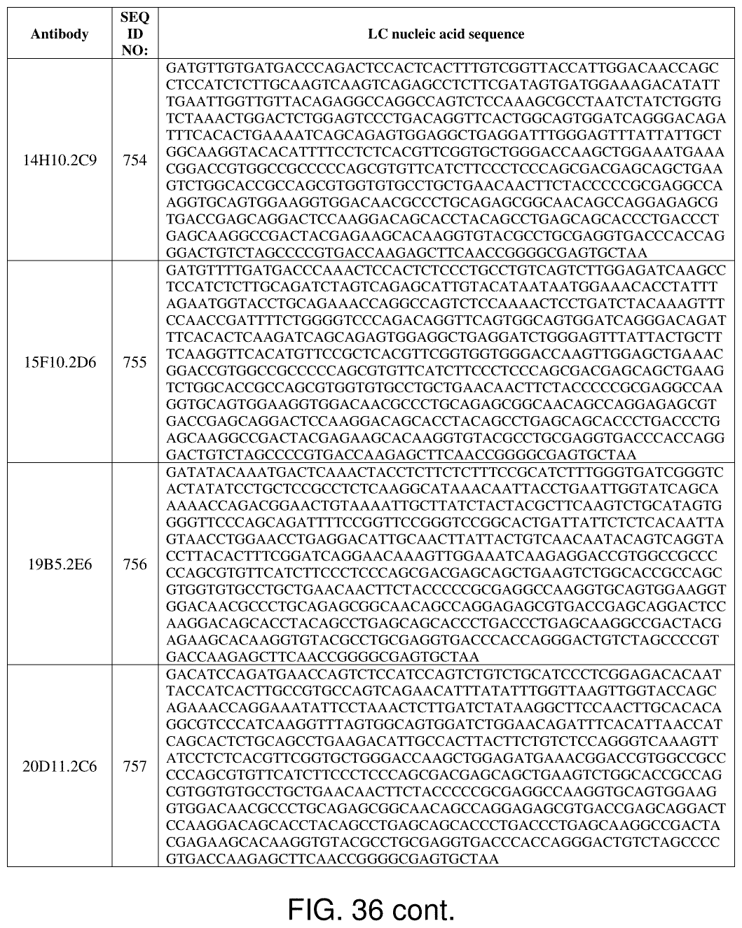

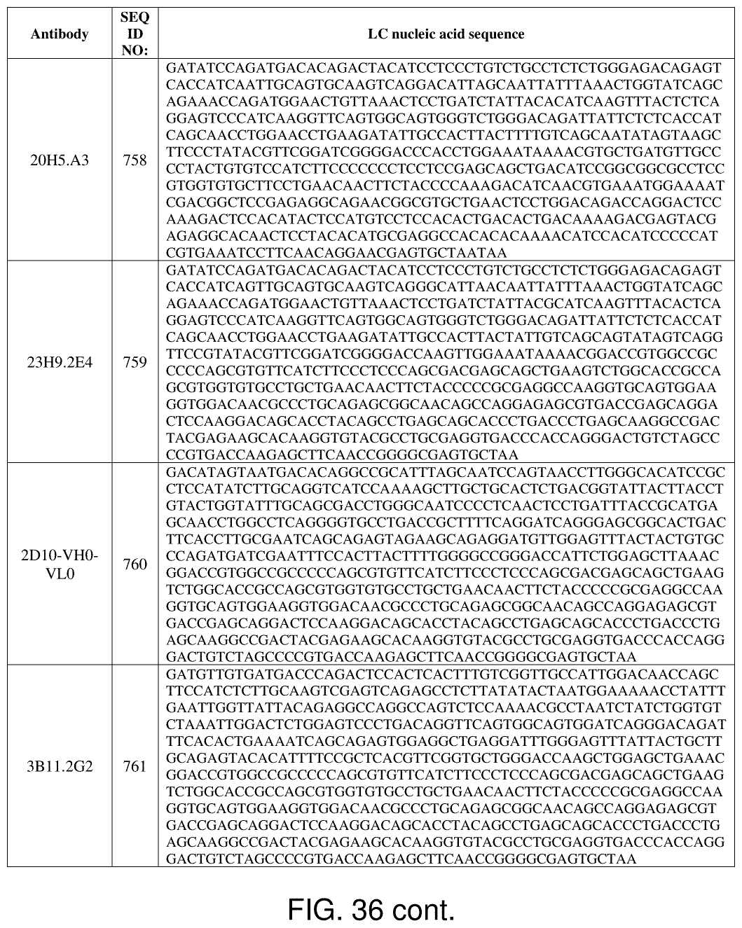

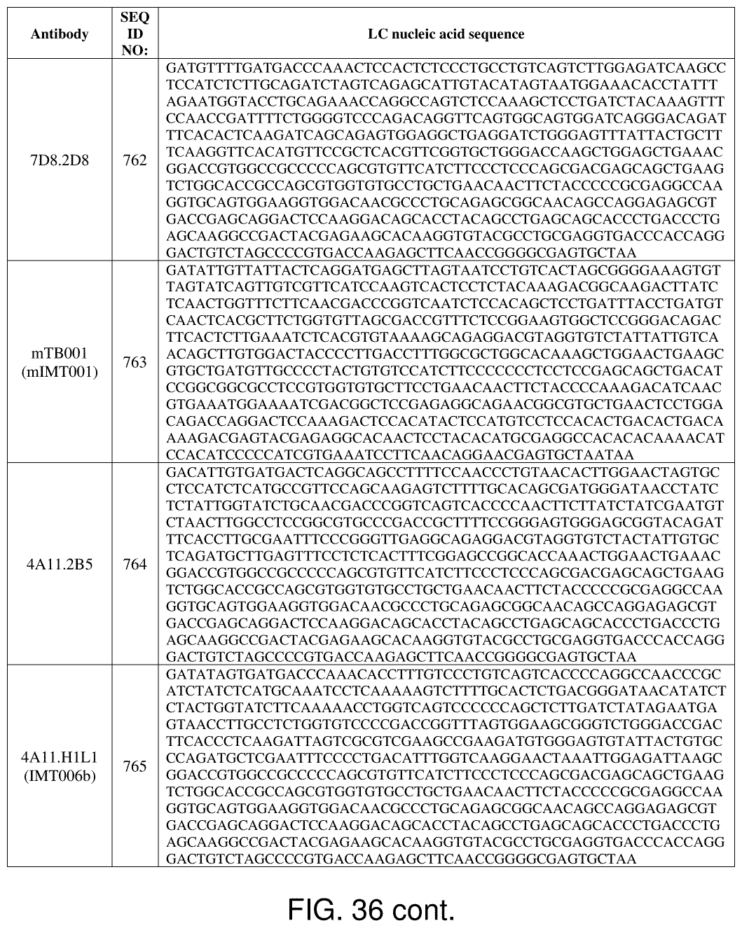

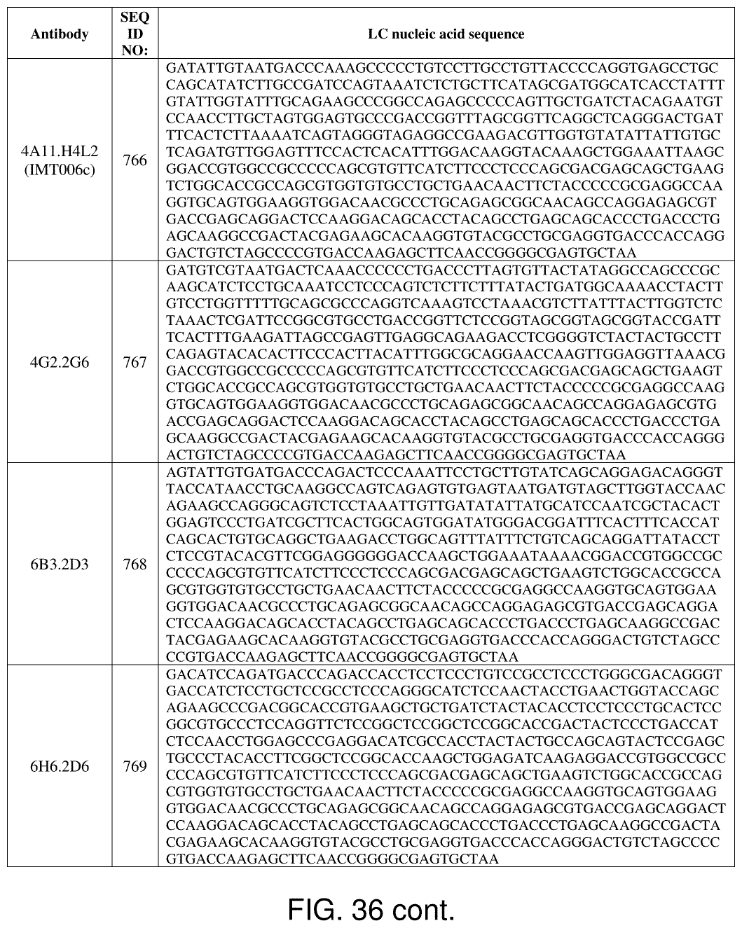

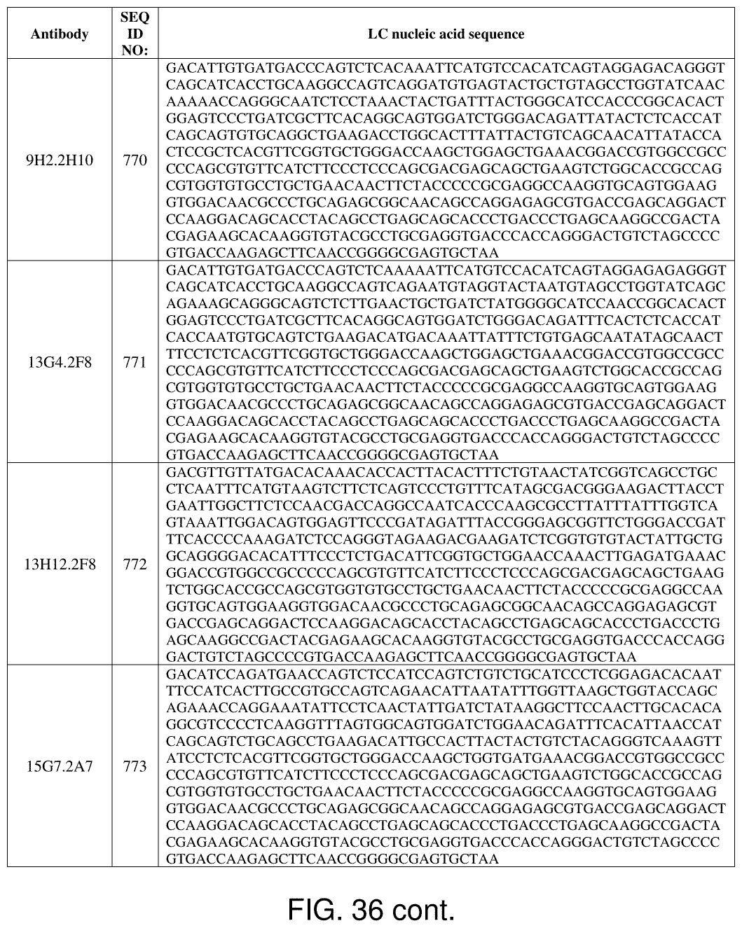

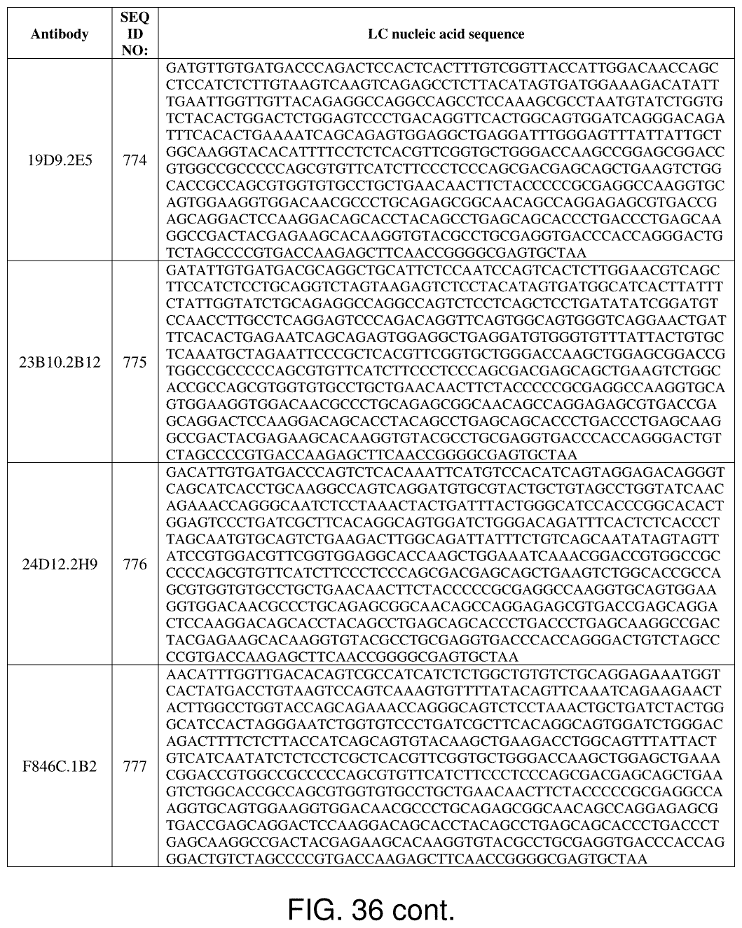

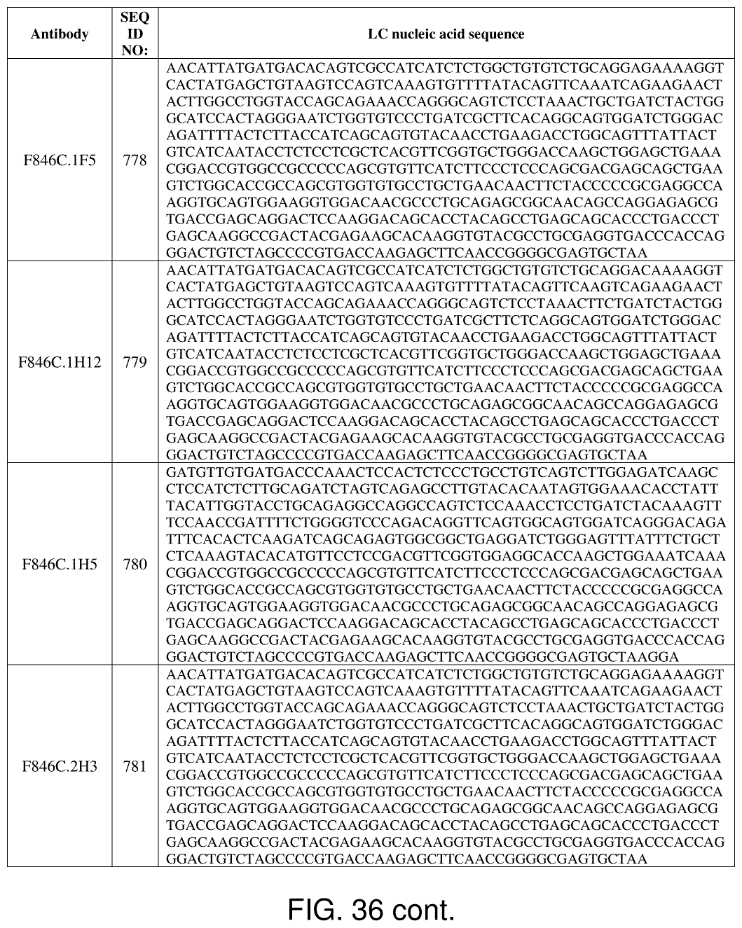

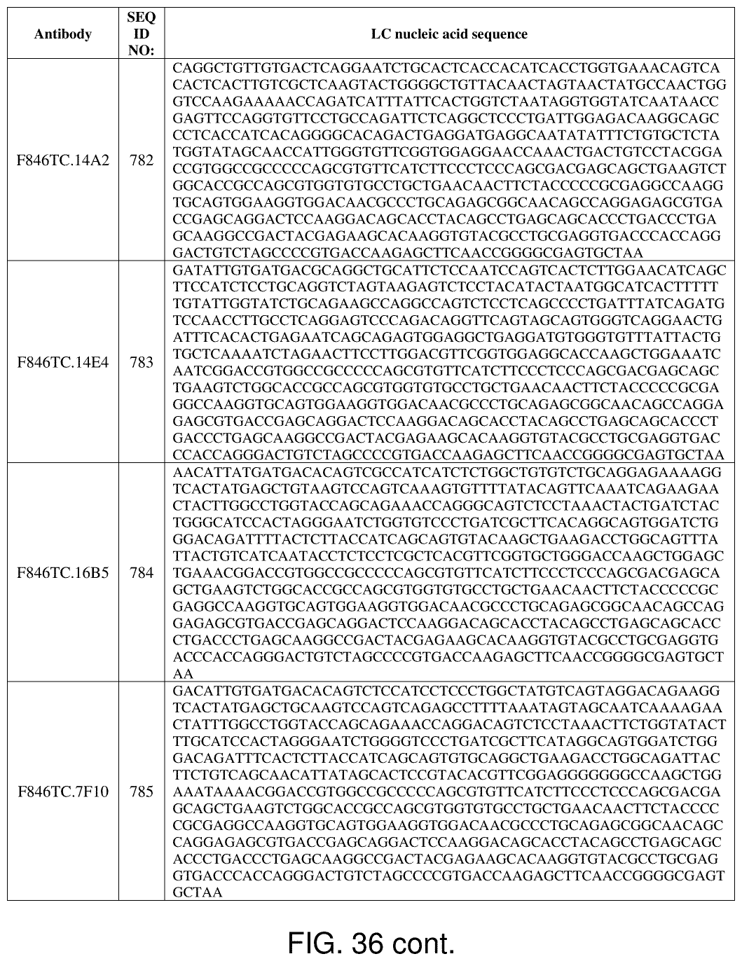

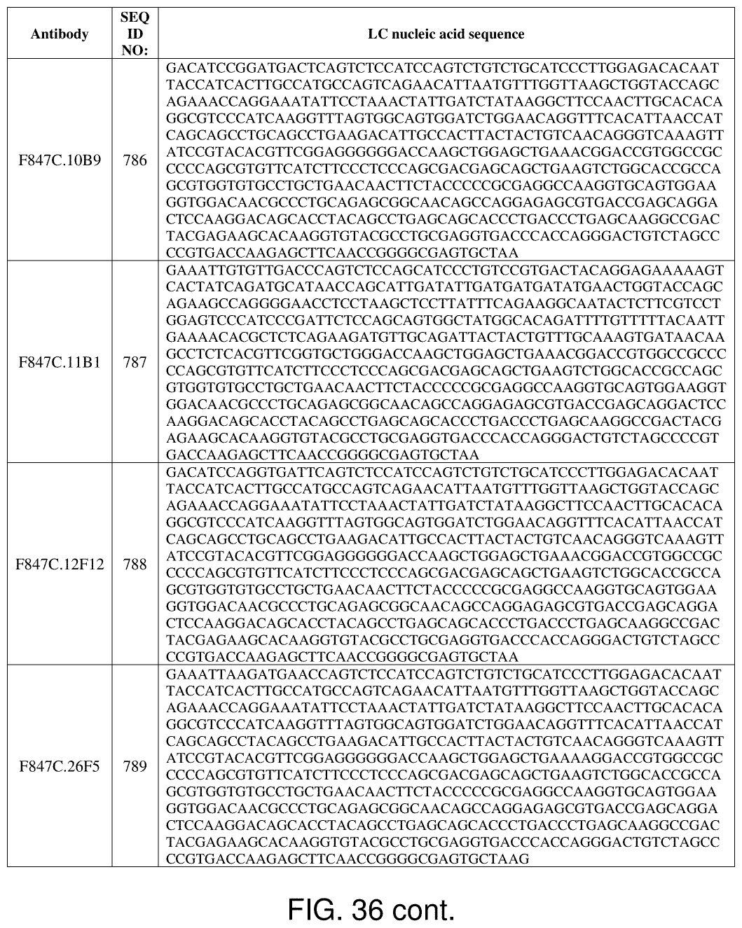

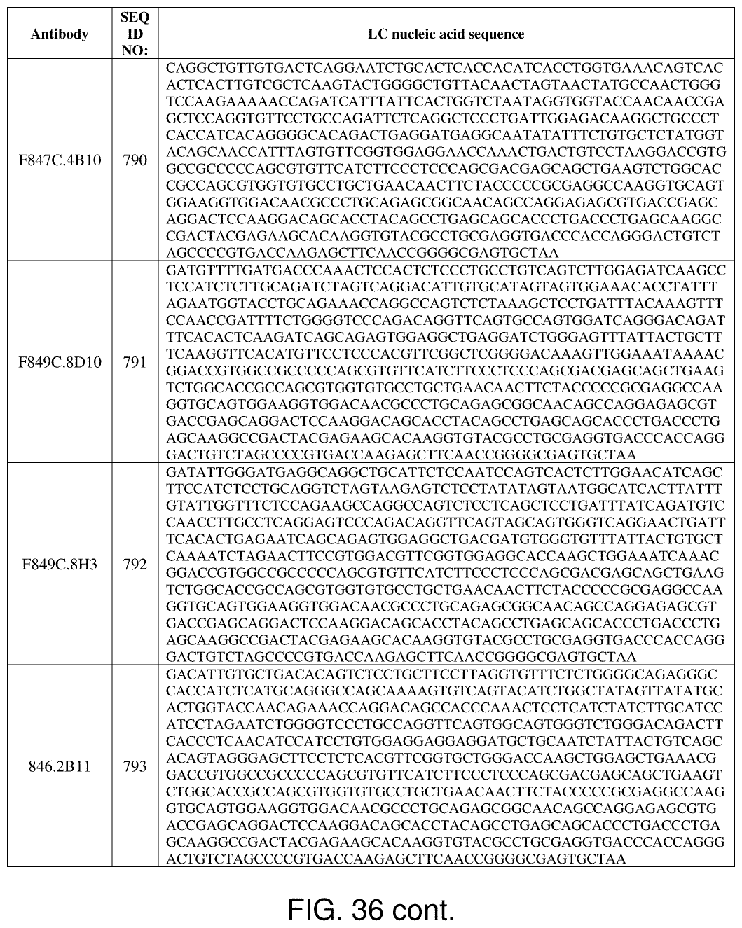

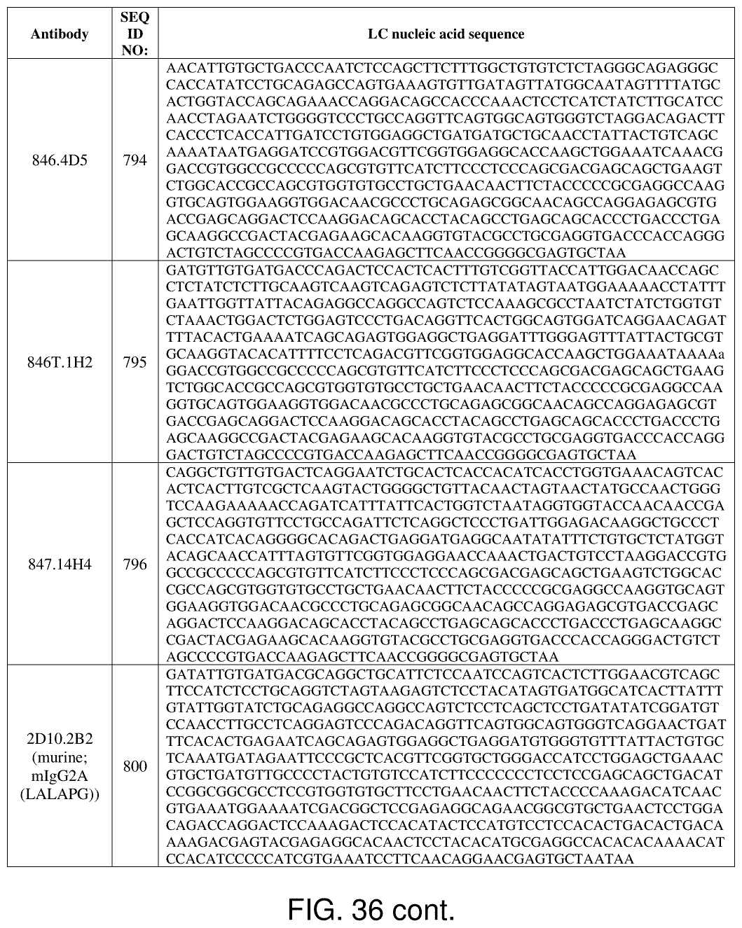

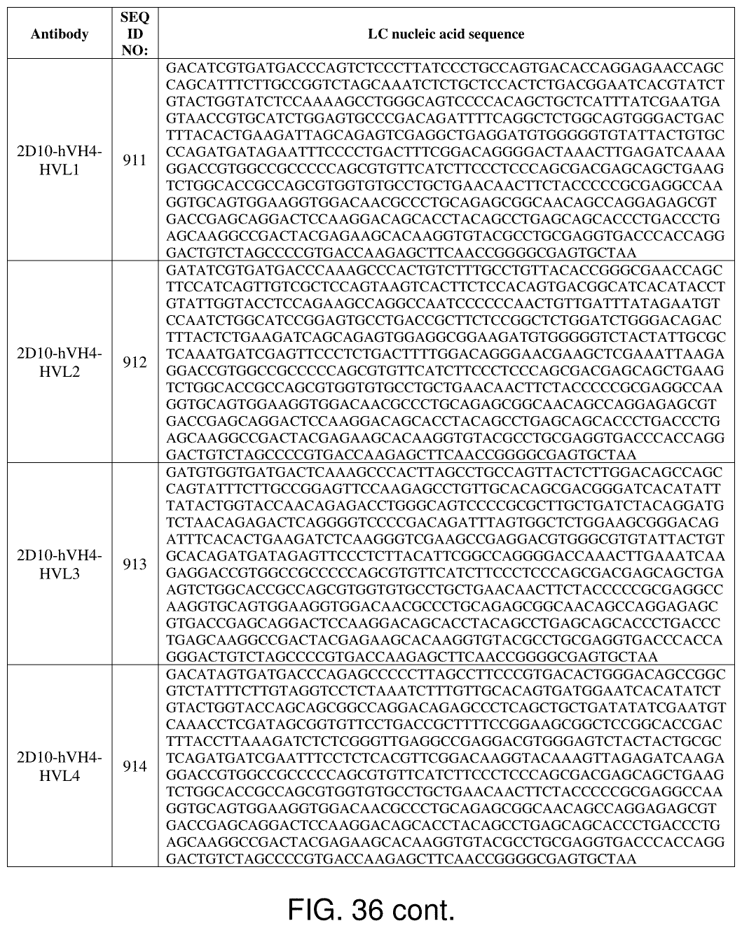

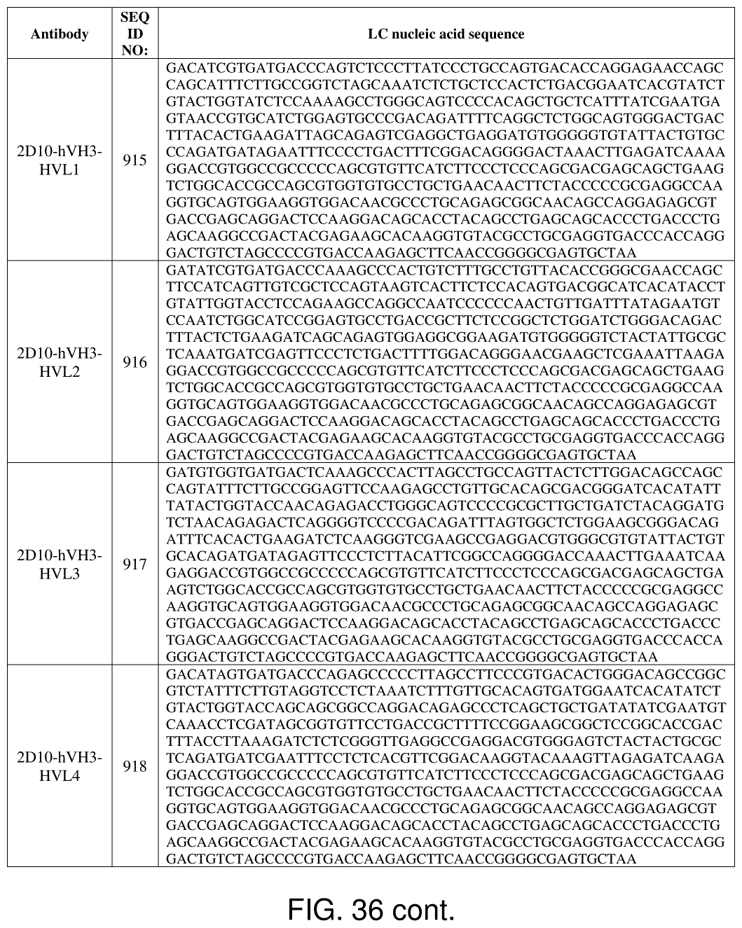

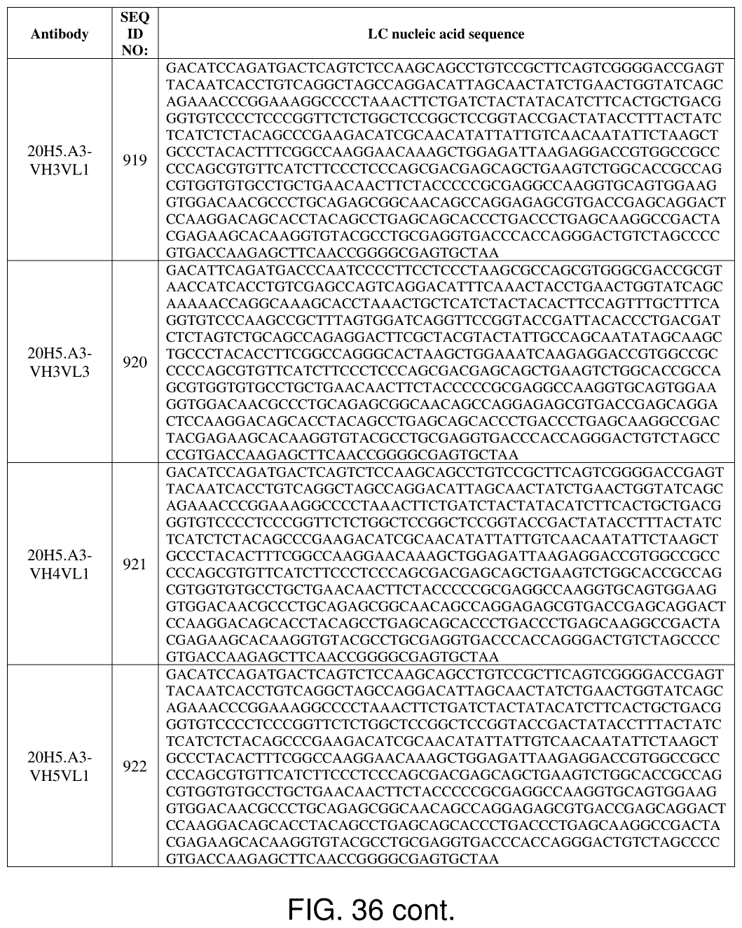

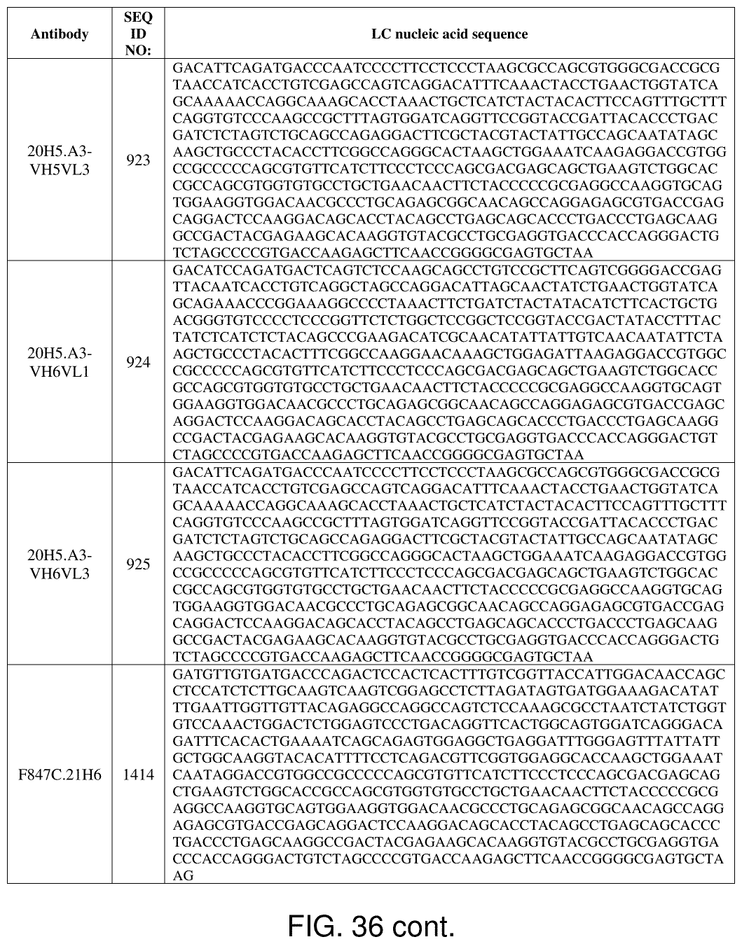

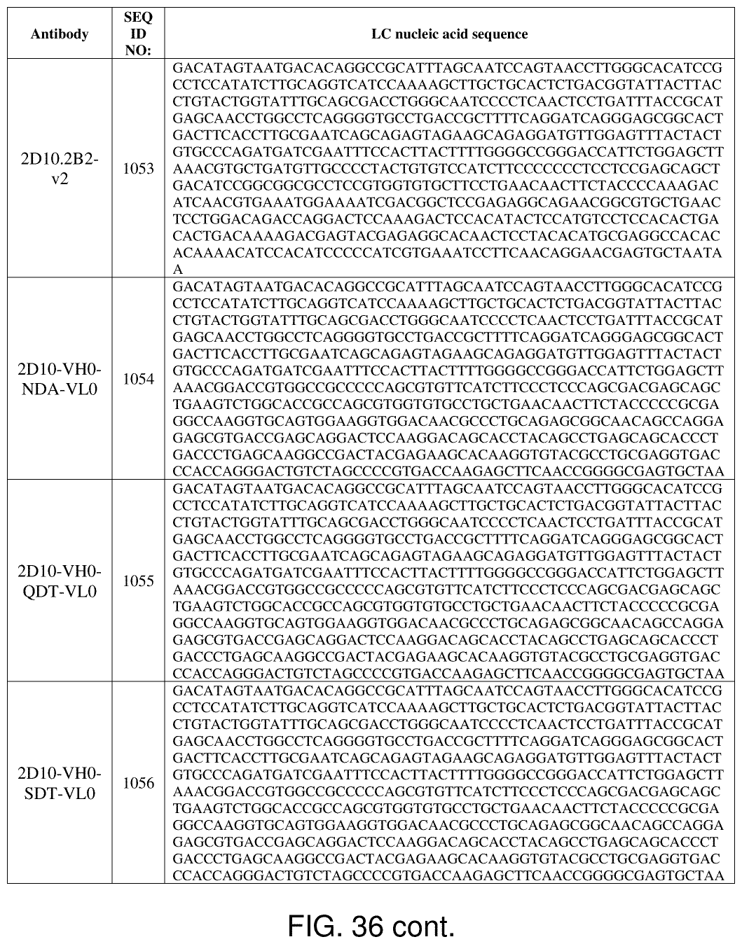

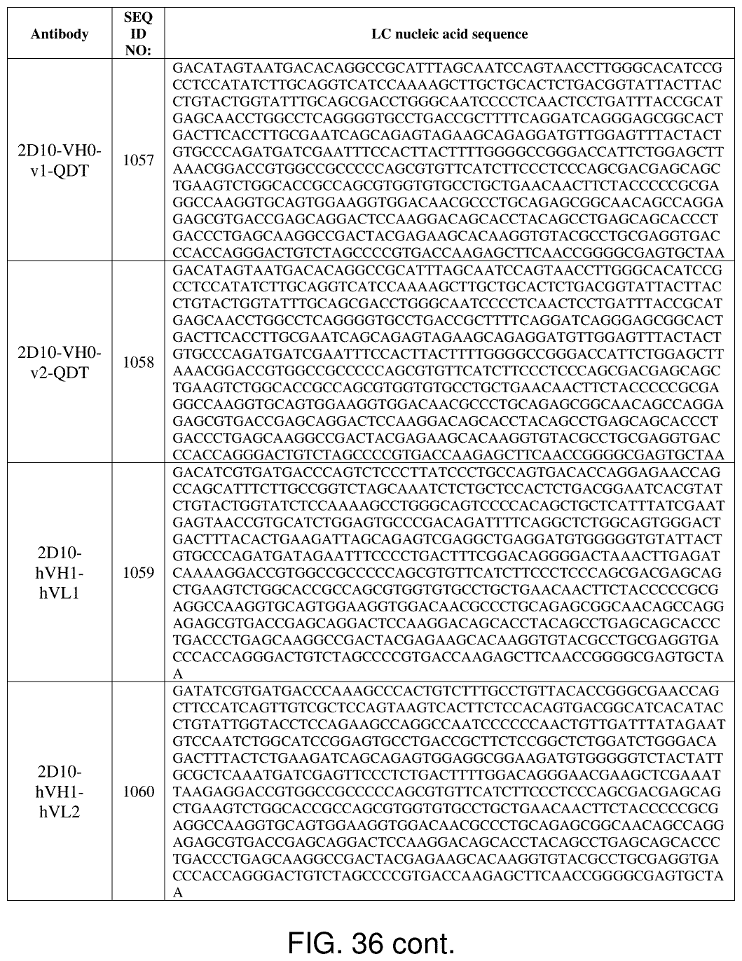

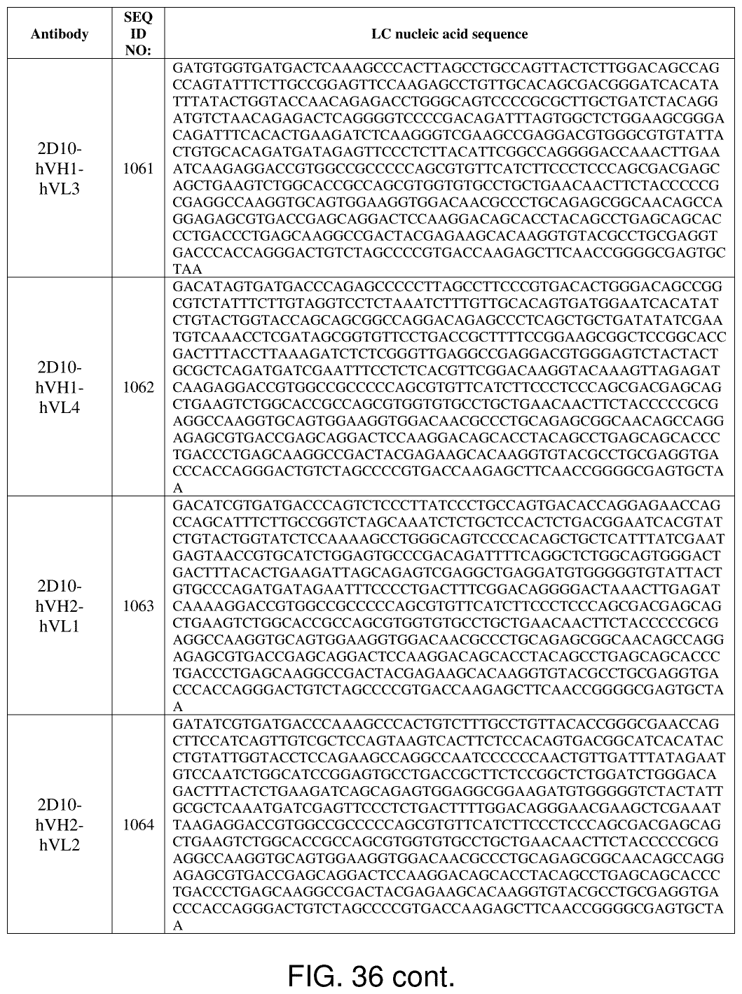

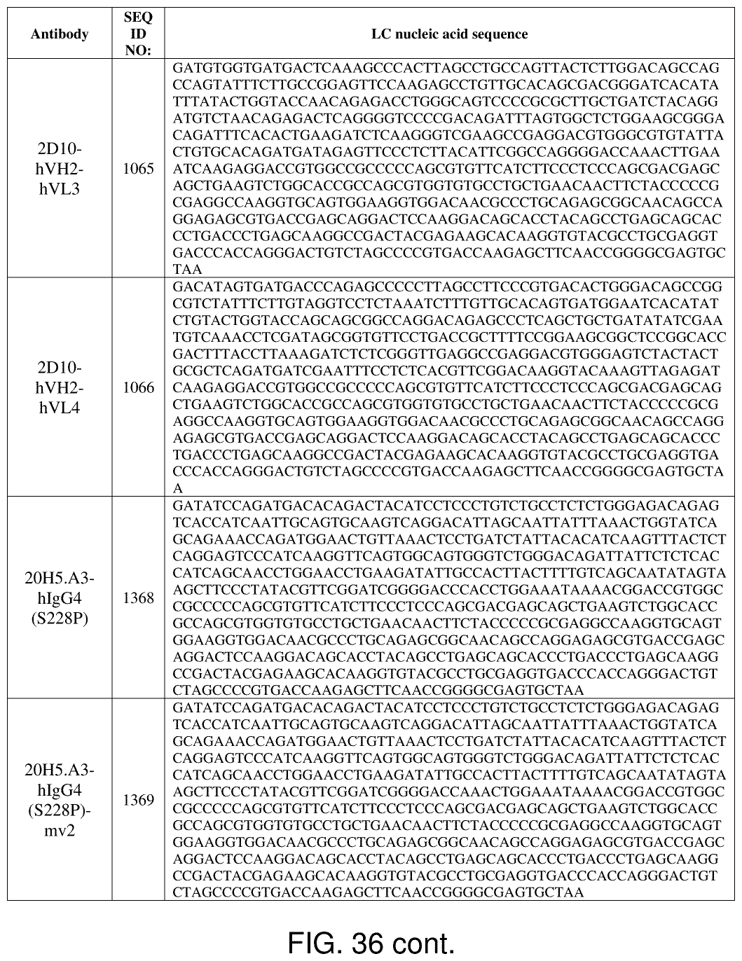

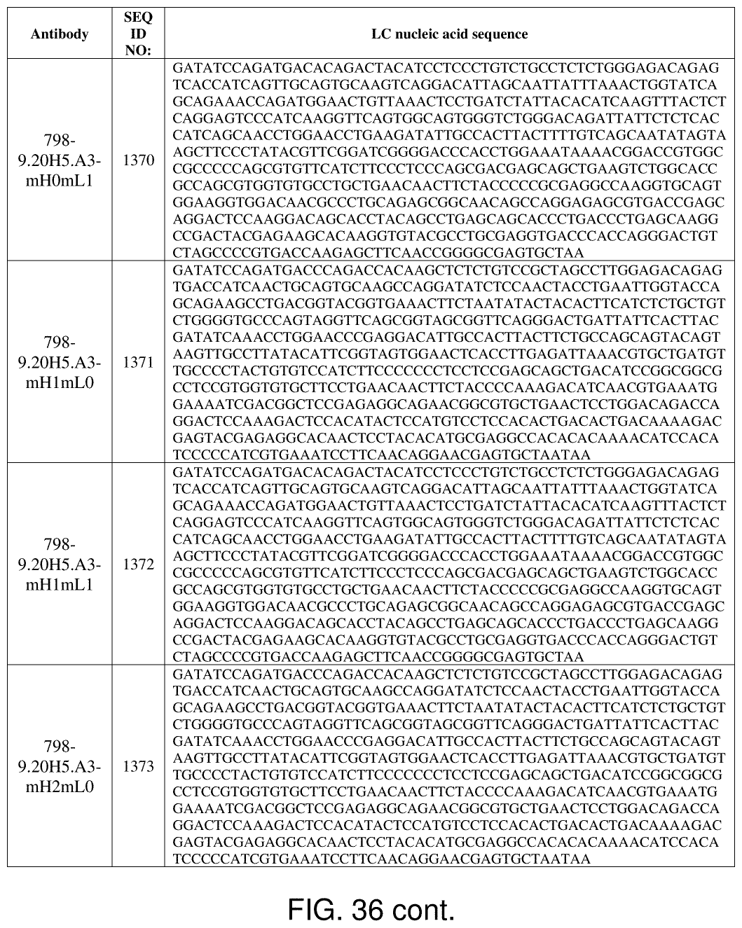

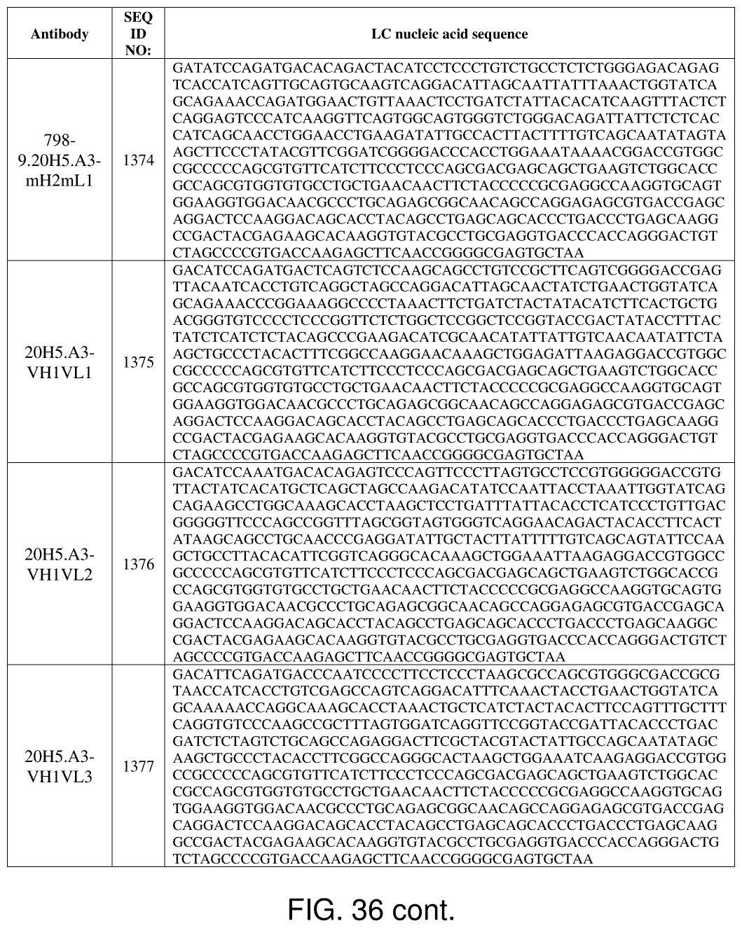

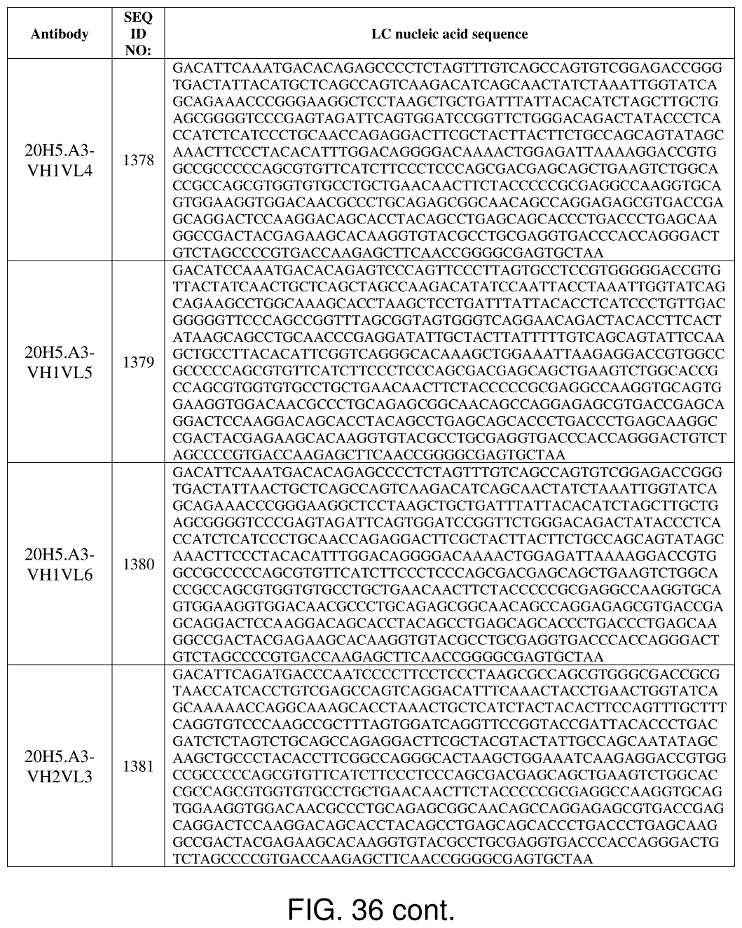

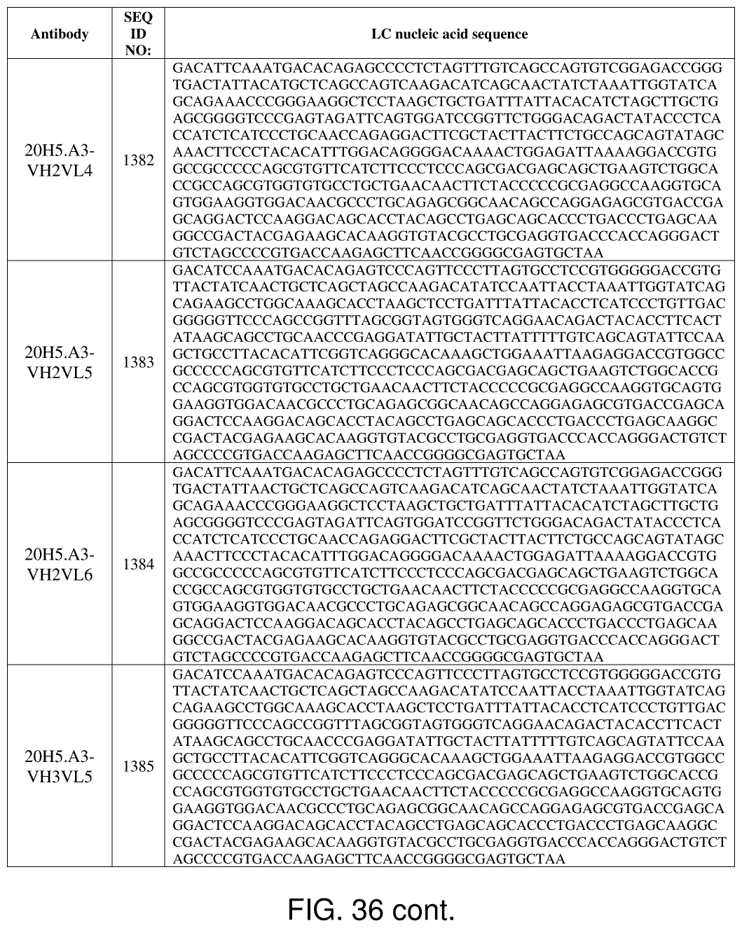

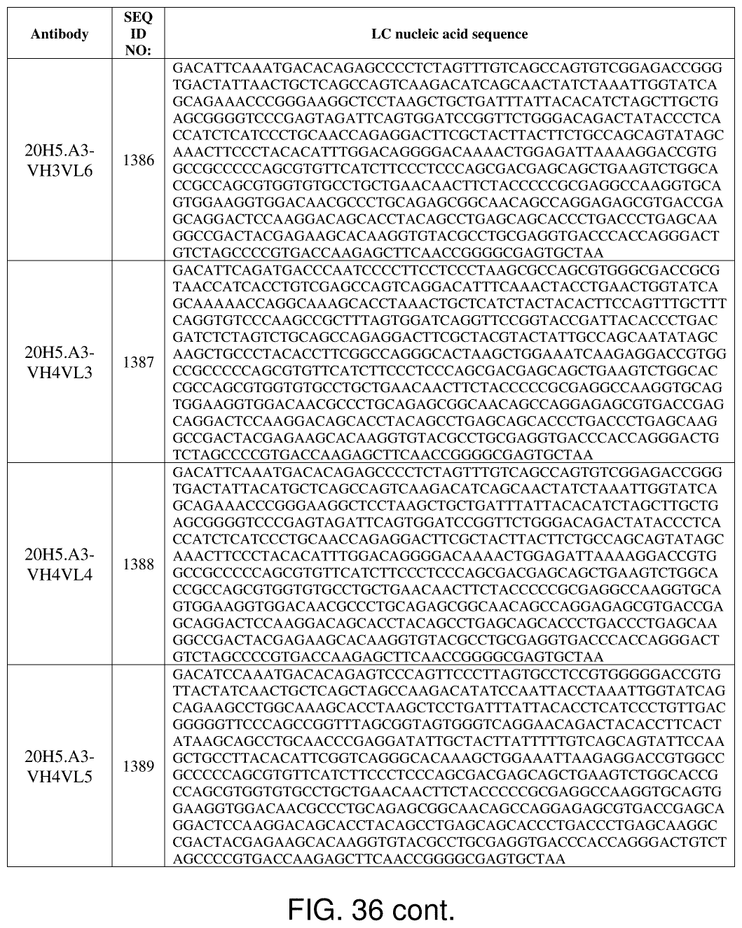

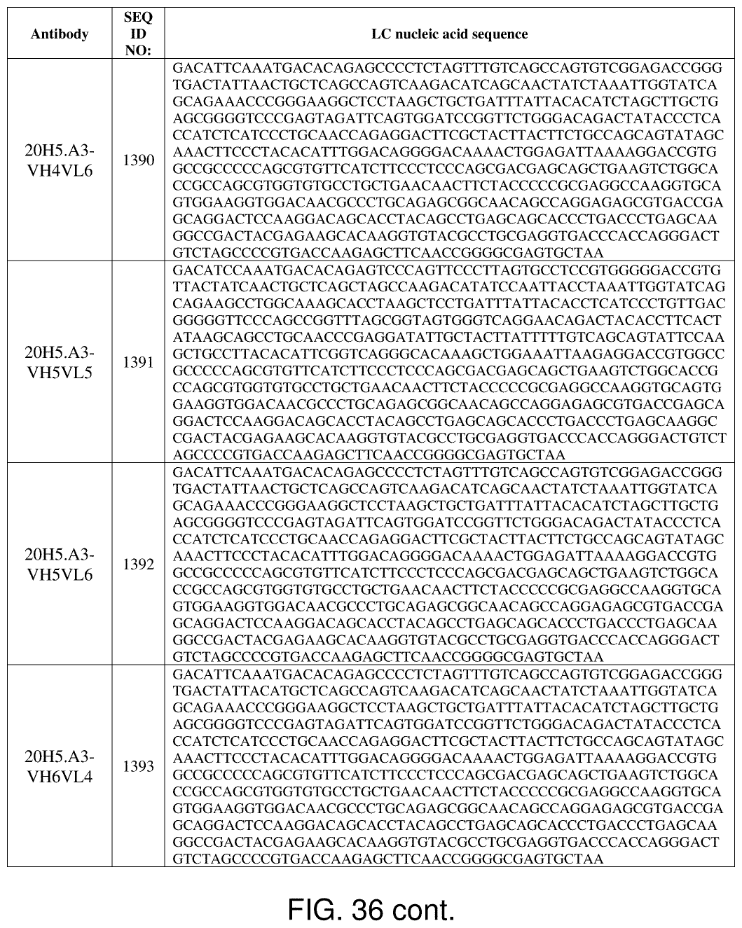

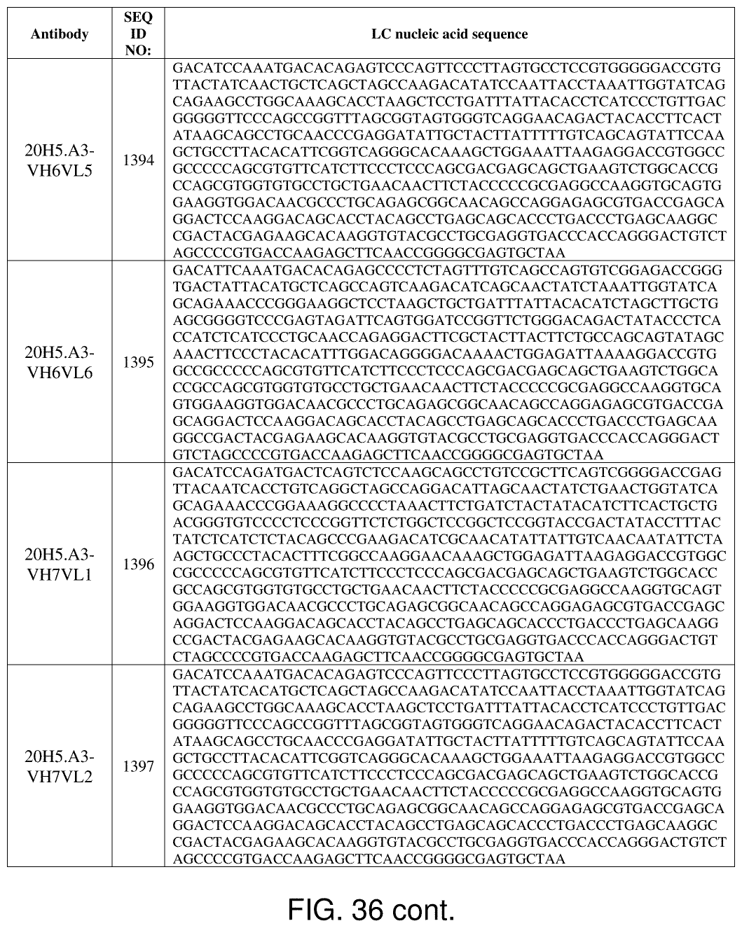

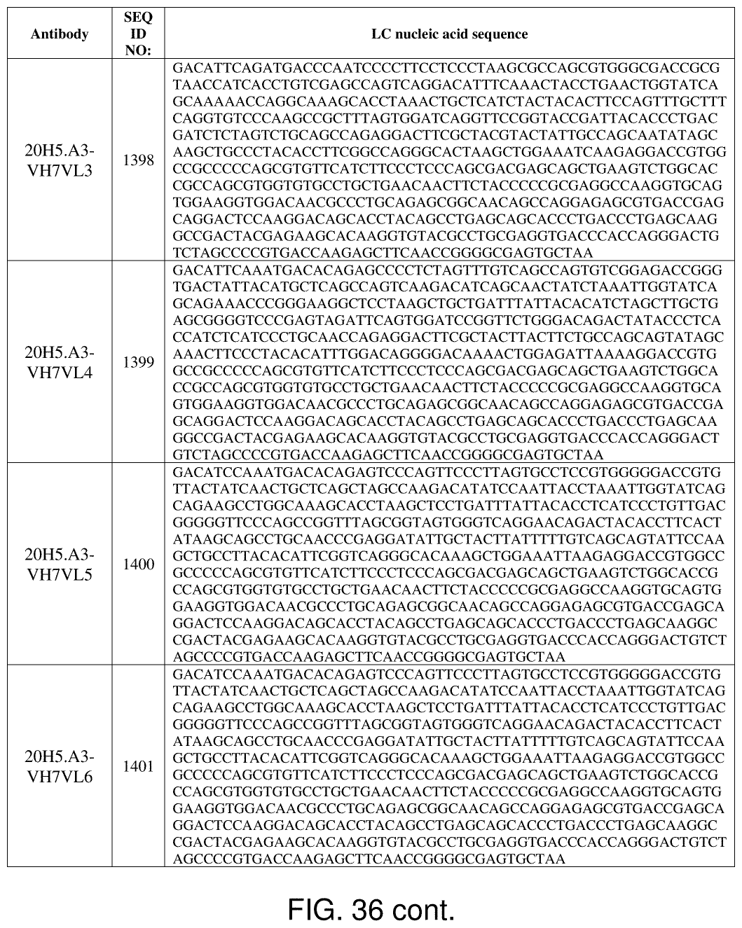

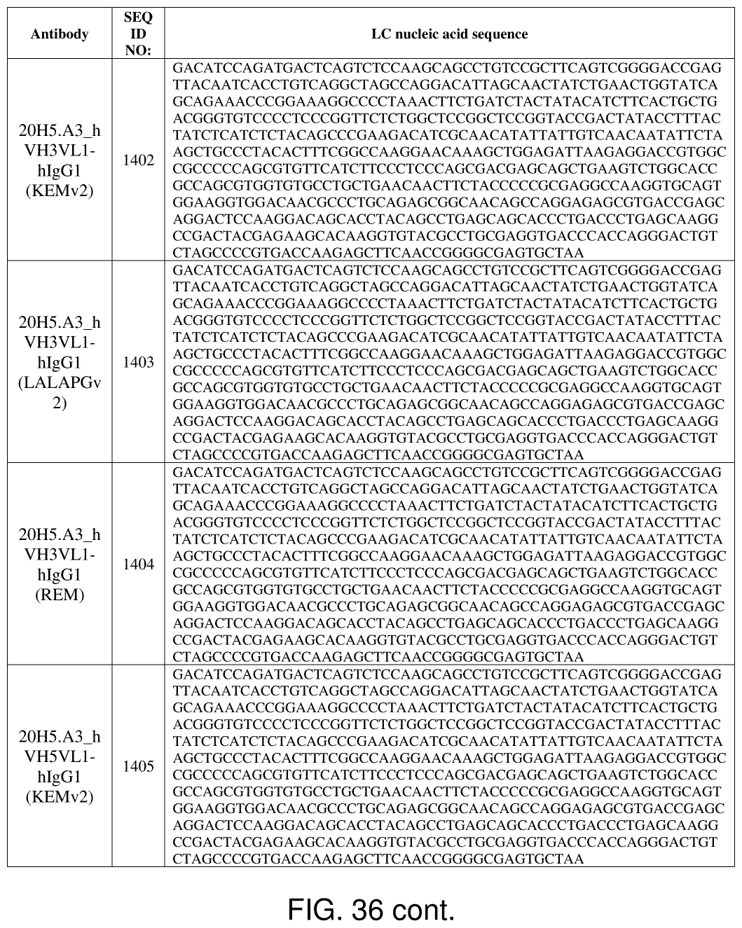

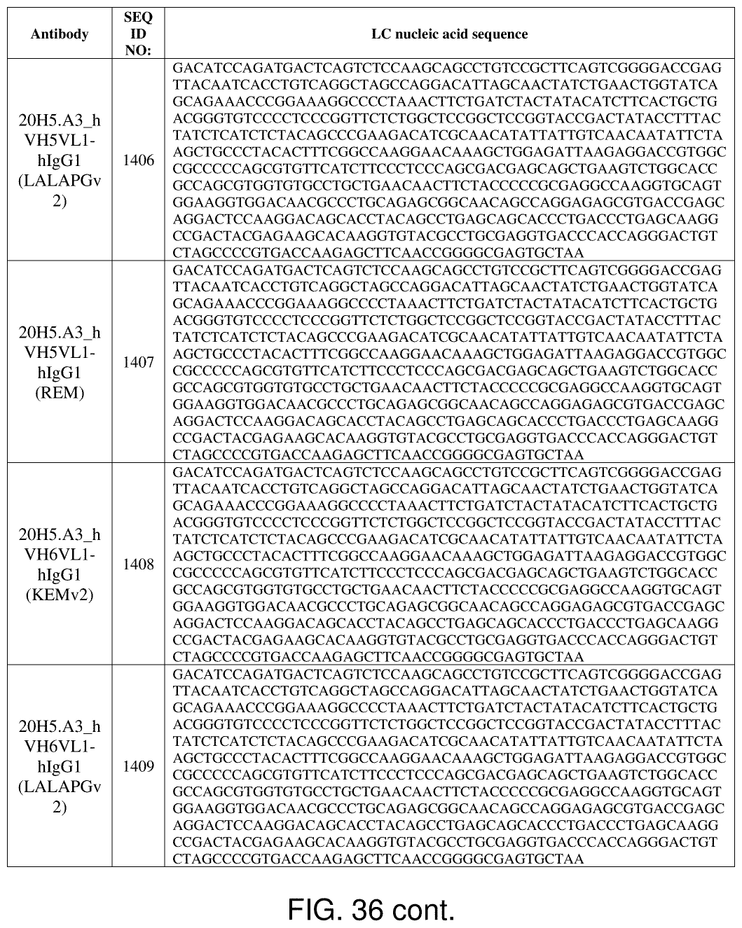

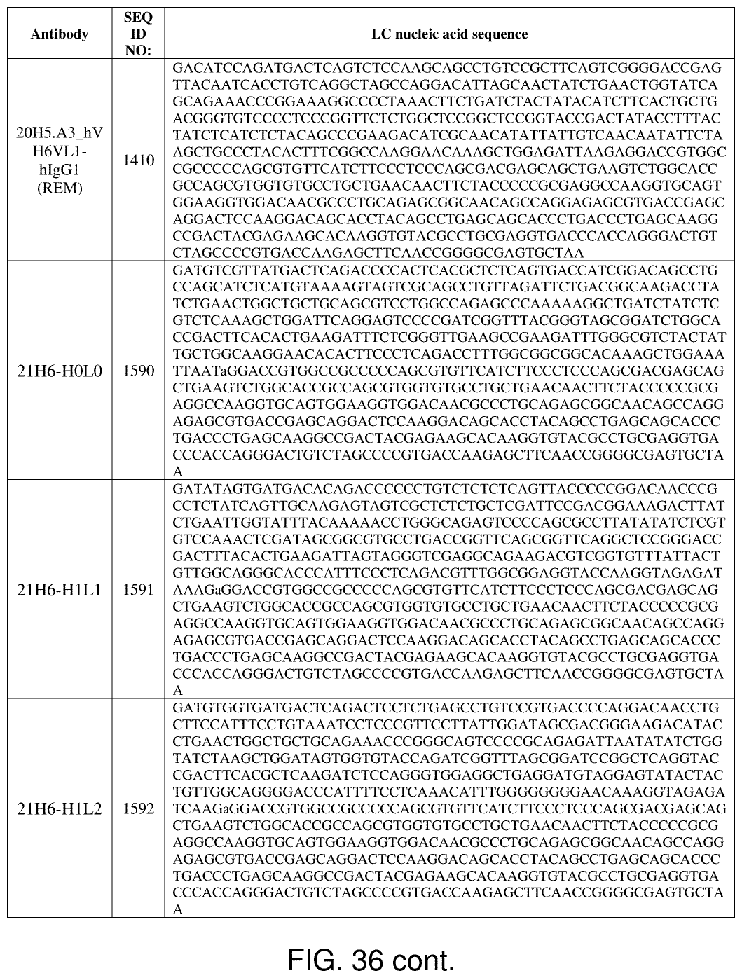

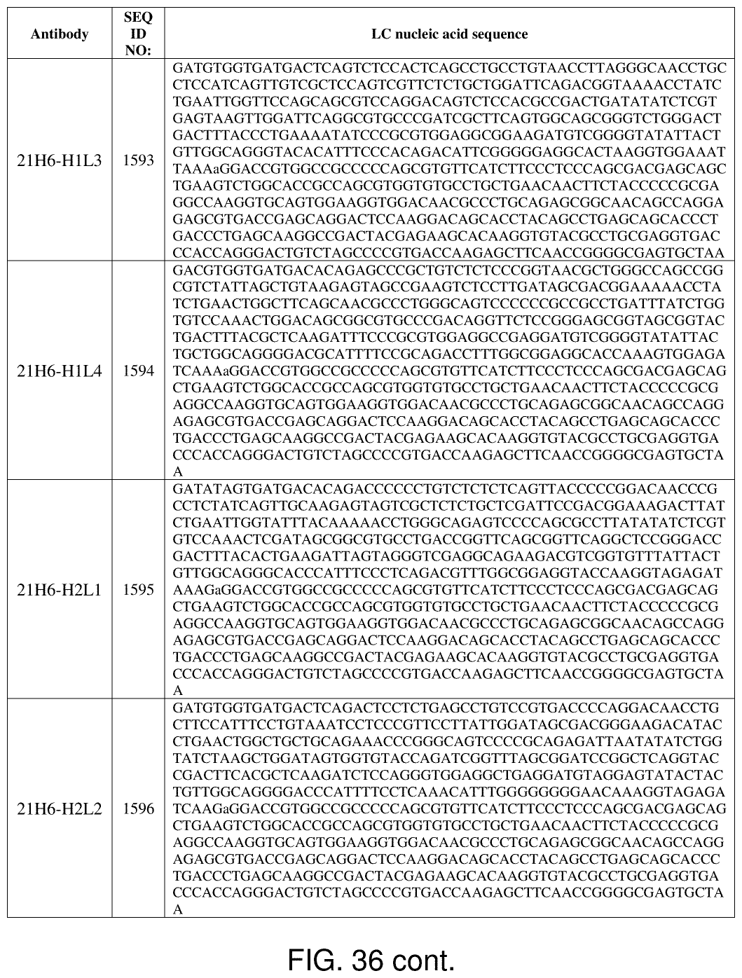

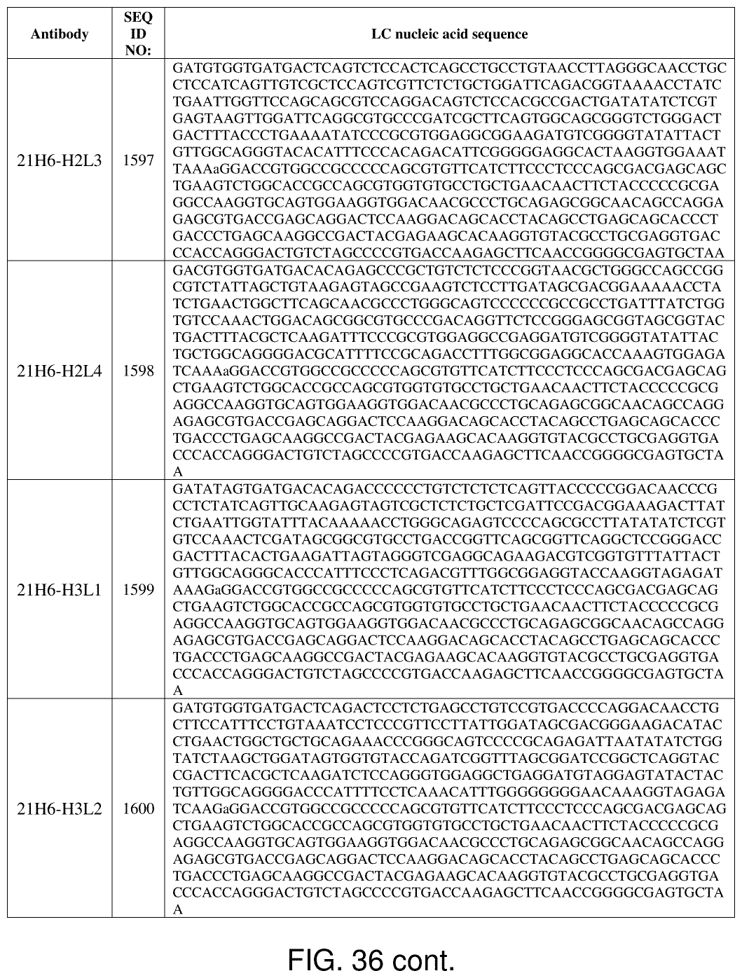

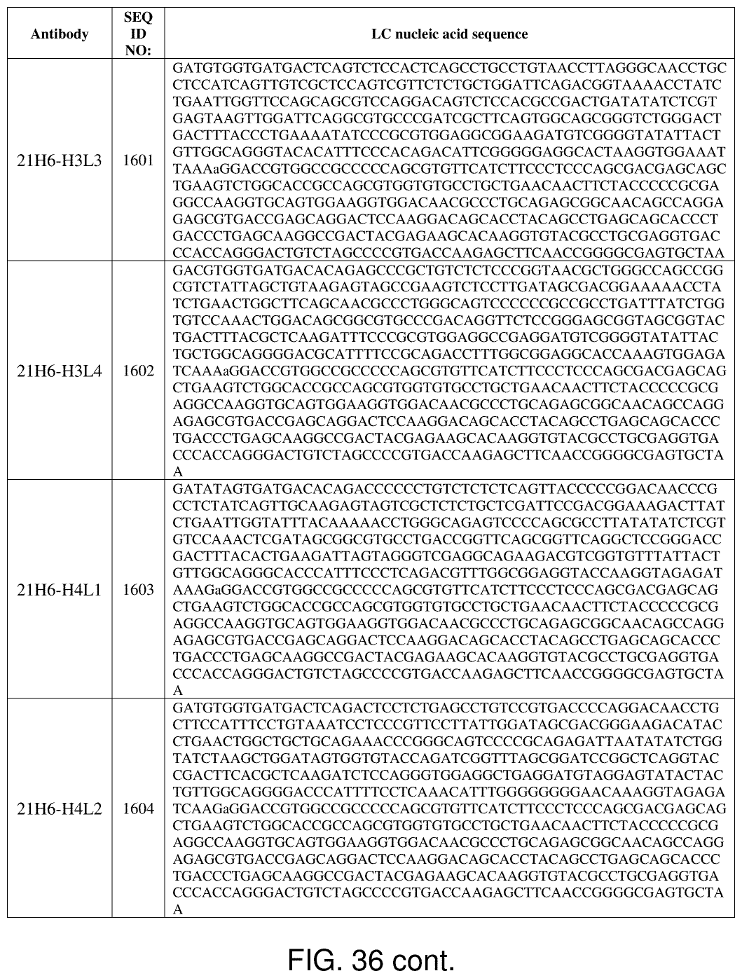

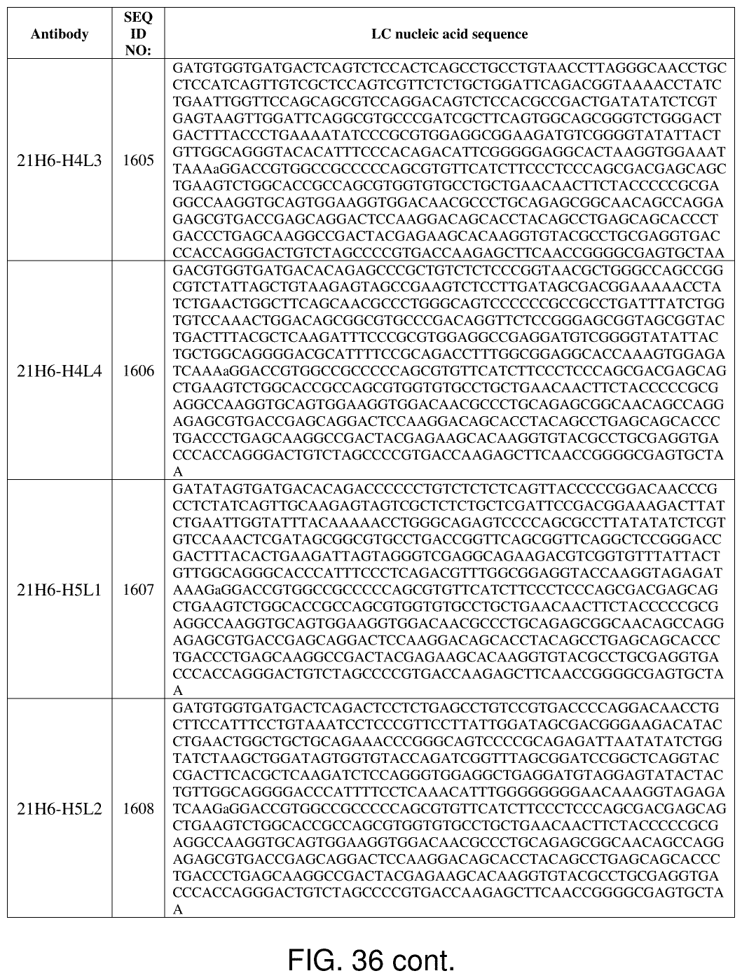

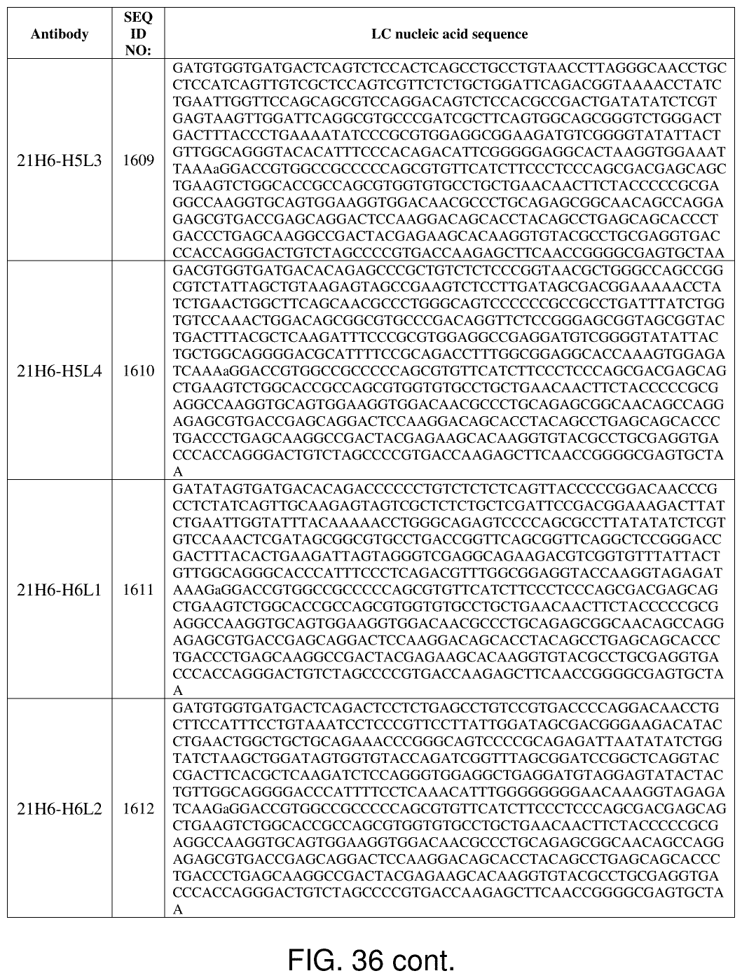

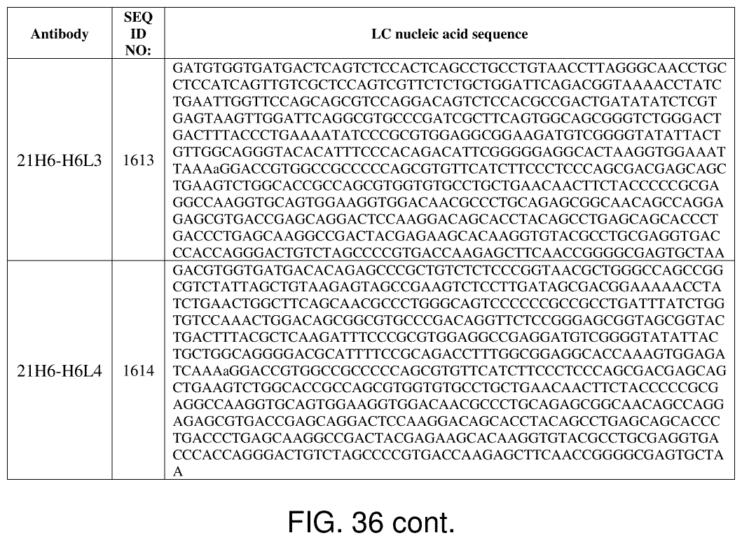

In some embodiments, nucleic acid sequences encoding for heavy chain variable regions are depicted in FIG. 33 (SEQ ID NOs: 539-620, 797, 866-880, 1011-1024, 1239-1281, 1515-1539). In some embodiments, nucleic acid sequences encoding for light chain variable regions are depicted in FIG. 34 (SEQ ID NOs: 621-702, 798, 881-895, 1025-1038, 1282-1324, 1540-1564). In some embodiments, nucleic acid sequences encoding for heavy chains are depicted in FIG. 35 (SEQ ID NO: 703-749, 799, 896-910, 1039-1052, 1325-1367, 1565-1589). In some embodiments, nucleic acid sequences encoding for light chains are depicted in FIG. 36 (SEQ ID NO: 750-796, 800, 911-925, 1053-1066, 1368-1410, 1590-1614).

Any one of the anti-Gal3 antibodies or binding fragments thereof described herein can be prepared by recombinant DNA technology, synthetic chemistry techniques, or a combination thereof. For instance, sequences encoding the desired components of the anti-Gal3 antibodies, including light chain CDRs and heavy chain CDRs are typically assembled cloned into an expression vector using standard molecular techniques know in the art. These sequences may be assembled from other vectors encoding the desired protein sequence, from PCR-generated fragments using respective template nucleic acids, or by assembly of synthetic oligonucleotides encoding the desired sequences. Expression systems can be created by transfecting a suitable cell with an expressing vector which comprises an anti-Gal3 antibody of interest or binding fragment thereof.

Nucleotide sequences corresponding to various regions of light or heavy chains of an existing antibody can be readily obtained and sequenced using convention techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection, which offers a diverse collection of well-characterized hybridoma cell lines. Alternatively, antibody nucleotides can be obtained from immunized or non-immunized rodents or humans, and form organs such as spleen and peripheral blood lymphocytes. Specific techniques applicable for extracting and synthesizing antibody nucleotides are described in Orlandi et al. (1989) Proc. Natl. Acad. Sci. U.S.A. 86: 3833-3837; Larrick et al. (1989) Biochem. Biophys. Res. Commun. 160:1250-1255; Sastry et al. (1989) Proc. Natl. Acad. Sci., U.S.A. 86: 5728-5732; and U.S. Pat. No. 5,969,108.

Polynucleotides encoding anti-Gal3 antibodies or binding fragments thereof can also be modified, for example, by substituting the coding sequence for human heavy and light chain constant regions in place of the homologous non-human sequences. In that manner, chimeric antibodies are prepared that retain the binding specificity of the original anti-Gal3 antibody or binding fragment thereof.

Also disclosed herein are methods of producing an anti-Gal3 antibody or binding fragment thereof. In some embodiments, the methods comprise expressing a nucleic acid that encodes for the anti-Gal3 antibody or binding fragment thereof in a cell and isolating the expressed anti-Gal3 antibody or binding fragment thereof from the cell. In some embodiments, the methods further comprise concentrating the anti-Gal3 antibody or binding fragment thereof to a desired concentration. In some embodiments, the cell is a mammalian cell, insect cell, or bacterial cell. In some embodiments, the anti-Gal3 antibody or binding fragment thereof is any one of the anti-Gal3 antibodies or binding fragments disclosed herein. Specific procedures of expressing antibodies in a cell and isolation of the expressed antibodies are conventionally known and can be practiced by one skilled in the art.

### Antibody Production

In some cases, anti-Gal3 antibodies or binding fragments thereof are raised by standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund's, Freund's complete, oil-in-water emulsions, etc.). When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH.

Polyclonal or monoclonal anti-Gal3 antibodies or binding fragments thereof can be produced from animals which have been genetically altered to produce human immunoglobulins. A transgenic animal can be produced by initially producing a “knock-out” animal which does not produce the animal's natural antibodies, and stably transforming the animal with a human antibody locus (e.g., by the use of a human artificial chromosome). In such cases, only human antibodies are then made by the animal. Techniques for generating such animals, and deriving antibodies therefrom, are described in U.S. Pat. Nos. 6,162,963 and 6,150,584, each incorporated fully herein by reference in its entirety. Such antibodies can be referred to as human xenogenic antibodies.

Alternatively, anti-Gal3 antibodies or binding fragments thereof can be produced from phage libraries containing human variable regions. See U.S. Pat. No. 6,174,708, incorporated fully herein by reference in its entirety.

In some aspects of any of the embodiments disclosed herein, an anti-Gal3 antibody or binding fragment thereof is produced by a hybridoma.

For monoclonal anti-Gal3 antibodies, hybridomas may be formed by isolating the stimulated immune cells, such as those from the spleen of the inoculated animal. These cells can then be fused to immortalized cells, such as myeloma cells or transformed cells, which are capable of replicating indefinitely in cell culture, thereby producing an immortal, immunoglobulin-secreting cell line. The immortal cell line utilized can be selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

In addition, the anti-Gal3 antibody or binding fragment thereof may be produced by genetic engineering.

Anti-Gal3 antibodies or binding fragments thereof disclosed herein can have a reduced propensity to induce an undesired immune response in humans, for example, anaphylactic shock, and can also exhibit a reduced propensity for priming an immune response which would prevent repeated dosage with an antibody therapeutic or imaging agent (e.g., the human-anti-murine-antibody “HAMA” response). Such anti-Gal3 antibodies or binding fragments thereof include, but are not limited to, humanized, chimeric, or xenogenic human anti-Gal3 antibodies or binding fragments thereof.

Chimeric anti-Gal3 antibodies or binding fragments thereof can be made, for example, by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art and may be achieved by standard means (as described, e.g., in U.S. Pat. No. 5,624,659, incorporated fully herein by reference).

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In some examples, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

Humanized antibodies can be engineered to contain human-like immunoglobulin domains and incorporate only the complementarity-determining regions of the animal-derived antibody. This can be accomplished by carefully examining the sequence of the hyper-variable loops of the variable regions of a monoclonal antigen binding unit or monoclonal antibody and fitting them to the structure of a human antigen binding unit or human antibody chains. See, e.g., U.S. Pat. No. 6,187,287, incorporated fully herein by reference.

Methods for humanizing non-human antibodies are well known in the art. “Humanized” antibodies are antibodies in which at least part of the sequence has been altered from its initial form to render it more like human immunoglobulins. In some versions, the heavy (H) chain and light (L) chain constant (C) regions are replaced with human sequence. This can be a fusion polypeptide comprising a variable (V) region and a heterologous immunoglobulin C region. In some versions, the complementarity determining regions (CDRs) comprise non-human antibody sequences, while the V framework regions have also been converted to human sequences. See, for example, EP 0329400. In some versions, V regions are humanized by designing consensus sequences of human and mouse V regions and converting residues outside the CDRs that are different between the consensus sequences.

In principle, a framework sequence from a humanized antibody can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a human antibody (HuAb) is to the original murine antibody (muAb), the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The disclosure thus encompasses HuAbs with different variable (V) regions. It is within the skill of one in the art to determine suitable V region sequences and to optimize these sequences. Methods for obtaining antibodies with reduced immunogenicity are also described in U.S. Pat. No. 5,270,202 and EP 699,755, each hereby incorporated by reference in its entirety.

Humanized antibodies can be prepared by a process of analysis of the parental sequences and various conceptual humanized products using three dimensional models of the parental and humanized sequences. Three-dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved.

A process for humanization of subject antigen binding units can be as follows. The best-fit germline acceptor heavy and light chain variable regions are selected based on homology, canonical structure and physical properties of the human antibody germlines for grafting. Computer modeling of mVH/VL versus grafted hVH/VL is performed and prototype humanized antibody sequence is generated. If modeling indicated a need for framework back-mutations, second variant with indicated FW changes is generated. DNA fragments encoding the selected germline frameworks and murine CDRs are synthesized. The synthesized DNA fragments are subcloned into IgG expression vectors and sequences are confirmed by DNA sequencing. The humanized antibodies are expressed in cells, such as 293F and the proteins are tested, for example in MDM phagocytosis assays and antigen binding assays. The humanized antigen binding units are compared with parental antigen binding units in antigen binding affinity, for example, by FACS on cells expressing the target antigen. If the affinity is greater than 2-fold lower than parental antigen binding unit, a second round of humanized variants can be generated and tested as described above.

As noted above, an anti-Gal3 antibody or binding fragment thereof can be either “monovalent” or “multivalent.” Whereas the former has one binding site per antigen-binding unit, the latter contains multiple binding sites capable of binding to more than one antigen of the same or different kind. Depending on the number of binding sites, antigen binding units may be bivalent (having two antigen-binding sites), trivalent (having three antigen-binding sites), tetravalent (having four antigen-binding sites), and so on.

Multivalent anti-Gal3 antibodies or binding fragments thereof can be further classified on the basis of their binding specificities. A “monospecific” anti-Gal3 antibody or binding fragment thereof is a molecule capable of binding to one or more antigens of the same kind. A “multispecific” anti-Gal3 antibody or binding fragment thereof is a molecule having binding specificities for at least two different antigens. While such molecules normally will only bind two distinct antigens (i.e. bispecific anti-Gal3 antibodies), antibodies with additional specificities such as trispecific antibodies are encompassed by this expression when used herein. This disclosure further provides multispecific anti-Gal3 antibodies. Multispecific anti-Gal3 antibodies or binding fragments thereof are multivalent molecules capable of binding to at least two distinct antigens, e.g., bispecific and trispecific molecules exhibiting binding specificities to two and three distinct antigens, respectively.

In some embodiments, the methods further provide for screening for or identifying antibodies or binding fragments thereof capable of disrupting an interaction between Gal3 and a target protein, such as an insulin receptor or glucose transporter. A non-limiting example of such a method is described in Example 1. In some aspects, the method may comprise: (a) contacting Gal3 protein with an antibody or binding fragment thereof that selectively binds to Gal3, thereby forming a Gal3-antibody complex; (b) contacting the Gal3-antibody complex with the target protein; (c) removing unbound target protein; and (d) detecting the target protein bound to the Gal3-antibody complex, wherein the antibody or binding fragment thereof is capable of disrupting an interaction of Gal3 and the target protein when the target protein is not detected in (d). In some cases, the method comprises an immunoassay. In some cases, the immunoassay is an enzyme-linked immunosorbent assay (ELISA).

### Host Cells

In some embodiments, the present disclosure provides host cells expressing any one of the anti-Gal3 antibodies or binding fragments thereof disclosed herein. A subject host cell typically comprises a nucleic acid encoding any one of the anti-Gal3 antibodies or binding fragments thereof disclosed herein.

The disclosure provides host cells transfected with the polynucleotides, vectors, or a library of the vectors described above. The vectors can be introduced into a suitable prokaryotic or eukaryotic cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell.

For most animal cells, any of the above-mentioned methods is suitable for vector delivery. Preferred animal cells are vertebrate cells, preferably mammalian cells, capable of expressing exogenously introduced gene products in large quantity, e.g. at the milligram level. Non-limiting examples of preferred cells are NIH3T3 cells, COS, HeLa, and CHO cells.

Once introduced into a suitable host cell, expression of the anti-Gal3 antibodies or binding fragments thereof can be determined using any nucleic acid or protein assay known in the art. For example, the presence of transcribed mRNA of light chain CDRs or heavy chain CDRs, or the anti-Gal3 antibody or binding fragment thereof can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of a polynucleotide that encodes the anti-Gal3 antibody or binding fragment thereof.

Expression of the vector can also be determined by examining the expressed anti-Gal3 antibody or binding fragment thereof. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

### Payload

In some embodiments, any anti-Gal3 antibody disclosed herein further comprises a payload. In some cases, the payload comprises a small molecule, a protein or functional fragment thereof, a peptide, or a nucleic acid polymer.

In some cases, the number of payloads conjugated to the anti-Gal3 antibody (e.g., the drug-to-antibody ratio or DAR) is about 1:1, one payload to one anti-Gal3 antibody. In some cases, the ratio of the payloads to the anti-Gal3 antibody is about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some cases, the ratio of the payloads to the anti-Gal3 antibody is about 2:1. In some cases, the ratio of the payloads to the anti-Gal3 antibody is about 3:1. In some cases, the ratio of the payloads to the anti-Gal3 antibody is about 4:1. In some cases, the ratio of the payloads to the anti-Gal3 antibody is about 6:1. In some cases, the ratio of the payloads to the anti-Gal3 antibody is about 8:1. In some cases, the ratio of the payloads to the anti-Gal3 antibody is about 12:1.

In some embodiment, the payload is a small molecule. In some instances, the small molecule is a cytotoxic payload. Exemplary cytotoxic payloads include, but are not limited to, microtubule disrupting agents, DNA modifying agents, or Akt inhibitors.

In some embodiments, the payload comprises a microtubule disrupting agent. Exemplary microtubule disrupting agents include, but are not limited to, 2-methoxyestradiol, auristatin, chalcones, colchicine, combretastatin, cryptophycin, dictyostatin, discodermolide, dolastain, eleutherobin, epothilone, halichondrin, laulimalide, maytansine, noscapinoid, paclitaxel, peloruside, phomopsin, podophyllotoxin, rhizoxin, spongistatin, taxane, tubulysin, vinca alkaloid, vinorelbine, or derivatives or analogs thereof.

In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5,208,020, 5,416,064, 7,276,497, and 6,716,821 or U.S. Publication Nos. 2013029900 and US20130323268.

In some embodiments, the payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 15 analog is cemadotin or tasidotin.

In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714, and 8,871,720.

In some embodiments, the payload comprises a DNA modifying agent. In some embodiments, the DNA modifying agent comprises DNA cleavers, DNA intercalators, DNA transcription inhibitors, or DNA cross-linkers. In some instances, the DNA cleaver comprises bleomycine A2, calicheamicin, or derivatives or analogs thereof. In some instances, the DNA intercalator comprises doxorubicin, epirubicin, PNU-159682, duocarmycin, pyrrolobenzodiazepine, oligomycin C, daunorubicin, valrubicin, topotecan, or derivatives or analogs thereof. In some instances, the DNA transcription inhibitor comprises dactinomycin. In some instances, the DNA cross-linker comprises mitomycin C.

In some embodiments, the DNA modifying agent comprises amsacrine, anthracycline, camptothecin, doxorubicin, duocarmycin, enediyne, etoposide, indolinobenzodiazepine, netropsin, teniposide, or derivatives or analogs thereof.

In some embodiments, the anthracycline is doxorubicin, daunorubicin, epirubicin, idarubicin, mitomycin-C, dactinomycin, mithramycin, nemorubicin, pixantrone, sabarubicin, or valrubicin.

In some embodiments, the analog of camptothecin is topotecan, irinotecan, silatecan, cositecan, exatecan, lurtotecan, gimatecan, belotecan, rubitecan, or SN-38.

In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120. In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

In some embodiments, the payload comprises an Akt inhibitor. In some cases, the Akt inhibitor comprises ipatasertib (GDC-0068) or derivatives thereof.

In some embodiments, the payload comprises a polymerase inhibitor, including, but not limited to polymerase II inhibitors such as a-amanitin, and poly(ADP-ribose) polymerase (PARP) inhibitors. Exemplary PARP inhibitors include, but are not limited to Iniparib (BSI 201), Talazoparib (BMN-673), Olaparib (AZD-2281), Olaparib, Rucaparib (AG014699, PF-01367338), Veliparib (ABT-888), CEP 9722, MK 4827, BGB-290, or 3-aminobenzamide.

In some embodiments, the payload comprises a detectable moiety. As used herein, a “detectable moiety” may comprise an atom, molecule, or compound that is useful in diagnosing, detecting or visualizing a location and/or quantity of a target molecule, cell, tissue, organ, and the like. Detectable moieties that can be used in accordance with the embodiments herein include, but are not limited to, radioactive substances (e.g. radioisotopes, radionuclides, radiolabels or radiotracers), dyes, contrast agents, fluorescent compounds or molecules, bioluminescent compounds or molecules, enzyme and enhancing agents (e.g. paramagnetic ions), or specific binding moieties such as streptavidin, avidin, or biotin. In addition, some nanoparticles, for example quantum dots or metal nanoparticles can be suitable for use as a detectable moiety.

Exemplary radioactive substances that can be used as detectable moieties in accordance with the embodiments herein include, but are not limited to, ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Exemplary paramagnetic ions substances that can be used as detectable markers include, but are not limited to ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the detectable marker is a radioactive metal or paramagnetic ion, in some embodiments, the marker can be reacted with a reagent having a long tail with one or more chelating groups attached to the long tail for binding these ions. The long tail can be a polymer such as apolylysine, polysaccharide, or other derivatized or derivatizable chain having pendant groups to which may be bound to a chelating group for binding the ions. Examples of chelating groups that may be used according to the embodiments herein include, but are not limited to, ethylenediaminetetraacetic acid (EDTA), diethylenetriaminepentaacetic acid (DTPA), DOTA, NOTA, NOGADA, NETA, deferoxamine (DfO), porphyrins, polyamines, crown ethers, bis-thiosemicarbazones, polyoximes, and like groups. The chelate can be linked to the antigen binding construct by a group which allows formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antigen binding constructs and carriers described herein. Macrocyclic chelates such as NOTA, NOGADA, DOTA, and TETA are of use with a variety of metals and radiometals including, but not limited to, radionuclides of gallium, yttrium and copper, respectively. Other ring-type chelates such as macrocyclic polyethers, which are of interest for stably binding radionuclides, such as Radium-223 for RAIT may be used. In certain embodiments, chelating moieties may be used to attach a PET imaging agent, such as an Aluminum-¹⁸F complex, to a targeting molecule for use in PET analysis.

Exemplary contrast agents that can be used as detectable moieties in accordance with the embodiments of the disclosure include, but are not limited to, barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof.

Bioluminescent and fluorescent compounds or molecules and dyes that can be used as detectable moieties in accordance with the embodiments of the disclosure include, but are not limited to, fluorescein, fluorescein isothiocyanate (FITC), OREGON GREEN™ rhodamine, Texas red, tetrarhodimine isothiocynate (TRITC), Cy3, Cy5, and the like), fluorescent markers (e.g., green fluorescent protein (GFP), phycoerythrin, and the like), autoquenched fluorescent compounds that are activated by tumor-associated proteases, enzymes (e.g., luciferase, horseradish peroxidase, alkaline phosphatase, and the like), nanoparticles, biotin, digoxigenin or combinations thereof.

Enzymes that can be used as detectable moieties in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

In some embodiments, the payload is a nanoparticle. The term “nanoparticle” refers to a microscopic particle whose size is measured in nanometers, e.g., a particle with at least one dimension less than about 100 nm. Nanoparticles can be used as detectable substances because they are small enough to scatter visible light rather than absorb it. For example, gold nanoparticles possess significant visible light extinction properties and appear deep red to black in solution. As a result, compositions comprising antigen binding constructs conjugated to nanoparticles can be used for the in vivo imaging of T-cells in a subject. At the small end of the size range, nanoparticles are often referred to as clusters. Metal, dielectric, and semiconductor nanoparticles have been formed, as well as hybrid structures (e.g. core-shell nanoparticles). Nanospheres, nanorods, and nanocups are just a few of the shapes that have been grown. Semiconductor quantum dots and nanocrystals are examples of additional types of nanoparticles. Such nanoscale particles can be used as payloads to be conjugated to any one of the anti-Gal3 antibodies disclosed herein.

In some embodiments, the payload comprises an immunomodulatory agent. Useful immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, streptokinase, or rapamycin.

In some embodiments, the payload comprises an immune modulator. Exemplary immune modulators include, but are not limited to, gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, xanthines, stem cell growth factors, lymphotoxins, hematopoietic factors, tumor necrosis factor (TNF) (e.g., TNFα), interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “S1 factor,” erythropoietin and thrombopoietin, or a combination thereof.

In some embodiments, the payload comprises an immunotoxin. Immunotoxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

In some instances, the payload comprises a nucleic acid polymer. In such instances, the nucleic acid polymer comprises short interfering nucleic acid (siNA), short interfering RNA (siRNA), double-stranded RNA (dsRNA), micro-RNA (miRNA), short hairpin RNA (shRNA), an antisense oligonucleotide. In other instances, the nucleic acid polymer comprises an mRNA, encoding, e.g., a cytotoxic protein or peptide or an apoptotic triggering protein or peptide. Exemplary cytotoxic proteins or peptides include a bacterial cytotoxin such as an alpha-pore forming toxin (e.g., cytolysin A from E. coli), a beta-pore-forming toxin (e.g., α-Hemolysin, PVL—panton Valentine leukocidin, aerolysin, clostridial Epsilon-toxin, Clostridium perfringens enterotoxin), binary toxins (anthrax toxin, edema toxin, C. botulinum C2 toxin, C spirofome toxin, C. perfringens iota toxin, C. difficile cyto-lethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT). Exemplary apoptotic triggering proteins or peptides include apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2). In additional instances, the nucleic acid polymer comprises a nucleic acid decoy. In some instances, the nucleic acid decoy is a mimic of protein-binding nucleic acids such as RNA-based protein-binding mimics. Exemplary nucleic acid decoys include transactivating region (TAR) decoy and Rev response element (RRE) decoy.

In some cases, the payload is an aptamer. Aptamers are small oligonucleotide or peptide molecules that bind to specific target molecules. Exemplary nucleic acid aptamers include DNA aptamers, RNA aptamers, or XNA aptamers which are RNA and/or DNA aptamers comprising one or more unnatural nucleotides. Exemplary nucleic acid aptamers include ARC19499 (Archemix Corp.), REG1 (Regado Biosciences), and ARC1905 (Ophthotech).

Nucleic acids in accordance with the embodiments described herein optionally include naturally occurring nucleic acids, or one or more nucleotide analogs or have a structure that otherwise differs from that of a naturally occurring nucleic acid. For example, 2′-modifications include halo, alkoxy, and allyloxy groups. In some embodiments, the 2′-OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids having a variety of different nucleotide analogs, modified backbones, or non-naturally occurring internucleoside linkages are utilized in accordance with the embodiments described herein. In some cases, nucleic acids include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, 0(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display enhanced properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Different nucleotide modifications and/or backbone structures may exist at various positions in the nucleic acid. Such modifications include morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof.

Any of the anti-Gal3 antibodies disclosed herein may be conjugated to one or more (e.g. 1, 2, 3, 4, 5, 6, 7, 8, 9, or 10, or more) payloads described herein.

### Conjugation Chemistry

In some instances, the payload is conjugated to an anti-Gal3 antibody described herein by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910.

In some instances, the payload is conjugated to an anti-Gal3 antibody described herein by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the payload is conjugated to an anti-Gal3 antibody described herein by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the payload is conjugated to an anti-Gal3 antibody described herein by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some instances, the enzyme-catalyzed process comprises microbial transglutaminase (mTG). In some cases, the payload is conjugated to the anti-Gal3 antibody utilizing a microbial transglutaminze catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some instances, the payload is conjugated to an anti-Gal3 antibody by a method as described in PCT Publication No. WO2014/140317, which utilizes a sequence-specific transpeptidase and is hereby expressly incorporated by reference in its entirety.

In some instances, the payload is conjugated to an anti-Gal3 antibody described herein by a method as described in U.S. Patent Publication Nos. 2015/0105539 and 2015/0105540.

### Linker

In some instances, a linker described herein comprises a natural or synthetic polymer, consisting of long chains of branched or unbranched monomers, and/or cross-linked network of monomers in two or three dimensions. In some instances, the linker includes a polysaccharide, lignin, rubber, or polyalkylen oxide (e.g., polyethylene glycol).

In some instances, the linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is built up from monomers of another polymer. In some instances, the linker comprises polyalkylene oxide. In some instances, the linker comprises PEG. In some instances, the linker comprises polyethylene imide (PEI) or hydroxy ethyl starch (HES).

In some cases, the polyalkylene oxide (e.g., PEG) is a polydispers or monodispers compound. In some instances, polydispers material comprises disperse distribution of different molecular weight of the material, characterized by mean weight (weight average) size and dispersity. In some instances, the monodisperse PEG comprises one size of molecules. In some embodiments, the linker is poly- or monodispersed polyalkylene oxide (e.g., PEG) and the indicated molecular weight represents an average of the molecular weight of the polyalkylene oxide, e.g., PEG, molecules.

In some embodiments, the linker comprises a polyalkylene oxide (e.g., PEG) and the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, the polyalkylene oxide (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight.

In some instances, the linker is a discrete PEG, optionally comprising from 2 to 60, from 2 to 50, or from 2 to 48 repeating ethylene oxide units. In some cases, the linker comprises a dPEG comprising about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units.

In some embodiments, the linker is a polypeptide linker. In some instances, the polypeptide linker comprises at least 2, 3, 4, 5, 6, 7, 8, 10, 15, 20, 25, 30, 35, 40, 45, 50, 60, 70, 80, 90, 100, or more amino acid residues. In some instances, the polypeptide linker comprises at least 2, 3, 4, 5, 6, 7, 8, or more amino acid residues. In some instances, the polypeptide linker comprises at most 2, 3, 4, 5, 6, 7, 8, or less amino acid residues. In some cases, the polypeptide linker is a cleavable polypeptide linker (e.g., either enzymatically or chemically). In some cases, the polypeptide linker is a non-cleavable polypeptide linker. In some instances, the polypeptide linker comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some instances, the polypeptide linker comprises a peptide such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some cases, the polypeptide linker comprises L-amino acids, D-amino acids, or a mixture of both L- and D-amino acids.

In some instances, the linker comprises a homobifuctional linker. Exemplary homobifuctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[D-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such as1-(p-Azidosalicylamido)-4-(iodoacetamido)butane (AsIB), N-[4-(p-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(p-azidosalicylamido)butylamine (AsBA), and arginine-reactive and photoreactive cross-linkers such as p-azidophenyl glyoxal (APG).

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker comprises PAMAM dendrimers.

In some embodiments, the linker is a traceless linker or a linker in which after cleavage does not leave behind a linker moiety (e.g., an atom or a linker group) to the antibody or payload. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

### Kit/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include an anti-Gal3 antibody as disclosed herein, host cells for producing one or more antibodies described herein, and/or vectors comprising nucleic acid molecules that encode the antibodies described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

Some embodiments provided herein are described by way of the following provided numbered arrangements and also provided as possible combinations or overlapping embodiments:

1. An anti-Gal3 antibody or binding fragment thereof comprising (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein

the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 32, 37, or 66;

the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 801, 951, 952, 77, or 108;

the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 953, 954, 802, 118, or 164;

the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 171, 178, or 215;

the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 222, 229, or 225; and

the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the amino acid sequence of SEQ ID NO: 257, 256, or 291.

2. The anti-Gal3 antibody or binding fragment thereof of arrangement 1, wherein the heavy chain variable region comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 806-820, 955-968, 1067-1109, or 1415-1439.

3. The anti-Gal3 antibody or binding fragment thereof of arrangement 1 or 2, wherein the light chain variable region comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 821-835, 969-982, 1110-1152, or 1440-1464.

4. The anti-Gal3 antibody or binding fragment thereof of any one of arrangements 1-3, wherein the anti-Gal3 antibody or binding fragment thereof comprises a heavy chain, wherein the heavy chain comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 836-850, 983-996,1411, 1153-1195, or 1465-1489.

5. The anti-Gal3 antibody or binding fragment thereof of any one of arrangements 1-4, wherein the anti-Gal3 antibody or binding fragment thereof comprises a light chain, wherein the light chain comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 851-865, 997-1010,1196-1238,1412 or 1490-1514.

6. A nucleic acid comprising a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the nucleic acid sequences of SEQ ID NOs: 866-925, 1011-1066, 1239-1410, or 1515-1614.

7. A method of enhancing glucose transporter (GLUT) translocation in a cell, comprising:

contacting the cell with an anti-Gal3 antibody or binding fragment thereof,

wherein binding of the anti-Gal3 antibody or binding fragment thereof to Gal3 in the cell inhibits Gal3-mediated blocking of GLUT4 translocation.

8. A method of enhancing glucose transporter (GLUT) translocation in a cell, comprising:

contacting the cell with a preincubated complex of Gal3 and an anti-Gal3 antibody or binding fragment thereof.

9. The method of arrangement 8, wherein the preincubated complex of Gal3 and the anti-Gal3 antibody or binding fragment thereof is prepared with Gal3 and the anti-Gal3 antibody or binding fragment thereof at a mass ratio of or of about 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, or 1:2.5, or any mass ratio within a range defined by any two of the aforementioned mass ratios.

10. The method of arrangement 8 or 9, wherein the preincubated complex of Gal3 and the anti-Gal3 antibody or binding fragment is prepared with Gal3 at a concentration of or of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 μg/mL, or any concentration within a range defined by any two of the aforementioned concentrations, and the anti-Gal3 antibody or binding fragment thereof at a concentration of or of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL, or any concentration within a range defined by any two of the aforementioned concentrations.

11. The method of any one of arrangements 7-10, wherein the glucose transporter is glucose transporter 1 (GLUT1) and/or glucose transporter 4 (GLUT4).

12. The method of any one of arrangements 7-11, wherein the method is performed in vitro or in vivo.

13. The method of any one of arrangements 7-12, wherein GLUT translocation in the cell is enhanced by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% after contacting with the anti-Gal3 antibody or binding fragment thereof relative to a cell that is not contacted with the anti-Gal3 antibody or binding fragment thereof.

14. The method of any one of arrangements 7-13, wherein the anti-Gal3 antibody or binding fragment thereof selectively binds to an N-terminal domain of Gal3.

15. The method of any one of arrangements 7-14, wherein the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof.

16. The method of any one of arrangements 7-15, wherein the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein

the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70;

the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952;

the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954;

the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220;

the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 221-247; and

the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296.

17. The method of arrangement 16, wherein the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 23.

18. The method of arrangement 16 or 17, wherein the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 955-968, 1067-1109, 1415-1439.

19. The method of any one of arrangements 16-18, wherein the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 969-982, 1110-1152, 1440-1464.

20. The method of any one of arrangements 7-19, wherein the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489.

21. The method of any one of arrangements 7-20, wherein the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514.

22. The method of any one of arrangements 7-21, wherein the anti-Gal3 antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

23 The method of any one of arrangements 7-22, wherein the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

24. The method of any one of arrangements 7-22, wherein the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof.

25. The method of any one of arrangements 7-22, wherein the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

26. A method of improving insulin sensitivity in a subject in need thereof, comprising:

administering to the subject an anti-Gal3 antibody or binding fragment thereof,

wherein binding of the anti-Gal3 antibody or binding fragment thereof to Gal3 in the subject inhibits Gal3-mediated blocking of glucose transporter (GLUT) translocation in the subject, thereby improving insulin sensitivity in the subject.

27. A method of improving insulin sensitivity in a subject in need thereof, comprising:

administering to the subject a preincubated complex of Gal3 and an anti-Gal3 antibody or binding fragment thereof, thereby improving insulin sensitivity in the subject.

28. The method of arrangement 27, wherein the preincubated complex of Gal3 and the anti-Gal3 antibody or binding fragment thereof is prepared with Gal3 and the anti-Gal3 antibody or binding fragment thereof at a mass ratio of or of about 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, or 1:2.5, or any mass ratio within a range defined by any two of the aforementioned mass ratios.

29. The method of arrangement 27 or 28, wherein the preincubated complex of Gal3 and the anti-Gal3 antibody or binding fragment is prepared with Gal3 at a concentration of or of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 μg/mL, or any concentration within a range defined by any two of the aforementioned concentrations, and the anti-Gal3 antibody or binding fragment thereof at a concentration of or of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL, or any concentration within a range defined by any two of the aforementioned concentrations.

30. The method of any one of arrangements 26-29, wherein the GLUT is glucose transporter 1 (GLUT1) and/or glucose transporter 4 (GLUT4).

31. The method of any one of arrangements 26-30, further comprising identifying the subject as needing improvement in insulin sensitivity prior to the administering step.

32. The method of any one of arrangements 26-31, further comprising detecting an improvement in insulin sensitivity in the subject following the administering step.

33. The method of arrangement 32, wherein detecting the improvement in insulin sensitivity in the subject is done by measuring blood sugar levels, measuring blood insulin levels, glucose tolerance testing, or hyperinsulinemic euglycemic clamp.

34. The method of arrangement 32 or 33, wherein the insulin sensitivity in the subject is improved by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% relative to the insulin sensitivity of the subject prior to the administering step.

35. The method of any one of arrangements 26-34, wherein the anti-Gal3 antibody or binding fragment thereof selectively binds to an N-terminal domain of Gal3.

36. The method of any one of arrangements 26-35, wherein the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof.

37. The method of any one of arrangements 26-36, wherein the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein

the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70;

the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952;

the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954;

the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220;

the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 221-247; and

the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296.

38. The method of arrangement 37, wherein the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 23.

39. The method of arrangement 37 or 38, wherein the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 955-968, 1067-1109, 1415-1439.

40. The method of any one of arrangements 37-39, wherein the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 969-982, 1110-1152, 1440-1464.

41. The method of any one of arrangements 26-40, wherein the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489.

42. The method of any one of arrangements 26-41, wherein the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514.

43. The method of any one of arrangements 26-42, wherein the anti-Gal3 antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

44. The method of any one of arrangements 26-43, wherein the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

45. The method of any one of arrangements 26-43, wherein the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof.

46. The method of any one of arrangements 26-43, wherein the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

47. A method of treating a disease associated with insulin resistance in a subject in need thereof, comprising:

administering to the subject an anti-Gal3 antibody or binding fragment thereof,

thereby treating the disease associated with insulin resistance in the subject.

48. A method of treating a disease associated with insulin resistance in a subject in need thereof, comprising:

administering to a subject a preincubated complex of Gal3 and an anti-Gal3 antibody or binding fragment thereof, thereby treating the disease associated with insulin resistance in the subject.

49. The method of arrangement 48, wherein the preincubated complex of Gal3 and the anti-Gal3 antibody or binding fragment thereof is prepared with Gal3 and the anti-Gal3 antibody or binding fragment thereof at a mass ratio of or of about 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, or 1:2.5, or any mass ratio within a range defined by any two of the aforementioned mass ratios.

50. The method of arrangement 48 or 49, wherein the preincubated complex of Gal3 and the anti-Gal3 antibody or binding fragment is prepared with Gal3 at a concentration of or of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 μg/mL, or any concentration within a range defined by any two of the aforementioned concentrations, and the anti-Gal3 antibody or binding fragment thereof at a concentration of or of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL, or any concentration within a range defined by any two of the aforementioned concentrations.

51. The method of any one of arrangements 47-50, wherein the disease associated with insulin resistance comprises diabetes mellitus, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular disease, cardiac hypertrophy, myocardial ischemia, hypertension, pancreatic cancer associated diabetes (PCDM) or cancer.

52. The method of any one of arrangements 47-51, further comprising identifying the subject as needing treatment of the disease associated with insulin resistance prior to the administering step.

53. The method of any one of arrangements 47-52, further comprising detecting an improvement in the disease associated with insulin resistance following the administering step.

54. The method of arrangement 53, wherein detecting an improvement in the disease associated with insulin resistance comprises detecting an improvement in insulin sensitivity in the subject.

55. The method of arrangement 54, wherein detecting the improvement in insulin sensitivity in the subject is done by measuring blood sugar levels, measuring blood insulin levels, glucose tolerance testing, or hyperinsulinemic euglycemic clamp.

56. The method of arrangement 54 or 55, wherein the insulin sensitivity in the subject is improved by at least 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% relative to the insulin sensitivity of the subject prior to the administering step.

57. The method of any one of arrangements 47-56, wherein the anti-Gal3 antibody or binding fragment thereof selectively binds to an N-terminal domain of Gal3.

58. The method of any one of arrangements 47-57, wherein the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof.

59. The method of any one of arrangements 47-58, wherein the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein

the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70;

the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952;

the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954;

the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220;

the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 221-247; and

the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296.

60. The method of arrangement 59, wherein the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 23.

61. The method of arrangement 59 or 60, wherein the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 955-968, 1067-1109, 1415-1439.

62. The method of any one of arrangements 59-61, wherein the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 969-982, 1110-1152, 1440-1464.

63. The method of any one of arrangements 47-62, wherein the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489.

64. The method of any one of arrangements 47-63, wherein the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514.

65. The method of any one of arrangements 47-64, wherein the anti-Gal3 antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10.2B2-v2, 2D10-VH0-NDA-VL0, 2D10-VH0-QDT-VL0, 2D10-VH0-SDT-VL0, 2D10-VH0-v1-QDT, 2D10-VH0-v2-QDT, 2D10-hVH1-hVL1, 2D10-hVH1-hVL2, 2D10-hVH1-hVL3, 2D10-hVH1-hVL4, 2D10-hVH2-hVL1, 2D10-hVH2-hVL2, 2D10-hVH2-hVL3, 2D10-hVH2-hVL4, 20H5.A3-hIgG4(S228P), 20H5.A3-hIgG4(S228P)-mv2, 798-9.20H5.A3-mH0mL1, 798-9.20H5.A3-mH1mL0, 798-9.20H5.A3-mH1mL1, 798-9.20H5.A3-mH2mL0, 798-9.20H5.A3-mH2mL1, 20H5.A3-VH1VL1, 20H5.A3-VH1VL2, 20H5.A3-VH1VL3, 20H5.A3-VH1VL4, 20H5.A3-VH1VL5, 20H5.A3-VH1VL6, 20H5.A3-VH2VL3, 20H5.A3-VH2VL4, 20H5.A3-VH2VL5, 20H5.A3-VH2VL6, 20H5.A3-VH3VL5, 20H5.A3-VH3VL6, 20H5.A3-VH4VL3, 20H5.A3-VH4VL4, 20H5.A3-VH4VL5, 20H5.A3-VH4VL6, 20H5.A3-VH5VL5, 20H5.A3-VH5VL6, 20H5.A3-VH6VL4, 20H5.A3-VH6VL5, 20H5.A3-VH6VL6, 20H5.A3-VH7VL1, 20H5.A3-VH7VL2, 20H5.A3-VH7VL3, 20H5.A3-VH7VL4, 20H5.A3-VH7VL5, 20H5.A3-VH7VL6, 20H5.A3_hVH3VL1-hIgG1 (KEMv2), 20H5.A3_hVH3VL1-hIgG1 (LALAPGv2), 20H5.A3_hVH3VL1-hIgG1 (REM), 20H5.A3_hVH5VL1-hIgG1 (KEMv2), 20H5.A3_hVH5VL1-hIgG1 (LALAPGv2), 20H5.A3_hVH5VL1-hIgG1 (REM), 20H5.A3_hVH6VL1-hIgG1 (KEMv2), 20H5.A3_hVH6VL1-hIgG1 (LALAPGv2), 20H5.A3_hVH6VL1-hIgG1 (REM), 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

66. The method of any one of arrangements 47-65, wherein the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

67. The method of any one of arrangements 47-65, wherein the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof.

68. The method of any one of arrangements 47-65, wherein the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1. Identification of Gal3-Binding Antibodies with and without Gal3-INSR Blocking Activity

To identify Gal3-binding antibodies with the capacity to block the assembly of Gal3 and INSR, an immunization campaign was run in mice. Balb/C, FVB, and CD-1F mice were inoculated at 7 day intervals with 50 μg of Gal3 protein fused to a linker-spaced 6-histidine tag, Gal3-ECD-His, (Acro GA3-H5129; Lot #819-43PS1-5E) in combination with a TLR agonist adjuvant mix (50 μg MPL, 20 μg CpG, 10 μg Poly(I:C) and 10 μg R848) for 3 repetitions, followed by an inoculation with 50 μg of Gal3-His alone administered subcutaneously to the inguinal, back of the neck and base of the tail sites as well as hock and intraperitoneal sites. Animals were sacrificed in accordance with IACUC protocol and spleen, femurs, and lymph nodes (axillary, accessory axillary, mediastinal, superficial inguinal, iliac, sacral and popliteal) were harvested. A single cell suspension of immunized lymph node (LN), spleen and bone marrow cells were obtained using 2 sterile frosted glass slides in a tissue culture petri dish with 15 mL DMEM. Bone marrow was extracted from femurs via end-cap flushing with a 5 mL syringe fitted with an 18-gauge needle. Cells from 3 animals were pelleted with 5 minutes of centrifugation at 1200 RPM, resuspended in 10 mL of DMEM (GIBCO 10564-011) and nucleated cells were enumerated by hemocytometer count. Cells were pelleted at 1200 RPM and were resuspended in SC-Buffer (PBS, 2% FBS and 1 mM EDTA), and plasma cells were isolated with an EasySep™ Mouse CD138 Positive Selection Kit (StemCell Technologies) with the manufacturer recommended protocol. Enriched CD138-positive cells were pelleted with 5 minutes of centrifugation at 1200 RPM, resuspended in 50 mL electrofusion buffer (Eppendorf 940-00-220-6) and were enumerated. Separately, SP2/0-mIL6 myeloma cells (ATCC CRL2016) were pelleted with 5 minutes of centrifugation at 1200 RPM, resuspended in 50 mL electrofusion buffer and were enumerated. Myeloma cells and CD138-positive plasma cells were combined at a 1:1 ratio, volume was expanded to 50 mL with electrofusion buffer, cells were pelleted with 5 minutes of centrifugation at 1200 RPM and supernatant was discarded. After a repeated step of washing and pelleting in electrofusion buffer, cells were resuspended in electrofusion buffer to a concentration of 10×10\hat{ }6 cells/mL, up to 9 mL of cell suspension was added to a BTX electrofusion chamber, and cells were fused with an 800V electrofusion protocol. Fused cells were rested for 5 minutes, transferred to a tissue culture dish containing 40 mL medium MM (DMEM, 15% FBS, 1% glutamax and 1% Pen/Strep), incubated for 1 hour at 37° C., 8% CO₂, resuspended with a pipette, pelleted with 5 minutes of centrifugation at 1200 RPM, resuspended in ClonaCell HY Liquid HAT Selection Medium (StemCell Technologies), and plated in 96-well tissue culture flat bottomed plates. After 10 days, supernatants were sampled and evaluated for binding to isolated Gal3 by ELISA. 50 μl of 0.1 μg/mL Gal3-ECD-His, (Acro GA3-H5129; Lot #819-43PS1-5E) resuspended in diluent (PBS with 0.5% BSA) was added to each well for 45 minutes, supernatant was discarded and plates were washed with phosphate buffered saline (PBS) with 0.05% Tween20. 50 μl of 1:5 dilution of hybridoma supernatant in diluent was added to each well for 1 hour, followed by 5 successive 300 μl washes with PBS/0.05% Tween20, after which a 1:3000 dilution of goat anti-mouse Fc-specific antibody conjugated to horseradish peroxidase (Novex A16090) in 50 μl of diluent was added to each well for 1 hour followed by 5 successive 300 μl washes with PBS/0.05% Tween20. Following washing, 50 μl of ABTS (Novex #00-202-4) was added to each well for 20-30 minutes, prior to readout on a spectrophotometer (Molecular Devices) at absorbance of 405 nm.

Positively scoring wells were evaluated for the ability to block association of Gal3 and INSR. To identify Gal3-targeted antibodies with the ability to block the interaction of Gal3 and INSR, purified Gal3 and INSR proteins were incubated in the presence of Gal3-immunization hybridoma supernatants described above, or without antibody, and protein interaction was evaluated by ELISA. Human Galectin-3 protein (Acro Biosystems, GA3-H5129) was diluted in PBS (Corning, 21-030-CM) to a concentration of 3 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]). The plate was then blocked for an hour with 2% BSA (EMD Millipore, 126609) in PBST at room temperature with gentle rocking. Thereafter, the 2% BSA in PBST was discarded and antibody or inhibitor (3-fold dilutions beginning at 20 μg/ml, 60 μg/ml, or 180 μM) in 2% BSA in PBST was added to the wells. Afterwards, 6 μg/ml of human Insulin Receptor (Sino Biological, 11086-H08H) in 2% BSA in PBST was added to the antibody or inhibitor in the wells in a 1:1 ratio. The plate was incubated for an hour at room temperature with gentle rocking. Thereafter, the plate was washed three times with PBST, and 0.3 μg/ml of human INSR Biotinylated Antibody (R&D Systems, BAF1544) in 2% BSA in PBST was added to the wells. The plate was incubated for an hour with gentle rocking and then washed three times with PBST. Avidin-HRP (1:2000) was then added to the wells. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

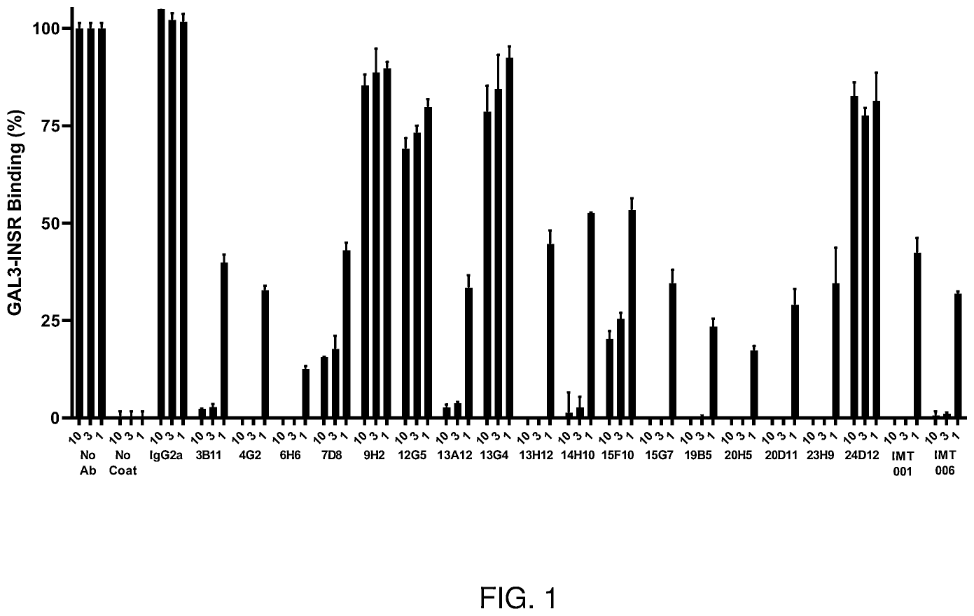

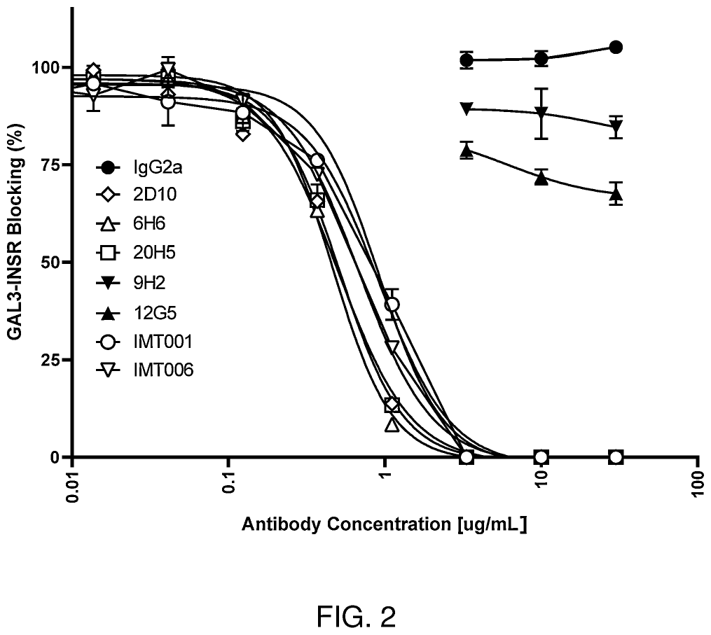

Several hGal3-binding antibodies with the ability to strongly block the binding of Gal3 to INSR were identified, depicted in FIG. 1 and FIG. 2, including 6H6.2D6, 20H5.A3, 20D11.2C6, 4G2.2G6, 13H12.2F8, 19B5.2E6, 15G7.2A7, 23H9.2E4, 19D9.2E5, 2D10.2B2, 4A11.2B5, 14H10.2C9, 3B11.2G2, 13A12.2E5, which reduced the binding of Gal3 and INSR at a concentration of 3 μg/mL to less than 5% of an unblocked sample. Additionally, monoclonal antibodies with reduced capacity to inhibit Gal3-INSR binding at 3 μg/mL to 15-25% of levels of an unblocked sample were identified, including 7D8.2D8 and 15F10.2D6. Finally, monoclonal antibodies with the capacity to minimally impact Gal3-INSR binding by 30% or less at 3 μg/ml were identified, including 9H2.2H1, 12G5.D7, 13G4.2F8, and 24D12.2H9.

To evaluate whether the ability to block Gal3 assembly with INSR was a function of antibody affinity to Gal3, the affinity of monoclonal Gal3 antibodies to Gal3 was evaluated by SPR. Kinetics experiments were performed on BiacoreT200 at 25° C. in high performance mode. Ligand proteins, purified antibodies were captured onto a CM5 chip coupled with anti-human Fc or anti-mouse Fc antibody, three antibodies at a time onto flow cell #2, 3, and 4, respectively, while flow cell #1 was used as reference. The analyte Galectin-3 in HBS-EP buffer was injected over all four flow cells at concentrations of 100, 50, 25, 12.5, 6.25, 3.125 and 0 nM at a flow rate of 30 μL/min. The complex was allowed to associate and dissociate for 240 and 300 seconds, respectively. The surfaces were regenerated with a 30 second injection of 10 mM Glycine pH 1.7 (flow rate 30 μL/min). The data were fit to a simple 1:1 interaction model using the global data analysis option available within BiacoreT200 Evaluation software V2.0.

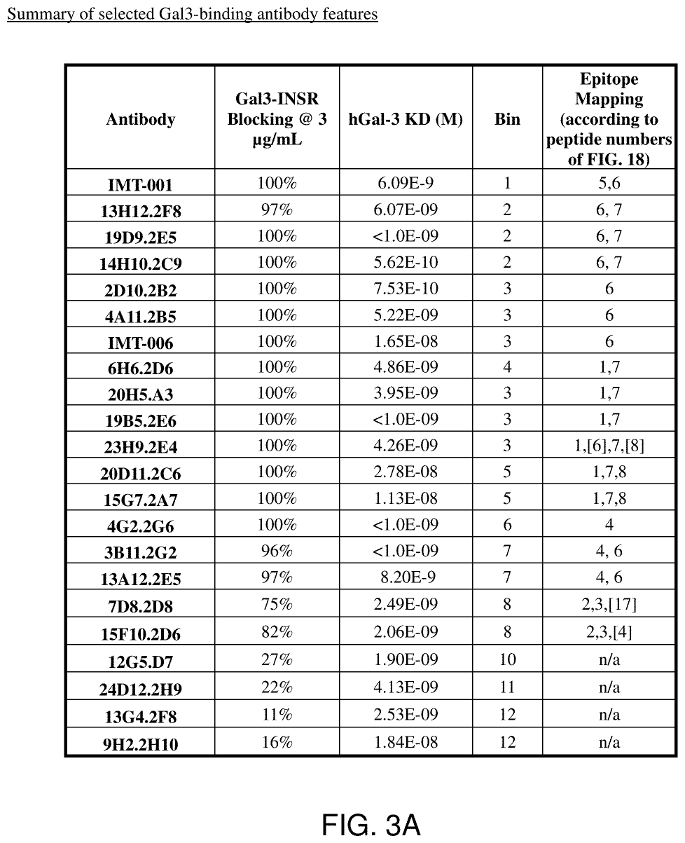

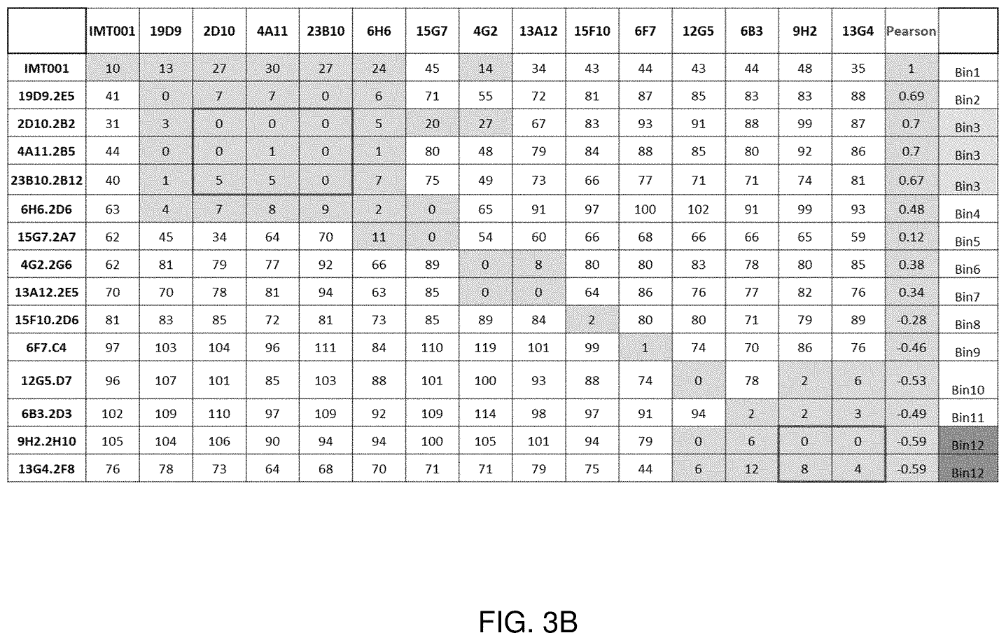

The affinity of Gal3 monoclonal antibodies was confirmed to be greater than 30 nM for all antibodies studied (FIG. 3A). Importantly, 15G7, an antibody which reduced assembly of Gal3 and INSR by greater than 99% at 3 μg/mL exhibited an affinity of 27.8 nM for Gal3, indicating that the ability to block the assembly of Gal3 and INSR is possible with antibodies with affinity at or below this level. In contrast, Gal3-targeted antibodies which poorly block Gal3-INSR assembly, 13G4.2F8, 9H2.2H1, 24D12.2H9, and 12G5.D7, exhibited affinities of 18.4, 2.53, 4.13, and 1.9 nM, respectively. Thus, antibody affinity to Gal3 of less than 10 nM is not sufficient to predict the ability to block assembly of Gal3 and INSR.

### Example 2. Gal3-Targeted Antibodies with and without Gal3-INSR Blocking Activity Bind to Distinct Epitopes of Gal3

To identify the epitopes to which Gal3 antibodies with and without Gal3-INSR blocking activity bound, a library of 24 amino acid peptides representing portions of Gal3, depicted in FIG. 18, was produced and the ability of each peptide to bind Gal3 antibodies was evaluated by ELISA. At least 2 μg/ml of hGal3 peptide in 50 μl of PBS or 0.1 μg/ml of full-length human Gal3 protein (GenScript) and human Galectin-3 protein (Acro Biosystems, GA3-H5129) were diluted in PBS (Corning, 21-030-CM) to concentrations of at least 2 μg/ml or 0.1 μg/ml, respectively, and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]). The plate was then blocked for an hour with 2% BSA (EMD Millipore, 126609) in PBST at room temperature with gentle rocking. Thereafter, the 2% BSA in PBST was discarded and human Galectin-3 hybridoma supernatants or antibodies were diluted in 2% BSA in PBST to concentrations of at least 0.1 μg/ml and added to the wells. The plate was incubated for an hour at room temperature with gentle rocking and then washed three times with PBST. Afterwards, Goat Anti-Mouse IgG-HRP (Jackson ImmunoResearch, 115-036-1461) or Goat Anti-Rat IgG HRP (Abcam, ab205720) diluted in 2% BSA in PBST (1:4000) were added to the wells. The plate was incubated for 30 minutes to 1 hour at room temperature with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

Binding of Gal3-binding antibodies to the peptide array was observed at multiple locations, with the majority of binding observed in Peptides 1-8 (SEQ ID NOs: 3-10), summarized in FIG. 3A. Significantly, all Gal3-binding antibodies with strong INSR-Gal3 blocking activity exhibited the ability to bind to peptides 4, 6, or 7, corresponding to peptide sequences in the N-terminal domain of Gal3. Specifically, six separate Gal3-binding antibodies with strong Gal3-INSR blocking activity (6H6.2D6, 20H5.A3, 20D11.2C6, 19B5.2E6, 15G7.2A7, and 23H9.2E4) all bound peptide 1 of Gal3, corresponding to amino acids 1-20 of Gal3, ADNFSLHDALSGSGNPNPQG (SEQ ID NO: 3). Conversely, no Gal3-targeted antibodies with poor Gal3-INSR blocking activity were observed to bind peptide 1. Taken together, these data indicate that binding to Gal3 peptide 1 is predictive of the ability to block the interaction of Gal3 with INSR. Similarly, two separate Gal3-binding antibodies with strong Gal3-INSR blocking activity (4G2.2G6 and 3B11.2G2) bound peptide 4 of Gal3, corresponding to amino acids 31-50 of Gal3, GAGGYPGASYPGAYPGQAPP (SEQ ID NO: 6). Conversely, no Gal3-targeted antibodies with poor Gal3-INSR blocking activity were observed to bind peptide 4. Taken together, these data indicate that binding to Gal3 peptide 4 is predictive of the ability to block the interaction of Gal3 with INSR. Further, seven Gal3-binding antibodies with strong Gal3-INSR blocking activity (13H12.2F8, 19D9.2E5, 14H10.2C9, 2D10.2B2, 4A11.2B5, 3B11.2D2, and 13A12.2E5) all bound peptide 6 of Gal3, corresponding to amino acids 51-70 of Gal3, GAYPGQAPPGAYPGAPGAYP (SEQ ID NO: 8). Conversely, no Gal3-targeted antibodies with poor Gal3-INSR blocking activity were observed to bind peptide 6. Taken together, these data indicate that binding to Gal3 peptide 6 is predictive of the ability to block the interaction of Gal3 with INSR. Additionally, eleven Gal3-binding antibodies with Gal3-INSR blocking activity (6H6.2D6, 20H5.A3, 20D11.2C6, 13H12.2F8, 19B5.2E6, 23H9.2E4, 15G7.2A7, 19D9.2E5, 14H10.2C9, 7D8.2D8, and 15F10.2D6) all bound peptide 7 of Gal3, corresponding to amino acids 61-80 of Gal3, AYPGAPGAYPGAPAPGVYPG (SEQ ID NO: 9). Conversely, no Gal3-targeted antibodies with poor Gal3-INSR blocking activity were observed to bind peptide 7. Taken together, these data indicate that binding to Gal3 peptide 7 is predictive of the ability to block the interaction of Gal3 with INSR. In total, these data indicate the utility of anti-Gal3 antibodies to Gal3 peptides 1, 4, 6, and 7 as predictive of the ability to block the interaction of Gal3 and INSR.

As illustrated in FIG. 18, peptides 4, 6, and 7 share repeated amino acid sequences comprised of proline-glycine (PG) and tyrosine-proline-glycine (YPG), suggesting a common feature that may explain the ability of Gal3-targeted antibodies to bind to multiple Gal3 peptides. Further, the amino acid sequence glycine-x-tyrosine-proline-glycine (GxYPG), where x may be the amino acids alanine (A), glycine (G), or valine (V), is shared in peptides 4, 6, and 7, each of which possess two such sequences separated by 3 amino acids. Accordingly, the presence of two GxYPG sequences in close apposition is likely predictive of the ability to bind Gal3-targeted antibodies with the ability to block Gal3 and INSR. Additionally, the Grantham distance of alanine, glycine, and valine is Ala-Val: 64, Ala-Gly: 60, Val-Gly: 109, thereby predicting that amino acids with similarly low Grantham distances may similarly be able to substitute at the variable region, including proline and threonine.

### Example 3. Gal3-INSR Antibodies with Blocking Activity Compete for Binding to Gal3

To determine whether Gal3-binding antibodies with Gal3-INSR blocking activity bind to the same or overlapping regions of the Gal3 molecule, antibody binning assays were performed to assess the ability of antibodies to simultaneously bind Gal3. Amine-reactive probes were loaded onto a Gator biosensor (Probe Life, Palo Alto, Calif.), equilibrated in dH20 for 60 seconds, dipped into 100 μl EDC 0.2M/NHS 0.05M activation buffer for 30 seconds, then dipped into a solution of 20 μg/μl human Gal3-His in 10 mM NaOAc buffer, pH 5 until binding was saturated, and quenched in 1 M ethanolamine pH 8.5 for 300 seconds. Following Gal3-His loading, tips were dipped in 20 μg/mL saturating antibody, then successively dipped into 5 μg/mL competing antibody. As shown in FIG. 3A-B, antibodies with competitive binding profiles were assigned bins and associations to blocking activity were made.

Separate bins of competitive antibody binding patterns to Gal3 were established. Significantly, strong associations between bin and blocking Gal3-INSR blocking activity were observed. All antibodies from bins 1, 2, 3, 4, 5, 6, and 7 strongly inhibited Gal3 binding to INSR, summarized in FIG. 3A. In contrast, antibodies in bin 8 were somewhat weaker blockers of Gal3 blocking to INSR, despite strong affinity to Gal3. Antibodies in bin 10, 11, and 12 uniformly did not have the ability to significantly inhibit the association of Gal3 and INSR. Thus, the competitive binding bins of 1, 2, 3, 4, 5, 6, and 7, are useful to predict the ability of Gal3-binding antibodies to block the assembly of Gal3 and INSR.

### Example 4. Gal3-INSR Blocking Antibody TB001 (IMT001-4) Reduces Weight Gain, Insulin Resistance, Glucose Insensitivity, Liver Steatosis, and Liver Dysfunction in High-Fat Diet Fed Diabetic Mice

To evaluate whether Gal3-INSR blockade by Gal3-targeted antibodies functionally impacted diabetes biology, a mouse model of type II diabetes was employed. Male C57BL6J mice were purchased from Jackson Lab and fed with high fat diet (HFD, 60% kcal from fat, Research Diet, Catalog #12492i) starting from 7-week old for 8 weeks, 10 or 16 weeks. Age-matched male C57BL6J mice fed with standard chow diet were used as control mice. The mice were housed in standard facilities and disposable standard cages with filter tops at room temperature with a 06:00-18:00 day-night cycle. Mice were fed with standard chow diet or HFD ad libitum except when fasted as indicated in the experiments. Glucose tolerance test (GTT) and insulin tolerance test (ITT) were done at 15 weeks and 16 weeks old (8 weeks and 9 weeks after HFD), respectively, to confirm the insulin resistant and glucose intolerant phenotype. For GTT, mice were fasted for 6 hours by transferring mice to clean cage with no food or faeces but with drinking water and then injected with 2 g/kg glucose (Sigma) and blood was drawn to measure glucose levels at 0, 15, 30, 60, 90, and 120 minutes after glucose injection. For ITT, mice were fasted for 6 hours, and then injected with 0.75 IU/kg body weight of Humalin-R (Eli Lilly) intraperitoneally. Blood from the tail was measured for glucose content using HemoCue Glucose 201 Analyzer at 0, 15, 30, 45, 60 minutes. Based on area under the curve (AUC) from ITT, HFD fed mice were randomized into two groups: human IgG4 isotype group and IMT001-4 treated group. At 17 weeks of age, mice were given antibody treatments by intraperitoneal injection twice a week for 5 doses before ITT and GTT were done at 19 weeks old and 20 weeks old. Human IgG4 isotype and IMT001-4 were dosed at 10 mg/kg with 100 μl/mouse and BM was dosed at 10 mg/ml with 100 μl/mouse. Treatments continued until the mice were sacked at 21 weeks old. The mice body weight was monitored one week after the mice arrived at the facility. Body weight was measured once a week using a balance. Mouse blood was collected using cardiac puncture. Liver, gastrocnemius muscle, and posterior subcutaneous white adipose fat were collected and weighed.

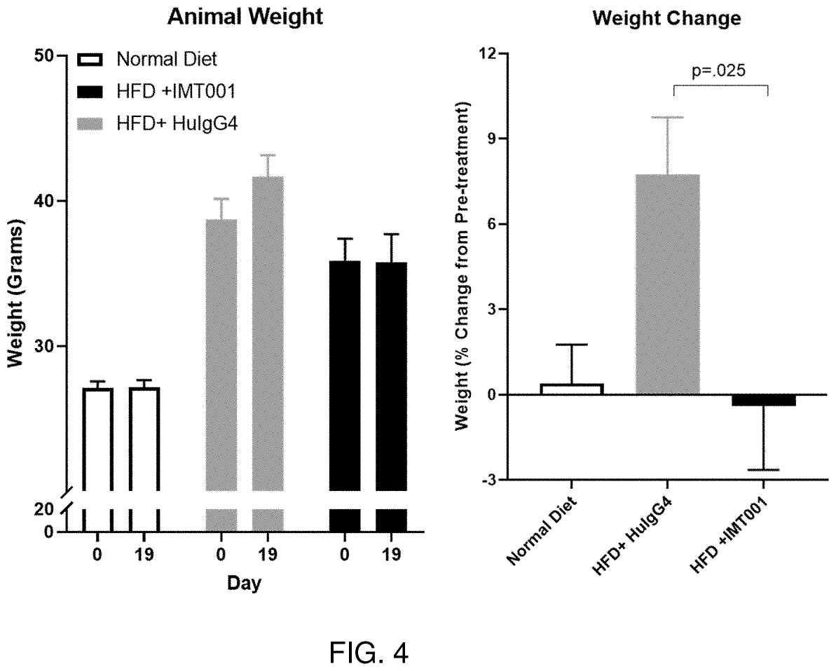

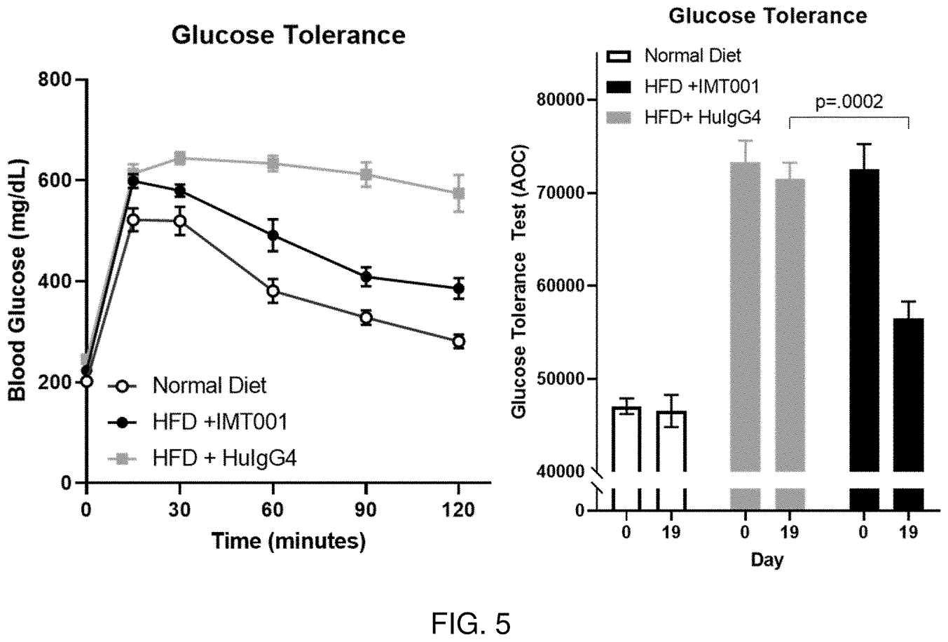

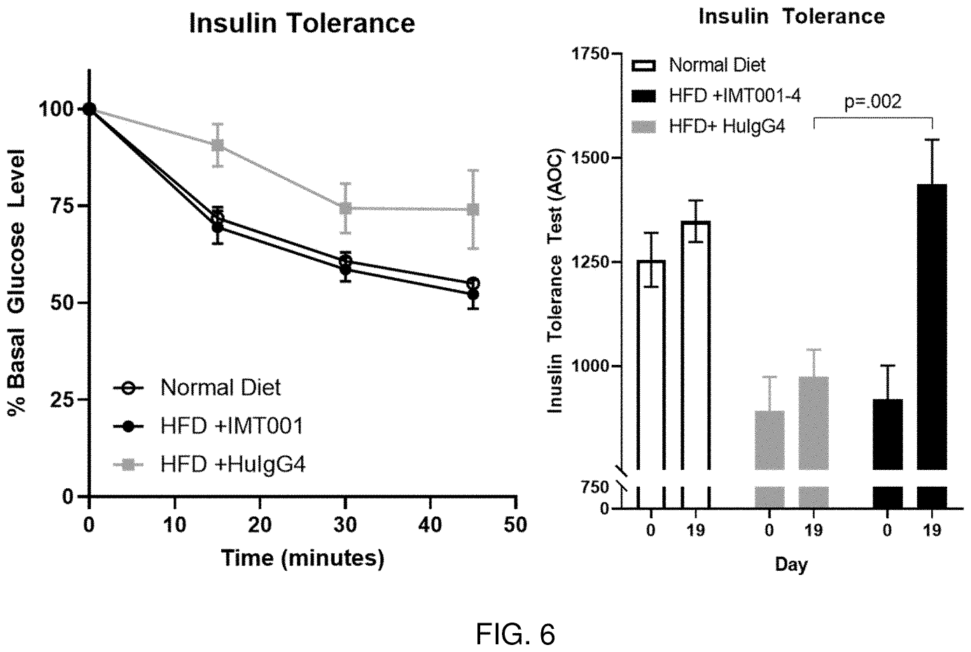

High-fat diet (HFD) fed mice were confirmed to have elevated basal glucose levels, and gained weight more rapidly than control-diet fed animals. Significantly, whereas HFD-fed mice treated with isotype control exhibited significant weight gain of over 7% body mass over the 19 day study, HFD-fed animals treated with IMT001-4 did not gain any appreciable weight (FIG. 4). Further, whereas isotype-control treated HFD-fed mice exhibited delayed response to glucose challenge, HFD-fed animals treated with IMT001-4 cleared glucose significantly more rapidly, in a manner more similar to mice fed with a normal diet (FIG. 5). Further, whereas HFD-fed animals treated with isotype control exhibited delayed glucose reduction in response to insulin challenge, IMT001-4 treated HFD-fed mice exhibited glucose clearance indistinguishable from non-diabetic mice fed a normal diet (FIG. 6). Collectively, these data support the finding that the Gal3-INSR blocking antibody IMT001-4 resolves multiple aspects of diabetes, including insulin resistance, glucose insensitivity, and weight gain.

To further evaluate the ability of IMT001-4 to impact the pathological sequalae of diabetes, liver sections from animals treated as in FIGS. 3-5 were examined for signs of fat accumulation, e.g., steatosis. Briefly, liver specimens were fixed in 4% paraformaldehyde for 24 hours, placed in 70% EtOH for 3 days, embedded into paraffin and 10 μm sections were cut and mounted to glass slides and stained with hematoxylin and eosin and visualized on a Revolve microscope at 40× magnification. Images were evaluated by ImageJ to quantitate the extent of steatosis.

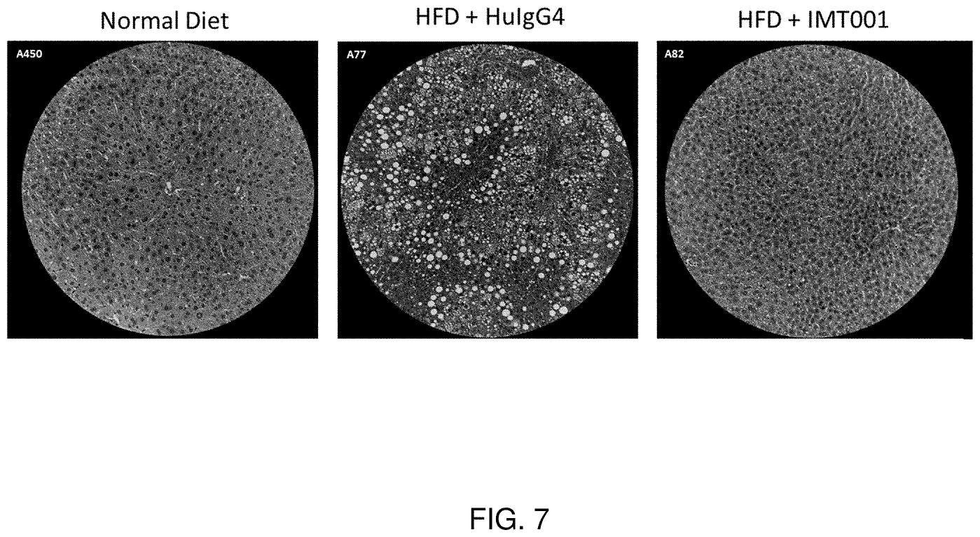

Moderate levels of steatosis were observed in HFD-fed animals treated with control antibody, exemplified by the presence of circular-non stained regions in H&E stained sections (FIG. 7). In contrast, HFD-fed animals treated with IMT001-4 exhibited negligible levels of steatosis and were difficult to distinguish from livers of non-diabetic animals fed with a normal diet. Visual observations were confirmed by image quantification, which demonstrated a complete reversion of disease state in IMT001-4 treated animals relative to isotype-control treated animals. Accordingly, the Gal3-targeted Gal3-INSR blocking antibody, IMT001-4, has utility in reducing liver steatosis.

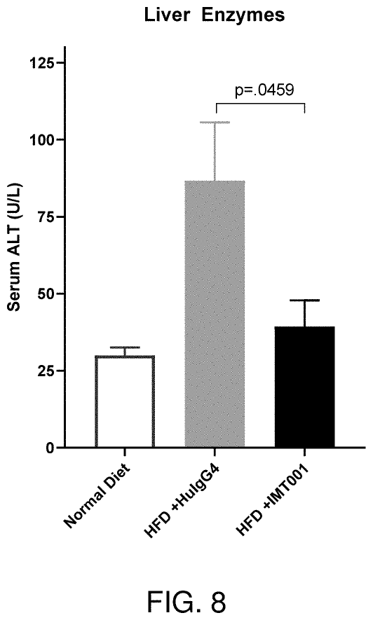

To further characterize the liver disease observed in histological samples, serum markers of liver dysfunction were evaluated in animals treated as above by ELISA. Briefly, mouse plasma samples were obtained from whole blood sample in lithium heparin collection tubes. 100 μl of serum was dispensed into the rotor of a VetScan VS2 Blood Chemistry Analyzer (Abaxis) through the sample port. ALT levels were determined as per the manufacturer's specification. Consistent with the observed elevated levels of steatosis observed in isotype-control treated HFD-fed diabetic mice relative to normal-diet fed non-diabetic mice, isotype-control HFD-fed diabetic mice exhibited nearly a 3-fold increase in ALT relative to normal-diet fed non-diabetic mice (FIG. 8). Strikingly, ALT levels were significantly decreased in IMT001-4 treated HFD-fed mice relative to HFD-fed isotype-control treated mice, to levels on-par with normal-diet fed non-diabetic mice. Accordingly, the Gal3-targeted Gal3-INSR blocking antibody, IMT001-4, has utility in reducing serum liver enzymes.

### Example 5: Anti-GAL3 Antibodies have Therapeutic Use in a Model of Advanced Type II Diabetes

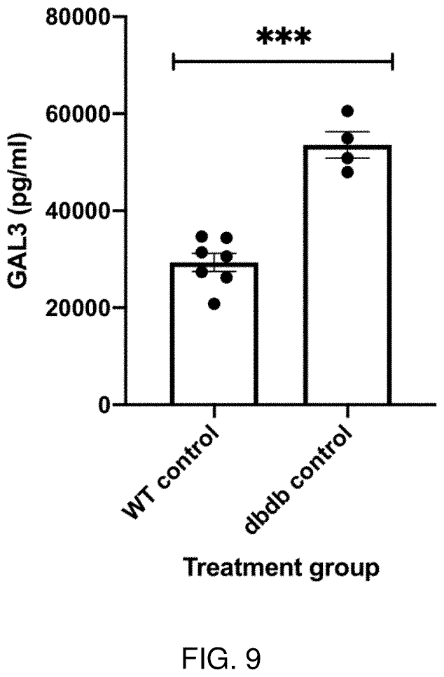

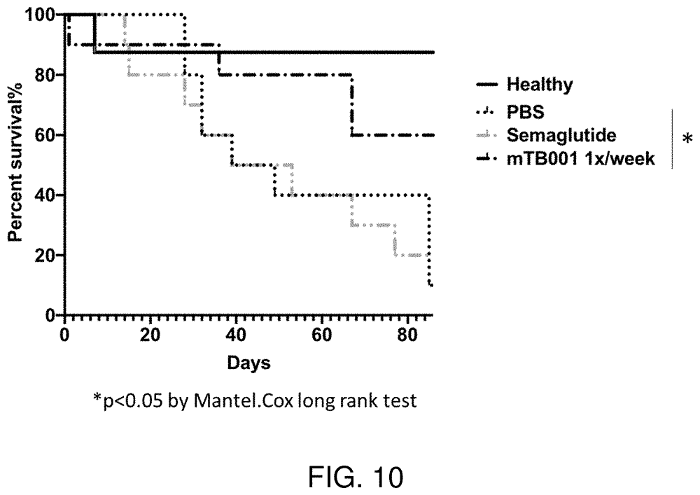

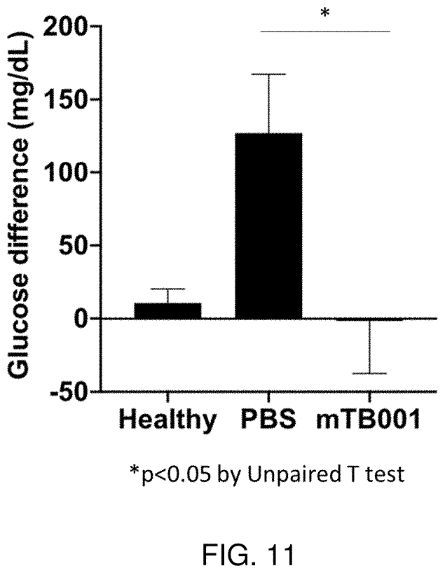

In order to investigate the effect of therapeutic anti-GAL-3 antibodies on a severe case of type II diabetes, a mouse model using High Fat Diet (HFD)-fed db/db mice was implemented. Four-weeks-old male Bks-db mice (000642) obtained from Jackson lab were fed with 60% kcal HFD (Research Diet, 12492i) for eight weeks. Control animals (male C57BL6/J (000662), Jackson lab) were fed normal chow diet. db/db animals had increased levels circulating Gal-3 (FIG. 9). Animals were randomized based on the body composition measured by employing EchoMRI-500™ (EchoMRI). The HFD-fed db/db animals were treated with negative control (PBS), positive control (Semaglutide), or anti-GAL3 TB001 antibodies Q1W (once weekly). Wild type animals were used as a control (Healthy) group. The animals were dosed for twelve weeks. TB001 (anti-GAL3) treatment significantly prolonged survival of db/db animals (FIG. 10). The prolonged survival of the TB001-treated animals was also associated with a lack of change in the levels of fasting blood glucose compared to PBS-treated groups as depicted in FIG. 11. The fasting blood glucose was measured as described in Example 6. Briefly, the mice were fasted for 4-6 hours in clean cages with water and no food. Blood from the tail was diluted 2.5 times before using HemoCue Glucose 201 Analyzer and cuvettes (Mercedes Scientific 110706) to measure the blood glucose level.

### Example 6: Anti-GAL3 Antibodies have Therapeutic Use in a Mouse Model of Type I Diabetes

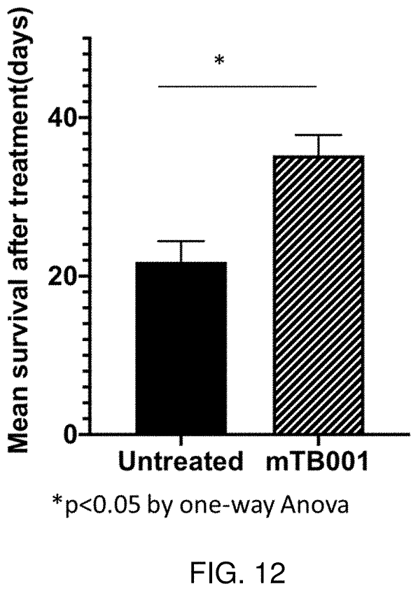

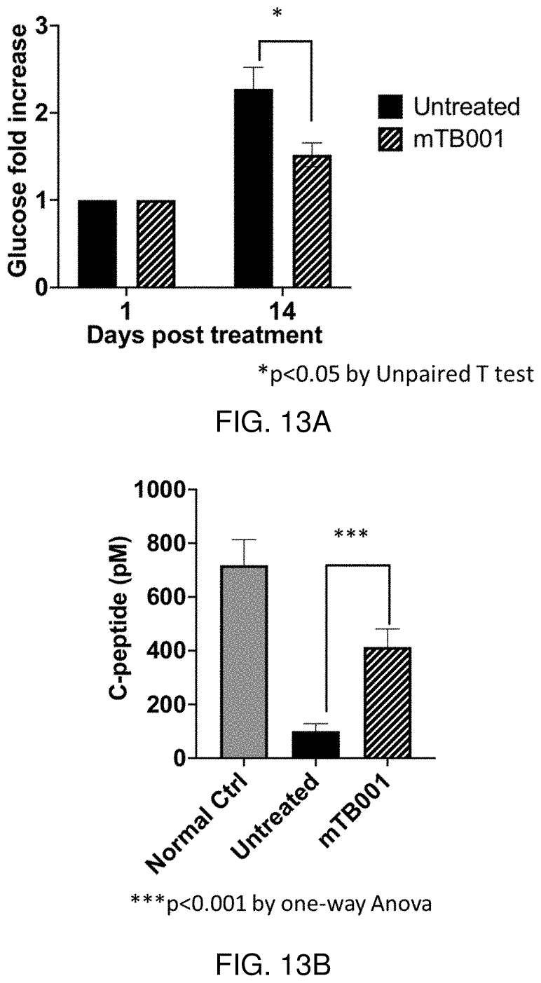

To assess the therapeutic potential of anti-GAL3 antibody in Type I diabetes, a mouse model of diabetes type I was used. Briefly, seven-weeks-old female NOD/ShiLtJ mice (Jackson lab (001976)) and control group animals NOR/LtJ (Jackson Lab (002050)) were housed in standard disposable cages with filter tops at room temperature with a 6:00-18:00 day-night cycle. Mice were allowed to rest for a week before initiating glucose monitoring. The antibody treatment was initiated when glucose levels reach 250-400 (mg/dL) for two consecutive days. To establish glucose levels, the blood samples were collected from tail vein of the experimental animals following housing in clean cages with water and no food for 4-6 hours. The collected samples were analyzed by HemoCue Glucose 201 Analyzer and cuvettes (Mercedes Scientific 110706). The animals were dosed twice a week (10 mg/kg) with anti-GAL3 antibodies (mTB001) or PBS control. The animals were sacrificed when the glucose levels reached >1000 (mg/dL) or until they exhibited morbidity symptoms (i.e. hunched posture, hypothermia, and hypoactivity). At the end stage, the plasma samples were collected from symptomatic animals. C-peptide levels were determined by Alpco Mouse C-peptide ELISA kit (Alpco, 80-CPTMS-E01). As depicted in FIG. 12, anti-GAL3 treatment prolonged survival of NOD/ShiLtJ animals and restored beta-cell function by stabilizing the levels of blood glucose (FIG. 13A) and restoring C-peptide levels (FIG. 13B). Therefore, anti-Gal3 treatment decreases the symptoms of both Type II and Type I diabetes.

### Example 7: Anti-GAL3 Antibodies have Therapeutic Use in a Mouse Model of Chronic Inflammatory Bowel Disease (IBD)

A mouse model of chronic inflammatory bowel disease was implemented to study the effect of Gal-3 antibodies. Briefly, seven-weeks-old male C57BL/6J mice were housed in standard disposable cages with filter tops at room temperature with a 6:00-8:00 day-night cycle. Mice were allowed to rest for one week before initiating the treatment.

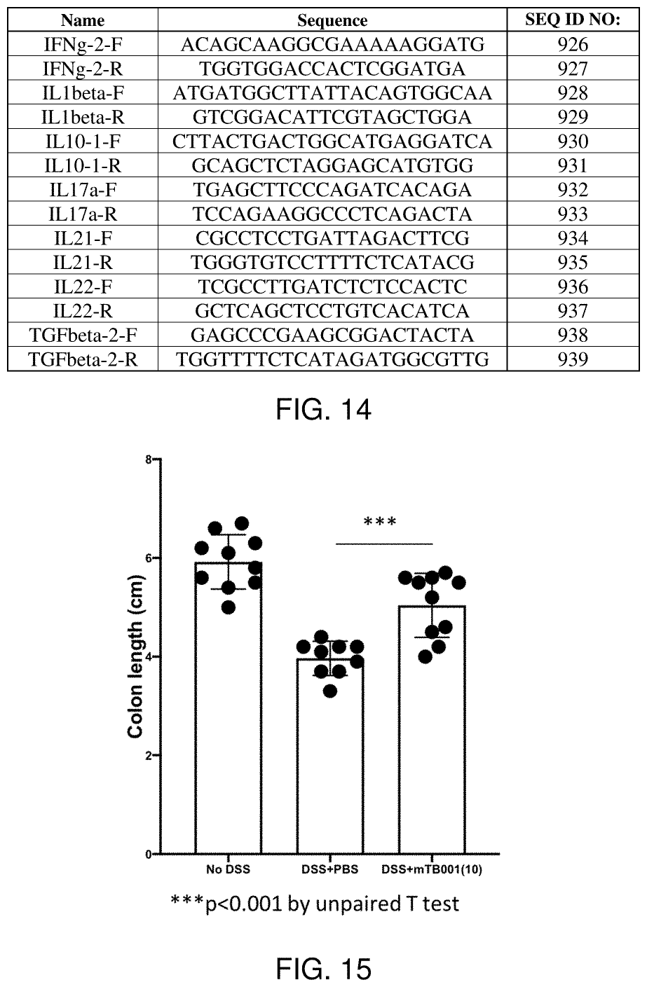

Mice were randomized into the four treatment groups: no dextran sulfate sodium salt (DSS), 3% DSS in water+PBS, 3% DSS+mTB001 (10 mg/kg), 3% DSS+mTB001 (1 mg/kg). DSS water mixture was given for five consecutive days followed by seven days of normal water for a total of three DSS cycles and two normal water cycles. Body weight, stool consistency, gross bleeding score, and Hemoccult score (Fisher, SK-61130) were monitored daily. At necropsy, blood, colon, jejunum, ileum, duodenum, and spleen were preserved and flash frozen. Plasma Inflammatory cytokines were measured using Multi-spot Assay Systems (MSD) Proinflammatory Panel 1 (mouse) kit (MSD, K15048D). RT-qPCR was performed on frozen colon samples. Briefly, the colon tissue was disrupted using Qiazol lysis buffer (Qiagen, 79306) and the Qiagen Tissue Lyser II (Qiagen, 85300). RNA was extracted using RNeasy mini kit (Qiagen, 74104). cDNA was synthesized with iScript Reverse Transcription Supermix (BioRad, 1708841) at BioRad C1000 touch thermal cycler (BioRad, 1851138). RT-qPCR was performed using SsoAdvanced Universal SYBR green supermix (BioRad, 1725272) at CFX384 Touch Real-Time PCR detection system (BioRad, 1955485) using the primers in FIG. 14 (SEQ ID NO: 926-936).

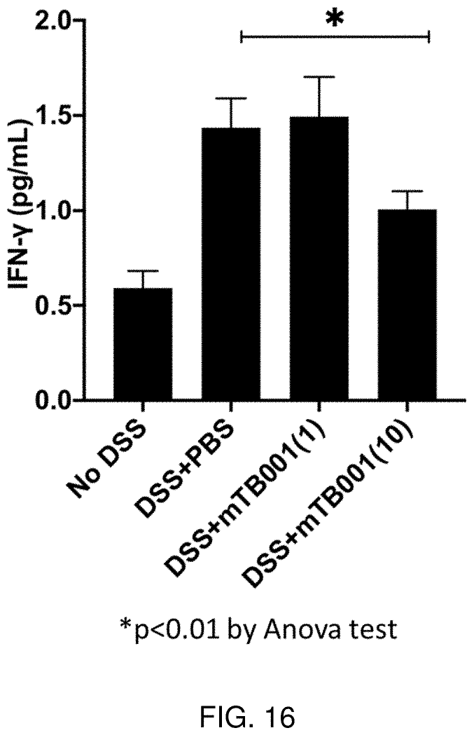

Anti-galectin-3 treatment restored DSS-induced reduction in the colon length (FIG. 15) and reduced DSS-induced inflammation determined by the circulating levels of IFN-gamma (FIG. 16). In addition, mRNA levels of several pro-inflammatory genes (e.g. IFN-gamma, IL17a, IL-1beta, IL-21, IL-22) were reduced in response to TB001 treatment.

### Example 8: Binning and Peptide Binding Assay of Additional Exemplary Anti-Gal3 Antibodies

A large-scale antibody binning assay was performed on exemplary anti-Gal3 antibodies.

The epitope binning assay was done in a sandwich format on the high-throughput SPR-based Carterra LSA unit (CarterraBio, Salt Lake City, Utah). First, the purified antibodies were diluted to 10 μg/ml concentration in 10 mM NaOAc (pH 5.0) and then were covalently coupled via amine group to HC200M chip activated by EDC and S-NHS to immobilize antibodies to different positions of a 384-spot array. One hundred thirty-eight binning cycles were run on the array of immobilized antibodies. In each cycle, first, human Gal3 (AcroBio GA3-H5129) was injected over the entire array to bind to different antibodies (primary antibody), followed by one antibody (secondary antibody) selected among the panel of 150 anti-GAL3 antibodies tested. At the end of each cycle, the array was regenerated by 10 mM Glycine (pH 2.0) to remove bound antigen and secondary antibody from the array. The data analysis was done using the Epitope software by CarterraBio.

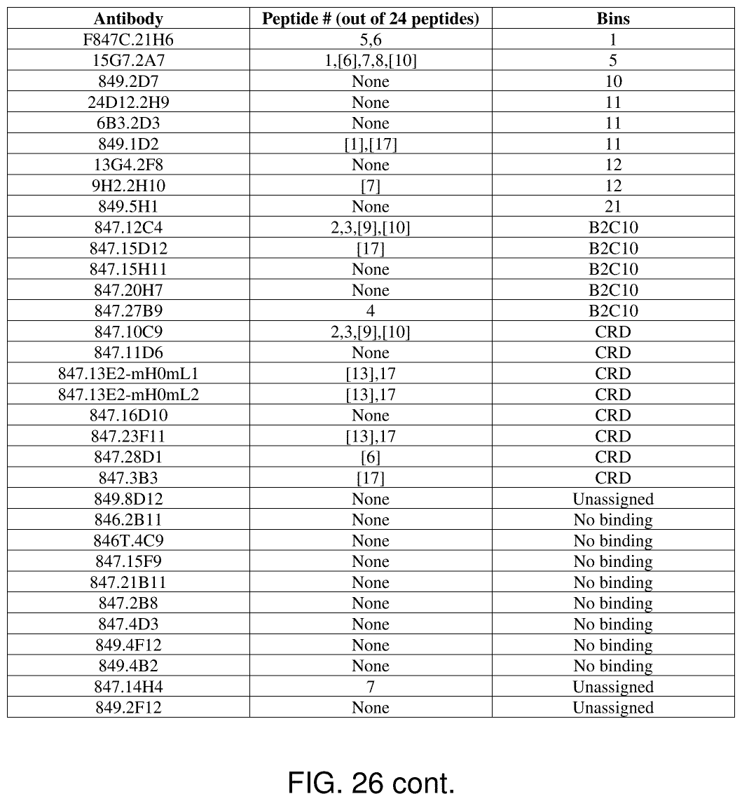

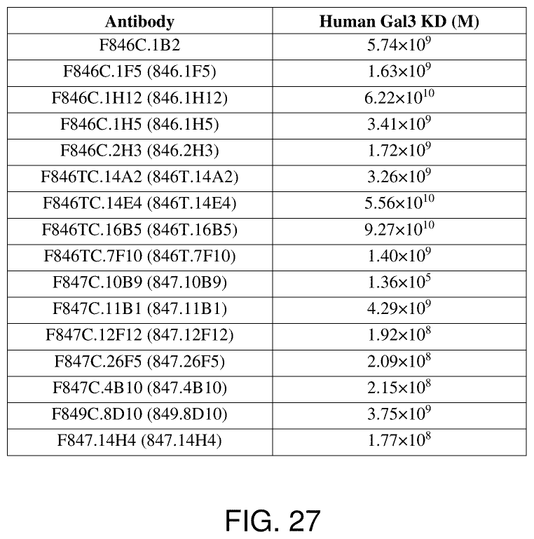

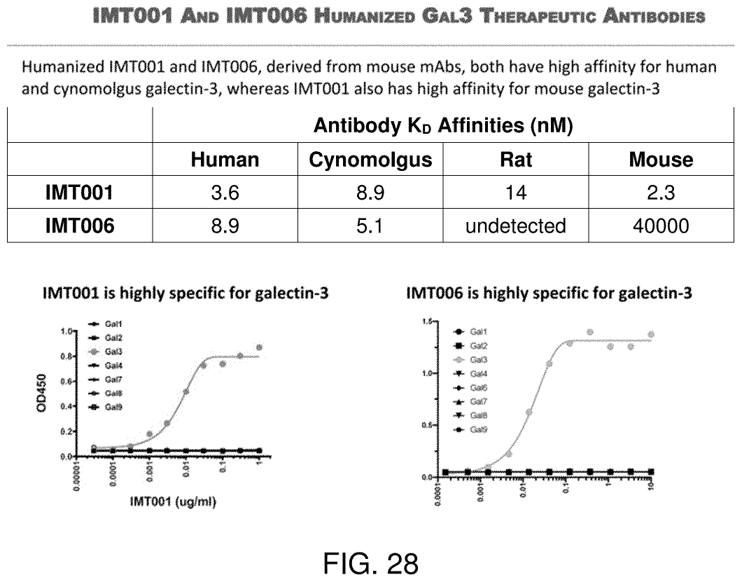

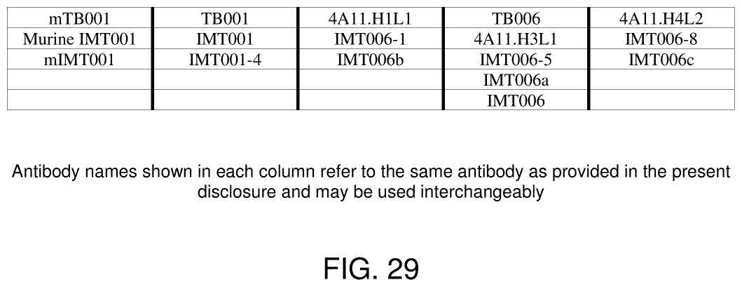

Binning results are shown in FIG. 26. In total, 49 distinct bins were identified for 120 anti-GAL3 antibodies that demonstrate binding to hGAL3 (30 antibodies out of 150 tested did not bind hGAL3 when immobilized on HC200M chip and were thus excluded from further analysis). Antibodies that strongly blocked the association of GAL3 and APP695 belonged to a number of distinct bins defined below. This highlights the utility of these bins as predictors of GAL3 binding activity.

Clones IMT001 (TB001) and F847C.21H6 exhibited mutual competitive binding for Gal3, but did not prevent binding of the rest of the clones, thus defining bin 1. Clones IMT006 (TB006), 19B5.2E6, 20H5.A3, 23H9.2E4, 2D10.2B2 exhibited mutual competitive binding for Gal3, but did not prevent binding of the rest of the clones, thus defining bin 3. Clones 20D11.2C6 and 15G7.2A7 exhibited mutual competitive binding for Gal3, but did not prevent binding of the rest of the clones, thus defining bin 5. Clones 3B11.2G2, 13A12.2E5 exhibited mutual competitive binding for Gal3, but did not prevent binding of the rest of the clones, thus defining bin 7. Clones 14H10.2C9, 15F10.2D6, 7D8.2D8, F846TC.14E4, F846TC.7F10, F849C.8D10 exhibited mutual competitive binding for Gal3, but did not prevent binding of the rest of the clones, thus defining bin 8. Clones 12G5.D7 and 849.2D7 exhibited mutual competitive binding for Gal3, but did not prevent binding of the rest of the clones, thus defining bin 10. Clones 24D12.2H9, 6B3.2D3, 849.1D2 exhibited mutual competitive binding for Gal3, but did not prevent binding of the rest of the clones, thus defining bin 11. Clones 13G4.2F8 and 9H2.2H10 exhibited mutual competitive binding for Gal3, but did not prevent binding of the rest of the clones, thus defining bin 12. Clones F846C.1B2, F846C.1F5, F846C.1H12, F846C.2H3, F846TC.16B5 exhibited mutual competitive binding for Gal3, but did not prevent binding of the rest of the clones, thus defining bin 17. In addition, clone 849.5H1 did not compete for binding to Gal3 with other antibodies tested, therefore defining bin 21.

Antibodies 847.12C4, 847.15D12, 847.15H11, 847.20H7, and 847.27B9 exhibited mutual competitive binding and competed with binding with the commercially available anti-Gal3 antibody B2C10 for hGAL3, but did not prevent binding of the rest of the clones, thus defining bin “B2C10”. B2C10 has been epitope mapped to bind to the first 18 amino acids of Gal3. Antibodies 847.10C9, 847.11D6, 847.13E2-mH0mL1, 847.13E2-mH0mL2, 847.16D10, 847.23F11, 847.28D1, and 847.3B3 exhibited mutual competitive binding to the C-terminal carbohydrate-recognition domain (CRD), but did not prevent binding of the rest of the clones, thus defining bin “CRD”. Antibodies F846TC.14A2, F847C.10B9, F847C.11B1, F847C.12F12, F847C.4B10, F849C.8H3, 849.8D12, F846C.1H5, 847.14H4, F847C.26F5, 849.2F12, were either not tested (“n/a”) or not assigned (“Unassigned”) to a bin. Antibodies 846.2B11, 846T.4C9, 847.15F9, 847.21B11, 847.2B8, 847.4D3, 849.4F12, and 849.4B2 were determined to not bind to hGAL3 under the conditions tested.

Gal3 peptide binding activity of additional exemplary anti-Gal3 antibodies was assessed with the same peptides as described in FIG. 18 (SEQ ID NOs: 3-26). Human Gal3 peptides (LifeTein, custom order) and human Gal3 proteins (R&D Systems, 8259-GA; TrueBinding, QCB200349) were diluted in PBS (Corning, 21-030-CM) to concentrations of at least 100 μg/mL (peptides) or 1 μg/mL (proteins), and added to the wells of several 96-well ELISA plates (Thermo Fisher, 44-2404-21). After incubating the plates at 4° C. overnight, the plates were washed three times with PBST (PBS with 0.05% Tween 20 [VWR, 0777]). The plates were then blocked for an hour with 2% BSA (EMD Millipore, 126609) in PBST at room temperature with gentle rocking. Thereafter, the 2% BSA in PBST was discarded and Gal3-binding antibodies (reformatted hIgG4 [S228P]) were diluted in 2% BSA in PBST to concentrations of 5 μg/mL and added to the wells. The plates were incubated for an hour at room temperature with gentle rocking and then washed three times with PBST. Afterwards, Peroxidase AffiniPure F(ab′)₂ Fragment Goat Anti-Human IgG (H+L) polyclonal antibody (Jackson ImmunoResearch, 109-036-003), was diluted in 2% BSA in PBST (1:4000) and added to the wells. The plates were incubated for 1 hour at room temperature with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

Peptide binding results are depicted in FIG. 26. Peptide numbering is based on FIG. 18. Binding of Gal3-binding antibodies to the peptide array was observed at multiple locations, with the majority of binding observed in peptides 1-8 and some binding to peptides 13 and 17.

13 separate Gal3-binding antibodies (19B5.2E6, 20D11.2C6, 20H5.A3, 23H9.2E4, F846C.1H5, F846TC.14A2, F846TC.7F10, F847C.10B9, F847C.12F12, F847C.26F5, F847C.4B10, 15FG7.2A7, 849.1D2) all bound peptide 1 of Gal3, corresponding to amino acids ADNFSLHDALSGSGNPNPQG (SEQ ID NO: 3) of Gal3.

7 separate Gal3-binding antibodies (15F10.2D6, 7D8.2D8, F846TC.14E4, F849C.8D10, F849C.8H3, 847.12C4, 847.10C9) bound peptide 2 of Gal3, corresponding to amino acids SGSGNPNPQGWPGAWGNQPA (SEQ ID NO: 4) of Gal3.

5 separate Gal3-binding antibodies (15F10.2D6, 7D8.2D8, F849C.8D10, 847.12C4, 847.10C9) bound peptide 3 of Gal3, corresponding to amino acids WPGAWGNQPAGAGGYPGASY (SEQ ID NO: 5) of Gal3.

4 separate Gal3-binding antibodies (13A12.2E5, 15F10.2D6, 3B11.2G2, 847.27B9) bound peptide 4 of Gal3, corresponding to amino acids GAGGYPGASYPGAYPGQAPP (SEQ ID NO: 6) of Gal3.

4 separate Gal3-binding antibodies (TB001 (IMT001), F846C.1B2, F846C.1H12, F847C.21H6) bound peptide 5 of Gal3, corresponding to amino acids PGAYPGQAPPGAYPGQAPPG (SEQ ID NO: 7) of Gal3.

15 separate Gal3-binding antibodies (TB001 (IMT001), TB006 (IMT006), 13A12.2E5, 14H10.2C9, 23H9.2E4, 2D10.2B2, 3B11.2G2, F846C.1B2, F846C.1F5, F846C.1H12, F846C.2H3, F846TC.16B5, F847C.21H6, 15FG7.2A7, 847.28D1) bound peptide 6 of Gal3, corresponding to amino acids GAYPGQAPPGAYPGAPGAYP (SEQ ID NO: 8) of Gal3.

13 separate Gal3-binding antibodies (14H10.2C9, 19B5.2E6, 20D11.2C6, 20H5.A3, 23H9.2E4, F846C.1B2, F846TC.14A2, F847C.10B9, F847C.12F12, F847C.26F5, 15FG7.2A7, 9H2.2H10, 847.14H4) all bound peptide 7 of Gal3, corresponding to amino acids AYPGAPGAYPGAPAPGVYPG (SEQ ID NO: 9) of Gal3.

4 separate Gal3-binding antibodies (20D11.2C6, 23H9.2E4, F846TC.14A2, 15G7.2A7) all bound peptide 8 of Gal3, corresponding to amino acids GAPAPGVYPGPPSGPGAYPS (SEQ ID NO: 10) of Gal3.

2 separate Gal3-binding antibodies (847.12C4, 847.10C9) bound peptide 9 of Gal3, corresponding to amino acids PPSGPGAYPSSGQPSATGAY (SEQ ID NO: 11) of Gal3.

3 separate Gal3-binding antibodies (15G7.2A7, 847.12C4, 847.10C9) all bound peptide 10 of Gal3, corresponding to amino acids SGQPSATGAYPATGPYGAPA (SEQ ID NO: 12) of Gal3.

3 separate Gal3-binding antibodies (847.13E2-mH0mL1, 847.13E2-mH0mL2, 847.23F11) all bound peptide 13 of Gal3, corresponding to amino acids LPGGVVPRMLITILGTVKPN (SEQ ID NO: 15) of Gal3.

13 separate Gal3-binding antibodies (7D8.2D8, F846C.1F5, F846C.1H12, F846C.2H3, F846TC.16B5, F847C.11B1, F849C.8H3, 849.1D2, 847.15D12, 847.13E2-mH0mL1, 847.13E2-mH0mL2, 847.23F11, 847.3B3) all bound peptide 17, corresponding to amino acids RFNENNRRVIVCNTKLDNNW (SEQ ID NO: 19) of Gal3.

19 separate Gal-3 binding antibodies (849.2D7, 24D12.2H9, 6B3.2D3, 13G4.2F8, 849.5H1, 847.15H11, 847.20H7, 847.11D6, 847.16D10, 849.8D12, 846.2B11, 846T.4C9, 847.15F9, 847.21B11, 847.2B8, 847.4D3, 849.4F12, 849.4B2, 849.2F12) were not found to bind to any of the tested Gal3 peptides under the conditions used in this Example.

As illustrated in FIG. 18, peptides 4, 5, 6, and 7 share repeated amino acid sequences comprised of proline-glycine (PG) and tyrosine-proline-glycine (YPG), indicating a common feature that may explain the ability of Gal3-targeted antibodies to bind to multiple Gal3 peptides. Further, the amino acid sequence glycine-x-tyrosine-proline-glycine (GxYPG), where x may be the amino acids alanine (A), glycine (G), or valine (V), is shared in peptides 4, 6, and 7, each of which possess two such sequences separated by 3 amino acids. Accordingly, the presence of two GxYPG sequences in close apposition is likely predictive of the ability to bind Gal3-targeted antibodies. Additionally, the Grantham distance of alanine, glycine, and valine is Ala-Val: 64, Ala-Gly: 60, Val-Gly: 109, thereby predicting that amino acids with similarly low Grantham distances may similarly be able to substitute at the variable region, including proline and threonine.

### Example 9. Humanized Anti-Gal3 Antibodies have High Affinity for Gal3 of Different Species

Cross reactivity of exemplary anti-Gal3 antibodies TB001 and TB006 to human or mouse Gal3 were tested. Kinetics experiments were performed on a Carterra LSA at 25° C. An HC30M chip was immobilized with recombinant Protein A/G. TB001 (IMT001;

IMT001-4), TB006 (IMT006; IMT006-5), and a negative control antibody Synagis (10 μg/mL each diluted in HBSTE buffer (HEPES based saline with Tween-20 and EDTA) with 0.5 mg/mL BSA) were captured onto different spots in the 384-spot array. Then, a dilution series of Gal3 (2, 4, 8, 16, 32, 64, and 128 nM) in HBSTE with 0.5 mg/mL BSA was injected to the whole array for 300 seconds to allow association followed by dissociation for 240 seconds. The kinetics constants were calculated by the software NextGenKIT.

Separately, the affinity kinetics of TB001 and TB006 to cynomolgus Gal3 (cynoGal3) was measured on a Biacore T200 at 25° C. A CM5 chip coated with anti-mouse Fc/anti-human Fc mixture was used to load purified antibody TB001 or TB006 at 10 μg/mL in HBS-EP+ buffer (HEPES based saline with polysorbate 20 and EDTA) with 0.5 mg/mL BSA for 180 seconds, and then a dilution series of His-enterokinase cleavage site (EK)-cynoGal3 (TrueBinding in-house antigen) in HBS-EP+ with 0.5 mg/mL BSA starting from 100 nM, 1:2 dilution for 7 points for 200 seconds, followed by dissociation for 300 seconds. In each cycle, duplicates were made by loading the same antibody to flow cells FC2 and FC4, and then analyte was flowed to all 4 flow cells. The detection was done pairwise, FC2-FC1 and FC4-FC3, to subtract non-specific binding of analyte to the surface. A cycle of 0 nM analyte was run prior to a dilution series of analyte. The 0 nM raw data were subtracted from each non-zero concentration data to minimize machine drifts. The data were analyzed by Biacore Evaluation software version 2.0. The binding affinities were characterized by fitting the kinetic sensograms to a monovalent binding model (1:1 binding).

Separately, the affinity kinetics of TB001 and TB006 to rat Gal3 was measured on a Biacore T200 at 25° C. A CM5 chip was coated with goat anti-human Fc antibody on all 4 flow cells, and was used to load purified antibodies TB001, TB006, and Synagis (negative control), which all use an hIgG4 backbone. In each sample cycle, duplicates of the same antibody in HBS-EP+ with 0.5 mg/mL BSA at 10 μg/mL were captured to FC2 and FC4 by flowing at 10.0 μL/min for 120 seconds, and then diluted His-EK-ratGal3 in HBS-EP+ with 0.5 mg/mL BSA was injected to all 4 flow cells at 30.0 μL/min for 180 seconds, followed by dissociation for 240 seconds. A dilution series of His-EK-ratGal3 started from 300 nM, and was 1:3 diluted for 6 points. FC1 and FC3 were used as reference flow cells, and their sensograms were subtracted from FC2 and FC4 respectively to reduce non-specific binding background. Also, an additional sample cycle at 0 nM analyte was run, and its sensogram was subtracted from those of non-zero nM cycles to address machine drifts and bulk shifts. The data were analyzed by Biacore Evaluation software version 2.0. The binding affinities were characterized by fitting the kinetic sensograms to a monovalent binding model (1:1 binding).

The affinity of Gal3 binding to antibody was determined by acquiring real time Ligand:Analyte binding kinetics data and fitting the data with a 1:1 monovalent binding model. The kinetic evaluation procedure determines association and dissociation constants by fitting the experimental data to a 1:1 interaction model between analyte A and ligand B:

Kₐ: A+B→AB; and K_{d}: A+B←AB, where Kₐ is the association rate constant (M⁻¹s⁻¹) and the K_{d} is the dissociation rate constant (s⁻¹).

The net rate of complex formation during injection is given by:

d[AB]/dt=Kₐ[A][B]−K_{d}[AB]

and the rate of dissociation after the end of the injection is:

d[AB]/dt=−K_{d}[AB].

The affinity KD is the result of K_{d} divided by Kₐ.

Humanized TB001 (IMT001) and TB006 (IMT006a), which were derived from mouse mAbs, both have high affinity for human (IMT001: 3.6 nM, IMT006a: 8.9 nM) and cynomolgus (IMT001: 8.9 nM, IMT006a: 5.1 nM) Gal3 (FIG. 28). IMT001 also has high affinity for mouse Gal3 (IMT001: 2.3 nM, IMT006a: 40000 nM) and rat Gal3 (IMT001: 14 nM, IMT006a: undetected).

### Example 10. Antibodies that Bind to GAL3 NTD and CRD have Differential Activities in Regulating GAL3-Mediated GLUT4 Translocation

Insulin leads to glucose uptake by stimulating the translocation of the glucose transporter GLUT4 from intracellular stores to the plasma membrane. It was tested if Gal3 can interfere with GLUT4 translocation and if anti-Gal3 antibodies can block this effect.

Rat L6 GLUT4myc myoblasts (Kerafast #ESK202-FP) express a Myc-tagged GLUT4 that can be detected with an anti-Myc antibody. These cells were grown in a 48-well plate in MEM u growth media (Gibco, #12-571-063)+10% fetal bovine serum (FBS; Corning #35-016-CV) until 90-95% confluence was reached. Cells were then washed in PBS and serum starved for 2 hours in MEM u growth media without FBS. Thirty (30) μg/mL recombinant human Gal3 (TrueBinding QCB200347) was pre-incubated with 60 μg/mL anti-Gal3 antibodies for 30 minutes before being added to the serum-starved cells for 1 hour at 37° C. At the end of incubation, half of the cells were treated with 100 nM insulin (Sigma-Aldrich #I5523-50MG) for 20 minutes while the other half acted as basal controls. After 20 minutes, cells were washed quickly with ice cold PBS twice, then incubated with 4% paraformaldehyde (Boster Bio #AR1068) for 10 minutes on ice followed by 10 minutes at room temperature. The cells were washed three times with PBS, then blocked with 0.5 mL 5% goat serum (Vector Laboratories #101098-382) in PBS for 15 minutes. After the blocking agent was aspirated from the cells, 1 μg/ml anti-Myc polyclonal antibody (Sigma-Aldrich #C-3956) in 5% goat serum was added for 1 hour. One well did not receive the primary antibody and was used later for background subtraction. Primary antibody was aspirated away, and then cells washed 5 times with PBS. Next, cells (including background controls) were incubated with 1:1000 goat anti rabbit antibody secondary antibody (Abcam #Ab97051) in 5% Goat serum for 30 minutes at room temperature. After washing, cells were incubated with 400 μL of TMB (Thermo Scientific #PI34029) for 5 minutes, then 200 μL of 1N HCL (Avantor Performance #JT5620-2) was added to stop the reaction. 100 μL of the sample was transferred to a 96 well plate with duplicates transferred for each condition. Samples were read at 450 nm on a SpectraMax plate reader. After subtracting background control values for cells that did not receive primary antibody, the GLUT4 Translocation Insulin Stimulation Index was calculated as the fold change of cells treated with Gal3 alone or treated with Gal3 following pre-incubation with anti-Gal3 antibodies with insulin (Stimulated) to that of without insulin (Basal). The fold changes were plotted on GraphPad Prism and presented as mean (SEM) of the technical replicates. Statistical significance was determined using unpaired T-tests comparing samples treated with Gal3 and insulin with all other conditions.

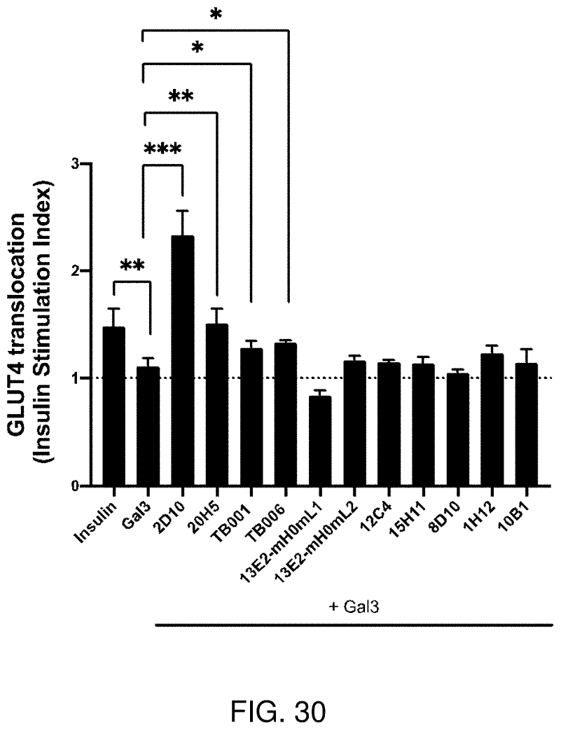

FIG. 30 shows that insulin stimulation elevates the surface level of Myc-tagged GLUT4 that can be detected and that Gal3 co-treatment blocks that increase. Antibodies that bind the N-terminal domain (NTD) of Gal3 (e.g. TB001, TB006, 2D10-VH0-VL0, 20H5.A3) reduce the ability of Gal3 to block GLUT4 translocation. Antibodies that bind to the carbohydrate recognition binding domain (CRD) of Gal3 (e.g. 13E2) enhance the ability of Gal3 to block GLUT4 translocation.

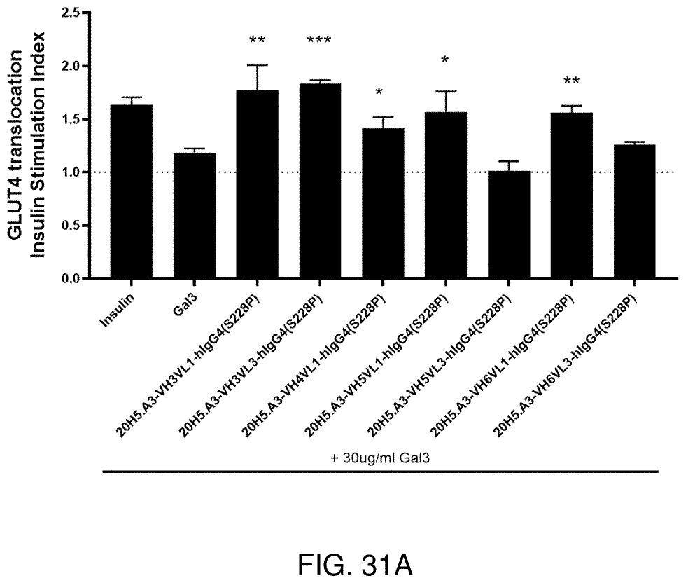

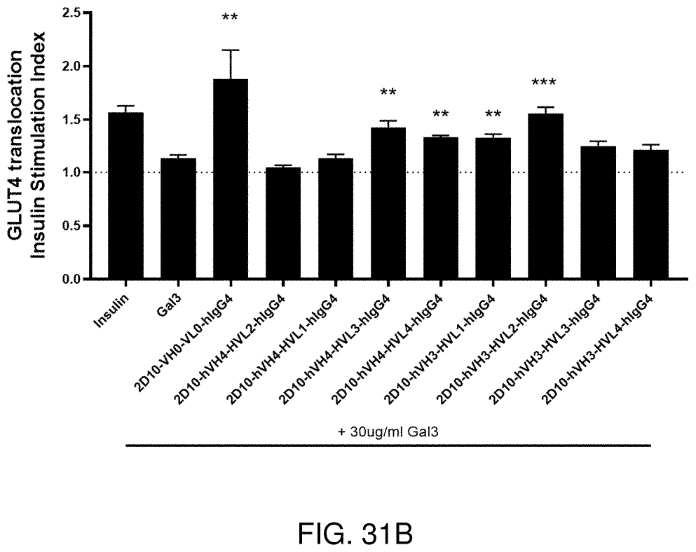

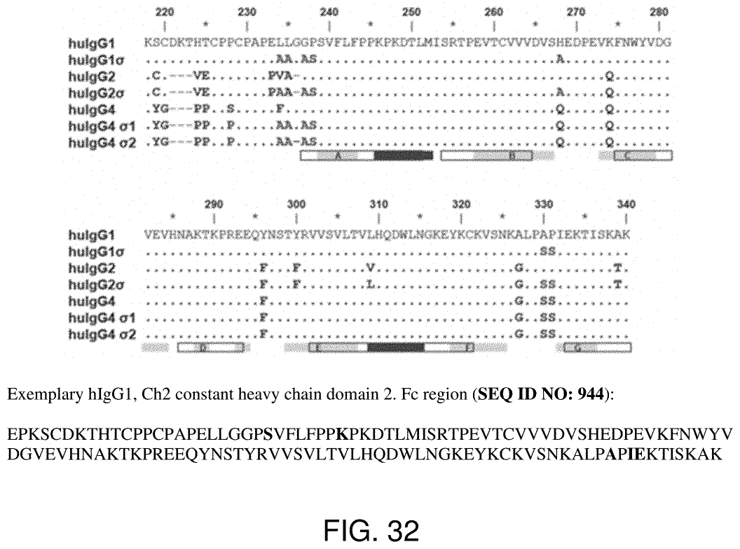

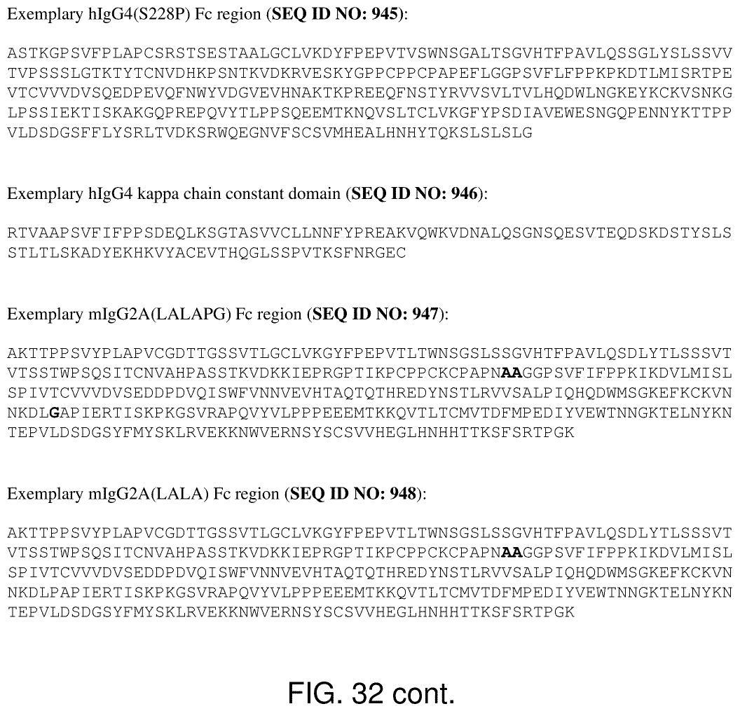

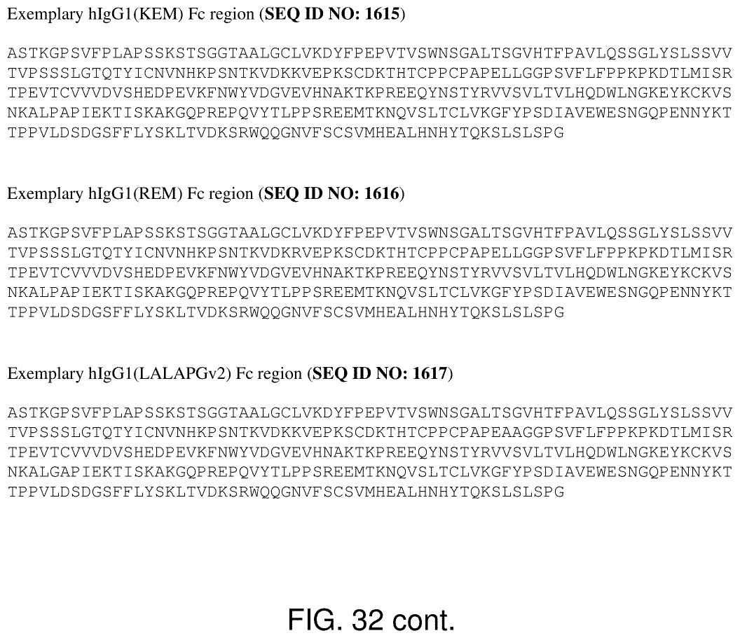

FIG. 31A shows that variants of 20H5 (20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3) can reduce the ability of Gal3 to block insulin-dependent GLUT4 translocation. FIG. 31B shows that variants of 2D10-VH0-VL0 (2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4) can reduce the ability of Gal3 to block GLUT4 translocation. It is believed that the variation in blocking between the antibodies is due to variations in the antibodies' aggregation status, rather than the effectiveness of the specific CDRs for the antibodies.

### Example 11. Anti-Gal3 Antibodies Block Gal3-Induced Apoptosis in Pancreatic Islet Cells

Death of insulin-secreting pancreatic islet beta cells can lead to diabetes. It can be tested if Gal3 can induce beta cell apoptosis and if anti-Gal3 antibodies can block this effect.

The INS-1 832/13 cell line (Sigma-Aldrich #SCC207) is a rat beta cell line that produces insulin. These cells will be grown in a 48-well plate in RPMI-1640 with glucose (Gibco #11875-093)+10% FBS (Corning #35-016-CV) until 90-95% confluence is reached. Cells will then be washed in PBS and serum starved for 2 hours in RPMI without glucose (Gibco #11879-020) and without FBS added. 30 μg/mL recombinant human Gal3 (TrueBinding QCB200347) will be pre-incubated with or without 60 μg/mL anti-Gal3 antibodies for 30 minutes before being added to the serum-starved cells for 1-4 hour at 37 C. At the end of the time course, cells will be lysed and analyzed for apoptosis with a Caspase 3 Assay (Sigma Aldrich #CASP3F). The fold change in fluorescence of cells that will be treated with Gal3 alone or Gal3 incubated with anti-Gal3 antibodies compared to those that will be unstimulated will be tested to ascertain change in Gal-3 induced apoptosis. The fold changes are plotted on GraphPad Prism and show the mean (+SEM) of the technical replicates. Statistical significance will be determined using an ordinary one-way ANOVA.

### Example 12: Anti-Gal3 Antibodies for Use in the Treatment of Diabetes

Patients present with diabetes mellitus type I or type II. One or more anti-Gal3 antibodies or binding fragments thereof disclosed herein are administered to the patients enterally, orally, intranasally, parenterally, intracranially, subcutaneously, intramuscularly, intradermally, or intravenously.

The anti-Gal3 antibodies or binding fragments thereof are administered as doses in at an amount of 1 ng (or in the alternative: 0.1, 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans). The doses are administered every 1 day (or in the alternative: every 2, 3, 4, 5, 6, 7, 8, 9, 10, 11, 12, 24, 36, or 48 days or any time within a range defined by any two of the aforementioned times).

A treatment of the diabetes or symptoms associated with diabetes is observed in the patients following administration of the anti-Gal3 antibodies or binding fragments thereof. Administration of the anti-Gal3 antibodies or binding fragments may be performed in conjunction with another therapy for diabetes, for example, insulin, an insulin derivative or mimetic thereof, insulin aspart, insulin glulisine, insulin lispro, insulin isophane, insulin degludec, insulin detemir, insulin zinc, insulin glargine, vanadium, biguanides, metformin, phenformin, buformin, thiazolidinediones, rosiglitazone, pioglitazone, troglitazone, tolimidone, sulfonylureas, tolbutamide, acetohexamide, tolazamide, chlorpropamide, glipizide, glibenclamide, glimepiride, gliclazide, glyclopyramide, gliquidone, meglitinides, repaglinide, nateglinide, alpha-glucosidase inhibitors, miglitol, acarbose, voglibose, incretins, glucagon-like peptide 1, glucagon-like peptide agonists, exenatide, liraglutide, taspoglutide, lixisenatide, semaglutide, dulaglutide, gastric inhibitory peptide, dipeptidyl peptidase-4 inhibitors, vildagliptin, sitagliptin, saxagliptin, linagliptin, alogliptin, septagliptin, teneligpliptin, gemigliptin, pramlintide, dapagliflozin, canagliflozin, empagliflozin, or remogliflozin.

### Example 13: Anti-Gal3 Antibodies for Use in the Treatment of Insulin Resistance

Patients present with insulin resistance, which may be associated with one or more diseases, including but not limited to diabetes mellitus, chronic hyperinsulinemia, dysmetabolic syndrome, type A insulin resistance syndrome, type B insulin resistance syndrome, gestational diabetes, acanthosis nigricans, polycystic ovary syndrome (PCOS), obesity, muscle wasting, cardiovascular diseases, cardiac hypertrophy, myocardial ischemia, hypertension, pancreatic cancer associated diabetes (PCDM), or cancers. One or more anti-Gal3 antibodies or binding fragments thereof disclosed herein are administered to the patients enterally, orally, intranasally, parenterally, intracranially, subcutaneously, intramuscularly, intradermally, or intravenously.

The anti-Gal3 antibodies or binding fragments thereof are administered as doses in an amount of 1 ng (or in the alternative: 0.1, 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans). The doses are administered every 1 day (or in the alternative: every 2, 3, 4, 5, 6, 7, 8, 9, 10, 11, 12, 24, 36, or 48 days or any time within a range defined by any two of the aforementioned times).

A treatment of the insulin resistance or symptoms associated with insulin resistance is observed in the patients following administration of the anti-Gal3 antibodies or binding fragments thereof. Administration of the anti-Gal3 antibodies or binding fragments may be performed in conjunction with another therapy for insulin resistance, for example, insulin, an insulin derivative or mimetic thereof, insulin aspart, insulin glulisine, insulin lispro, insulin isophane, insulin degludec, insulin detemir, insulin zinc, insulin glargine, vanadium, biguanides, metformin, phenformin, buformin, thiazolidinediones, rosiglitazone, pioglitazone, troglitazone, tolimidone, sulfonylureas, tolbutamide, acetohexamide, tolazamide, chlorpropamide, glipizide, glibenclamide, glimepiride, gliclazide, glyclopyramide, gliquidone, meglitinides, repaglinide, nateglinide, alpha-glucosidase inhibitors, miglitol, acarbose, voglibose, incretins, glucagon-like peptide 1, glucagon-like peptide agonists, exenatide, liraglutide, taspoglutide, lixisenatide, semaglutide, dulaglutide, gastric inhibitory peptide, dipeptidyl peptidase-4 inhibitors, vildagliptin, sitagliptin, saxagliptin, linagliptin, alogliptin, septagliptin, teneligpliptin, gemigliptin, pramlintide, dapagliflozin, canagliflozin, empagliflozin, or remogliflozin.

### Example 14: Quantification of Affinity for Exemplary Anti-Gal3 Antibodies to Gal3 of Different Species

The protocol disclosed in Example 9 was generally followed.

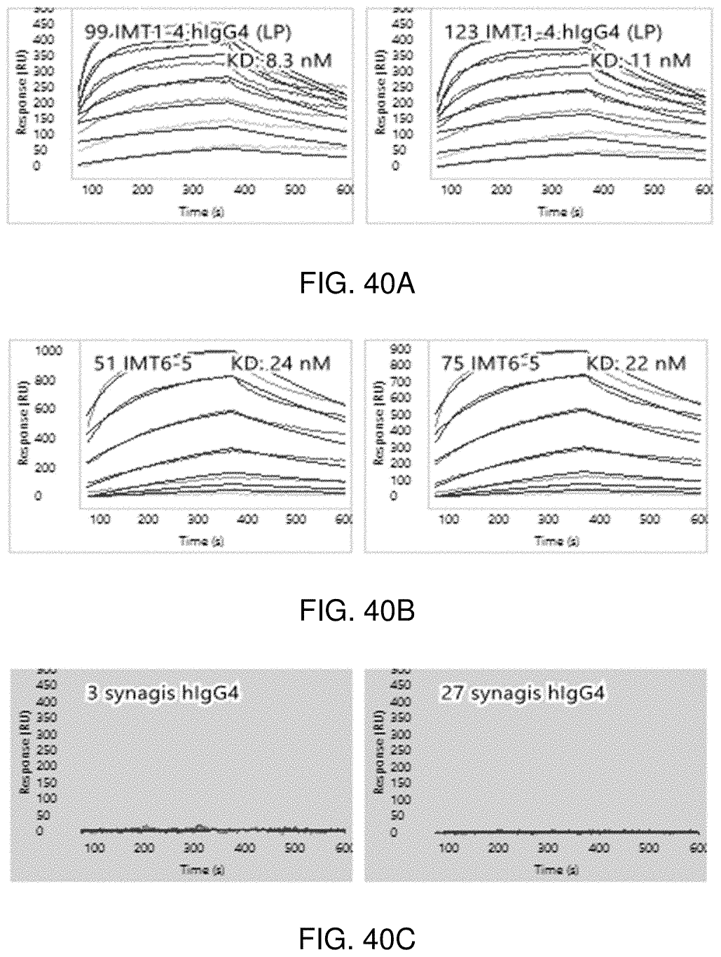

TB001 showed binding to human Gal3 at an average affinity of 9.7 nM (FIG. 40A), while TB006 binds to human Gal3 at an average affinity of 23 nM (FIG. 40B). Synagis did not bind to human Gal3.

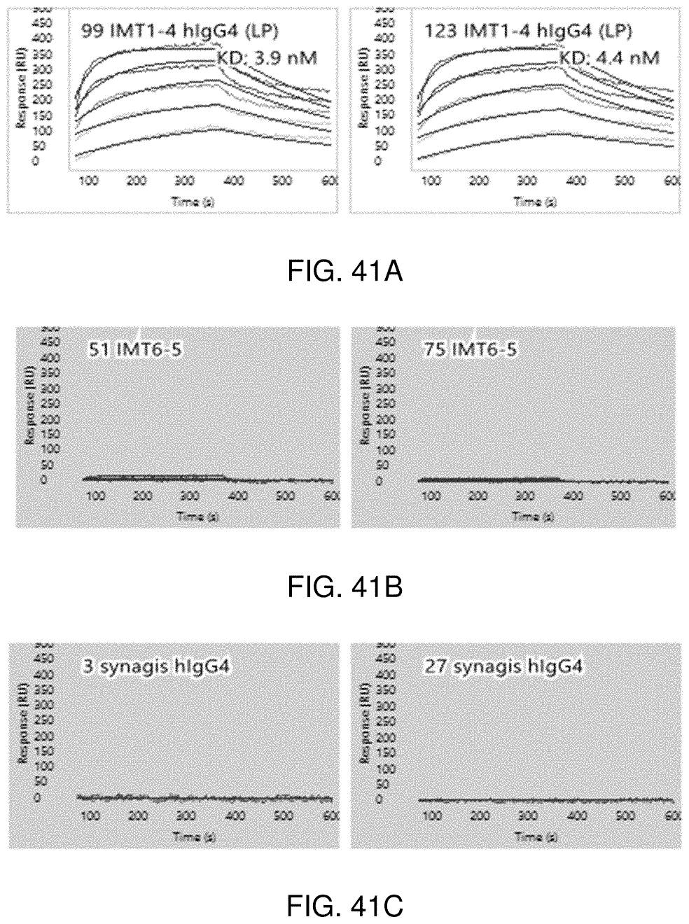

TB001 showed binding to mouse Gal3 at an average affinity of 4.2 nM (FIG. 41A), while TB006 or Synagis did not show any binding activity to mouse Gal3 (FIG. 41B-C).

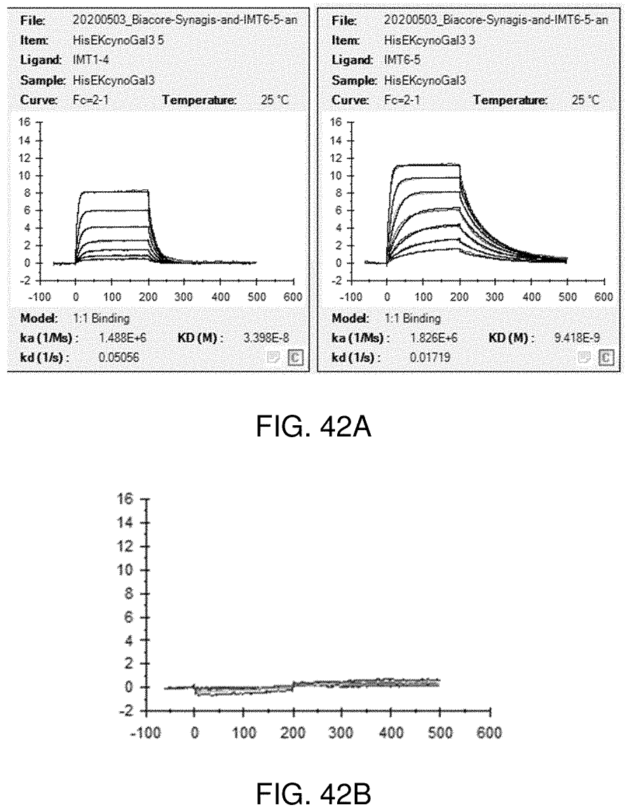

TB001 showed binding to His-EK-cynoGal3 at an affinity of 34 nM (FIG. 42A, left panel), while TB006 binds to His-EK-cynoGal3 at an affinity of 9.4 nM (FIG. 42A, right panel). Synagis did not bind to His-EK-cynoGal3 (FIG. 42B).

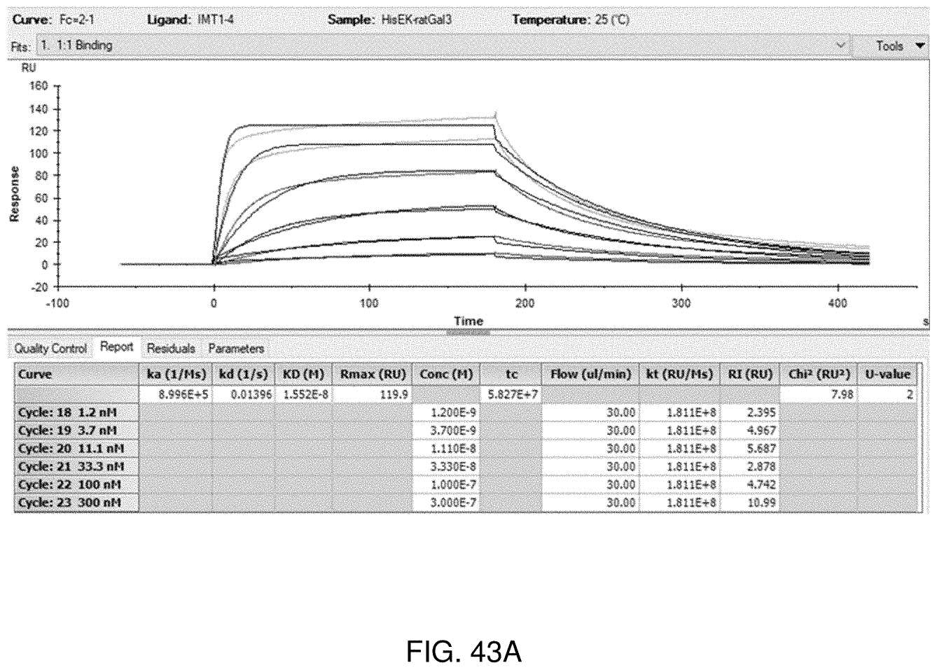

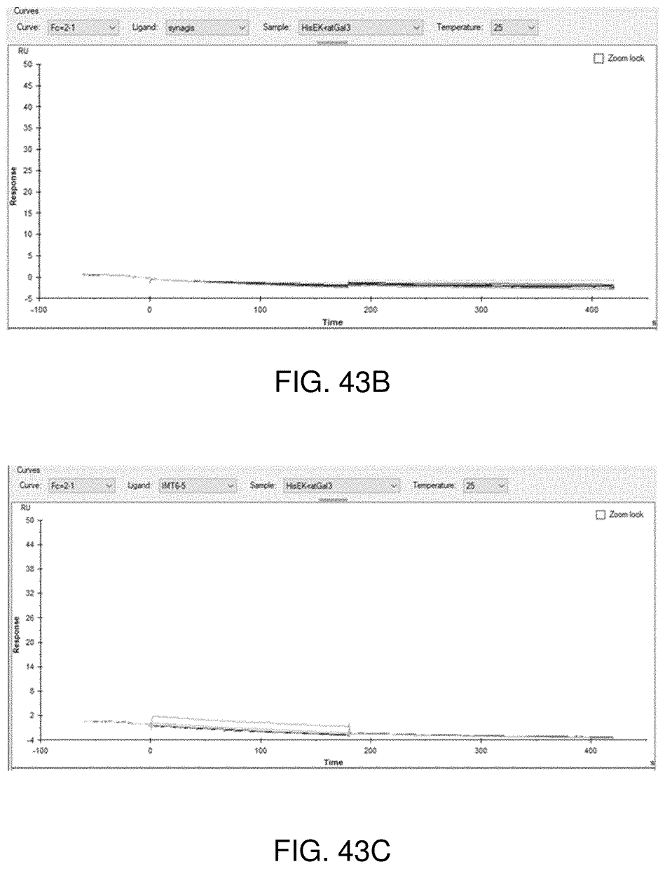

TB001 showed binding to His-EK-ratGal3 at an average affinity of 14 nM (representative sensogram depicted in FIG. 43A), while TB006 or Synagis did not bind to His-EK-ratGal3.

The resulting data showed similar binding profiles of TB001 and TB006 to human Gal3 and cynomolgus Gal3. Meanwhile, TB001 displayed stronger binding activity to rat and mouse Gal3 in comparison to the binding activity of TB006.

### Example 15: Screening for Additional Anti-Gal3 Antibodies that Induce Insulin-Independent GLUT4 Translocation

Additional anti-Gal3 antibodies were screened for ability to induce insulin-independent GLUT4 translocation in L6 GLUT4myc myoblasts according to the protocol provided in Example 10, but modified such that the cells were not treated with insulin. In these experiments, 30 μg/mL (approximately 1 μM) of human Gal3 was incubated with 60 μg/mL (approximately 0.4 μM) of candidate antibodies or MOPC21 control antibody, and cells were treated without insulin.

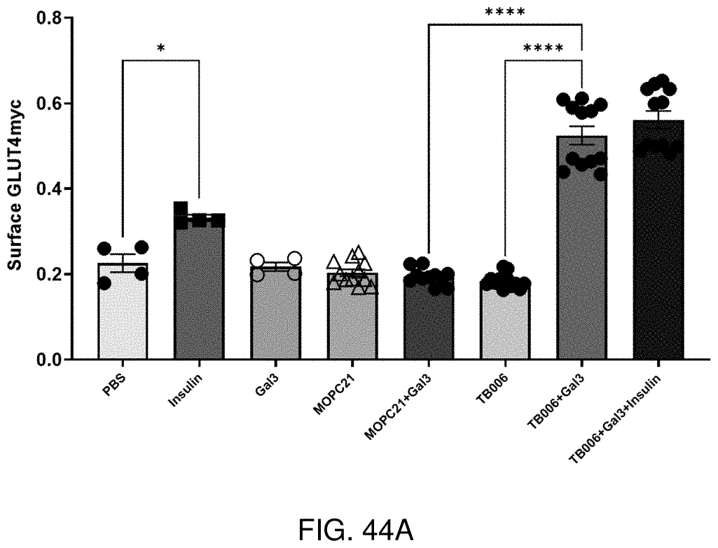

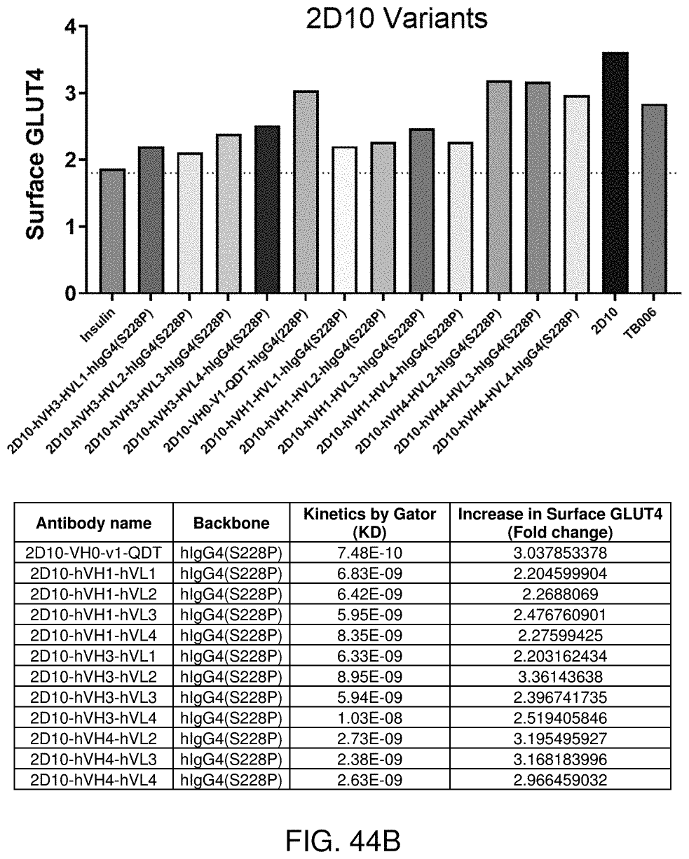

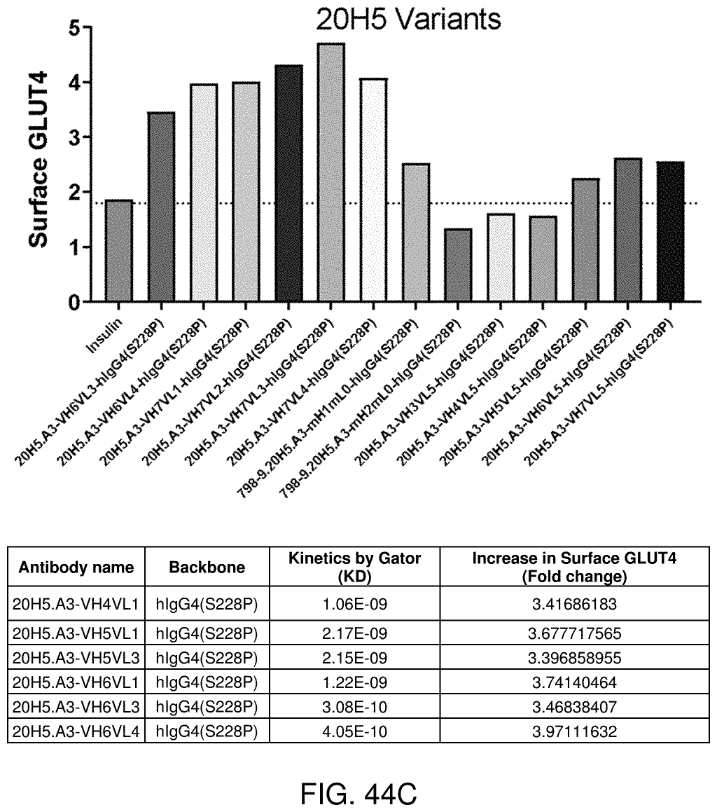

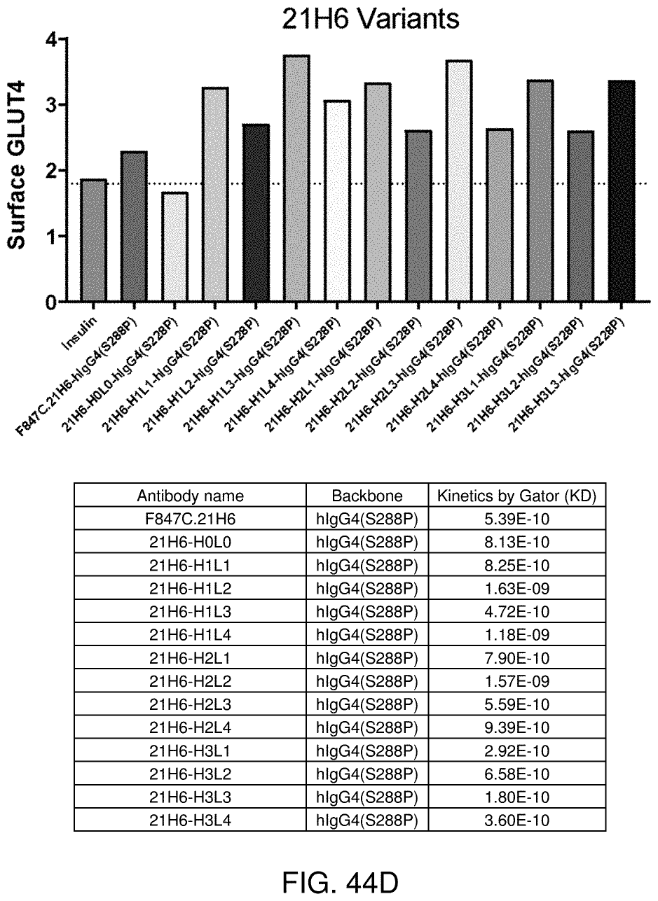

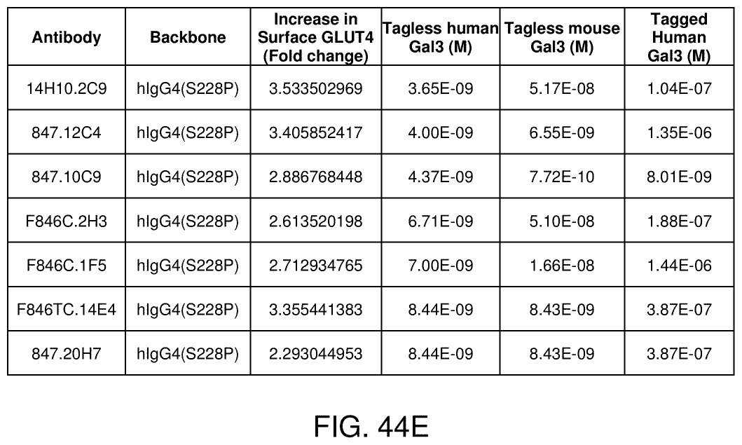

The preincubated TB006 and Gal3 complex increased surface GLUT4 levels independent of insulin in tested L6 GLUT4myc cells (FIG. 44A). Interestingly, TB006 alone did not result in comparable induction of GLUT4 translocation, suggesting that the antibody-Gal3 complex exhibits an insulin-independent effect relative to antibody alone.

The 2D10.2B2 (2D10) antibody and variants thereof disclosed herein were screened for the ability to induce insulin-independent GLUT4 translocation according to the same protocol provided in Example 10, but modified such that the cells were not treated with insulin. The 2D10 variants have the same heavy chain and light chain CDRs and generally vary in framework sequences. Exemplary variants tested (and their corresponding VH and VL) are shown in Table 1. Each of the 2D10 variants increased surface GLUT4 levels independent of insulin in tested L6 GLUT4myc cells (FIG. 44B).

TABLE 1
Tested 2D10 variants

| | Heavy chain | Light chain |
|---|---|---|
| Antibody | variable region | variable region |
| 2D10-VH0-v1-QDT | SEQ ID NO: 959 | SEQ ID NO: 973 |
| 2D10-hVH1-hVLl | SEQ ID NO: 961 | SEQ ID NO: 975 |
| 2D10-hVH1-hVL2 | SEQ ID NO: 962 | SEQ ID NO: 976 |
| 2D10-hVH1-hVL3 | SEQ ID NO: 963 | SEQ ID NO: 977 |
| 2D10-hVH1-hVL4 | SEQ ID NO: 964 | SEQ ID NO: 978 |
| 2D10-hVH2-hVLl | SEQ ID NO: 965 | SEQ ID NO: 979 |
| 2D10-hVH2-hVL2 | SEQ ID NO: 966 | SEQ ID NO: 980 |
| 2D10-hVH2-hVL3 | SEQ ID NO: 967 | SEQ ID NO: 981 |
| 2D10-hVH2-hVL4 | SEQ ID NO: 968 | SEQ ID NO: 982 |
| 2D10-hVH3-hVLl | SEQ ID NO: 810 | SEQ ID NO: 825 |
| 2D10-hVH3-hVL2 | SEQ ID NO: 811 | SEQ ID NO: 826 |
| 2D10-hVH3-hVL3 | SEQ ID NO: 812 | SEQ ID NO: 827 |
| 2D10-hVH3-hVL4 | SEQ ID NO: 813 | SEQ ID NO: 828 |
| 2D10-hVH4-hVLl | SEQ ID NO: 806 | SEQ ID NO: 821 |
| 2D10-hVH4-hVL2 | SEQ ID NO: 807 | SEQ ID NO: 822 |
| 2D10-hVH4-hVL3 | SEQ ID NO: 808 | SEQ ID NO: 823 |
| 2D10-hVH4-hVL4 | SEQ ID NO: 809 | SEQ ID NO: 824 |

Variants of the 20H5.A3 (20H5) antibody disclosed herein were screened for the ability to induce insulin-independent GLUT4 translocation according to the same protocol provided in Example 10, but modified such that the cells were not treated with insulin. The 20H5 variants have the same heavy chain and light chain CDRs and generally vary in the framework sequences. Exemplary variants tested (and their corresponding VH and VL) are shown in Table 2. The 20H5 variants exhibited variability in increasing surface GLUT4 levels independent of insulin, with some variants reducing GLUT4 levels, in tested L6 GLUT4myc cells (FIG. 44C).

TABLE 2
Tested 20H5 variants

| | Heavy chain | Light chain |
|---|---|---|
| Antibody | variable region | variable region |
| 20H5.A3-VH3VL1 | SEQ ID NO: 814 | SEQ ID NO: 829 |
| 20H5.A3-VH3VL3 | SEQ ID NO: 815 | SEQ ID NO: 830 |
| 20H5.A3-VH4VL1 | SEQ ID NO: 816 | SEQ ID NO: 831 |
| 20H5.A3-VH4VL3 | SEQ ID NO: 1086 | SEQ ID NO: 1129 |
| 20H5.A3-VH4VL4 | SEQ ID NO: 1087 | SEQ ID NO: 1130 |
| 2OH5.A3-VH5VL1 | SEQ ID NO: 817 | SEQ ID NO: 832 |
| 2OH5.A3-VH5VL3 | SEQ ID NO: 818 | SEQ ID NO: 833 |
| 2OH5.A3-VH6VL1 | SEQ ID NO: 819 | SEQ ID NO: 834 |
| 20H5.A3-VH6VL3 | SEQ ID NO: 820 | SEQ ID NO: 835 |
| 20H5.A3-VH6VL4 | SEQ ID NO: 1092 | SEQ ID NO: 1135 |

Variants of the F847C.21H6 (21H6) antibody disclosed herein were screened for the ability to induce insulin-independent GLUT4 translocation according to the same protocol provided in Example 10, but modified such that the cells were not treated with insulin. The 21H6 variants have the same heavy chain and light chain CDRs and generally vary in the framework sequences. Exemplary variants tested (and their corresponding VH and VL) are shown in Table 3. The 2116 variants, other than 2116-H0L0, increased surface GLUT4 levels independent of insulin in tested L6 GLUT4myc cells (FIG. 44D).

TABLE 3
Tested 21H6 variants

| | Heavy chain | Light chain |
|---|---|---|
| Antibody | variable region | variable region |
| F847C.21H6 | SEQ ID NO: 367 | SEQ ID NO: 440 |
| 21H6-H0L0 | SEQ ID NO: 1415 | SEQ ID NO: 1440 |
| 21H6-H1L1 | SEQ ID NO: 1416 | SEQ ID NO: 1441 |
| 21H6-H1L2 | SEQ ID NO: 1417 | SEQ ID NO: 1442 |
| 21H6-H1L3 | SEQ ID NO: 1418 | SEQ ID NO: 1443 |
| 21H6-H1L4 | SEQ ID NO: 1419 | SEQ ID NO: 1444 |
| 21H6-H2L1 | SEQ ID NO: 1420 | SEQ ID NO: 1445 |
| 21H6-H2L2 | SEQ ID NO: 1421 | SEQ ID NO: 1446 |
| 21H6-H2L3 | SEQ ID NO: 1422 | SEQ ID NO: 1447 |
| 21H6-H2L4 | SEQ ID NO: 1423 | SEQ ID NO: 1448 |
| 21H6-H3L1 | SEQ ID NO: 1424 | SEQ ID NO: 1449 |
| 21H6-H3L2 | SEQ ID NO: 1425 | SEQ ID NO: 1450 |
| 21H6-H3L3 | SEQ ID NO: 1426 | SEQ ID NO: 1451 |
| 21H6-H3L4 | SEQ ID NO: 1427 | SEQ ID NO: 1452 |

In addition to these antibodies, other anti-Gal3 antibodies disclosed herein were tested for their ability to induce insulin-independent GLUT4 translocation according to the same protocol provided in Example 10, but modified such that the cells were not treated with insulin (FIG. 44E).

### Example 16: The Gal3 N-Terminal and C-Terminal Domain are Important for Inducing GLUT4 Translocation

Insulin-independent GLUT4 translocation induced by complexed Gal3 and anti-Gal3 antibody in L6 GLUT4myc myoblasts was tested according to the protocol provided in Example 10 but using different mutants of Gal3. The human Gal3 mutants that were tested include: hGal3 R186S (mutation that blocks glycan binding), amino acids 65-250 of hGal3, hGal3 P64H (mutation that makes Gal3 susceptible to cleavage by MMP), amino acids 2-112 of hGal3 (N-terminal domain), and amino acids 111-250 of hGal3 (C-terminal carbohydrate recognition domain). The Gal3 mutants were pre-incubated with TB006 to form a Gal3-antibody complex prior to myoblast treatment.

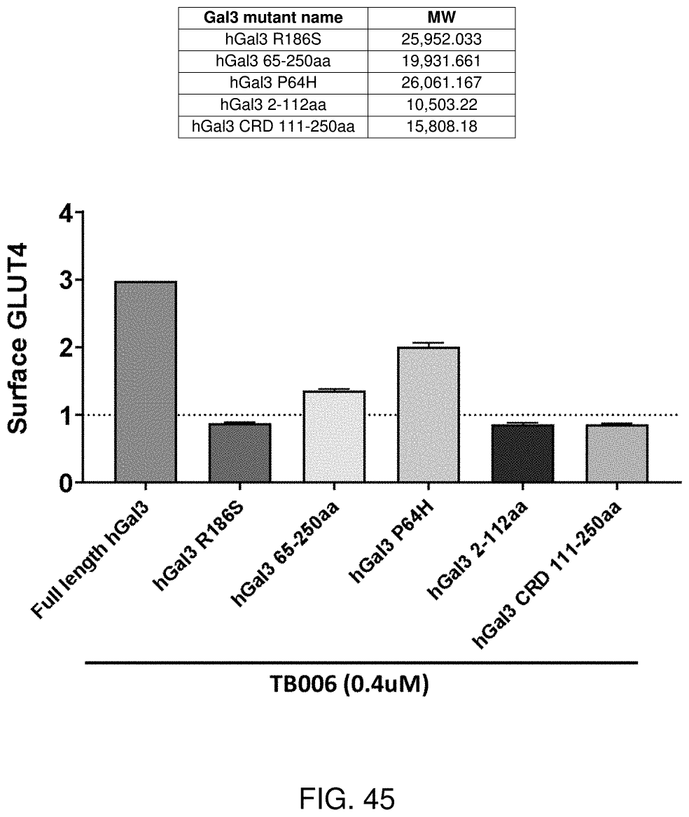

FIG. 45 shows the ability of TB006 complexed with the different Gal3 mutants to induce insulin-independent GLUT4 translocation. The 2-112 amino acid N-terminal truncation and the 111-250 amino acid C-terminal truncation both exhibit significantly reduced GLUT4 translocation, suggesting that both domains of the Gal3 protein are important for insulin-independent GLUT4 translocation activity. Similar reduction of ability was observed for the Gal3 R186S mutant, suggesting that glycan binding by Gal3 is also involved in GLUT4 translocation.

### Example 17: Gal3 Binds to the Glucose Transporters GLUT1 and GLUT4 and Anti-Gal3 Antibodies Block this Binding

Human Gal3 protein was diluted in PBS to a concentration of 4 μg/mL and coated on a 96-well ELISA plate by adding 80 μL per well. Additional wells were coated with a two-fold serial dilution of Gal3 in PBS (2 μg/mL and 1 μg/mL) or no Gal3 coat control. After incubating the plate at 4° C. overnight, the plate was washed with 300 μL PBST three times, followed by a blocking step with 150 μL of 2% BSA in PBST per well and incubated for an hour at room temperature (RT) with gentle rocking. The existing blocking solution was then discarded from the plate. Binding solutions were prepared by diluting GLUT1 and GLUT4 proteins tagged with FLAG in separate buffers of 2% BSA in PBST to a concentration of 4 μg/mL. The GLUT1 and GLUT4 dilutions were then applied to the plate by adding 60 μL per well column-wise for each Gal3 coat concentration condition, and then two-fold serially diluted (2 μg/mL and 1 μg/mL) in 2% BSA in PBST. The plate was incubated for an hour at RT with gentle rocking, and then washed with 300 μL PBST three times. Afterwards, HRP-tagged anti-FLAG antibodies were diluted to 1:4000 in 2% BSA in PBST, and 25 μL was added to all of the wells. The plate was incubated for an hour at RT with gentle rocking, and then washed with 300 μL PBST three times. To develop the plate, 50 μL of ABTS substrate was added to each well and incubated until a sufficiently high signal was achieved. The plate was read in a plate reader at an absorbance of 405 nm. Data was graphed using GraphPad Prism 8.0 software (GraphPad Software, Inc.).

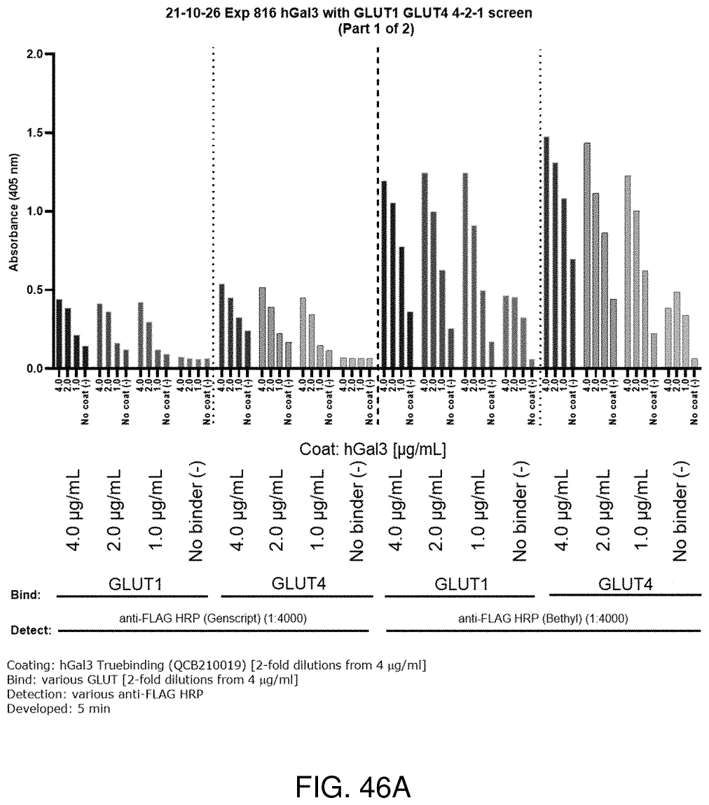

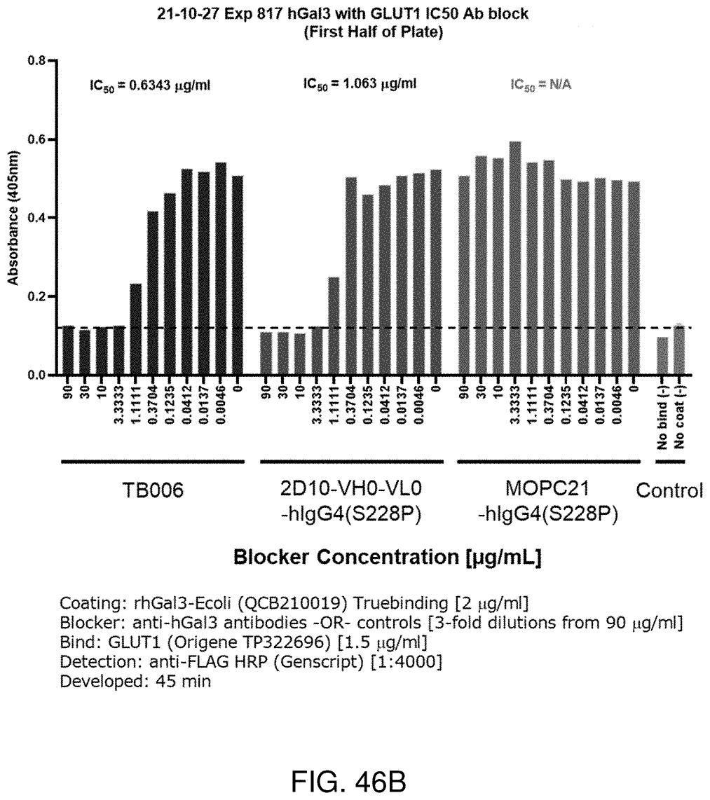

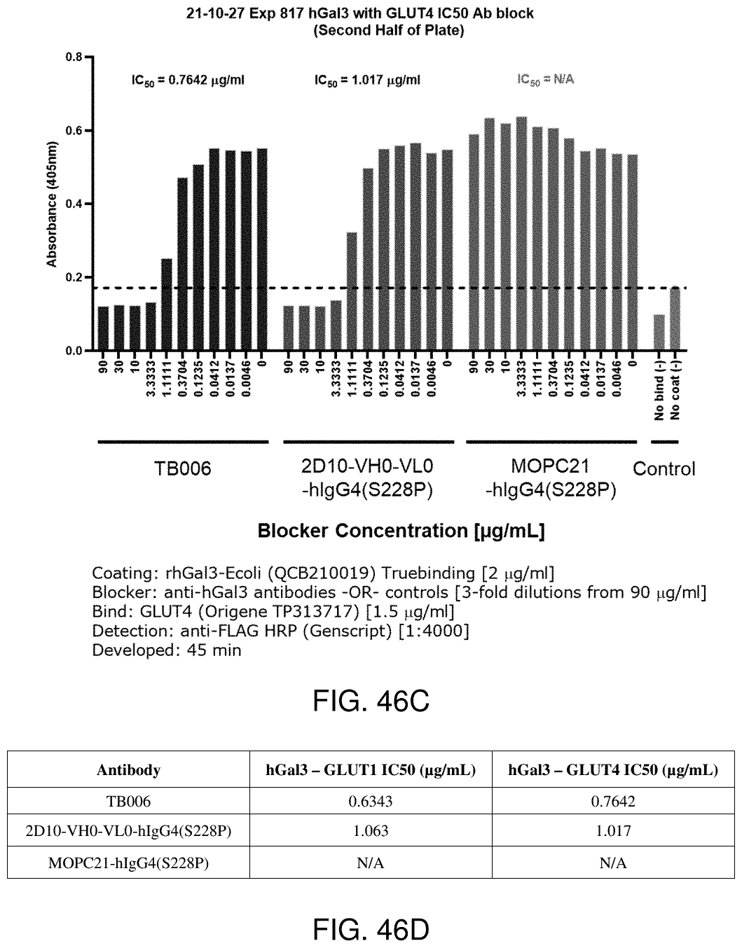

FIG. 46A depicts the results of the Gal3 with GLUT1 or GLUT4 binding ELISA. Both GLUT1 and GLUT4 bound to Gal3 in a concentration-dependent manner.

ELISA was also used to quantify IC50s of exemplary anti-Gal3 antibodies TB006 and 2D10-VH0-VL0 for disrupting the interaction between Gal3 and GLUT1 or GLUT4. Human Gal3 protein was diluted in PBS to a concentration of 2 μg/mL and used to coat a 96-well ELISA plate with 40 μL per well. After incubating the plate at 4° C. overnight, the plate was washed three times with 300 μL PBST. A blocking step was performed with 150 μL of 2% BSA in PBST per well and incubated for an hour at RT with gentle rocking. The existing blocking solution was then discarded form the plate. Antibodies TB006, 2D10-VH0-VL0 (2D10), or MOPC21 hIgG4 isotype control were diluted to 180 μg/mL in 2% BSA in PBST, then applied to the plate by adding 45 μL to each well in the leftmost column and serially diluted 3-fold rightwards across the plate. 30 μL of 3 μg/mL of FLAG-tagged GLUT1 or GLUT4 protein diluted in 2% BSA in PBST was then added to the appropriate wells containing antibody or control solution. The plate was incubated for an hour at RT with gentle rocking and then washed three times with 300 μL PBST. Afterwards, HRP-tagged anti-FLAG antibody was diluted to 1:4000 in 2% BSA in PBST, and 25 μL was added to all of the wells. The plate was incubated for an hour at RT with gentle rocking, and then washed with 300 μL PBST three times. To develop the plate, 50 μL of ABTS substrate was added to each well and incubated until a sufficiently high signal was achieved. The plate was read with a plate reader at an absorbance of 405 nm. Data was graphed using GraphPad Prism 8.0 software and IC50 values were generated using manufacturer instructions.

The ELISA results for GLUT1 (FIG. 46B) and GLUT4 (FIG. 46C) indicate a consistent concentration-dependent ability for anti-Gal3 antibodies TB006 and 2D10 to inhibit the binding of these glucose transporters to Gal3. MOPC21 isotype control failed to block the Gal3 binding to either GLUT1 or GLUT4. FIG. 46D show the calculated IC50 for the conditions tested.

### Example 18: GLUT4 Binds to the C-Terminal Domain of Gal3 as Determined by Hydrogen-Deuterium Exchange

Hydrogen-deuterium exchange mass spectrometry (HDX-MS) can be used to determine the binding site between antibody and antigen. The protein interaction decreases deuterium uptake by the two proteins at their binding position, and thus causes a mass shift between bound and unbound samples. The mass shift can be detected using mass spectrometry. In the data process, the deuterium uptake difference (bound minus unbound) at the residue level is visualized using a heatmap. In a protein sequence, the residues with negative mass shift indicate that this may be the binding site, and the heatmap shows the intensity of the binding interaction on different residues in a protein sequence.

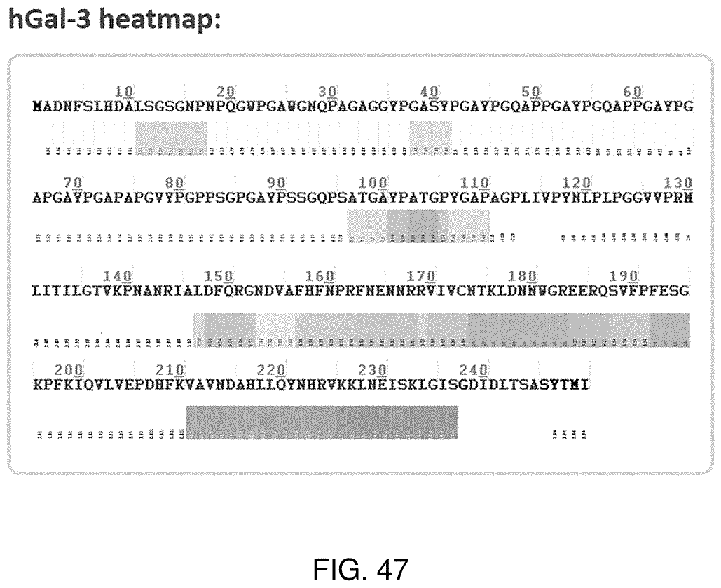

FIG. 47 depicts a heatmap of HDX-MS to determine the binding region of hGal3 with GLUT4. An Arg 228-Val 292 truncated form of GLUT4 that is soluble under buffer conditions was used. Binding as detected by less deuterium uptake was found mainly on the C-terminal domain of Gal3. Three major binding sites were determined as amino acids 101-106, 148-195, and 211-237 as shown in FIG. 47. The amino acid 211-237 binding region was determined to have the strongest binding affinity among these.

### Example 19: Anti-Gal3 Antibodies are Internalized into Cells Only in the Presence of Gal3

It was tested whether anti-Gal3 antibodies were internalized into skeletal muscle cells. L6 myoblasts were seeded at a density of 10000 cells per well in a black clear bottom 96 well plate in L6 complete medium. The next day, the cells were serum starved using serum-free medium in the morning for 2 hours before proceeding with the following internalization experiment. Exemplary anti-Gal3 antibodies TB006 and 2D10, or MOPC21 isotype control, were conjugated with fluorophore using the Xenon pHrodo iFL Red Human IgG Labeling Reagent (Thermo Fisher). The fluorophore-conjugated antibodies were then mixed with Gal3 protein (2 μg/mL or 10 μg/mL Gal3) or without Gal3 as control, and this mixture was added to the myoblasts. After 3 hours of incubation, fluorescent images of the cells were taken.

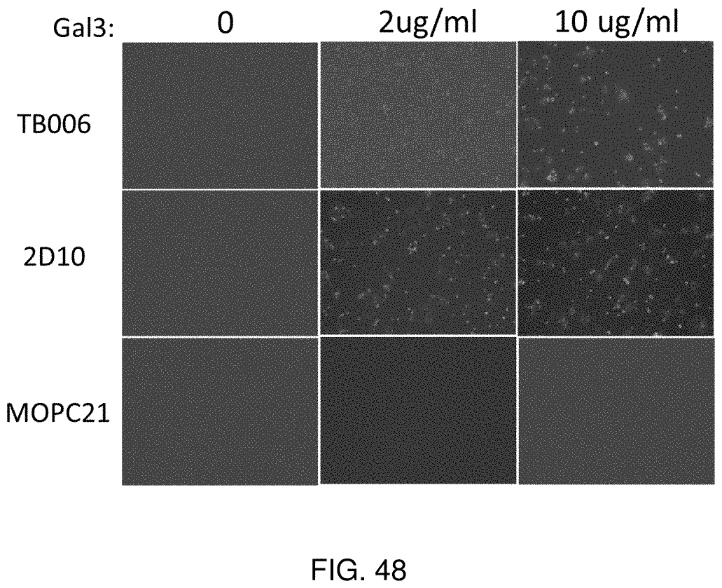

FIG. 48 depicts fluorescent images of the myoblasts contacted with the anti-Gal3 antibody and Gal3 complexes. Internalization of the tested anti-Gal3 antibodies were observed only when the antibodies were first incubated with Gal3 (i.e. the null Gal3 control did not result in antibody internalization). The MOPC21 isotype control was not observed to internalize into the myoblasts under any condition.

### Example 20: Anti-Gal3 Antibodies Rescue Gal3-Induced Glucose Intolerance in Mice

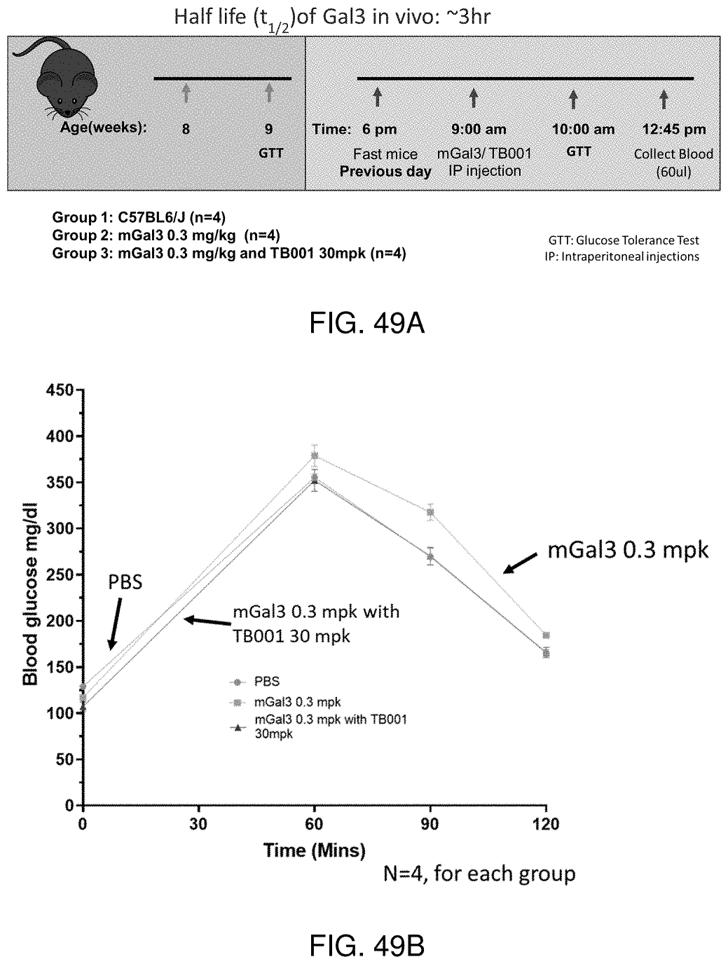

FIG. 49A depicts a schematic for testing the effects of anti-Gal3 antibody administration on Gal3-mediated glucose intolerance in vivo in mice. C57BL6/J mice were fasted for a day, and then injected intraperitoneally with 0.3 mg/kg mouse Gal3 with or without 30 mg/kg TB001, or with PBS as control. Mice were injected with glucose as generally described in Example 4 at 1 hour after injection of Gal3 and/or TB001. Blood was collected from the mice at 0, 30, 60, 90, and 120 minutes after the glucose injection for blood glucose measurement.

FIG. 49B shows the blood glucose levels of the mice at the tested times after glucose injection, indicating that mice injected with Gal3 alone exhibited elevated blood glucose levels compared to PBS control mice, and that the mice injected with Gal3 followed by injection with the anti-Gal3 antibody TB001 exhibited a restoration of blood glucose levels more similar to the PBS injected control mice.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

A Sequence Listing in electronic format is submitted herewith. Some of the sequences provided in the Sequence Listing may be designated as Artificial Sequences by virtue of being non-naturally occurring fragments or portions of other sequences, including naturally occurring sequences. Some of the sequences provided in the Sequence Listing may be designated as Artificial Sequences by virtue of being combinations of sequences from different origins, such as humanized antibody sequences.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. An anti-Gal3 antibody or binding fragment thereof comprising (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence that is SEQ ID NO: 32, 37, or 66;
the V_{H}-CDR2 comprises an amino acid sequence that is SEQ ID NO: 801, 951, 952, 77, or 108;
the V_{H}-CDR3 comprises an amino acid sequence that is SEQ ID NO: 953, 954, 802, 118, or 164;
the V_{L}-CDR1 comprises an amino acid sequence that is SEQ ID NO: 171, 178, or 215;
the V_{L}-CDR2 comprises an amino acid sequence having that is SEQ ID NO: 222, 229, or 225; and
the V_{L}-CDR3 comprises an amino acid sequence that is SEQ ID NO: 257, 256, or 291.

2.-6. (canceled)

7. A method of enhancing glucose transporter (GLUT) translocation in a cell, comprising:
contacting the cell with an anti-Gal3 antibody or binding fragment thereof,
wherein binding of the anti-Gal3 antibody or binding fragment thereof to Gal3 in the cell inhibits Gal3-mediated blocking of GLUT4 translocation.

8.-10. (canceled)

11. The method of claim 7, wherein the glucose transporter is glucose transporter 1 (GLUT1) and/or glucose transporter 4 (GLUT4).

12. (canceled)

13. The method of claim 7, wherein GLUT translocation in the cell is enhanced by at least 50% after contacting with the anti-Gal3 antibody or binding fragment thereof relative to a cell that is not contacted with the anti-Gal3 antibody or binding fragment thereof.

14. The method of claim 7, wherein the anti-Gal3 antibody or binding fragment thereof selectively binds to an N-terminal domain of Gal3.

15. The method of claim 7, wherein the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof.

16.-17. (canceled)

18. The method of claim 7, wherein the heavy chain variable region comprises a sequence having at least 80% identity to the sequence selected from SEQ ID NOs: 1067-1109, 1415-1439.

19. The method of claim 7, wherein the light chain variable region comprises a sequence having at least 80% identity to the sequence selected from SEQ ID NOs: 969-982, 1110-1152, 1440-1464.

20.-22. (canceled)

23. The method of claim 7, wherein the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

24. The method of claim 7, wherein the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof.

25. The method of claim 7, wherein the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

26. A method of improving insulin sensitivity in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated blocking of glucose transporter (GLUT) translocation in the subject, thereby improving insulin sensitivity in the subject.

27.-29. (canceled)

30. The method of claim 26, wherein the GLUT is glucose transporter 1 (GLUT1) and/or glucose transporter 4 (GLUT4).

31.-33. (canceled)

34. The method of claim 26, wherein the insulin sensitivity in the subject is improved by at least 50% relative to the insulin sensitivity of the subject prior to the administering step.

35. The method claim 26, wherein the anti-Gal3 antibody or binding fragment thereof selectively binds to an N-terminal domain of Gal3.

36. The method of claim 26, wherein the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof.

37.-38. (canceled)

39. The method of claim 26, wherein the heavy chain variable region comprises a sequence having at least 80% identity to the sequence selected from SEQ ID NOs: 955-968, 1067-1109, 1415-1439.

40. The method of claim 26, wherein the light chain variable region comprises a sequence having at least 80% identity to the sequence selected from SEQ ID NOs: 969-982, 1110-1152, 1440-1464.

41.-43. (canceled)

44. The method of claim 26, wherein the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

45. The method of claim 26, wherein the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof.

46. The method of claim 26, wherein the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.

47. A method of treating a disease associated with insulin resistance in a subject in need thereof, comprising:
administering to the subject an anti-Gal3 antibody or binding fragment thereof,
thereby treating the disease associated with insulin resistance in the subject.

48.-55. (canceled)

56. The method of claim 47, wherein the insulin sensitivity in the subject is improved by at least 50% relative to the insulin sensitivity of the subject prior to the administering step.

57. The method of claim 47, wherein the anti-Gal3 antibody or binding fragment thereof selectively binds to an N-terminal domain of Gal3.

58. The method of claim 47, wherein the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof.

59.-60. (canceled)

61. The method of claim 47, wherein the heavy chain variable region comprises a sequence having at least 80% identity to the sequence selected from SEQ ID NOs: 955-968, 1067-1109, 1415-1439.

62. The method of claim 47, wherein the light chain variable region comprises a sequence having at least 80% identity to the sequence selected from SEQ ID NOs: 969-982, 1110-1152, 1440-1464.

63.-65. (canceled)

66. The method of claim 47, wherein the anti-Gal3 antibody or binding fragment thereof is selected from the group consisting of: 20H5.A3, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

67. The method of claim 47, wherein the anti-Gal3 antibody or binding fragment thereof is selected from the group consisting of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof.

68. The method of claim 47, wherein the anti-Gal3 antibody or binding fragment thereof is selected from the group consisting of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof.
